(19) United States
(12) Patent Application Publication
(10) Pub. No.: US 2024/0246932 A1
(43) Pub. Date: Jul. 25, 2024

(54) ARYL 3-OXOPIPERAZINE CARBOXAMIDES AND HETEROARYL 3-OXOPIPERAZINE CARBOXAMIDES AS NAV1.8 INHIBITORS

(71) Applicant: Merck Sharp & Dohme LLC, Rahway, NJ (US)

(72) Inventors: Ashok Arasappan, Bridgewater, NJ (US); Ian M. Bell, Lansdale, PA (US); Jason M. Cox, Rancho Santa Fe, CA (US); Michael J. Kelly, III, Paoli, PA (US); Mark Eric Layton, Harleysville, PA (US); Hong Liu, Hillsborough, NJ (US); Jian Liu, Norristown, PA (US); Akshay A. Shah, Robbinsville, NJ (US); Michael D. VanHeyst, Harleysville, PA (US)

(73) Assignee: Merck Sharp & Dohme LLC, Rahway, NJ (US)

(21) Appl. No.: 18/557,667

(22) PCT Filed: May 2, 2022
(86) PCT No.: PCT/US2022/027262

§ 371 Date: Oct. 27, 2023

Related U.S. Application Data
(60) Provisional application No. 63/185,608, filed on May 7, 2021.

(51) Int. Cl. C07D 401/12; A61K 31/496; C07D 401/14; C07D 403/12; C07D 413/12; C07D 417/12; C07D 417/14; C07D 471/04

(52) U.S. Cl. CPC C07D 401/12; A61K 31/496; C07D 401/14; C07D 403/12; C07D 413/12; C07D 417/12; C07D 417/14; C07D 471/04

(57) ABSTRACT

Novel compounds of the structural formula (I), and the pharmaceutically acceptable salts thereof, are inhibitors of Nav1.8 channel activity and may be useful in the treatment, prevention, management, amelioration, control and suppression of diseases mediated by Nav1.8 channel activity. The compounds of the present invention may be useful in the treatment, prevention or management of pain disorders, cough disorders, acute itch disorders, and chronic itch disorders.

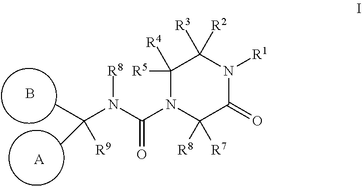

### BACKGROUND OF THE INVENTION

Voltage-gated sodium channels (VGSC) mediate the selective influx of sodium ions in excitable cells and play a central role in initiating and propagating action potentials (Yu et al., Genome Biology 4:207 (2003)). Voltage-gated sodium channels are ubiquitous in the central and peripheral nervous system where they play a central role in the initiation and propagation of action potentials, and also in skeletal and cardiac muscle where the action potential triggers cellular contraction (Goldin et al., Ann N Y Acad Sci. 1999 Apr. 30; 868:38-50). Alterations in VGSC function or their expression can profoundly affect normal cell excitability (Huang et al., J Neurosci. 2013 Aug. 28; 33 (35): 14087-97; Emery et al., J Neurosci. 2015 May 20; 35(20): 7674-81; Kist et al., PLOS One. 2016 Sep. 6; 11(9):e0161789; and Schreiber et al., World J Diabetes. 2015 Apr. 15; 6(3):432-44).

Voltage-gated sodium channels are multimeric complexes characterized by one α-subunit, which forms an ion-conducting aqueous pore, and at least one β-subunit that modifies the kinetics and voltage-dependence of the channel gating. Nine different α-subunits have been identified and characterized in mammalian voltage-gated sodium channels, including Naᵥ1.8, also known as SNS, PN3 or Naᵥ1.8 (Goldin et al., Neuron. 2000 November; 28 (2):365-8). Expression of sodium channels can be tissue specific. Naᵥ1.8 voltage-gated sodium ion channels are expressed primarily in sensory neurons, which are responsible for conveying information from the periphery (e.g. skin, muscle and joints) to the central nervous system via the spinal cord. Sodium channels are integral to this process as sodium channel activity is required for initiation and propagation of action potentials triggered by noxious stimuli (thermal, mechanical and chemical) activating peripheral nociceptors (Catterall et al., Nat Chem Biol. 2017 Apr. 13; 13(5):455-463). An increase in VGSC protein level at the cell surface or an alteration in activity of the VGSC channels can result in disease states such as migraine, neurodegeneration following ischemia, epilepsies, and chronic neuropathic and inflammatory pain states. Gain of function mutations in Naᵥ1.7, Naᵥ1.8, and Naᵥ1.9 manifest in a variety of pain syndromes where patients experience spontaneous pain without an external stimulus (Bennett et al., Lancet Neurol. 2014 June; 13(6):587-99; Huang et al., J Neurosci. 2013 Aug. 28; 33(35): 14087-97; Kist et al., PLOS One. 2016 Sep. 6; 11(9):e0161789; Emery et al., J Neurosci. 2015 May 20; 35(20):7674-81; and Schreiber et al., World J Diabetes. 2015 Apr. 15; 6(3):432-44).

Naᵥ1.8 voltage-gated sodium ion channels are believed to play a role in various maladies, including neuropathic pain, chronic itch, and inflammatory pain perception (Belkouch et al., J Neuroinflammation. 2014 Mar. 7; 11:45; Coward et al., Pain. 2000 March; 85(1-2): 41-50; Yiangou et al., FEBS Lett. 2000 Feb. 11; 467(2-3):249-52; Black et al., Ann Neurol. 2008 December; 64(6):644-53; Bird et al., Br J Pharmacol. 2015 May; 172(10): 2654-70; Liu et al., Neuron. 2010 Nov. 4; 68(3):543-56; and Zhao et al., J Clin Invest. 2013).

Large portions of the voltage gated sodium ion channels are conserved among the various subtypes, therefore there is a potential for producing serious side effects when utilizing therapeutic agents that do not demonstrate subtype selectivity. Therefore, therapeutic agents suitable for use in addressing nociception, cough, or itch disorders, require specificity in their action, for example, discriminating between action upon Naᵥ1.5 sodium ion channels, thought to be important in regulation of cardiac function, and action upon Naᵥ1.8 sodium ion channels, thought to be central in inflammatory nociception, or itch and disorders arising from dysfunctional and/or upregulated Naᵥ1.8 sodium ion channels.

Accordingly, it is believed that inhibitors of Naᵥ1.8 voltage-gated sodium ion channel activity may useful to treat or prevent diseases, disorders and conditions involving Naᵥ1.8 receptors and/or stemming specifically from dysfunction of Naᵥ1.8 voltage-gated sodium ion channels (Han et al., J Neurol Neurosurg Psychiatry 2014 May; 85(5):499-505), including but not limited to, migraine, neurodegeneration following ischemia, epilepsy, inflammatory pain, spontaneous pain, acute pain, preoperative pain, perioperative pain, post-operative pain, neuropathic pain, chronic itch, and itch disorders.

There remains a need for potent Naᵥ1.8 sodium ion channel activity inhibitors with selective activity for Naᵥ1.8 sodium ion channels. As a result, the compounds of the present invention are useful for the treatment and prevention of diseases, disorders and conditions involving Naᵥ1.8 receptors and Naᵥ1.8 voltage-gated sodium ion channels.

The role of Nav1.8 sodium ion channels is discussed in: Bennett et al., Physical Medicine and Rehabilitation Clinics of North America, 2001, 12(2):447-459; Meissner et al., Br J Sports Med. 2018 May; 52(10):642-650; Legroux-Crespel et al., Neurology. 2016 Feb. 2; 86(5):473-83; and Flaxman et al., Lancet, 380:2163-2196 (2012).

Compounds useful to treat Naᵥ1.8 sodium ion channel related conditions are disclosed in: ACS Med. Chem. Lett. 2015, 6, 650; BJP 2015, 172, 2654; PNAS 2007, 104, 8520; J. Med. Chem. 2008, 51, 407; JPET 2008, 324, 1204; and Neuropharmacology 2010, 59, 201.

Naᵥ1.8 compounds are also disclosed in; WO 2009/049180, WO 2009/049181, WO 2009/049183, WO 2014/120808; WO 2014/120815; WO 2014/120820; WO 2015/010065; and WO 2015/089361; WO 2017/209322: U.S. Pat. Nos. 8,519,137; 9,051,270; 9,108,903; 9,163,042: U.S. Pat. No. 9,783,501; WO 2020/092667: WO2019/014352; WO2018/213426; U.S. Pat. No. 8,629,149; and WO2011/026240.

### SUMMARY OF THE INVENTION

The present invention relates to novel compounds of structural formula I:

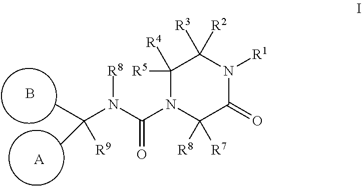

and pharmaceutically acceptable salts, hydrates, and solvates thereof. The compounds of structural formula I, and embodiments thereof, are inhibitors of Naᵥ1.8 sodium ion channel activity (or Naᵥ1.8 inhibitors) and may be useful in the treatment and prevention of diseases, disorders and conditions mediated by Naᵥ1.8 sodium ion channel activity, such as nociception, osteoarthritis, peripheral neuropathy, inherited erythromelalgia, multiple sclerosis, asthma, itch, atopy, allergic or contact dermatitis, renal failure, cholestasis, pruritus, acute itch, chronic itch, migraine, neurodegeneration following ischemia, epilepsy, pain, inflammatory pain, spontaneous pain, acute pain, acute pain due to fractures, musculoskeletal damage, pancreatitis and renal colic, peri-operative pain, post-operative pain, neuropathic pain, postherpetic neuralgia, trigeminal neuralgia, diabetic neuropathy, chronic lower back pain, phantom limb pain, sciatica, pain caused by 2° or 3° burn injury, optic neuritis, pain resulting from cancer and chemotherapy, chronic pelvic pain, pain syndromes, and complex regional pain syndromes. In one embodiment of the present invention, the condition, disease or disorder is a pain disorder, an acute pain disorder or chronic pain disorder. In another embodiment of the present invention, the condition, disease or disorder is an acute pain disorder.

The present invention also relates to pharmaceutical compositions comprising the compounds of the present invention and a pharmaceutically acceptable carrier.

The present invention also relates to methods for the treatment, management, prevention, alleviation, amelioration, suppression or control of disorders, diseases, and conditions that may be responsive to inhibition of Naᵥ1.8 sodium ion channel activity in a subject in need thereof by administering the compounds and pharmaceutical compositions of the present invention.

The present invention also relates to the use of compounds of the present invention for manufacture of a medicament useful in treating diseases, disorders and conditions that may be responsive to the inhibition of Naᵥ1.8 sodium ion channel activity.

The present invention is also concerned with treatment or prevention of these diseases, disorders and conditions by administering the compounds of the present invention in combination with a therapeutically effective amount of another agent that may be useful to treat the disease, disorder and condition. The invention is further concerned with processes for preparing the compounds of this invention.

### DETAILED DESCRIPTION OF THE INVENTION

The present invention is concerned with novel compounds of structural Formula I:

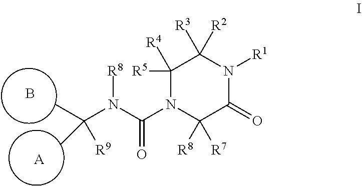

or pharmaceutically acceptable salts thereof, wherein
one of A and B is selected from the group consisting of:
(1) aryl, and
(2) heteroaryl,
wherein each aryl and heteroaryl is unsubstituted or substituted with one to five substituents selected from R^{a}, and the other of A and B is selected from the group consisting of:
(1) aryl, and
(2) heteroaryl,
wherein B is unsubstituted or substituted with one to five substituents selected from R^{b}, provided that when A is aryl then B is not aryl;
R¹ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)ₜC(O)R^{j},
(9) —(CH₂)ₜC(O)NR^{e}R^{j},
(10) —(CH₂)ₙNR^{e}C(O)R^{j},
(11) —(CH₂)ₙNR^{e}C(O)OR^{j},
(12) —(CH₂)ₙNR^{e}C(O)N(R^{e})₂,
(13) —(CH₂)ₙNR^{e}C(O)NR^{e}R^{j},
(14) —(CH₂)ₙNR^{e}S(O)ₘR^{j},
(15) —(CH₂)ₙNR^{e}S(O)ₘN(R^{e})₂,
(16) —(CH₂)ₙNR^{e}S(O)ₘNR^{e}R^{j}, and
(17) —(CH₂)ₙNR^{e}R^{j},
wherein each CH₂, alkyl, alkenyl, alkynyl, cycloalkyl and cycloheteroalkyl is unsubstituted or substituted with one to five substituents selected from R^{c};
R² is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{d},
wherein R² and R³ and the carbon atoms they are connected to can from a —C₃₋₅cycloalkyl ring,
and wherein R² and R⁴ and the carbon atoms they are connected to can from a —C₃₋₅cycloalkyl ring;
R³ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d};
R⁴ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{f}, and
wherein R⁴ and R⁵ and the carbon atoms they are connected to can from a —C₃₋₅cycloalkyl ring;
R⁵ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{f}, and
wherein R⁵ and R⁷ and the carbon atoms they are attached to may form a 4-, 5- or 6-membered saturated ring;
R⁶ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{g}, and wherein R⁶ and R⁷ and the carbon atoms they are connected to can from a —C₃₋₅cycloalkyl ring;
R⁷ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{g};
R⁸ is selected from the group consisting of:
(1) hydrogen,
(2) —C₁₋₆alkyl,
(3) —C₃₋₆cycloalkyl, and
(4) —C₂₋₆cycloheteroalkyl,
wherein each alkyl, cycloalkyl and cycloheteroalkyl is unsubstituted or substituted with one to five substituents selected from R^{e};
R⁹ is selected from the group consisting of:
(1) hydrogen,
(2) —C₁₋₆alkyl,
(3) —C₂₋₆alkenyl, and
(4) —C₂₋₆alkynyl,
wherein each alkyl, alkenyl and alkynyl is unsubstituted or substituted with one to five substituents selected from halogen;
each R^{a} is independently selected from the group consisting of:
(1) CN,
(2) oxo,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —C₂₋₆alkenyl-C₃₋₆cycloalkyl,
(17) —C₂₋₆alkenyl-C₂₋₆cycloheteroalkyl,
(18) —C₂₋₆alkenyl-aryl,
(19) —C₂₋₆alkenyl-heteroaryl,
(20) —C₂₋₆alkynyl-C₃₋₆cycloalkyl,
(21) —C₂₋₆alkynylC₂₋₆cycloheteroalkyl,
(22) —C₂₋₆alkynyl-aryl,
(23) —C₂₋₆alkynyl-heteroaryl,
(24) —OH,
(25) —(CH₂)ₚ—OC₁₋₆alkyl,
(26) —(CH₂)ₚ—OC₂₋₆alkenyl,
(27) —(CH₂)ₚ—OC₂₋₆alkynyl,
(28) —(CH₂)ₚ—OC₃₋₆cycloalkyl,
(29) —(CH₂)ₚ—OC₂₋₆cycloheteroalkyl,
(30) —(CH₂)ₚ—O-aryl,
(31) —(CH₂)ₚ—O-heteroaryl,
(32) —OC₁₋₆alkyl-C₃₋₆cycloalkyl,
(33) —OC₁₋₆alkyl-C₂₋₆cycloheteroalkyl,
(34) —OC₁₋₆alkyl-aryl,
(35) —OC₁₋₆alkyl-heteroaryl,
(36) —S(O)ᵣR^{h},
(37) —C₁₋₆alkyl-S(O)ᵣR^{h},
(38) —N(R^{k})₂,
(39) —C(O)R^{L}, and
(40) —NR^{k}R^{L}, wherein each R^{a} is unsubstituted or substituted with one to six substituents selected from halogen, CF₃, OH, C₁₋₆alkyl, and OC₁₋₆alkyl;
each R^{b} is independently selected from the group consisting of:
(1) CN,
(2) OXO,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —C₂₋₆alkenyl-C₃₋₆cycloalkyl,
(17) —C₂₋₆alkenyl-C₂₋₆cycloheteroalkyl,
(18) —C₂₋₆alkenyl-aryl,
(19) —C₂₋₆alkenyl-heteroaryl,
(20) —C₂₋₆alkynyl-C₃₋₆cycloalkyl,
(21) —C₂₋₆alkynyl-C₂₋₆cycloheteroalkyl,
(22) —C₂₋₆alkynyl-aryl,
(23) —C₂₋₆alkynyl-heteroaryl,
(24) —OH,
(25) —(CH₂)ₚ—OC₁₋₆alkyl,
(26) —(CH₂)ₚ—OC₂₋₆alkenyl,
(27) —(CH₂)ₚ—OC₂₋₆alkynyl,
(28) —(CH₂)ₚ—OC₃₋₆cycloalkyl,
(29) —(CH₂)ₚ—OC₂₋₆heterocycloalkyl,
(30) —(CH₂)ₚ—O-aryl,
(31) —(CH₂)ₚ—O-heteroaryl,
(32) —OC₁₋₆alkyl-C₃₋₆cycloalkyl,
(33) —OC₁₋₆alkyl-C₂₋₆heterocycloalkyl,
(34) —OC₁₋₆alkyl-aryl,
(35) —OC₁₋₆alkyl-heteroaryl,
(36) —S(O)ᵣRⁱ,
(37) —C₁₋₆alkyl-S(O)ᵣRⁱ,
(38) —N(R^{k})₂,
(39) —C(O)R^{L}, and
(40) —NR^{k}R^{L},
wherein each R^{b} is unsubstituted or substituted with one to six substituents selected from halogen, CF₃, OCF₃, CN, CH₂CF₃, CF₂CH₃, —C₁₋₆alkyl, and O—C₁₋₆alkyl;
R^{c} is selected from:
(1) —C₁₋₆alkyl,
(2) OH,
(3) halogen, and
(4) —OC₁₋₆alkyl,
wherein alkyl is unsubstituted or substituted with one to three halogens;
R^{d} is selected from:
(1) —C₁₋₆alkyl,
(2) OH,
(3) halogen, and
(4) —OC₁₋₆alkyl,
wherein alkyl is unsubstituted or substituted with one to three halogens;
R^{e} is selected from:
(1) hydrogen, and
(2) C₁₋₆alkyl;
R^{f} is selected from:
(1) —C₁₋₆alkyl,
(2) OH,
(3) halogen, and
(4) —OC₁₋₆alkyl,
wherein alkyl is unsubstituted or substituted with one to three halogens;
R^{g} is selected from:
(1) —C₁₋₆alkyl,
(2) OH,
(3) halogen, and
(4) —OC₁₋₆alkyl,
wherein alkyl is unsubstituted or substituted with one to three halogens;
R^{h} is selected from:
(1) hydrogen,
(2) C₁₋₆alkyl,
(3) C₃₋₆cycloalkyl,
(4) aryl, and
(5) heteroaryl;
Rⁱ is selected from:
(1) hydrogen,
(2) C₁₋₆alkyl,
(3) C₃₋₆cycloalkyl,
(4) aryl, and
(5) heteroaryl;
R^{j} is selected from:
(1) hydrogen,
(2) C₁₋₆alkyl,
(3) C₃₋₆alkenyl,
(4) C₃₋₆alkynyl,
(5) C₃₋₆cycloalkyl,
(6) C₂₋₅cycloheteroalkyl,
(7) aryl, and
(8) heteroaryl;
R^{k} is selected from:
(1) hydrogen, and
(2) C₁₋₆alkyl:
R^{L} is selected from:
(1) hydrogen,
(2) C₁₋₆alkyl,
(3) C₃₋₆cycloalkyl,
(4) aryl, and
(5) heteroaryl;
m is independently selected from 0, 1 and 2;
n is independently selected from 2, 3, 4, 5 and 6;
p is independently selected from 0, 1, 2 and 3;
q is independently selected from 0, 1, 2 and 3;
r is independently selected from 0, 1 and 2;
s is independently selected from 0, 1, 2, 3, 4, 5, and 6; and
t is is independently selected from 0, 1, 2, 3, 4, 5, and 6.

The invention has numerous embodiments, which are summarized below. The invention includes the compounds as shown, and also includes individual diastereoisomers, enantiomers, and epimers of the compounds, and mixtures of diastereoisomers and/or enantiomers thereof including racemic mixtures.

In one embodiment of the present invention, A is selected from the group consisting of: aryl, and heteroaryl, wherein each aryl and heteroaryl is unsubstituted or substituted with one to five substituents selected from R^{a}. In a class of this embodiment, A is selected from the group consisting of: phenyl, pyridine, pyrazole, oxazole, imidazopyridine, pyrimidine, and thiazole, wherein A is unsubstituted or substituted with one to five substituents selected from R^{a}.

In another embodiment of the present invention, A is selected from the group consisting of: aryl, and heteroaryl, wherein each aryl and heteroaryl is unsubstituted or substituted with one to five substituents selected from R^{a}. In a class of this embodiment, A is selected from the group consisting of: phenyl, pyridine, pyrazole, oxazole, and thiazolyl, wherein A is unsubstituted or substituted with one to five substituents selected from R^{a}. In another embodiment, A is selected from the group consisting of: phenyl, and pyridine, wherein phenyl and pyridine are unsubstituted or substituted with one to five substituents selected from R^{a}.

In another embodiment of the present invention, A is selected from the group consisting of: aryl, and heteroaryl, wherein each aryl and heteroaryl is unsubstituted or substituted with one to five substituents selected from R^{a}, provided that if A is aryl then B is not aryl. In a class of this embodiment, A is selected from the group consisting of: phenyl, pyridine, pyrazole, oxazole, imidazopyridine, pyrimidine, and thiazole, wherein A is unsubstituted or substituted with one to five substituents selected from R^{a}, provided that when A is phenyl then B is not phenyl.

In another embodiment, A is selected from the group consisting of: phenyl, and pyridine, wherein phenyl and pyridine are unsubstituted or substituted with one to five substituents selected from R^{a}, provided that when A is phenyl then B is not phenyl.

In another embodiment, A is selected from the group consisting of: phenyl, and pyridine, wherein phenyl and pyridine are substituted with one to five substituents selected from R^{a}, provided that when A is phenyl then B is not phenyl.

In another embodiment of the present invention, A is aryl, wherein aryl is unsubstituted or substituted with one to five substituents selected from R^{a}. In a class of this embodiment, A is phenyl, wherein phenyl is unsubstituted or substituted with one to five substituents selected from R^{a} In another class of this embodiment, A is phenyl, wherein phenyl is substituted with one to five substituents selected from R^{a}.

In another embodiment of the present invention, A is aryl, wherein aryl is unsubstituted or substituted with one to five substituents selected from R^{a}, provided that when A is aryl then B is not aryl. In a class of this embodiment, A is phenyl, wherein phenyl is unsubstituted or substituted with one to five substituents selected from R^{a}, provided that when A is phenyl then B is not phenyl. In another class of this embodiment, A is phenyl, wherein phenyl is substituted with one to five substituents selected from R^{a}, provided that when A is phenyl then B is not phenyl.

In another embodiment of the present invention, A is heteroaryl, wherein heteroaryl is unsubstituted or substituted with one to five substituents selected from R^{a}. In a class of this embodiment, A is selected from the group consisting of: pyridine, pyrazole, oxazole, and thiazole, wherein A is unsubstituted or substituted with one to five substituents selected from R^{a}. In another class of this embodiment, A is pyridine, wherein pyridine is unsubstituted or substituted with one to five substituents selected from R^{a}.

In one embodiment of the present invention, B is independently selected from the group consisting of: aryl, and heteroaryl, wherein B is unsubstituted or substituted with one to five substituents selected from R^{b}, provided that both A and B are not aryl. In a class of this embodiment, B is selected from the group consisting of: phenyl, pyridine, pyrimidine, pyrazole, thiazole, imidazo[1,2-a]pyridine, oxazole, benzofuran, benzoxazole, indazole, and thiazolopyridine, wherein B is unsubstituted or substituted with one to five substituents selected from R^{b}, provided that if B is phenyl, A is not aryl.

In another embodiment, B is heteroaryl, wherein heteroaryl is unsubstituted or substituted with one to five substituents selected from R^{b}. In a class of this embodiment, B is independently selected from the group consisting of: pyridine, pyrimidine, pyrazole, thiazole, imidazo[1,2-a]pyridine, oxazole, benzofuran, benzoxazole, indazole, and thiazolopyridine, wherein B is unsubstituted or substituted with one to five substituents selected from R^{b}. In another class of this embodiment, B is independently selected from the group consisting of: pyridine, pyrimidine, pyrazole, thiazole, and imidazo[1,2-a]pyridine, wherein B is unsubstituted or substituted with one to five substituents selected from R^{b}. In another class of this embodiment, B is independently selected from the group consisting of: pyridine, pyrazole, and thiazole, wherein B is unsubstituted or substituted with one to five substituents selected from R^{b}.

In another embodiment, B is aryl, wherein aryl is unsubstituted or substituted with one to five substituents selected from R^{b}, provided that A is not aryl. In a class of this embodiment, B is phenyl, wherein phenyl is unsubstituted or substituted with one to five substituents selected from R^{b}, provided that A is not aryl. In another class of this embodiment, B is phenyl, wherein phenyl is substituted with one to five substituents selected from R^{b}, provided that A is not aryl. In one embodiment of the present invention, R¹ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)ₜC(O)R^{j}, —(CH₂)ₜC(O)NR^{e}R^{j}, —(CH₂)ₙNR^{e}C(O)R^{j}, —(CH₂)ₙNR^{e}C(O)OR^{j}. —(CH₂)ₙNR^{e}C(O)N(R^{e})₂, —(CH₂)ₙNR^{e}C(O)NR^{e}R^{j}, —(CH₂)ₙNR^{e}S(O)ₘR^{j}, —(CH₂)ₙNR^{e}S(O)ₘN(R^{e})₂, —(CH₂)ₙNR^{e}S(O)ₘNR^{e}R^{j}, and —(CH₂)ₙNR^{e}R^{j}, wherein each CH₂, alkyl, alkenyl, alkynyl, cycloalkyl and cycloheteroalkyl is unsubstituted or substituted with one to five substituents selected from R^{c}.

In another embodiment of the present invention, R¹ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl and cycloheteroalkyl is unsubstituted or substituted with one to five substituents selected from R^{c}.

In another embodiment of the present invention, R¹ is selected from the group consisting of: hydrogen, —C₁₋₆alkyl, —C₃₋₆cycloalkyl, and —C₂₋₆cycloheteroalkyl, wherein each alkyl, cycloalkyl and cycloheteroalkyl is unsubstituted or substituted with one to five substituents selected from R^{c}.

In another embodiment of the present invention, R¹ is selected from the group consisting of: hydrogen, —C₁₋₆alkyl, and —C₃₋₆cycloalkyl, wherein each alkyl and cycloalkyl is unsubstituted or substituted with one to five substituents selected from R^{c}. In a class of this embodiment, R¹ is selected from the group consisting of: hydrogen, —CH₃, —CH₂CH₃, and cyclopropyl, wherein cyclopropyl is unsubstituted or substituted with one to five substituents selected from R^{c}. In another embodiment of the present invention, R¹ is hydrogen.

In another embodiment of the present invention, R¹ is —C₁₋₆alkyl, wherein each alkyl is unsubstituted or substituted with one to five substituents selected from R^{c}. In a class of this embodiment, R¹ is selected from the group consisting of: —CH₃, and —CH₂CH₃.

In another embodiment of the present invention, R¹ is —C₃₋₆cycloalkyl, wherein cycloalkyl is unsubstituted or substituted with one to five substituents selected from R^{c}. In a class of this embodiment, R¹ is cyclopropyl, wherein cycloalkyl is unsubstituted or substituted with one to five substituents selected from R^{c}.

In one embodiment of the present invention, R² is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)SC(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d}, wherein R² and R³ and the carbon atoms they are connected to can from a —C₃₋₅cycloalkyl ring, and wherein R² and R⁴ and the carbon atoms they are connected to can from a —C₃₋₅cycloalkyl ring.

In another embodiment of the present invention, R² is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d}, wherein R² and R³ and the carbon atoms they are connected to can from a —C₃₋₅cycloalkyl ring.

In another embodiment of the present invention, R² is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)SC(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d}, wherein R² and R⁴ and the carbon atoms they are connected to can from a —C₃₋₅cycloalkyl ring.

In another embodiment of the present invention, R² is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)SC(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d}.

In another embodiment of the present invention, R² is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{d}.

In another embodiment of the present invention, R² is selected from the group consisting of: hydrogen, —C₁₋₆alkyl, —C₂₋₆alkenyl and —C₃₋₆cycloalkyl, wherein each alkyl, alkenyl, and cycloalkyl is unsubstituted or substituted with one to five substituents selected from R^{d}.

In another embodiment of the present invention, R² is selected from the group consisting of: hydrogen, —C₁₋₆alkyl, and —C₃₋₆cycloalkyl, wherein each alkyl and cycloalkyl is unsubstituted or substituted with one to five substituents selected from R^{d}. In a class of this embodiment, R² is selected from the group consisting of: hydrogen, —CH₃, CH₂F, —CH₂CH₃, —CH(CH₃)₂, and cyclopropyl, wherein cyclopropyl is unsubstituted or substituted with one to five substituents selected from R^{d}. In another embodiment of the present invention, R² is hydrogen.

In another embodiment of the present invention, R² is —C₁₋₆alkyl, wherein alkyl is unsubstituted or substituted with one to five substituents selected from R^{d}. In a class of this embodiment, R² is selected from the group consisting of: —CH₃, —CF₃, CH₂F, —CH₂CH₃, and —CH(CH₃)₂. In another class of this embodiment, R² is selected from the group consisting of: —CH₃, and CH₂F.

In another embodiment of the present invention, R² is —C₃₋₆cycloalkyl, wherein each cycloalkyl is unsubstituted or substituted with one to five substituents selected from R^{d}. In a class of this embodiment, R² is cyclopropyl, wherein cyclopropyl is unsubstituted or substituted with one to five substituents selected from R^{d}.

In one embodiment of the present invention, R³ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{d}.

In another embodiment of the present invention, R³ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{d}.

In another embodiment of the present invention, R³ is selected from the group consisting of: hydrogen, —C₁₋₆alkyl, —C₂₋₆alkenyl, —C₃₋₆cycloalkyl, and —C₂₋₆cycloheteroalkyl, wherein each alkyl, alkenyl, cycloalkyl, and cycloheteroalkyl is unsubstituted or substituted with one to five substituents selected from R^{d}.

In another embodiment of the present invention, R³ is selected from the group consisting of: hydrogen, —C₁₋₆alkyl, —C₂₋₆alkenyl, and —C₃₋₆cycloalkyl, wherein each alkyl, alkenyl, and cycloalkyl is unsubstituted or substituted with one to five substituents selected from R^{d}.

In another embodiment of the present invention, R³ is selected from the group consisting of: hydrogen, —C₁₋₆alkyl, and —C₃₋₆cycloalkyl, wherein each alkyl and cycloalkyl is unsubstituted or substituted with one to five substituents selected from R^{d}. In a class of this embodiment, R³ is selected from the group consisting of: hydrogen, —CH₃, —CF₃, CH₂F, —CH₂CH₃, —CH(CH₃)₂, and cyclopropyl, wherein cyclopropyl is unsubstituted or substituted with one to five substituents selected from R^{d}.

In another embodiment of the present invention, R³ is selected from the group consisting of: hydrogen, and —C₁₋₆alkyl, wherein alkyl is unsubstituted or substituted with one to five substituents selected from R^{d}. In a class of this embodiment, R³ is selected from the group consisting of: hydrogen, —CH₃, and —CH₂F. In another embodiment of the present invention, R³ is hydrogen.

In another embodiment of the present invention, R³ is —C₁₋₆alkyl, wherein alkyl is unsubstituted or substituted with one to five substituents selected from R^{d}. In a class of this embodiment, R³ is selected from the group consisting of: —CH₃, CH₂F, —CH₂CH₃, and —CH(CH₃)₂. In another class of this embodiment, R³ is —CH₃, or —CH₂F.

In another embodiment of the present invention, R³ is —C₃₋₆cycloalkyl, wherein cycloalkyl is unsubstituted or substituted with one to five substituents selected from R^{d}. In a class of this embodiment, R³ is cyclopropyl, wherein cyclopropyl is unsubstituted or substituted with one to five substituents selected from R^{d}.

In one embodiment of the present invention, R⁴ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)SC(O)NR^{e}R^{j}, —(CH₂)_{S}NR^{e}C(O)R^{j}, —(CH₂)_{S}NR^{e}C(O)Or^{j}, —(CH₂)_{S}NR^{e}C(O)N(R^{e})₂, —(CH₂)_{S}NR^{e}C(O)Nr^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{f}, and wherein R⁴ and R⁵ and the carbon atoms they are connected to can from a —C₃₋₅cycloalkyl ring.

In another embodiment of the present invention, R⁴ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{f}.

In another embodiment of the present invention, R⁴ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{f}.

In another embodiment of the present invention, R⁴ is selected from the group consisting of: hydrogen, —C₁₋₆alkyl, —C₂₋₆alkenyl, —C₃₋₆cycloalkyl, and —C₂₋₆cycloheteroalkyl, wherein each alkyl, alkenyl, cycloalkyl, and cycloheteroalkyl is unsubstituted or substituted with one to five substituents selected from R^{f}.

In another embodiment of the present invention, R⁴ is selected from the group consisting of: hydrogen, —C₁₋₆alkyl, —C₂₋₆alkenyl, and —C₃₋₆cycloalkyl, wherein each alkyl, alkenyl, and cycloalkyl is unsubstituted or substituted with one to five substituents selected from R^{f}.

In another embodiment of the present invention, R⁴ is selected from the group consisting of: hydrogen, —C₁₋₆alkyl, and —C₃₋₆cycloalkyl, wherein alkyl and cycloalkyl is unsubstituted or substituted with one to five substituents selected from R^{f}. In a class of this embodiment, R⁴ is selected from the group consisting of: hydrogen, —CH₃, CH₂F, —CH₂CH₃, —CH(CH₃)₂, and cyclopropyl, wherein cyclopropyl is unsubstituted or substituted with one to five substituents selected from R^{f}.

In another embodiment of the present invention, R⁴ is hydrogen or —C₁₋₆alkyl, wherein alkyl is unsubstituted or substituted with one to five substituents selected from R^{f}. In a class of this embodiment, R⁴ is selected from the group consisting of: hydrogen, —CH₃, CH₂F, —CH₂CH₃, and —CH(CH₃)₂. In another embodiment of the present invention, R⁴ is hydrogen.

In another embodiment of the present invention, R⁴ is —C₁₋₆alkyl, wherein each alkyl is unsubstituted or substituted with one to five substituents selected from R^{f}. In a class of this embodiment, R⁴ is selected from the group consisting of: —CH₃, CH₂F, —CH₂CH₃, and —CH(CH₃)₂.

In another embodiment of the present invention, R⁴ is —C₃₋₆cycloalkyl, wherein cycloalkyl is unsubstituted or substituted with one to five substituents selected from R^{f}. In a class of this embodiment, R⁴ is cyclopropyl, wherein cyclopropyl is unsubstituted or substituted with one to five substituents selected from R^{f}.

In one embodiment of the present invention, R⁵ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)Or^{j}, —(CH₂)_{S}NR^{e}C(O)N(R^{e})₂, —(CH₂)_{S}NR^{e}C(O)Nr^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{f}, and wherein R⁵ and R⁷ and the carbon atoms they are attached to may form a 4-, 5- or 6-membered saturated ring.

In another embodiment of the present invention, R⁵ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)SC(O)NR^{e}R^{j}, —(CH₂)_{S}NR^{e}C(O)R^{j}, —(CH₂)_{S}NR^{e}C(O)OR, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{f}, and wherein R⁵ and R⁷ and the carbon atoms they are attached to may form a 5-membered saturated ring.

In another embodiment of the present invention, R⁵ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)OR^{j}, —(CH₂)_{S}NR^{e}C(O)N(R^{e})₂, —(CH₂)_{S}NR^{e}C(O)NR^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{f}.

In another embodiment of the present invention, R⁵ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{f}.

In another embodiment of the present invention, R⁵ is selected from the group consisting of: hydrogen, —C₁₋₆alkyl, —C₂₋₆alkenyl, —C₃₋₆cycloalkyl, and —C₂₋₆cycloheteroalkyl, wherein each alkyl, alkenyl, cycloalkyl, and cycloheteroalkyl is unsubstituted or substituted with one to five substituents selected from R^{f}.

In another embodiment of the present invention, R⁵ is selected from the group consisting of: hydrogen, —C₁₋₆alkyl, —C₂₋₆alkenyl, and —C₃₋₆cycloalkyl, wherein each alkyl, alkenyl, and cycloalkyl is unsubstituted or substituted with one to five substituents selected from R^{f}.

In another embodiment of the present invention, R⁵ is selected from the group consisting of: hydrogen, —C₁₋₆alkyl, and —C₃₋₆cycloalkyl, wherein alkyl and cycloalkyl is unsubstituted or substituted with one to five substituents selected from R^{f}. In a class of this embodiment, R⁵ is selected from the group consisting of: hydrogen, —CH₃, CH₂F, —CH₂CH₃, —CH(CH₃)₂, and cyclopropyl, wherein cyclopropyl is unsubstituted or substituted with one to five substituents selected from R^{f}.

In another embodiment of the present invention, R$ is selected from the group consisting of: hydrogen, and —C₁₋₆alkyl, wherein alkyl is unsubstituted or substituted with one to five substituents selected from R^{f}. In a class of this embodiment, R⁵ is selected from the group consisting of: hydrogen, —CH₃, CH₂F, —CH₂CH₃, and —CH(CH₃)₂. In another embodiment of the present invention, R⁵ is hydrogen.

In another embodiment of the present invention, R⁵ is —C₁₋₆alkyl, wherein alkyl is unsubstituted or substituted with one to five substituents selected from R^{f}. In a class of this embodiment, R⁵ is selected from the group consisting of: —CH₃, CH₂F, —CH₂CH₃, and —CH(CH₃)₂.

In another embodiment of the present invention, R⁵ is —C₃₋₆cycloalkyl, wherein cycloalkyl is unsubstituted or substituted with one to five substituents selected from R^{f}. In a class of this embodiment, R⁵ is cyclopropyl, wherein cyclopropyl is unsubstituted or substituted with one to five substituents selected from R^{f}.

In one embodiment of the present invention, R⁶ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)NR^{e}R^{j}, —(CH₂)_{S}NR^{e}C(O)R^{j}, —(CH₂)_{S}NR^{e}C(O)Or^{j}, —(CH₂)_{S}NR^{e}C(O)N(R^{e})₂, —(CH₂)_{S}NR^{e}C(O)Nr^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{g}, and wherein R⁶ and R⁷ and the carbon atoms they are connected to can from a —C₃₋₅cycloalkyl ring.

In another embodiment of the present invention, R⁶ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)Nr^{e}R^{j}, —(CH₂)_{S}NR^{e}C(O)R^{j}, —(CH₂)_{S}NR^{e}C(O)Or^{j}, —(CH₂)_{S}NR^{e}C(O)N(R^{e})₂, —(CH₂)_{S}NR^{e}C(O)Nr^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{g}.

In another embodiment of the present invention, R⁶ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{g}.

In another embodiment of the present invention, R⁶ is selected from the group consisting of: hydrogen, —C₁₋₆alkyl, —C₂₋₆alkenyl, —C₃₋₆cycloalkyl, and —C₂₋₆cycloheteroalkyl, wherein each alkyl, alkenyl, cycloalkyl, and cycloheteroalkyl is unsubstituted or substituted with one to five substituents selected from R^{g}.

In another embodiment of the present invention, R⁶ is selected from the group consisting of: hydrogen, —C₁₋₆alkyl, —C₂₋₆alkenyl, and —C₃₋₆cycloalkyl, wherein each alkyl, alkenyl, and cycloalkyl is unsubstituted or substituted with one to five substituents selected from R^{g}.

In another embodiment of the present invention, R⁶ is selected from the group consisting of: hydrogen, —C₁₋₆alkyl, and —C₃₋₆cycloalkyl, wherein each alkyl and cycloalkyl is unsubstituted or substituted with one to five substituents selected from R^{g}. In a class of this embodiment, R⁶ is selected from the group consisting of: hydrogen, —CH₃, —CH₂CH₃, CH(CH₃)₂ and cyclopropyl, wherein cyclopropyl is unsubstituted or substituted with one to five substituents selected from R^{g}. In another class of this embodiment, R⁶ is selected from the group consisting of: hydrogen, —CH₃, —CH₂CH₃ and cyclopropyl, wherein cyclopropyl is unsubstituted or substituted with one to five substituents selected from R^{g}.

In another class of this embodiment, R⁶ is selected from the group consisting of: hydrogen, —CH₃, and cyclopropyl, wherein cyclopropyl is unsubstituted or substituted with one to five substituents selected from R^{g}.

In another embodiment of the present invention, R⁶ is selected from the group consisting of: hydrogen, and —C₁₋₆alkyl, wherein alkyl is unsubstituted or substituted with one to five substituents selected from R^{g}. In a class of this embodiment, R⁶ is selected from the group consisting of: hydrogen, —CH₃, —CH₂CH₃, —CH(CH₃)₂, and —CH₂F. In another class of this embodiment, R⁶ is selected from the group consisting of: hydrogen, —CH₃, and —CH₂CH₃. In another embodiment of the present invention, R⁶ is hydrogen.

In another embodiment of the present invention, R⁶ is selected from the group consisting of: —C₁₋₆alkyl, and —C₃₋₆cycloalkyl, wherein alkyl and cycloalkyl is unsubstituted or substituted with one to five substituents selected from R^{g}. In a class of this embodiment, R⁶ is selected from the group consisting of: —CH₃, —CH₂CH₃, —CH(CH₃)₂, —CH₂F and cyclopropyl, wherein cyclopropyl is unsubstituted or substituted with one to five substituents selected from R^{g}.

In another embodiment of the present invention, R⁶ is —C₁₋₆alkyl, wherein alkyl is unsubstituted or substituted with one to five substituents selected from R^{g}. In a class of this embodiment, R⁶ is selected from the group consisting of: —CH₃, —CH₂CH₃, —CH(CH₃)₂, and —CH₂F. In another class of this embodiment, R⁶ is selected from the group consisting of: —CH₃, and —CH₂CH₃. In another class of this embodiment, R⁶ is —CH₃.

In another embodiment of the present invention, R⁶ is —C₃₋₆cycloalkyl, wherein cycloalkyl is unsubstituted or substituted with one to five substituents selected from R^{g}. In a class of this embodiment, R⁶ is cyclopropyl, wherein cyclopropyl is unsubstituted or substituted with one to five substituents selected from R^{g}.

In one embodiment of the present invention, R⁷ is selected from the group consisting of: hydrogen, —C₁₋₆alkyl, —C₂₋₆alkenyl, —C₂₋₆alkynyl, —C₃₋₆cycloalkyl, —C₂₋₆cycloheteroalkyl, —C₁₋₆alkyl-O—C₁₋₆alkyl-, —(CH₂)_{S}C(O)R^{j}, —(CH₂)_{S}C(O)Nr^{e}R^{j}, —(CH₂)_{S}NR^{e}C(O)R^{j}, —(CH₂)_{S}NR^{e}C(O)Or^{j}, —(CH₂)_{S}NR^{e}C(O)N(R^{e})₂, —(CH₂)_{S}NR^{e}C(O)Nr^{e}R^{j}, —(CH₂)_{S}NR^{e}S(O)ₘR^{j}, —(CH₂)_{S}NR^{e}S(O)ₘN(R^{e})₂, —(CH₂)_{S}NR^{e}S(O)ₘNR^{e}R^{j}, and —(CH₂)_{S}NR^{e}R^{j}, wherein each CH₂, alkyl, alkenyl, alkynyl, cycloalkyl, and cycloheteroalkyl is unsubstituted or substituted with one to five substituents selected from R^{g}.

In another embodiment of the present invention, R⁷ is selected from the group consisting of: hydrogen, —C₁₋₆alkyl, —C₂₋₆alkenyl, —C₂₋₆alkynyl, —C₃₋₆cycloalkyl, and —C₂₋₆ cycloheteroalkyl, wherein each alkyl, alkenyl, alkynyl, cycloalkyl, and cycloheteroalkyl is unsubstituted or substituted with one to five substituents selected from R^{g}.

In another embodiment of the present invention, R⁷ is selected from the group consisting of: hydrogen, —C₁₋₆alkyl, —C₂₋₆alkenyl, —C₃₋₆cycloalkyl, —C₂₋₆cycloheteroalkyl, wherein each alkyl, alkenyl, cycloalkyl, and cycloheteroalkyl is unsubstituted or substituted with one to five substituents selected from R^{g}.

In another embodiment of the present invention, R⁷ is selected from the group consisting of: hydrogen, —C₁₋₆alkyl, —C₂₋₆alkenyl, and —C₃₋₆cycloalkyl, wherein each alkyl, alkenyl, and cycloalkyl is unsubstituted or substituted with one to five substituents selected from R^{g}.

In another embodiment of the present invention, R⁷ is selected from the group consisting of: hydrogen, —C₁₋₆alkyl, and —C₃₋₆cycloalkyl, wherein each alkyl and cycloalkyl is unsubstituted or substituted with one to five substituents selected from R^{g}. In a class of this embodiment, R⁷ is selected from the group consisting of: hydrogen, —CH₃, —CH₂CH₃, —CH(CH₃)₂, —CH₂F, and cyclopropyl, wherein cyclopropyl is unsubstituted or substituted with one to five substituents selected from R^{g}. In another class of this embodiment, R⁷ is selected from the group consisting of: hydrogen, —CH₃, —CH₂CH₃, and cyclopropyl, wherein cyclopropyl is unsubstituted or substituted with one to five substituents selected from R^{g}. In another class of this embodiment, R⁷ is selected from the group consisting of: hydrogen, —CH₃, and cyclopropyl, wherein cyclopropyl is unsubstituted or substituted with one to five substituents selected from R^{g}. In another embodiment of the present invention, R⁷ is hydrogen.

In another embodiment of the present invention, R⁷ is selected from the group consisting of: hydrogen, and —C₁₋₆alkyl, wherein alkyl is unsubstituted or substituted with one to five substituents selected from R^{g}. In a class of this embodiment, R⁷ is selected from the group consisting of: hydrogen, —CH₃, —CH₂CH₃, —CH(CH₃)₂, and —CH₂F. In another class of this embodiment, R⁷ is selected from the group consisting of: hydrogen, —CH₃, and —CH₂CH₃. In another class of this embodiment, R⁷ is selected from the group consisting of: hydrogen, and —CH₃.

In another embodiment of the present invention, R⁷ is selected from the group consisting of: —C₁₋₆alkyl, and —C₃₋₆cycloalkyl, wherein each alkyl and cycloalkyl is unsubstituted or substituted with one to five substituents selected from R^{g}. In a class of this embodiment, R⁷ is selected from the group consisting of: —CH₃, —CH₂CH₃, —CH(CH₃)₂, —CH₂F, and cyclopropyl, wherein cyclopropyl is unsubstituted or substituted with one to five substituents selected from R^{g}. In another class of this embodiment, R⁷ is selected from the group consisting of: —CH₃, —CH₂CH₃, and cyclopropyl, wherein cyclopropyl is unsubstituted or substituted with one to five substituents selected from R^{g}. In another class of this embodiment, R⁷ is selected from the group consisting of: —CH₃, and cyclopropyl, wherein cyclopropyl is unsubstituted or substituted with one to five substituents selected from R^{g}. In another class of this embodiment, R⁷ is selected from the group consisting of: —CH₃, and cyclopropyl, wherein cyclopropyl is unsubstituted.

In another embodiment of the present invention, R⁷ is selected from the group consisting of: —C₁₋₆alkyl, wherein alkyl is unsubstituted or substituted with one to five substituents selected from R^{g}. In a class of this embodiment, R⁷ is selected from the group consisting of: —CH₃, —CH₂CH₃, —CH(CH₃)₂, and —CH₂F. In another class of this embodiment, R⁷ is selected from the group consisting of: —CH₃, and —CH₂CH₃. In another class of this embodiment, R⁷ is —CH₃.

In another embodiment of the present invention, R⁷ is selected from the group consisting of: —C₃₋₆cycloalkyl, wherein cycloalkyl is unsubstituted or substituted with one to five substituents selected from R^{g}. In a class of this embodiment, R⁷ is cyclopropyl, wherein cyclopropyl is unsubstituted or substituted with one to five substituents selected from R^{g}.

In one embodiment of the present invention, R⁸ is selected from the group consisting of: hydrogen, —C₁₋₆alkyl, —C₃₋₆cycloalkyl, and —C₂₋₆cycloheteroalkyl, wherein each alkyl, cycloalkyl and cycloheteroalkyl is unsubstituted or substituted with one to five substituents selected from R^{e}.

In another embodiment of the present invention, R⁸ is selected from the group consisting of: hydrogen, and —C₁₋₆alkyl, wherein alkyl is unsubstituted or substituted with one to five substituents selected from R^{e}. In a class of this embodiment, R⁸ is selected from the group consisting of: hydrogen, and —CH₃. In another embodiment of the present invention, R⁸ is hydrogen.

In another embodiment of the present invention, R⁸ is —C₁₋₆alkyl, wherein each alkyl is unsubstituted or substituted with one to five substituents selected from R^{e}. In a class of this embodiment, R⁸ is —CH₃.

In one embodiment of the present invention, R⁹ is selected from the group consisting of: hydrogen, —C₁₋₆alkyl, —C₂₋₆alkenyl, and —C₂₋₆alkynyl, wherein each alkyl, alkenyl and alkynyl is unsubstituted or substituted with one to five substituents selected from halogen.

In another embodiment of the present invention, R⁹ is selected from the group consisting of: hydrogen, and —C₁₋₆alkyl, wherein each alkyl is unsubstituted or substituted with one to five substituents selected from halogen. In a class of this embodiment, R⁹ is selected from the group consisting of: hydrogen, —CH₃, and —CH₂CH₃. In another class of this embodiment, R⁹ is selected from the group consisting of: hydrogen, and —CH₃. In another embodiment of the present invention, R⁹ is hydrogen.

In another embodiment of the present invention, R⁹ is selected from the group consisting of: —C₁₋₆alkyl, wherein each alkyl is unsubstituted or substituted with one to five substituents selected from halogen. In a class of this embodiment of the present invention, R⁹ is selected from the group consisting of: —CH₃, and —CH₂CH₃.

In one embodiment of the present invention, each R^{a} is independently selected from the group consisting of: CN, oxo, halogen, —S(O)₂C₁₋₆alkyl, —C₁₋₆alkyl, —C₁₋₆alkenyl, —C₂₋₆ alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆ cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl C₂₋₆cycloheteroalkyl, —C₂₋₆alkynyl-aryl, —C₂₋₆ alkynyl-heteroaryl, —OH, —(CH₂)ₚ—OC₁₋₆alkyl, —(CH₂)ₚ—OC₂₋₆alkenyl, —(CH₂)ₚ—OC₂₋₆ alkynyl, —(CH₂)ₚ—OC₃₋₆cycloalkyl, —(CH₂)ₚ—OC₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆ alkyl-aryl, —OC₁₋₆alkyl-heteroaryl, —S(O)ᵣR^{h}, —C₁₋₆alkyl-S(O)ᵣR^{h}, —N(R^{k})₂, —C(O)R^{L}, and —NR^{k}R^{L}, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, oxo, halogen, —S(O)₂C₁₋₆alkyl, —C₁₋₆alkyl, —C₁₋₆alkenyl, —C₂₋₆alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl C₂₋₆cycloheteroalkyl, —C₂₋₆alkynyl-aryl, —C₂₋₆ alkynyl-heteroaryl, —OH, —(CH₂)ₚ—OC₁₋₆alkyl, —(CH₂)ₚ—OC₂₋₆alkenyl, —(CH₂)ₚ—OC₂₋₆ alkynyl, —(CH₂)ₚ—OC₃₋₆cycloalkyl, —(CH₂)ₚ—OC₂₋₆cycloheteroalkyl, —(CH₂)ₚ—O-aryl, and —(CH₂)ₚ—O-heteroaryl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, oxo, halogen, —S(O)₂C₁₋₆alkyl, —C₁₋₆alkyl, —C₁₋₆alkenyl, —C₂₋₆alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —OH, —(CH₂)ₚ—OC₁₋₆alkyl, —(CH₂)ₚ—OC₃₋₆cycloalkyl, and —(CH₂)ₚ—OC₂₋₆cycloheteroalkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, oxo, halogen, —S(O)₂C₁₋₆alkyl, —C₁₋₆alkyl, —C₁₋₆alkenyl, —C₂₋₆alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —OH, —OC₁₋₆alkyl, —OC₃₋₆cycloalkyl, and —OC₂₋₆cycloheteroalkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, oxo, halogen, —S(O)₂C₁₋₆alkyl, —C₁₋₆alkyl, —C₁₋₆alkenyl, —C₃₋₆cycloalkyl, —C₂₋₆cycloheteroalkyl, aryl, heteroaryl, —OH, —OC₁₋₆alkyl, —OC₃₋₆cycloalkyl, and —OC₂₋₆cycloheteroalkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, halogen, —C₁₋₆alkyl, —C₁₋₆alkenyl, —C₃₋₆cycloalkyl, —C₂₋₆ cycloheteroalkyl, aryl, heteroaryl, —OC₁₋₆alkyl, —OC₃₋₆cycloalkyl, and —OC₂₋₆ cycloheteroalkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, halogen, —C₁₋₆alkyl, —C₁₋₆alkenyl, —C₃₋₆cycloalkyl, aryl, —OC₁₋₆alkyl, and —OC₃₋₆cycloalkyl, wherein each R^{a} is unsubstituted or substituted with one to six substituents selected from halogen, CF₃, OH, C₁₋₆alkyl, and —OC₁₋₆alkyl. In a class of this embodiment, each R^{a} is independently selected from the group consisting of: CN, F, Cl, —CH₃, —CH(CH₃)₂, —C(CH₃)₃, —CF₃, —CHF₂, —CH₂CF₃, —CH(CH₃)CF₃, —CF₂CH₃, ═CH₂, cyclopropyl, phenyl, —OCF₃, —OCH₃, —OCHF₂, —OCH₂CF₃, and —O-cyclopropyl, wherein each cyclopropyl and phenyl is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: CN, halogen, —C₁₋₆alkyl, —OC₁₋₆alkyl, and —OC₃₋₆cycloalkyl, wherein each R^{a} is unsubstituted or substituted with one to six substituents selected from halogen, CF₃, OH, C₁₋₆alkyl, and —OC₁₋₆alkyl. In another class of this embodiment, each R^{a} is independently selected from the group consisting of: CN, F, Cl, —CH₃, —CH(CH₃)₂, —C(CH₃)₃, —CF₃, —CHF₂, —CH₂CF₃, —CH(CH₃)CF₃, —CF₂CH₃, —OCF₃, —OCH₃, —OCHF₂, —OCH₂CF₃, and —O— cyclopropyl, wherein cyclopropyl is unsubstituted or substituted with one to six substituents selected from: CN, F, Cl, CF₃, OH, CH₃, and —OCH₃. In another class of this embodiment, each R^{a} is independently selected from the group consisting of: F, Cl, —CH₃, —CF₃, —OCF₃, and —O— cyclopropyl, wherein cyclopropyl is unsubstituted or substituted with one to six substituents selected from F, Cl, CF₃, OH, CH₃, and —OCH₃.

In another embodiment of the present invention, each R^{a} is independently selected from the group consisting of: halogen, —C₁₋₆alkyl, —OC₁₋₆alkyl and —OC₃₋₆cycloalkyl, wherein each R^{a} is unsubstituted or substituted with one to six substituents selected from halogen, CF₃, OH, C₁₋₆alkyl, and —OC₁₋₆alkyl. In a class of this embodiment, each R^{a} is independently selected from the group consisting of: halogen, —C₁₋₆alkyl, —OC₁₋₆alkyl, and —OC₃₋₆cycloalkyl, wherein each R^{a} is unsubstituted or substituted with one to six substituents selected from halogen, CF₃, OH, CH₃, and —OCH₃. In another class of this embodiment, each R^{a} is independently selected from the group consisting of: F, Cl, —CH₃, —CH(CH₃)₂, —C(CH₃)₃, —CF₃, —CHF₂, —CH₂CF₃, —CH(CH₃)CF₃, —CF₂CH₃, —OCF₃, —OCH₃, —OCHF₂, —OCH₂CF₃, and —O-cyclopropyl, wherein cyclopropyl is unsubstituted or substituted with one to six substituents selected from F, Cl, CF₃, OH, CH₃, and —OCH₃. In another class of this embodiment, each R^{a} is independently selected from the group consisting of: F, Cl, —CH₃, —CF₃, —OCF₃, and —O-cyclopropyl, wherein cyclopropyl is unsubstituted or substituted with one to six substituents selected from F, C₁, CF₃, OH, CH₃, and —OCH₃. In another class of this embodiment, each R^{a} is independently selected from the group consisting of: F, Cl, —CF₃, —OCF₃, and —O-cyclopropyl, wherein cyclopropyl is unsubstituted or substituted with one to six substituents selected from F, Cl, CF₃, OH, CH₃, and —OCH₃.

In another embodiment of the present invention, R^{a} is independently selected from the group consisting of: CN, F, C., CF3, CHF2, cyclopropyl, 4-fluorophenyl, OCH2CF3, OCF3, OCHF2, and O-cyclopropyl, wherein cyclopropyl is unsubstituted or substituted with one to six substituents selected from F, Cl, CF₃, OH, CH₃, and —OCH₃.

In one embodiment of the present invention, each R^{b} is independently selected from the group consisting of: CN, oxo, halogen, —S(O)₂C₁₋₆alkyl, —C₁₋₆alkyl, —C₁₋₆alkenyl, —C₂₋₆alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆alkynyl-aryl, —C₂₋₆alkynyl-heteroaryl, —OH, —(CH₂)ₚ—OC₁₋₆alkyl, —(CH₂)ₚ—OC₂₋₆alkenyl, —(CH₂)ₚ—OC₂₋₆alkynyl, —(CH₂)ₚ—OC₃₋₆cycloalkyl, —(CH₂)ₚ—OC₂₋₆heterocycloalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆heterocycloalkyl, —OC₁₋₆alkyl-aryl, —OC₁₋₆alkyl-heteroaryl, —S(O)ᵣRⁱ, —C₁₋₆alkyl-S(O)ᵣRⁱ, —N(R^{k})₂, —C(O)R^{L}, and —NR^{k}R^{L}, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment of the present invention, each R^{b} is independently selected from the group consisting of: CN, oxo, halogen, —S(O)₂C₁₋₆alkyl, —C₁₋₆alkyl, —C₁₋₆alkenyl, —C₂₋₆ alkynyl, —C₃₋₆cycloalkyl, —C₂₋₆cycloheteroalkyl, aryl, heteroaryl, —C₁₋₆alkyl-aryl, (CH₂)₂-phenyl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆ alkynyl-aryl, —C₂₋₆alkynyl-heteroaryl, —OH, —(CH₂)ₚ—OC₁₋₆alkyl, —(CH₂)ₚ—OC₂₋₆alkenyl, —(CH₂)ₚ—OC₂₋₆alkynyl, —(CH₂)ₚ—OC₃₋₆cycloalkyl, —(CH₂)ₚ—OC₂₋₆heterocycloalkyl, —(CH₂)ₚ—O-aryl, and —(CH₂)ₚ—O-heteroaryl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment of the present invention, each R^{b} is independently selected from the group consisting of: CN, oxo, halogen, —S(O)₂C₁₋₆alkyl, —C₁₋₆alkyl, —C₁₋₆alkenyl, —C₂₋₆ alkynyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —OH, —(CH₂)ₚ—OC₁₋₆alkyl, —(CH₂)ₚ—OC₃₋₆cycloalkyl and —(CH₂)ₚ—OC₂₋₆heterocycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl. In another embodiment of the present invention, each R^{b} is independently selected from the group consisting of: CN, oxo, halogen, —S(O)₂C₁₋₆alkyl, —C₁₋₆alkyl, —C₁₋₆alkenyl, —C₃₋₆cycloalkyl, —C₂₋₆cycloheteroalkyl, aryl, heteroaryl, —OH, —OC₁₋₆alkyl, —OC₃₋₆cycloalkyl, and —OC₂₋₆heterocycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment of the present invention, each R^{b} is independently selected from the group consisting of: CN, halogen, —C₁₋₆alkyl, —C₁₋₆alkenyl, —C₃₋₆cycloalkyl, —C₂₋₆ cycloheteroalkyl, aryl, heteroaryl, —OC₁₋₆alkyl, —OC₃₋₆cycloalkyl, and —OC₂₋₆heterocycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment of the present invention, each R^{b} is independently selected from the group consisting of: CN, halogen, —C₁₋₆alkyl, —C₁₋₆alkenyl, —C₃₋₆cycloalkyl, aryl, —OC₁₋₆alkyl, and —OC₃₋₆cycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl. In a class of this embodiment of the present invention, each R^{b} is independently selected from the group consisting of: CN, halogen, —C₁₋₆alkyl, —C₁₋₆alkenyl, —C₃₋₆cycloalkyl, aryl, —OC₁₋₆alkyl, and —OC₃₋₆cycloalkyl, wherein each R^{b} is unsubstituted or substituted with one to six substituents selected from halogen, CF₃, OCF₃, CN, CH₂CF₃, CF₂CH₃, —CH₃, and —OCH₃.

In another class of this embodiment of the present invention, each R^{b} is independently selected from the group consisting of: CN, F, Cl, —CH₃, —CH(CH₃)₂, —CF₃, —CHF₂, —CH₂CF₃, —CH(CH₃)CF₃, —CF₂CH₃, —CH₂, cyclopropyl, phenyl, —OCF₃, —OCH₃, —OCHF₂, —OCH₂CF₃, and —O-cyclopropyl, wherein each cyclopropyl and phenyl is unsubstituted or substituted with one to six substituents selected from halogen, CF₃, OCF₃, CN, CH₂CF₃, CF₂CH₃, —CH₃, and —OCH₃. In another class of this embodiment of the present invention, each R^{b} is independently selected from the group consisting of: CN, F, Cl, —CH₃, —CH(CH₃)₂, —CF₃, —CHF₂, —CH₂CF₃, —CF₂CH₃, —CH₂, cyclopropyl, phenyl, —OCF₃, —OCH₃, —OCHF₂, —OCH₂CF₃, and —O— cyclopropyl, wherein each cyclopropyl and phenyl is unsubstituted or substituted with one to six substituents selected from halogen, CF₃, OCF₃, CN, CH₂CF₃, CF₂CH₃, —CH₃, and —OCH₃.

In another embodiment of the present invention, each R^{b} is independently selected from the group consisting of: halogen, —C₁₋₆alkyl, —C₁₋₆alkenyl, —C₃₋₆cycloalkyl, aryl, —OC₁₋₆alkyl, and —OC₃₋₆cycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl. In a class of this embodiment of the present invention, each R^{b} is independently selected from the group consisting of: halogen, —C₁₋₆alkyl, —C₁₋₆alkenyl, —C₃₋₆cycloalkyl, aryl, —OC₁₋₆alkyl, and —OC₃₋₆cycloalkyl, wherein each R^{b} is unsubstituted or substituted with one to six substituents selected from halogen, CF₃, OCF₃, CN, CH₂CF₃, CF₂CH₃, —CH₃, and —OCH₃. In another class of this embodiment of the present invention, each R^{b} is independently selected from the group consisting of: F, Cl, —CH₃, —CH(CH₃)₂, —CF₃, —CHF₂, —CH₂CF₃, —CH(CH₃)CF₃, —CF₂CH₃, ═CH₂, cyclopropyl, phenyl, —OCF₃, —OCH₃, —OCHF₂, —OCH₂CF₃, and —O-cyclopropyl, wherein each cyclopropyl and phenyl is unsubstituted or substituted with one to six substituents selected from halogen, CF₃, OCF₃, CN, CH₂CF₃, CF₂CH₃, —CH₃, and —OCH₃. In another class of this embodiment of the present invention, each R^{b} is independently selected from the group consisting of: F, Cl, —CH₃, —CH(CH₃)₂, —CF₃, —CHF₂, —CH₂CF₃, —CF₂CH₃, ═CH₂, cyclopropyl, phenyl, —OCF₃, —OCH₃, —OCHF₂, —OCH₂CF₃, and —O— cyclopropyl, wherein each cyclopropyl and phenyl is unsubstituted or substituted with one to five substituents selected from halogen, CF₃, OCF₃, CN, CH₂CF₃, CF₂CH₃, —CH₃, and —OCH₃.

In another embodiment of the present invention, each R^{b} is independently selected from the group consisting of: halogen, —C₁₋₆alkyl, and —OC₁₋₆alkyl, wherein each R^{b} is unsubstituted or substituted with one to six substituents selected from halogen, CF₃, OCF₃, CN, CH₂CF₃, CF₂CH₃, —CH₃, and —OCH₃. In a class of this embodiment, each R^{b} is independently selected from the group consisting of: F, C₁, —CH₃, —CH(CH₃)₂, —CF₃, —CHF₂, —CH₂CF₃, —CH(CH₃)CF₃, —CF₂CH₃, —OCF₃, —OCH₃, —OCHF₂, and —OCH₂CF₃. In another class of this embodiment, each R^{b} is independently selected from the group consisting of: F, C₁, —CH₃, —CH(CH₃)₂, —CF₃, —CHF₂, —CH₂CF₃, —CF₂CH₃, —OCF₃, —OCH₃, —OCHF₂, and —OCH₂CF₃. In another class of this embodiment, each R^{b} is independently selected from the group consisting of: F, Cl, —CF₃, —OCHF₂, and —OCH₂CF₃.

In one embodiment of the present invention, R^{c} is selected from: —C₁₋₆alkyl, OH, halogen, and —OC₁₋₆alkyl, wherein alkyl is unsubstituted or substituted with one to three halogens. In another embodiment of the present invention, R^{c} is selected from: —C₁₋₆alkyl, halogen, and —OC₁₋₆alkyl, wherein alkyl is unsubstituted or substituted with one to three halogens. In another embodiment of the present invention, R^{c} is selected from: —C₁₋₆alkyl, and halogen, wherein alkyl is unsubstituted or substituted with one to three halogens. In another embodiment of the present invention, R^{c} is —C₁₋₆alkyl, wherein alkyl is unsubstituted or substituted with one to three halogens. In another embodiment of the present invention, R^{c} is halogen.

In one embodiment of the present invention, R^{d} is selected from: —C₁₋₆alkyl, OH, halogen, and —OC₁₋₆alkyl, wherein alkyl is unsubstituted or substituted with one to three halogens. In another embodiment of the present invention, R^{d} is selected from: —C₁₋₆alkyl, halogen, and —OC₁₋₆alkyl, wherein alkyl is unsubstituted or substituted with one to three halogens. In another embodiment of the present invention, R^{d} is selected from: —C₁₋₆alkyl, and halogen, wherein alkyl is unsubstituted or substituted with one to three halogens. In another embodiment of the present invention, R^{d} is —C₁₋₆alkyl, wherein alkyl is unsubstituted or substituted with one to three halogens. In another embodiment of the present invention, R^{d} is halogen. In a class of this embodiment, R^{d} is F.

In one embodiment of the present invention, R^{e} is selected from: hydrogen, and C₁₋₆alkyl. In another embodiment of the present invention, R^{e} is hydrogen. In another embodiment of the present invention, R^{e} is C₁₋₆alkyl.

In one embodiment of the present invention, R^{f} is selected from: —C₁₋₆alkyl, OH, halogen, and —OC₁₋₆alkyl, wherein alkyl is unsubstituted or substituted with one to three halogens. In another embodiment of the present invention, R^{f} is selected from: —C₁₋₆alkyl, halogen, and —OC₁₋₆alkyl, wherein alkyl is unsubstituted or substituted with one to three halogens. In another embodiment of the present invention, R^{f} is selected from: —C₁₋₆alkyl, and halogen, wherein alkyl is unsubstituted or substituted with one to three halogens. In another embodiment of the present invention, R^{f} is —C₁₋₆alkyl, wherein alkyl is unsubstituted or substituted with one to three halogens. In another embodiment of the present invention, R^{f} is halogen. In a class of this embodiment, R^{f} is F.

In one embodiment of the present invention, R^{g} is selected from: —C₁₋₆alkyl, OH, halogen, and —OC₁₋₆alkyl, wherein alkyl is unsubstituted or substituted with one to three halogens. In another embodiment of the present invention, R^{g} is selected from: —C₁₋₆alkyl, halogen, and —OC₁₋₆alkyl, wherein alkyl is unsubstituted or substituted with one to three halogens. In another embodiment of the present invention, R^{g} is selected from: —C₁₋₆alkyl, and halogen, wherein alkyl is unsubstituted or substituted with one to three halogens. In another embodiment of the present invention, R^{g} is —C₁₋₆alkyl, wherein alkyl is unsubstituted or substituted with one to three halogens. In another embodiment of the present invention, R^{g} is halogen. In a class of this embodiment, R^{g} is F.

In one embodiment of the present invention, R^{h} is selected from: hydrogen, C₁₋₆alkyl, C₃₋₆cycloalkyl, aryl, and heteroaryl. In another embodiment of the present invention, R^{h} is selected from: hydrogen, C₁₋₆alkyl, and C₃₋₆cycloalkyl. In another embodiment of the present invention, R^{h} is selected from: hydrogen, and —C₁₋₆alkyl. In another embodiment of the present invention, R^{h} is hydrogen. In another embodiment of the present invention, R^{h} is C₁₋₆alkyl.

In one embodiment of the present invention, Rⁱ is selected from: hydrogen, C₁₋₆alkyl, C₃₋₆cycloalkyl, aryl, and heteroaryl. In another embodiment of the present invention, Rⁱ is selected from: hydrogen, C₁₋₆alkyl, and C₃₋₆cycloalkyl. In another embodiment of the present invention, Rⁱ is selected from: hydrogen, and —C₁₋₆alkyl. In another embodiment of the present invention, Rⁱ is hydrogen. In another embodiment of the present invention, Rⁱ is C₁₋₆alkyl.

In one embodiment of the present invention, Rⁱ is selected from: hydrogen, C₁₋₆alkyl, C₃₋₆alkenyl, C₃₋₆alkynyl, C₃₋₆cycloalkyl, C₂₋₅cycloheteroalkyl, aryl, and heteroaryl.

In another embodiment of the present invention, R^{j} is selected from: hydrogen, C₁₋₆alkyl, C₃₋₆cycloalkyl, C₂₋₅cycloheteroalkyl, aryl, and heteroaryl.

In another embodiment of the present invention, R^{j} is selected from: hydrogen, C₁₋₆alkyl, C₃₋₆cycloalkyl, and C₂₋₅cycloheteroalkyl. In another embodiment of the present invention, R^{j} is selected from: hydrogen, C₁₋₆alkyl, and C₃₋₆cycloalkyl. In another embodiment of the present invention, R^{j} is selected from: hydrogen, and C₁₋₆alkyl. In another embodiment of the present invention, R^{j} is hydrogen. In another embodiment of the present invention, R^{j} is C₁₋₆alkyl.

In one embodiment of the present invention, R^{k} is selected from: hydrogen, and C₁₋₆alkyl. In another embodiment of the present invention, R^{k} is hydrogen. In another embodiment of the present invention, R^{k} is C₁₋₆alkyl.

In one embodiment of the present invention, R^{L} is selected from: hydrogen, C₁₋₆alkyl, C₃₋₆cycloalkyl, aryl, and heteroaryl. In another embodiment of the present invention, R^{L} is selected from: hydrogen, C₁₋₆alkyl, and C₃₋₆cycloalkyl. In another embodiment of the present invention, R^{L} is selected from: hydrogen, and —C₁₋₆alkyl. In another embodiment of the present invention, R^{L} is hydrogen. In another embodiment of the present invention, R^{L} is C₁₋₆alkyl.

In one embodiment of the present invention, m is 0, 1 or 2. In another embodiment, m is 0 or 1. In another embodiment, m is 0 or 2. In another embodiment, m is 0. In another embodiment, m is 1. In another embodiment, m is 2.

In one embodiment of the present invention, n is 2, 3, 4, 5 or 6. In another embodiment, n is 2, 3, 4, or 5. In another embodiment, n is 2, 3, or 4. In another embodiment, n is 2 or 3. In another embodiment, n is 2 or 4. In another embodiment, n is 2, 3, 4, or 5. In another embodiment, n is 3. In another embodiment, n is 4. In another embodiment, n is 5. In another embodiment, n is 6.

In one embodiment of the present invention, p is 0, 1, 2 or 3. In another embodiment, p is 0, 1 or 2. In another embodiment, p is 0, 1 or 3. In another embodiment, p is 1, 2 or 3. In another embodiment, p is 1 or 2. In another embodiment, p is 1 or 3. In another embodiment, p is 0 or 1. In another embodiment, p is 0 or 2. In another embodiment, p is 0 or 3. In another embodiment, p is 0. In another embodiment, p is 1. In another embodiment, p is 2. In another embodiment, p is 3.

In one embodiment of the present invention, q is 0, 1, 2 or 3. In another embodiment, q is 0, 1 or 2. In another embodiment, q is 0, 1 or 3. In another embodiment, q is 1, 2 or 3. In another embodiment, q is 1 or 2. In another embodiment, q is 1 or 3. In another embodiment, q is 0 or 1. In another embodiment, q is 0 or 2. In another embodiment, q is 0 or 3. In another embodiment, q is 0. In another embodiment, q is 1. In another embodiment, q is 2. In another embodiment, q is 3.

In one embodiment of the present invention, r is 0, 1 or 2. In another embodiment, r is 0 or 1. In another embodiment, r is 0 or 2. In another embodiment, r is 0. In another embodiment, r is 1. In another embodiment, r is 2.

In one embodiment of the present invention, s is 0, 1, 2, 3, 4, 5 or 6. In another embodiment, s is 0, 1, 2, 3, 4, or 5. In another embodiment, s is 1, 2, 3, 4, 5 or 6. In another embodiment, s is 1, 2, 3, 4 or 5. In another embodiment, s is 0, 1, 2, 3, or 4. In another embodiment, s is 1, 2, 3, or 4. In another embodiment, s is 0, 1, 2, or 3. In another embodiment, s is 1, 2, or 3. In another embodiment, s is 0, 1 or 2. In another embodiment, s is 1 or 2. In another embodiment, s is 0. In another embodiment, s is 1. In another embodiment, s is 2. In another embodiment, s is 3. In another embodiment, s is 4. In another embodiment, s is 5. In another embodiment, s is 6.

In one embodiment of the present invention, t is 0, 1, 2, 3, 4, 5 or 6. In another embodiment, t is 0, 1, 2, 3, 4, or 5. In another embodiment, t is 1, 2, 3, 4, 5 or 6. In another embodiment, t is 1, 2, 3, 4 or 5. In another embodiment, t is 0, 1, 2, 3, or 4. In another embodiment, t is 1, 2, 3, or 4. In another embodiment, t is 0, 1, 2, or 3. In another embodiment, tis 1, 2, or 3. In another embodiment, t is 0, 1 or 2. In another embodiment, t is 1 or 2. In another embodiment, t is 0. In another embodiment, t is 1. In another embodiment, t is 2. In another embodiment, t is 3. In another embodiment, t is 4. In another embodiment, t is 5. In another embodiment, t is 6.

In another embodiment of the present invention, the invention relates to compounds of structural formula Ia:

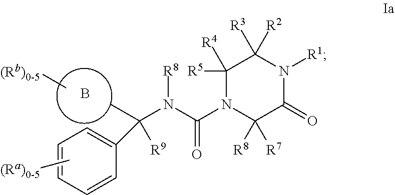

or a pharmaceutically acceptable salt thereof.

In another embodiment of the present invention, the invention relates to compounds of structural formula Ib:

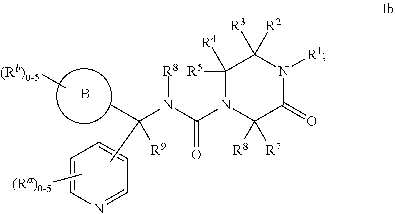

or a pharmaceutically acceptable salt thereof.

In a class of this embodiment, the pyridyl ring is:

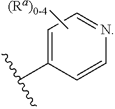

In another class of this embodiment, the pyridyl ring is:

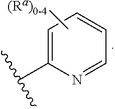

In another class of this embodiment, the pyridyl ring is:

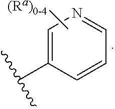

In another embodiment of the present invention, the invention relates to compounds of structural formula Ic:

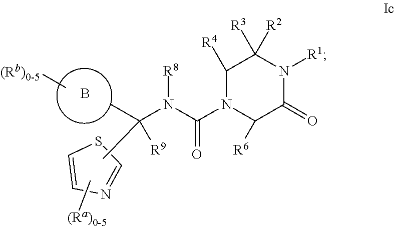

or a pharmaceutically acceptable salt thereof.

In another embodiment of the present invention, the invention relates to compounds of structural formula Id:

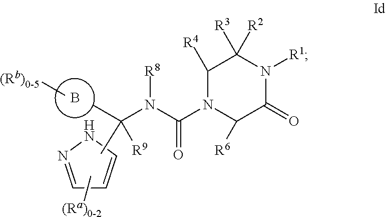

or a pharmaceutically acceptable salt thereof.

In another embodiment of the present invention, the invention relates to compounds of structural formula Ie:

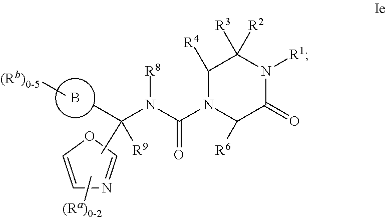

or a pharmaceutically acceptable salt thereof.

In another embodiment of the present invention, the invention relates to compounds of structural formula If:

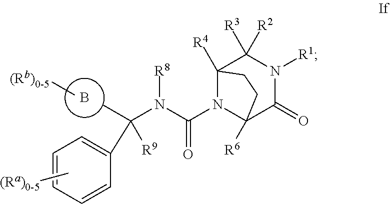

or a pharmaceutically acceptable salt thereof.

In another embodiment of the present invention, the invention relates to compounds of structural formula Ig:

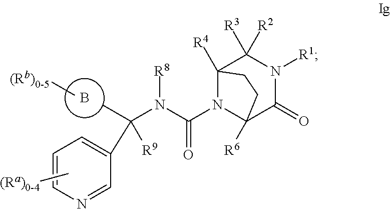

or a pharmaceutically acceptable salt thereof.

In a class of this embodiment, the pyridyl ring is:

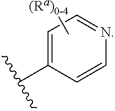

In another class of this embodiment, the pyridyl ring is:

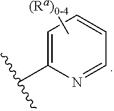

In another class of this embodiment, the pyridyl ring is:

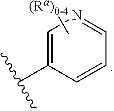

In another embodiment of the present invention, the invention relates to compounds of structural formula Ih:

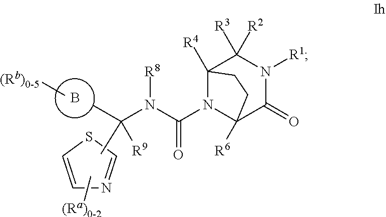

or a pharmaceutically acceptable salt thereof.

In another embodiment of the present invention, the invention relates to compounds of structural formula Ii:

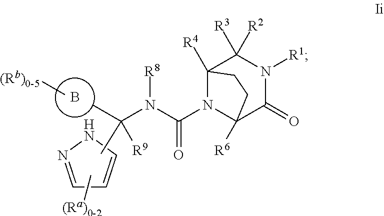

or a pharmaceutically acceptable salt thereof.

In another embodiment of the present invention, the invention relates to compounds of structural formula Ij:

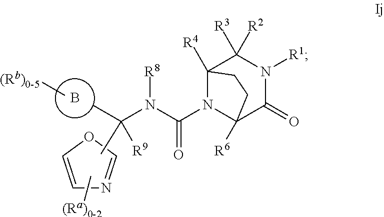

or a pharmaceutically acceptable salt thereof.

The compound of structural formula I, includes the compounds of structural formulas Ia, Ib, Ic, Id, Ie, If, Ig, Ih, and Ij, and pharmaceutically acceptable salts, hydrates and solvates thereof.

Another embodiment of the present invention relates to compounds of structural formula I wherein:
A is selected from the group consisting of:
(1) aryl, and
(2) heteroaryl,
wherein each aryl and heteroaryl is unsubstituted or substituted with one to five substituents selected from R^{a}; and
B, R¹, R², R³, R⁴, R⁵, R⁶, R⁷, R⁸, R⁹, R^{a}, R^{b}, R^{c}, R^{d}, R^{e}, R^{f}, R^{g}, R^{h}, Rⁱ, R^{j}, R^{k}, R^{L}, m, n, p, q, r, s and t are as defined above;
or a pharmaceutically acceptable salt thereof.

Another embodiment of the present invention relates to compounds of structural formula I wherein:
A is selected from the group consisting of:
(1) phenyl,
(2) pyridine,
(3) pyrazole,
(4) oxazole, and
(5) thiazole,
wherein A is unsubstituted or substituted with one to five substituents selected from R^{a}; B is independently selected from the group consisting of:
(1) aryl, and
(2) heteroaryl,
wherein B is unsubstituted or substituted with one to five substituents selected from R^{b};
R¹ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{c};
R² is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R³ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R⁴ is selected from the group consisting of:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁵ is selected from the group consisting of:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁶ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁷ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁸ is selected from the group consisting of:
(1) hydrogen, and
(2) —C₁₋₆alkyl,
wherein alkyl is unsubstituted or substituted with one to five substituents selected from R^{e};
R⁹ is selected from the group consisting of:
(1) hydrogen, and
(2) —C₁₋₆alkyl,
wherein each alkyl is unsubstituted or substituted with one to five substituents selected from halogen;
each R^{a} is independently selected from the group consisting of:
(1) CN,
(2) oxo,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —OH,
(17) —OC₁₋₆alkyl,
(18) —OC₃₋₆cycloalkyl, and
(19) —OC₂₋₆cycloheteroalkyl,
wherein each R^{a} is unsubstituted or substituted with one to six substituents selected from halogen, CF₃, OH, C₁₋₆alkyl, and —OC₁₋₆alkyl;
each R^{b} is independently selected from the group consisting of:
(1) CN,
(2) OXO,
(3) halogen,
(4) —S(O)₂C₁₋₆alkyl,
(5) —C₁₋₆alkyl,
(6) —C₁₋₆alkenyl,
(7) —C₃₋₆cycloalkyl,
(8) —C₂₋₆cycloheteroalkyl,
(9) aryl,
(10) heteroaryl,
(11) —OH,
(12) —OC₁₋₆alkyl,
(13) —OC₃₋₆cycloalkyl, and
(14) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl; and R^{c}, R^{d}, R^{e}, R^{f}, R^{g}, R^{h}, Rⁱ, R^{j}, R^{k}, R^{L}, m, n, p, q, r, s and t are as defined above;
or a pharmaceutically acceptable salt thereof.

Another embodiment of the present invention relates to compounds of structural formula I wherein:
A is selected from the group consisting of:
(1) phenyl, and
(1) pyridine,
(2) wherein phenyl and pyridine are unsubstituted or substituted with one to five substituents selected from R^{a};
B is heteroaryl, wherein heteroaryl is unsubstituted or substituted with one to five substituents selected from R^{b};
R¹, R², R³, R⁴, and R⁵ are hydrogen;
R⁶ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁷ is selected from the group consisting of:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁸ and R⁹ are hydrogen;
each R^{a} is independently selected from the group consisting of:
(1) CN,
(2) halogen,
(3) —C₁₋₆alkyl,
(4) —C₁₋₆alkenyl,
(5) —C₃₋₆cycloalkyl, aryl,
(6) —OC₁₋₆alkyl, and
(7) —OC₃₋₆cycloalkyl,
wherein each R^{a} is unsubstituted or substituted with one to six substituents selected from halogen, CF₃, OH, C₁₋₆alkyl, and —OC₁₋₆alkyl;
each R^{b} is independently selected from the group consisting of:
(1) CN,
(2) halogen,
(3) —C₁₋₆alkyl,
(4) —C₁₋₆alkenyl,
(5) —C₃₋₆cycloalkyl,
(6) —C₂₋₆cycloheteroalkyl,
(7) aryl,
(8) heteroaryl,
(9) —OC₁₋₆alkyl,
(10) —OC₃₋₆cycloalkyl, and
(11) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl; and
R^{c}, R^{d}, R^{e}, R^{f}, R^{g}, R^{h}, Rⁱ, R^{j}, R^{k}, R^{L}, m, n, p, q, r, s and t and are as defined above; or a pharmaceutically acceptable salt thereof.

Illustrative, but non-limiting, examples of the compounds of the present invention that are useful as inhibitors of Naᵥ1.8 channel activity are the following compounds:
(1) (2R)—N—((R)(3-chloro-2,4-difluorophenyl)(6-(2,2,2-trifluoroethoxy)pyridin-3-yl)methyl)-2-methyl-3-oxopiperazine-1-carboxamide;
(2) (2R)—N—((S)(3-chloro-2,4-difluorophenyl)(6-(2,2,2-trifluoroethoxy)pyridin-3-yl)methyl)-2-methyl-3-oxopiperazine-1-carboxamide;
(3) N—((R or S)-(3-chloro-4-fluorophenyl)(6-(2,2,2-trifluoro-ethoxy)pyridin-3-yl)methyl)-3-oxopiperazine-1-carboxamide;
(4) N—((S or R)-(3-chloro-4-fluorophenyl)(6-(2,2,2-trifluoroethoxy)pyridin-3-yl)methyl)-3-oxopiperazine-1-carboxamide;
(5) (2R)—N—((R or S)-(3-chloro-4-fluorophenyl)(6-(2,2,2-trifluoroethoxy)pyridin-3-yl)methyl)-2-methyl-3-oxopiperazine-1-carboxamide;
(6) (2R)—N—((S or R)-(3-chloro-4-fluorophenyl)(6-(2,2,2-trifluoroethoxy)pyridin-3-yl)methyl)-2-methyl-3-oxopiperazine-1-carboxamide;
(7) (2R)—N—((R or S)-(3-chloro-2,4-difluorophenyl)(6-(trifluoromethoxy)pyridin-3-yl)methyl)-2-methyl-3-oxopiperazine-1-carboxamide;
(8) (2R)—N—((S or R)-(3-chloro-2,4-difluorophenyl)(6-(trifluoromethoxy)pyridin-3-yl)methyl)-2-methyl-3-oxopiperazine-1-carboxamide;
(9) (2R)—N—((R)-(4-chlorophenyl)(6-(2,2,2-trifluoroethoxy)pyridin-3-yl)methyl)-2-methyl-3-oxopiperazine-1-carboxamide;
(10) (2R)—N—((S)-(4-chlorophenyl)(6-(2,2,2-trifluoroethoxy)pyridin-3-yl)methyl)-2-methyl-3-oxopiperazine-1-carboxamide;
(11) (2R)—N—((R)-(3,4-difluorophenyl)(6-(2,2,2-trifluoroethoxy)pyridin-3-yl)methyl)-2-methyl-3-oxopiperazine-1-carboxamide;
(12) (2R)—N—((S)-(3,4-difluorophenyl)(6-(2,2,2-trifluoroethoxy)pyridin-3-yl)methyl)-2-methyl-3-oxopiperazine-1-carboxamide;
(13) (2R)—N—((R)-(3-chloro-4,5-difluorophenyl)(6-(2,2,2-trifluoroethoxy)pyridin-3-yl)methyl)-2-methyl-3-oxopiperazine-1-carboxamide;
(14) (2R)—N—((S)-(3-chloro-4,5-difluorophenyl)(6-(2,2,2-trifluoroethoxy)pyridin-3-yl)methyl)-2-methyl-3-oxopiperazine-1-carboxamide;
(15) N—((R)-(3-chloro-2,4-difluorophenyl)(6-(2,2,2-trifluoroethoxy) pyridin-3-yl)methyl)-3-oxopiperazine-1-carboxamide;
(16) N—((S)-(3-chloro-2,4-difluorophenyl)(6-(2,2,2-trifluoroethoxy) pyridin-3-yl)methyl)-3-oxopiperazine-1-carboxamide;
(17) (2R)—N—((R)-(4-chlorophenyl)(6-(trifluoromethyl)pyridin-2-yl)methyl)-2-methyl-3-oxopiperazine-1-carboxamide;
(18) (2R)—N—((S)-(4-chlorophenyl)(6-(trifluoromethyl)pyridin-2-yl)methyl)-2-methyl-3-oxopiperazine-1-carboxamide;
(19) (2R)—N—((R)-(3-chloro-2,4-difluorophenyl)(5-fluoro-6-(2,2,2-trifluoroethoxy)pyridin-3-yl)methyl)-2-methyl-3-oxopiperazine-1-carboxamide;
(20) (2R)—N—((S)-(3-chloro-2,4-difluorophenyl)(5-fluoro-6-(2,2,2-trifluoroethoxy)pyridin-3-yl)methyl)-2-methyl-3-oxopiperazine-1-carboxamide;
(21) (2R)—N—((R)-(3-chloro-4-fluorophenyl)(2-(trifluoromethyl)imidazo[1,2-a]pyridin-6-yl)methyl)-2-methyl-3-oxopiperazine-1-carboxamide;
(22) (2R)—N—((S)-(3-chloro-4-fluorophenyl)(2-(trifluoromethyl)imidazo[1,2-a]pyridin-6-yl)methyl)-2-methyl-3-oxopiperazine-1-carboxamide;
(23) (2R)—N—((R)-(3-chloro-2,4-difluorophenyl)(2-(2,2,2-trifluoroethoxy)pyrimidin-5-yl)methyl)-2-methyl-3-oxopiperazine-1-carboxamide;
(24) (2R)—N—((S)-(3-chloro-2,4-difluorophenyl)(2-(2,2,2-trifluoroethoxy)pyrimidin-5-yl)methyl)-2-methyl-3-oxopiperazine-1-carboxamide;
(25) (2R)—N—((R)-(3-chloro-2,4-difluorophenyl)(2-(2,2,2-trifluoroethoxy)thiazol-5-yl)methyl)-2-methyl-3-oxopiperazine-1-carboxamide;
(26) (2R)—N—((S)-(3-chloro-2,4-difluorophenyl)(2-(2,2,2-trifluoroethoxy)thiazol-5-yl)methyl)-2-methyl-3-oxopiperazine-1-carboxamide;
(27) (2R)—N—((R)-(3-chloro-2,4-difluorophenyl)(2-(difluoromethoxy)thiazol-5-yl)methyl)-2-methyl-3-oxopiperazine-1-carboxamide;
(28) (2R)—N—((S)-(3-chloro-2,4-difluorophenyl)(2-(difluoromethoxy)thiazol-5-yl)methyl)-2-methyl-3-oxopiperazine-1-carboxamide;
(29) (2R)—N—((R)-(3-chloro-2,4-difluorophenyl)(6-(trifluoromethyl)pyridin-3-yl)methyl)-2-methyl-3-oxopiperazine-1-carboxamide;
(30) (2R)—N—((S)-(3-chloro-2,4-difluorophenyl)(6-(trifluoromethyl)pyridin-3-yl)methyl)-2-methyl-3-oxopiperazine-1-carboxamide;
(31) (2R)—N—((R)-(3-chloro-4-fluorophenyl)(6-(trifluoromethyl)pyridin-3-yl)methyl)-2-methyl-3-oxopiperazine-1-carboxamide;
(32) (2R)—N—((S)-(3-chloro-4-fluorophenyl)(6-(trifluoromethyl)pyridin-3-yl)methyl)-2-methyl-3-oxopiperazine-1-carboxamide;
(33) (2R)—N—((R)-(4-chlorophenyl)(6-(trifluoromethyl)pyridin-3-yl)methyl)-2-methyl-3-oxopiperazine-1-carboxamide;
(34) (2R)—N—((S)-(4-chlorophenyl)(6-(trifluoromethyl)pyridin-3-yl)methyl)-2-methyl-3-oxopiperazine-1-carboxamide;
(35) (2R)—N—((R)-(3,4-dichlorophenyl)(6-(trifluoromethyl)pyridin-3-yl)methyl)-2-methyl-3-oxopiperazine-1-carboxamide;
(36) (2R)—N—((S)-(3,4-dichlorophenyl)(6-(trifluoromethyl)pyridin-3-yl)methyl)-2-methyl-3-oxopiperazine-1-carboxamide;
(37) (2R)—N—((R)-(4-fluoro-3-(trifluoromethoxy)phenyl)(6-(trifluoromethyl) pyridin-3-yl)methyl)-2-methyl-3-oxopiperazine-1-carboxamide;
(38) (2R)—N—((S)-(4-fluoro-3-(trifluoromethoxy)phenyl)(6-(trifluoromethyl) pyridin-3-yl)methyl)-2-methyl-3-oxopiperazine-1-carboxamide;
(39) (2R)—N—((R)-(5-chloro-6-(trifluoromethyl)pyridin-3-yl)(4-(trifluoromethoxy)-phenyl)methyl)-2-methyl-3-oxopiperazine-1-carboxamide;
(40) (2R)—N—((S)-(5-chloro-6-(trifluoromethyl)pyridin-3-yl)(4-(trifluoromethoxy)-phenyl)methyl)-2-methyl-3-oxopiperazine-1-carboxamide;
(41) (2R)—N—((R)-(5-chloro-6-(trifluoromethyl)pyridin-3-yl)(3-fluoro-4-(trifluoromethoxy)-phenyl)methyl)-2-methyl-3-oxopiperazine-1-carboxamide;
(42) (2R)—N—((S)-(5-chloro-6-(trifluoromethyl)pyridin-3-yl)(3-fluoro-4-(trifluoromethoxy)-phenyl)methyl)-2-methyl-3-oxo-piperazine-1-carboxamide;
(43) (2R)—N—((R)-(3-chloro-4-(trifluoromethoxy)phenyl)(5-chloro-6-(trifluoromethylpyridin-3-yl)methyl)-2-methyl-3-oxopiperazine-1-carboxamide;
(44) (2R)—N—((S)-(3-chloro-4-(trifluoromethoxy)phenyl)(5-chloro-6-(trifluoro-methyl)pyridin-3-yl)methyl)-2-methyl-3-oxopiperazine-1-carboxamide;
(45) (2R)—N—((R)-(4-chloro-3-cyanophenyl)(5-chloro-6-(trifluoromethyl) pyridin-3-yl)methyl)-2-methyl-3-oxopiperazine-1-carboxamide;
(46) (2R)—N—((R)-(3-chloro-4-cyanophenyl)(5-chloro-6-(trifluoromethyl) pyridin-3-yl)methyl)-2-methyl-3-oxopiperazine-1-carboxamide;
(47) (2R)—N—((S)-(3-chloro-4-cyanophenyl)(5-chloro-6-(trifluoromethyl) pyridin-3-yl)methyl)-2-methyl-3-oxopiperazine-1-carboxamide;
(48) (2R)—N—((R)-(5-chloro-6-(trifluoromethyl)pyridin-3-yl)(4-cyclopropoxy-3-fluorophenyl)methyl)-2-methyl-3-oxopiperazine-1-carboxamide;
(49) (2R)—N—((S)-(5-chloro-6-(trifluoromethyl)pyridin-3-yl)(4-cyclopropoxy-3-fluorophenyl)methyl)-2-methyl-3-oxopiperazine-1-carboxamide;
(50) (2R)—N—((R)-(3-chloro-4-fluorophenyl)(5-chloro-6-(trifluoromethyl) pyridin-3-yl)methyl)-2-methyl-3-oxopiperazine-1-carboxamide;
(51) (2R)—N—((S)-(3-chloro-4-fluorophenyl)(5-chloro-6-(trifluoromethyl) pyridin-3-yl)methyl)-2-methyl-3-oxopiperazine-1-carboxamide;
(52) (2R)—N—((R)-(3-chloro-2,4-difluorophenyl)(5-chloro-6-cyclopropyl pyridin-3-yl)methyl)-2-methyl-3-oxopiperazine-1-carboxamide;
(53) (2R)—N—((S)-(3-chloro-2,4-difluorophenyl)(5-chloro-6-cyclopropyl pyridin-3-yl)methyl)-2-methyl-3-oxopiperazine-1-carboxamide;
(54) (2R)—N—((R)-(3,4-dichlorophenyl)(2-(trifluoromethyl)pyrimidin-5-yl)methyl)-2-methyl-3-oxopiperazine-1-carboxamide;
(55) (2R)—N—((S)-(3,4-dichlorophenyl)(2-(trifluoromethyl)pyrimidin-5-yl)methyl)-2-methyl-3-oxopiperazine-1-carboxamide;
(56) (2R)—N—((R)-(3,4-dichloro-2-fluorophenyl)(6-(trifluoro-methyl)pyridin-3-yl)methyl)-2-methyl-3-oxopiperazine-1-carboxamide;
(57) (2R)—N—((S)-(3,4-dichloro-2-fluorophenyl)(6-(trifluoro-methyl)pyridin-3-yl)methyl)-2-methyl-3-oxopiperazine-1-carboxamide;
(58) (2R)—N—((R)-(3-chloro-2,4-difluorophenyl)(2-(trifluoro-methyl)pyrimidin-5-yl)methyl)-2-methyl-3-oxopiperazine-1-carboxamide;
(59) (2R)—N—((S)-(3-chloro-2,4-difluorophenyl)(2-(trifluoro-methyl)pyrimidin-5-yl)methyl)-2-methyl-3-oxopiperazine-1-carboxamide;
(60) (2R)—N—((R)-(3-chloro-2,4-difluorophenyl)(6-(difluoro-methoxy)pyridin-3-yl)methyl)-2-methyl-3-oxopiperazine-1-carboxamide;
(61) (2R)—N—((S)-(3-chloro-2,4-difluorophenyl)(6-(difluoro-methoxy)pyridin-3-yl)methyl)-2-methyl-3-oxopiperazine-1-carboxamide;
(62) (2R)—N—((R)-(3-chloro-2,4-difluorophenyl)(6-(difluoro-methyl)pyridin-3-yl)methyl)-2-methyl-3-oxopiperazine-1-carboxamide;
(63) (2R)—N—((S)-(3-chloro-2,4-difluorophenyl)(6-(difluoro-methyl)pyridin-3-yl)methyl)-2-methyl-3-oxopiperazine-1-carboxamide;
(64) (2R)—N—((R)-(3-chloro-2,4-difluorophenyl)(6-cyclo-propylpyridin-3-yl)methyl)-2-methyl-3-oxopiperazine-1-carboxamide;
(65) (2R)—N—((S)-(3-chloro-2,4-difluorophenyl)(6-cyclo-propylpyridin-3-yl)methyl)-2-methyl-3-oxopiperazine-1-carboxamide;
(66) (2R)—N—((R)-(3-chloro-2,4-difluorophenyl)(5-fluoro-6-(trifluoromethyl)pyridin-3-yl)methyl)-2-methyl-3-oxopiperazine-1-carboxamide;
(67) (2R)—N—((S)-(3-chloro-2,4-difluorophenyl)(5-fluoro-6-(trifluoromethyl)pyridin-3-yl)methyl)-2-methyl-3-oxopiperazine-1-carboxamide;
(68) (2R)—N—((R)-(3-chloro-2,4-difluorophenyl)(5-chloro-6-(trifluoromethyl)pyridin-3-yl)methyl)-2-methyl-3-oxopiperazine-1-carboxamide;
(69) (2R)—N—((S)-(3-chloro-2,4-difluorophenyl)(5-chloro-6-(trifluoromethyl)pyridin-3-yl)methyl)-2-methyl-3-oxopiperazine-1-carboxamide;
(70) N—((R)-(3-chloro-2,4-difluorophenyl)(5-chloro-6-(trifluoro-methyl)pyridin-3-yl)methyl)-3-oxopiperazine-1-carboxamide;
(71) N—((S)-(3-chloro-2,4-difluorophenyl)(5-chloro-6-(trifluoro-methyl)pyridin-3-yl)methyl)-3-oxopiperazine-1-carboxamide;
(72) N—(R)-(3-chloro-2,4-difluorophenyl)(5-fluoro-6-(trifluoro-methyl)pyridin-3-yl)methyl)-(R or S)-2-cyclopropyl-3-oxopiperazine-1-carboxamide;
(73) N—((S)-(3-chloro-2,4-difluorophenyl)(5-fluoro-6-(trifluoro-methyl)pyridin-3-yl)methyl)-(S or R)-2-cyclopropyl-3-oxopiperazine-1-carboxamide;
(74) N—((R)-(3-chloro-2,4-difluorophenyl)(6-(trifluoromethyl)pyridin-3-yl)methyl)-(R)-2-cyclopropyl-3-oxopiperazine-1-carboxamide;
(75) N—((S)-(3-chloro-2,4-difluorophenyl)(6-(trifluoromethyl)pyridin-3-yl)methyl)-(S)-2-cyclopropyl-3-oxopiperazine-1-carboxamide;
(76) N—((R)-(3-chloro-2,4-difluorophenyl)(6-(trifluoromethyl)pyridin-3-yl)methyl)-(R or S)-2-isopropyl-3-oxo-piperazine-1-carboxamide;
(77) N—((S)-(3-chloro-2,4-difluorophenyl)(6-(trifluoromethyl)pyridin-3-yl)methyl)-(S or R)-2-isopropyl-3-oxo-piperazine-1-carboxamide;
(78) N—((R)-(3-chloro-2,4-difluorophenyl)(6-(trifluoromethyl)pyridin-3-yl)methyl)-(R or S)-2-ethyl-3-oxopiperazine-1-carboxamide;
(79) N—((S)-(3-chloro-2,4-difluorophenyl)(6-(trifluoromethyl)pyridin-3-yl)methyl)-(R or S)-2-ethyl-3-oxopiperazine-1-carboxamide;
(80) N—((R)-(3-chloro-2,4-difluorophenyl)(2-(trifluoromethyl)pyrimidin-5-yl)methyl)-(R or S)-2-cyclopropyl-3-oxopiperazine-1-carboxamide;
(81) N—((S)-(3-chloro-2,4-difluorophenyl)(2-(trifluoromethyl)pyrimidin-5-yl)methyl)-(S or R)-2-cyclopropyl-3-oxopiperazine-1-carboxamide;
(82) N—((R)-(3-chloro-4-fluorophenyl)(6-(trifluoromethyl)pyridin-2-yl)methyl)-3-oxopiperazine-1-carboxamide;
(83) N—((S)-(3-chloro-4-fluorophenyl)(6-(trifluoromethyl)pyridin-2-yl)methyl)-3-oxopiperazine-1-carboxamide;
(84) N—((R)-(3-chloro-4-fluorophenyl)(6-(trifluoromethyl)pyridin-2-yl)methyl)-2,2-dimethyl-3-oxopiperazine-1-carboxamide;
(85) N—((S)-(3-chloro-4-fluorophenyl)(6-(trifluoromethyl)pyridin-2-yl)methyl)-2,2-dimethyl-3-oxopiperazine-1-carboxamide;
(86) (2R)—N—((R)-(3-chloro-4-fluorophenyl)(6-(trifluoro-methyl)pyridin-2-yl)methyl)-2-methyl-3-oxopiperazine-1-carboxamide;
(87) (2R)—N—((S)-(3-chloro-4-fluorophenyl)(6-(trifluoro-methyl)pyridin-2-yl)methyl)-2-methyl-3-oxopiperazine-1-carboxamide;
(88) (2S)—N—((R)-(3-chloro-4-fluorophenyl)(6-(trifluoro-methyl)pyridin-2-yl)methyl)-2-methyl-3-oxopiperazine-1-carboxamide;
(89) (2S)—N—((S)-(3-chloro-4-fluorophenyl)(6-(trifluoro-methyl)pyridin-2-yl)methyl)-2-methyl-3-oxopiperazine-1-carboxamide;
(90) (3S)—N—((R)-(3-chloro-4-fluorophenyl)(6-(trifluoro-methyl)pyridin-2-yl)methyl)-3-methyl-5-oxopiperazine-1-carboxamide;
(91) (3R)—N—((S)-(3-chloro-4-fluorophenyl)(6-(trifluoro-methyl)pyridin-2-yl)methyl)-3-methyl-5-oxopiperazine-1-carboxamide;
(92) (2R)—N—((R)-(3-chloro-4-fluorophenyl)(6-(trifluoro-methyl)pyridin-2-yl)methyl)-2-(fluoromethyl)-5-oxopiperazine-1-carboxamide;
(93) (2S)—N—((S)-(3-chloro-4-fluorophenyl)(6-(trifluoro-methyl)pyridin-2-yl)methyl)-2-(fluoromethyl)-5-oxopiperazine-1-carboxamide;
(94) (2R)—N—((R)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide;
(95) (2R)—N—((S)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide;
(96) (2R)—N—((R)-(3,4-difluorophenyl)(5-fluoro-6-(trifluoro-methyl)pyridin-2-yl)methyl)-2-methyl-3-oxopiperazine-1-carboxamide;
(97) (2R)—N—((S)-(3,4-difluorophenyl)(5-fluoro-6-(trifluoro-methyl)pyridin-2-yl)methyl)-2-methyl-3-oxopiperazine-1-carboxamide;
(98) (2R)—N—((R)-(5-fluoro-6-(trifluoro-methyl)pyridin-2-yl)(6-(2,2,2-trifluoro-ethoxy)pyridin-3-yl)methyl)-2-methyl-3-oxopiperazine-1-carboxamide;
(99) (2R)—N—((S)-(5-fluoro-6-(trifluoro-methyl)pyridin-2-yl)(6-(2,2,2-trifluoro-ethoxy)pyridin-3-yl)methyl)-2-methyl-3-oxopiperazine-1-carboxamide;
(100) (2R)—N—((R)-(5-fluoro-6-(trifluoromethyl)pyridin-2-yl)(6-(trifluoro-methoxy)pyridin-3-yl)methyl)-2-methyl-3-oxopiperazine-1-carboxamide;
(101) (2R)—N—((S)-(5-fluoro-6-(trifluoro-methyl)pyridin-2-yl)(6-(trifluoromethoxy)pyridin-3-yl)methyl)-2-methyl-3-oxopiperazine-1-carboxamide;
(102) (2R)—N—((R)-(4-chloro-3-cyanophenyl)(5-fluoro-6-(trifluoromethyl) pyridin-2-yl)methyl)-2-methyl-3-oxopiperazine-1-carboxamide;
(103) (2R)—N—((S)-(4-chloro-3-cyanophenyl)(5-fluoro-6-(trifluoromethyl) pyridin-2-yl)methyl)-2-methyl-3-oxopiperazine-1-carboxamide;
(104) (2R)—N—((R)-(4-chloro-3-fluorophenyl)(5-fluoro-6-(trifluoromethyl) pyridin-2-yl)methyl)-2-methyl-3-oxopiperazine-1-carboxamide;
(105) (2R)—N—((S)-(4-chloro-3-fluoro-phenyl)(5-fluoro-6-(trifluoromethyl) pyridin-2-yl)methyl)-2-methyl-3-oxopiperazine-1-carboxamide;
(106) N—((R)-(4-chloro-3-fluorophenyl)(5-fluoro-6-(trifluoromethyl) pyridin-2-yl)methyl)-3-oxopiperazine-1-carboxamide;
(107) N—((S)-(4-chloro-3-fluorophenyl)(5-fluoro-6-(trifluoromethyl) pyridin-2-yl)methyl)-3-oxopiperazine-1-carboxamide;
(108) (2R)—N—((R)-(5-fluoro-6-(trifluoromethyl)pyridin-2-yl)(4-(trifluoromethoxy)-phenyl)methyl)-2-methyl-3-oxopiperazine-1-carboxamide;
(109) (2R)—N—((S)-(5-fluoro-6-(trifluoromethyl)pyridin-2-yl)(4-(trifluoromethoxy)-phenyl)methyl)-2-methyl-3-oxopiperazine-1-carboxamide;
(110) (2R)—N—((R)-(3-fluoro-4-(trifluoromethoxy)phenyl)(5-fluoro-6-(trifluoro-methyl)pyridin-2-yl)methyl)-2-methyl-3-oxopiperazine-1-carboxamide;
(111) (2R)—N—((S)-(3-fluoro-4-(trifluoromethoxy)phenyl)(5-fluoro-6-(trifluoro-methyl)pyridin-2-yl)methyl)-2-methyl-3-oxopiperazine-1-carboxamide;
(112) (2R)—N—((R)-(5-fluoro-6-(trifluoromethyl)pyridin-2-yl)(4-(2,2,2-trifluoro-ethoxy)phenyl)methyl)-2-methyl-3-oxopiperazine-1-carboxamide;
(113) (2R)—N—((S)-(5-fluoro-6-(trifluoromethyl)pyridin-2-yl)(4-(2,2,2-trifluoro-ethoxy)phenyl)methyl)-2-methyl-3-oxopiperazine-1-carboxamide;
(114) (2R)—N—((R)-(5-fluoro-6-(trifluoromethyl)pyridin-2-yl)(3-(trifluoromethoxy)-phenyl)methyl)-2-methyl-3-oxopiperazine-1-carboxamide;
(115) (2R)—N—((S)-(5-fluoro-6-(trifluoromethyl)pyridin-2-yl)(3-(trifluoromethoxy)-phenyl)methyl)-2-methyl-3-oxopiperazine-1-carboxamide;
(116) (2R)—N—((R)-(4-cyclopropoxy-3-fluorophenyl)(5-fluoro-6-(trifluoromethyl) pyridin-2-yl)methyl)-2-methyl-3-oxopiperazine-1-carboxamide;
(117) (2R)—N—((S)-(4-cyclopropoxy-3-fluorophenyl)(5-fluoro-6-(trifluoromethyl) pyridin-2-yl)methyl)-2-methyl-3-oxopiperazine-1-carboxamide;
(118) (2R)—N—((R)-(3-chloro-2,4-difluorophenyl)(5-fluoro-6-(trifluoromethyl)pyridin-2-yl)methyl)-2-methyl-3-oxopiperazine-1-carboxamide;
(119) (2R)—N—((S)-(3-chloro-2,4-difluorophenyl)(5-fluoro-6-(trifluoromethyl)pyridin-2-yl)methyl)-2-methyl-3-oxopiperazine-1-carboxamide;
(120) (2R)—N—((R)-(5-cyano-6-(trifluoromethyl)pyridin-2-yl)(3-fluoro-4-(trifluoromethoxy)-phenyl)methyl)-2-methyl-3-oxopiperazine-1-carboxamide;
(121) (2R)—N—((S)-(5-cyano-6-(trifluoromethyl)pyridin-2-yl)(3-fluoro-4-(trifluoromethoxy)-phenyl)methyl)-2-methyl-3-oxopiperazine-1-carboxamide;
(122) (2R)—N—((R)-(3-chloro-2,4-difluorophenyl)(2-(trifluoromethyl)thiazol-5-yl)methyl)-2-methyl-3-oxopiperazine-1-carboxamide;
(123) (2R)—N—((S)-(3-chloro-2,4-difluorophenyl)(2-(trifluoromethyl)thiazol-5-yl)methyl)-2-methyl-3-oxopiperazine-1-carboxamide;
(124) (2R)—N—((R)-(3-chloro-4-(trifluoromethoxy)phenyl)(1-(trifluoromethyl)-1H-pyrazol-4-yl)methyl)-2-methyl-3-oxopiperazine-1-carboxamide;
(125) (2R)—N—((S)-(3-chloro-4-(trifluoromethoxy)phenyl)(1-(trifluoromethyl)-1H-pyrazol-4-yl)methyl)-2-methyl-3-oxopiperazine-1-carboxamide;
(126) (2R)—N—((R)-(3-chloro-4-(trifluoromethoxy)phenyl)(2-(trifluoromethyl) oxazol-4-yl)methyl)-2-methyl-3-oxopiperazine-1-carboxamide;
(127) (2R)—N—((S)-(3-chloro-4-(trifluoromethoxy)phenyl)(2-(trifluoromethyl) oxazol-4-yl)methyl)-2-methyl-3-oxopiperazine-1-carboxamide;
(128) (2R)—N—((R)-(3-chloro-2,4-difluorophenyl)(1-(4-fluorophenyl)-1H-pyrazol-4-yl)methyl)-2-methyl-3-oxopiperazine-1-carboxamide;
(129) (2R)—N—((S)-(3-chloro-2,4-difluorophenyl)(1-(4-fluorophenyl)-1H-pyrazol-4-yl)methyl)-2-methyl-3-oxopiperazine-1-carboxamide;
(130) (2R)—N—((R)-(3-chloro-4-fluorophenyl)(1-(difluoromethyl)-1H-pyrazol-3-yl)methyl)-2-methyl-3-oxopiperazine-1-carboxamide;
(131) (2R)—N—((S)-(3-chloro-4-fluorophenyl)(1-(difluoromethyl)-1H-pyrazol-3-yl)methyl)-2-methyl-3-oxopiperazine-1-carboxamide;
(132) (2R)—N—((R)-(3-chloro-4-fluorophenyl)(1-(2,2,2-trifluoroethyl)-1H-pyrazol-4-yl)methyl)-2-methyl-3-oxopiperazine-1-carboxamide;
(133) (2R)—N—((S)-(3-chloro-4-fluorophenyl)(1-(2,2,2-trifluoroethyl)-1H-pyrazol-4-yl)methyl)-2-methyl-3-oxopiperazine-1-carboxamide;
(134) (2R)—N—((R)-(4-chlorophenyl)(2-(trifluoromethyl)pyrimidin-4-yl)methyl)-2-methyl-3-oxopiperazine-1-carboxamide;
(135) (2R)—N—((S)-(4-chlorophenyl)(2-(trifluoromethyl)pyrimidin-4-yl)methyl)-2-methyl-3-oxopiperazine-1-carboxamide;
(136) (2R)—N—((R)-(3-chloro-4-fluorophenyl)(2-(trifluoromethyl)pyrimidin-5-yl)methyl)-2-methyl-3-oxopiperazine-1-carboxamide;
(137) (2R)—N—((S)-(3-chloro-4-fluorophenyl)(2-(trifluoromethyl)pyrimidin-5-yl)methyl)-2-methyl-3-oxopiperazine-1-carboxamide;
(138) (2R)—N—((R)-(3-chloro-4-fluorophenyl)(5-fluoro-6-(2,2,2-trifluoroethoxy)pyridin-2-yl)methyl)-2-methyl-3-oxopiperazine-1-carboxamide;
(139) (2R)—N—((S)-(3-chloro-4-fluorophenyl)(5-fluoro-6-(2,2,2-trifluoroethoxy)pyridin-2-yl)methyl)-2-methyl-3-oxopiperazine-1-carboxamide;
(140) (2R)—N—((R)-(3-chloro-4-fluorophenyl)(2-(2,2,2-trifluoroethoxy)pyridin-4-yl)methyl)-2-methyl-3-oxopiperazine-1-carboxamide;
(141) (2R)—N—((S)-(3-chloro-4-fluorophenyl)(2-(2,2,2-trifluoroethoxy)pyridin-4-yl)methyl)-2-methyl-3-oxopiperazine-1-carboxamide;
(142) (2R)—N—((R)-(3-chloro-4-fluorophenyl)(6-(difluoromethoxy)pyridin-2-yl)methyl)-2-methyl-3-oxopiperazine-1-carboxamide;
(143) (2R)—N—((S)-(3-chloro-4-fluorophenyl)(6-(difluoromethoxy)pyridin-2-yl)methyl)-2-methyl-3-oxopiperazine-1-carboxamide;
(144) (2R)—N—((R)-(3-chloro-4-fluorophenyl)(6-(difluoromethoxy)pyridin-3-yl)methyl)-2-methyl-3-oxopiperazine-1-carboxamide;
(145) (2R)—N—((S)-(3-chloro-4-fluorophenyl)(6-(difluoromethoxy)pyridin-3-yl)methyl)-2-methyl-3-oxopiperazine-1-carboxamide;
(146) (2R)—N—((R)-(3-chloro-2,4-difluorophenyl)(2-(difluoromethoxy)pyrimidin-5-yl)methyl)-2-methyl-3-oxopiperazine-1-carboxamide;
(147) (2R)—N—((S)-(3-chloro-2,4-difluorophenyl)(2-(difluoromethoxy)pyrimidin-5-yl)methyl)-2-methyl-3-oxopiperazine-1-carboxamide;
(148) (2R)—N—((R)-(3-chloro-2,4-difluorophenyl)(6-(1,1-difluoroethyl)pyridin-3-yl)methyl)-2-methyl-3-oxopiperazine-1-carboxamide;
(149) (2R)—N—((S)-(3-chloro-2,4-difluorophenyl)(6-(1,1-difluoroethyl)pyridin-3-yl)methyl)-2-methyl-3-oxopiperazine-1-carboxamide;
(150) x(2R)—N—((R)-(5-chloro-6-(trifluoromethyl)pyridin-2-yl)(4-cyanophenyl)methyl)-2-methyl-3-oxopiperazine-1-carboxamide;
(151) (2R)—N—((S)-(5-chloro-6-(trifluoromethyl)pyridin-2-yl)(4-cyanophenyl)methyl)-2-methyl-3-oxopiperazine-1-carboxamide;
(152) (2R)—N—((R)-(3-chloro-2,4-difluorophenyl)(5-chloro-6-(trifluoromethyl)pyridin-2-yl)methyl)-2-methyl-3-oxopiperazine-1-carboxamide;
(153) (2R)—N—((S)-(3-chloro-2,4-difluorophenyl)(5-chloro-6-(trifluoromethyl)pyridin-2-methyl-3-oxopiperazine-1-carboxamide;
(154) N—((R)-(3-chloro-2,4-difluorophenyl)(5-chloro-6-(trifluoromethyl)pyridin-2-yl)methyl)-3-oxopiperazine-1-carboxamide;
(155) N—((S)-(3-chloro-2,4-difluorophenyl)(5-chloro-6-(trifluoromethyl)pyridin-2-yl)methyl)-3-oxopiperazine-1-carboxamide;
(156) (2R)—N—((R)-(3-chloro-4-fluorophenyl)(5-chloro-6-(trifluoromethyl) pyridin-2-yl)methyl)-2-methyl-3-oxopiperazine-1-carboxamide;
(157) (2R)—N—((S)-(3-chloro-4-fluorophenyl)(5-chloro-6-(trifluoromethyl) pyridin-2-yl)methyl)-2-methyl-3-oxopiperazine-1-carboxamide;
(158) N—((R)-(3-chloro-4-fluorophenyl)(5-chloro-6-(trifluoromethyl) pyridin-2-yl)methyl)-3-oxopiperazine-1-carboxamide;
(159) N—((S)-(3-chloro-4-fluorophenyl)(5-chloro-6-(trifluoromethyl) pyridin-2-yl)methyl)-3-oxopiperazine-1-carboxamide;
(160) (2R)—N—((R)-(5-chloro-6-(trifluoromethyl)pyridin-2-yl)(6-(trifluoromethyl) pyridin-3-yl)methyl)-2-methyl-3-oxopiperazine-1-carboxamide;
(161) (2R)—N—((S)-(5-chloro-6-(trifluoromethyl)pyridin-2-yl)(6-(trifluoromethyl) pyridin-3-yl)methyl)-2-methyl-3-oxopiperazine-1-carboxamide;
(162) ((2R)—N—((R)-(5-chloro-6-(trifluoromethyl)pyridin-3-yl)(5-fluoro-6-(trifluoromethyl)pyridin-2-yl)methyl)-2-methyl-3-oxopiperazine-1-carboxamide;
(163) ((2R)—N—((S)-(5-chloro-6-(trifluoromethyl)pyridin-3-yl)(5-fluoro-6-(trifluoromethyl)pyridin-2-yl)methyl)-2-methyl-3-oxopiperazine-1-carboxamide;
(164) (2R)—N—((R)-(3-chloro-2,4-difluorophenyl)(5-chloro-6-(trifluoromethyl)pyridin-2-yl)methyl)-2-methyl-3-oxopiperazine-1-carboxamide;
(165) (2R)—N—((S)-(3-chloro-2,4-difluorophenyl)(5-chloro-6-(trifluoromethyl)pyridin-2-yl)methyl)-2-methyl-3-oxopiperazine-1-carboxamide;
(166) (2R)—N—((R)-(5-chloro-6-(trifluoromethyl)pyridin-3-yl)(2-(trifluoromethyl)thiazol-4-yl)methyl)-2-methyl-3-oxopiperazine-1-carboxamide;
(167) (2R)—N—((S)-(5-chloro-6-(trifluoromethyl)pyridin-3-yl)(2-(trifluoromethyl)thiazol-4-yl)methyl)-2-methyl-3-oxopiperazine-1-carboxamide;
(168) (2R)—N—((R)-(3-chloro-4-fluorophenyl)(3-(trifluoromethyl)-1H-pyrazol-5-yl)methyl)-2-methyl-3-oxopiperazine-1-carboxamide;
(169) (2R)—N—((S)-(3-chloro-4-fluorophenyl)(3-(trifluoromethyl)-1H-pyrazol-5-yl)methyl)-2-methyl-3-oxopiperazine-1-carboxamide;
(170) (2R)—N—((R)-(3-chloro-4-fluorophenyl)(1-methyl-3-(trifluoromethyl)-1H-pyrazol-5-yl)methyl)-2-methyl-3-oxopiperazine-1-carboxamide;
(171) (2R)—N—((S)-(3-chloro-4-fluorophenyl)(1-methyl-3-(trifluoromethyl)-1H-pyrazol-5-yl)methyl)-2-methyl-3-oxopiperazine-1-carboxamide;
(172) (2R)-2-methyl-3-oxo-N—((R)-(4-(trifluoromethoxy)phenyl)(3-(trifluoromethyl)-1H-pyrazol-5-yl)methyl)piperazine-1-carboxamide; and
(173) (2R)-2-methyl-3-oxo-N—((S)-(4-(trifluoro methoxy)phenyl)(3-(trifluoromethyl)-1H-pyrazol-5-yl)methyl)piperazine-1-carboxamide;
   or a pharmaceutically acceptable salt thereof.

Illustrative, but non-limiting, examples of the compounds of the present invention that are useful as inhibitors of Naᵥ1.8 channel activity are the following compounds:
(1) (2R)—N—((R)(3-chloro-2,4-difluorophenyl)(6-(2,2,2-trifluoroethoxy)pyridin-3-yl)methyl)-2-methyl-3-oxopiperazine-1-carboxamide;
(2) (2R)—N—((S)(3-chloro-2,4-difluorophenyl)(6-(2,2,2-trifluoroethoxy)pyridin-3-yl)methyl)-2-methyl-3-oxopiperazine-1-carboxamide;
(3) (2R)—N—((R or S)-(3-chloro-2,4-difluorophenyl)(5-fluoro-6-(trifluoromethyl)pyridin-3-yl)methyl)-2-methyl-3-oxopiperazine-1-carboxamide;
(4) (2R)—N—((R)-(5-fluoro-6-(2,2,2-trifluoroethoxy)pyridin-3-yl)(5-fluoro-6-(trifluoro-methyl)-pyridin-2-yl)methyl)-2-methyl-3-oxopiperazine-1-carboxamide;
(5) (2R)—N—((S)-(5-fluoro-6-(2,2,2-trifluoroethoxy)pyridin-3-yl)(5-fluoro-6-(trifluoro-methyl)pyridin-2-yl)methyl)-2-methyl-3-oxopiperazine-1-carboxamide;
(6) (2R)—N—((R)-(3-chloro-2,4-difluorophenyl)(2-(trifluoromethyl)thiazol-5-yl)methyl)-2-methyl-3-oxopiperazine-1-carboxamide;
(7) (2R)—N—((S)-(3-chloro-2,4-difluorophenyl)(2-(trifluoromethyl)thiazol-5-yl)methyl)-2-methyl-3-oxopiperazine-1-carboxamide;
(8) (2R)—N—((R)-(3-chloro-4-fluorophenyl)(3-(trifluoromethyl)-1H-pyrazol-5-yl)methyl)-2-methyl-3-oxopiperazine-1-carboxamide; and
(9) (2R)—N—((S)-(3-chloro-4-fluorophenyl)(3-(trifluoromethyl)-1H-pyrazol-5-yl)methyl)-2-methyl-3-oxopiperazine-1-carboxamide;
   or a pharmaceutically acceptable salt thereof.

Although the specific stereochemistries described above are preferred, other stereoisomers, including diastereoisomers, enantiomers, epimers, and mixtures of these may also have utility in treating Naᵥ1.8 mediated diseases.

Synthetic methods for making the compounds are disclosed in the Examples shown below. Where synthetic details are not provided in the examples, the compounds are readily made by a person of ordinary skill in the art of medicinal chemistry or synthetic organic chemistry by applying the synthetic information provided herein. Where a stereochemical center is not defined, the structure represents a mixture of stereoisomers at that center. For such compounds, the individual stereoisomers, including enantiomers, diastereoisomers, and mixtures of these are also compounds of the invention.

### Definitions

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like. In one embodiment of the present invention, alkenyl is —C₁alkenyl or ═CH₂.

“Alkynyl” means carbon chains which contain at least one carbon-carbon triple bond, and which may be linear or branched, or combinations thereof, unless otherwise defined. Examples of alkynyl include ethynyl, propargyl, 3-methyl-1-pentynyl, 2-heptynyl and the like.

“Cycloalkyl” means a saturated monocyclic, bicyclic, spirocyclic or bridged carbocyclic ring, having a specified number of carbon atoms. Examples of cycloalkyl include cyclopropyl, cyclobutyl, cyclopentyl, cyclohexyl, cycloheptyl, and the like. In one embodiment, cycloalkyl is selected from: cyclopropane, cyclobutane, cyclopentane, and cyclohexane. In another embodiment, cycloalkyl is cyclopropane.

“Cycloheteroalkyl” means a saturated or partly unsaturated non-aromatic monocyclic, bicyclic, spirocyclic or bridged ring or ring system having a specified number of carbon atoms and containing at least one ring heteroatom selected from N, NH, S (including SO and SO₂) and O. The cycloheteroalkyl ring may be substituted on the ring carbons and/or the ring nitrogen or sulfur. Examples of cycloheteroalkyl include tetrahydrofuran, pyrrolidine, tetrahydrothiophene, azetidine, piperazine, piperidine, morpholine, oxetane and tetrahydropyran. In one embodiment of the present invention, cycloheteroalkyl is selected from: azetidine, piperidine, pyrrolidine, tetrahydropyran, and tetrahydrofuran.

“Aryl” means a monocyclic, bicyclic or tricyclic carbocyclic aromatic ring or ring system containing 6-14 carbon atoms, wherein at least one of the rings is aromatic. Examples of aryl include phenyl and naphthyl. In one embodiment of the present invention, aryl is phenyl. In another embodiment of the present invention, aryl is selected from phenyl and naphthalene.

“Heteroaryl” means a monocyclic, bicyclic or tricyclic ring or ring system containing 5-14 ring atoms and containing at least one ring heteroatom selected from N, NH, S (including SO and SO₂) and O, wherein at least one of the heteroatom containing rings is aromatic. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl, quinolyl, indolyl, isoquinolyl, quinazolinyl, dibenzofuranyl, and the like. In one embodiment of the present invention, heteroaryl is selected from: pyridine, pyrimidine, pyrazine, pyridazine, imidazole, pyrazole, thiazole, oxazole, benzofuran, benzoxazole, benzothiazole, indole, indazole, imidazopyridine, thiophene, and thiazolopyridine. In another embodiment of the present invention, heteroaryl is selected from pyridine and thiazole. In another embodiment, heteroaryl is selected from: pyridine, pyrazole, oxazole, and thiazole. In another embodiment, heteroaryl is pyridine. In another embodiment, heteroaryl is thiazole. In another embodiment, heteroaryl is pyrazole. In another embodiment, heteroaryl is oxazole. In another embodiment, heteroaryl is selected from: pyridine, pyrimidine, pyrazole, thiazole, imidazo[1,2-a]pyridine, oxazole, benzofuran, benzoxazole, indazole, and thiazolopyridine. In another embodiment, heteroaryl is selected from: oxazole, pyridine, pyrimidine, pyrazole, thiazole, and imidazo[1,2-a]pyridine. In another embodiment, heteroaryl is selected from: pyridine, pyrimidine, pyrazole, thiazole, and imidazo[1,2-a]pyridine. In another embodiment, heteroaryl is selected from: pyridine, pyrazole, and thiazole.

“Halogen” includes fluorine, chlorine, bromine and iodine. In one embodiment, halogen is fluorine, chorine or bromine. In another embodiment, halogen is fluorine or chlorine. In another embodiment, halogen is fluorine or bromine. In another embodiment, halogen is fluorine. In another embodiment, halogen is chlorine. In another embodiment, halogen is bromine.

“Me” represents methyl.

“Oxo” represents ═O.

“Saturated” means containing only single bonds.

“Unsaturated” means containing at least one double or triple bond. In one embodiment, unsaturated means containing at least one double bond. In another embodiment, unsaturated means containing one double bond. In another embodiment, unsaturated means containing at least one triple bond. In another embodiment, unsaturated means containing one triple bond.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to:

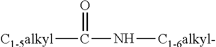

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of Formula I may contain one or more asymmetric centers and can thus occur as racemates and racemic mixtures, single enantiomers, diastereomeric mixtures and individual diastereomers. The present invention is meant to encompass all such isomeric forms of the compounds of Formula I.

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

In the compounds of general formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural formula I. For example, different isotopic forms of hydrogen (H) include protium (¹H), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

### Salts

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide, trifluoroacetate and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

The term “prodrug” means compounds that are rapidly transformed, for example, by hydrolysis in blood, in vivo to the parent compound, e.g., conversion of a prodrug of Formula I to a compound of Formula I, or to a salt thereof: a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. This invention includes prodrugs of the novel compounds of this invention.

Solvates, and in particular, the hydrates of the compounds of the present invention are included in the present invention as well.

### Utilities

The compound of the present invention are selective inhibitors of Naᵥ1.8 sodium ion channel activity or have selective activity as Naᵥ1.8 sodium ion channel blockers. In one embodiment, the compounds of the present invention exhibit at least 10-fold selectivity for Naᵥ1.8 sodium channels over Naᵥ1.5 sodium channels, and in some embodiments exhibit at least 100-fold selectivity for Naᵥ1.8 sodium channels over Naᵥ1.5 sodium channels based on functional potency (IC₅₀ values) for each channel in Qube® assay system.

The compounds of the present invention are potent inhibitors of Naᵥ1.8 channel activity. The compounds, and pharmaceutically acceptable salts thereof, may be efficacious in the treatment of diseases, disorders and conditions that are mediated by the inhibition of Naᵥ1.8 sodium ion channel activity and/or Naᵥ1.8 receptors.

Diseases, disorders or conditions mediated by Naᵥ1.8 sodium ion channel activity and/or Naᵥ1.8 receptors, include but are not limited to nociception, osteoarthritis, peripheral neuropathy, inherited erythromelalgia, multiple sclerosis, asthma, pruritus, acute itch, chronic itch, migraine, neurodegeneration following ischemia, epilepsy, inflammatory pain, spontaneous pain, acute pain, peri-operative pain, post-operative pain, neuropathic pain, postherpetic neuralgia, trigeminal neuralgia, diabetic neuropathy, chronic lower back pain, phantom limb pain, pain resulting from cancer and chemotherapy, chronic pelvic pain, pain syndromes, and complex regional pain syndromes.

One or more of these conditions or diseases may be treated, managed, prevented, reduced, alleviated, ameliorated or controlled by the administration of a therapeutically effective amount of a compound of the present invention, or a pharmaceutically acceptable salt thereof, to a patient in need of treatment. Also, the compounds of the present invention may be used for the manufacture of a medicament which may be useful for treating, preventing, managing, alleviating, ameliorating or controlling one or more of these conditions, diseases or disorders: nociception, osteoarthritis, peripheral neuropathy, inherited erythromelalgia, multiple sclerosis, asthma, pruritus, acute itch, chronic itch, migraine, neurodegeneration following ischemia, epilepsy, inflammatory pain, spontaneous pain, acute pain, peri-operative pain, post-operative pain, neuropathic pain, postherpetic neuralgia, trigeminal neuralgia, diabetic neuropathy, chronic lower back pain, phantom limb pain, pain resulting from cancer and chemotherapy, chronic pelvic pain, pain syndromes, and complex regional pain syndromes.

Preferred uses of the compounds may be for the treatment of one or more of the following diseases by administering a therapeutically effective amount to a patient in need of treatment. The compounds may be used for manufacturing a medicament for the treatment of one or more of these diseases:
1) pain conditions,
2) pruritic conditions, and
3) cough conditions.

In one embodiment of the present invention, the pain condition is an acute pain or chronic pain disorder. In another embodiment of the present invention, the the pain condition is an acute pain disorder.

The compounds of the present invention may be effective in treating nociception. Nociception or pain is essential for survival and often serves a protective function. However, the pain associated with surgical procedures and current therapies to relieve that pain, can delay recovery after surgery and increase the length of hospital stays. As many as 80% of surgical patients experience post-operative pain due to tissue damage, and damage to peripheral nerves and subsequent inflammation. Approximately 10-50% of surgical patients will develop chronic pain after surgery often because the nerve damage results in lasting neuropathic pain once the wound has healed.

The compounds of the present invention may be effective in treating osteoarthritis. Osteoarthritis is type of arthritis caused by inflammation, breakdown, and eventual loss of cartilage in the joints. The standards of care for pain associated with osteoarthritis are non-steroidal anti-inflammatory drugs (NSAIDs), for example celecoxib and diclofenac (reviewed in Zeng et al., 2018). Patients that do not respond to NSAID therapies are typically treated with low dose opiates, such as hydrocodone. Patients that are refractory to the above therapies will usually opt for total joint replacement.

The compounds of the present invention may be effective in treating peripheral neuropathy. Peripheral neuropathy is nerve damage caused by chronically high blood sugar and diabetes. It leads to numbness, loss of sensation, and sometimes pain in distal limbs such as feet, legs, or hands. It is the most common complication of diabetes. The standards of care for the treatment of painful diabetic neuropathy are gabapentinoids, for example gabapentin and pregabalin. Some patients will respond well to tricyclic antidepressants such as amitriptyline, while other patients get significant relief using SRI/NRI drugs such as duloxetine (Schreiber et al., World J Diabetes. 2015 Apr. 15; 6(3):432-44). Many options are available, however side-effects are common (e.g. dizziness, nausea) which limit their full potential.

The compounds of the present invention may be effective in treating inherited erythromelalgia. Inherited erythromelalgia (IEM) is a chronic pain syndrome which has been linked to mutations in several voltage-gated sodium channels, including Naᵥ1.8 (Kist et al., PLOS One. 2016 Sep. 6; 11(9):e0161789). Patients present with the classic “gloves and stocking” flare pattern on distal regions such as hands and feet, typically brought on with warm temperatures and exercise. Some patients find relief from the burning pain associated with flares by cold water immersion. Although medications that affect voltage-gated sodium channels (eg, lidocaine and mexiletine) show promise, there is no current standard of care to treat IEM.

The compounds of the present invention may be effective in treating neuropathic pain. Neuropathic pain is pain caused by damage or disease affecting the somatosensory nervous system. It has been demonstrated in human patients, as well as in animal models of neuropathic pain, that damage to primary afferent sensory neurons can lead to neuroma formation and spontaneous activity, as well as evoked activity in response to normally innocuous stimuli. (Colloca et al., Nat Rev Dis Primers. 2017 Feb. 16; 3: 17002; Coward et al., Pain. 2000 March; 85(1-2):41-50; Yiangou et al., FEBS Lett. 2000 Feb. 11; 467(2-3):249-52: Carter et al., Phys Med Rehabil Clin N Am. 2001 May; 12(2):447-59). Some nerve injuries result in an increase in Naᵥ1.8 expression, which is believed to be an underlying mechanism for pathological pain. (Black et al., Ann Neurol. 2008 December; 64(6):644-53; Bird et al., Br J Pharmacol. 2015 May; 172(10):2654-70). Injuries of the peripheral nervous system often result in neuropathic pain persisting long after an initial injury resolves. Examples of neuropathic pain include, but are not limited to, post herpetic neuralgia, trigeminal neuralgia, diabetic neuropathy, chronic lower back pain, lumbar radiculopathy, phantom limb pain, pain resulting from cancer and chemotherapy, chronic pelvic pain, complex regional pain syndrome and related neuralgias, and painful conditions that arise due to gain-of-function mutations in Naᵥ1.8 (Huang et al., J Neurosci. 2013 Aug. 28; 33(35): 14087-97; Kist et al., PLOS One. 2016 Sep. 6; 11(9):e0161789; Emery et al., J Neurosci. 2015 May 20; 35(20): 7674-81; and Schreiber et al., World J Diabetes. 2015 Apr. 15; 6(3):432-44.

The ectopic activity of normally silent sensory neurons is thought to contribute to the generation and maintenance of neuropathic pain, which is generally assumed to be associated with an increase in sodium channel activity in the injured nerve. (Wood et al., Curr Opin Pharmacol. 2001 February; 1(1): 17-21; Baker et al., TRENDS in Pharmacological Sciences, 2001, 22(1): 27-31). Standards of care for neuropathic pain vary considerably depending on the particular condition, but first line therapies are typically pregabalin, gabapentin, tricyclic antidepressants (e.g. amitriptyline), and SRI/NRI drugs (e.g. duloxetine). Patients refractory to these therapies are usually prescribed low dose opiates (e.g. hydrocodone).

The compounds of the present invention may be effective in treating multiple sclerosis. Recent evidence points to a potential role for Naᵥ1.8 in multiple sclerosis. Naᵥ1.8 expression in cerebellum has been identified in tissues taken from animal models of multiple sclerosis (EAE model) and in postmortem brains from patients suffering from multiple sclerosis (MS) (Shields et al., Ann Neurol. 2012 February; 71(2): 186-94; Black et al., Proc Natl Acad Sci USA. 2000 Oct. 10; 97(21): 11598-602). Also, two SCN10A polymorphisms showed significant association with MS (Roostaei et al., Neurology. 2016 Feb. 2; 86 (5):410-7). When Naᵥ1.8 is overexpressed in cerebellum, mice develop ataxic-related motor deficits which are ameliorated with oral delivery of a selective small molecule Nav1.8 antagonist (Shields et al., PLOS One. 2015 Mar. 6; 10(3)). These studies suggest that a Nav1.8 antagonist may be a useful therapy to treat symptoms related to multiple sclerosis.

The compounds of the present invention may be effective in treating asthma. Asthma is caused by airway inflammation in which a person's airways become hyper-responsive, narrow and swollen, which makes it difficult to breathe. These symptoms are typically triggered through an allergic reaction (Nair P et al., J Allergy Clin Immunol Pract. 2017 May-June; 5(3):649-659). In a preclinical model of asthma, deletion of Nav1.8-containing neurons, or inhibition of nerve fibers via small molecules reduces airway inflammation and immune cell infiltration (Talbot et al., Neuron. 2015 Jul. 15; 87(2):341-54). Selective Nav1.8 antagonists may be a useful therapy to prevent airway hypersensitivity caused by immune cell infiltration.

The compounds of the present invention may be effective in treating pruritus. Pruritus, also commonly known as itch, affects approximately 4% of the global population is an unpleasant sensation that elicits the desire or reflex to scratch, and is regarded as closely related to pain (Luo et al., Cell Mol Life Sci. 2015 September; 72 (17): 3201-23). Theories on the origin of itch implicate the subtle, low-frequency activation of nociceptors (pain-sensing neurons); however, it has been described that some afferents preferentially respond to histamine, which induces itch (Schmelz et al., J Neurosci. 1997 Oct. 15; 17(20):8003-8). At the same time, it has been found that histamine-responding neurons also respond to capsaicin which produces pain (McMahon et al., Trends in Neuroscience 1992, 15:497-501). Members of the transient receptor potential (TRP) family, and nerve growth factor (NGF) are both known to play a role in itch and pain, and clinically, both maladies are treated with therapeutic agents such as gabapentin and antidepressants. Therefore, it continues to be accepted that the underlying mechanisms of pain and itch are highly interwoven and complex, and distinguishing pan-selective or itch-selective pathways remains ambiguous (Ikoma et al., Nat Rev Neurosci. 2006 July; 7(7):535-47). A role for Nav1.8 in pruritis was studied using a mouse transgenically expressing a constitutively active form of the serine/threonine kinase BRAF was expressed in Nav1.8-expressing neurons. This resulted in enhanced pruriceptor excitability, and heightened evoked and spontaneous scratching behavior (Zhao et al., 2013). In skin, pruritogens are released from keratinocytes, lymphocytes, mast cells, and eosinophils during inflammation. These molecules act directly on free nerve endings which express Nav1.8 to induce itch (Riol-Blanco et al., Nature. 2014 Jun. 5; 510 (7503): 157-61). Chronic and acute itch can arise from many different insults, diseases and disorders, and may be classified as dermal or pruriceptive, neurogenic, neuropathic, or psychogenic: itch can arise from both systemic disorders, skin disorders, as well as physical or chemical insult to the dermis. Pathologically, conditions such as dry skin, eczema, psoriasis, varicella zoster, urticaria, scabies, renal failure, cirrhosis, lymphoma, iron deficiency, diabetes, menopause, polycythemia, uremia, and hyperthyroidism can cause itch, as can diseases of the nervous system such as tumors, multiple sclerosis, peripheral neuropathy, nerve compression, and delusions related to obsessive-compulsive disorders. Medicines such as opioids and chloroquine can also trigger itch (Ikoma et al., Nat Rev Neurosci. 2006 July; 7(7):535-47). Itching following burn is also an extremely serious clinical problem as it hampers the healing process, resulting in permanent scaring, and negatively impacting quality of life (Van Loey et al., Br J Dermatol. 2008 January; 158(1):95-100).

The invention also includes pharmaceutically acceptable salts of the compounds, and pharmaceutical compositions comprising the compounds and a pharmaceutically acceptable carrier.

The compounds, or pharmaceutically acceptable salts thereof, may be useful in treating pain conditions, pruritic conditions, and cough conditions.

A compound of the present invention, or a pharmaceutically acceptable salt thereof, may be used in the manufacture of a medicament for the treatment of pain conditions, pruritic conditions, and cough conditions in a human or other mammalian patient.

A method of treating a pain conditions comprises the administration of a therapeutically effective amount of a compound of the present invention, or a pharmaceutically acceptable salt thereof, or a pharmaceutical composition comprising the compound, to a patient in need of treatment. A method of treating a pruritic condition comprises the administration of a therapeutically effective amount of a compound of the present invention, or a pharmaceutically acceptable salt thereof, or a pharmaceutical composition comprising the compound, to a patient in need of treatment. A method of treating a cough condition comprises the administration of a therapeutically effective amount of a compound of the present invention, or a pharmaceutically acceptable salt thereof, or a pharmaceutical composition comprising the compound, to a patient in need of treatment. Other medical uses of the compounds of the present invention are described herein.

The term “pain condition” as used herein includes, but are not limited to, acute pain, peri-operative pain, pre-operative pain, post-operative pain, neuropathic pain, post herpetic neuralgia, trigeminal neuralgia, diabetic neuropathy, chronic lower back pain, phantom limb pain, chronic pelvic pain, vulvodynia, complex regional pain syndrome and related neuralgias, pain associated with cancer and chemotherapy, pain associated with HIV, and HIV treatment-induced neuropathy, nerve injury, root avulsions, painful traumatic mononeuropathy, painful polyneuropathy, erythromyelalgia, paroxysmal extreme pain disorder, small fiber neuropathy, burning mouth syndrome, central pain syndromes (potentially caused by virtually any lesion at any level of the nervous system), postsurgical pain syndromes (e.g., post mastectomy syndrome, post thoracotomy syndrome, stump pain)), bone and joint pain (osteoarthritis), repetitive motion pain, dental pain, myofascial pain (muscular injury, fibromyalgia), perioperative pain (general surgery, gynecological), chronic pain, dysmennorhea, pain associated with angina, inflammatory pain of varied origins (e.g. osteoarthritis, rheumatoid arthritis, rheumatic disease, teno-synovitis and gout), shoulder tendonitis or bursitis, gouty arthritis, and aolymyalgia rheumatica, primary hyperalgesia, secondary hyperalgesia, primary allodynia, secondary allodynia, or other pain caused by central sensitization, complex regional pain syndrome, chronic arthritic pain and related neuralgias acute pain, migraine, migraine headache, headache pain, cluster headache, non-vascular headache, traumatic nerve injury, nerve compression or entrapment, and neuroma pain,

The term “pruritic condition” or “pruritic disorder” as used herein includes, but is not limited to, conditions with an unpleasant sensation that provokes the desire to scratch, such as chronic itch.

The term “cough condition” or “cough disorder” as used herein includes, but is not limited to, chronic cough, neuropathic cough or cough due to neurological conditions.

Treatment of a disease, disorder or condition mediated by Naᵥ1.8 sodium ion channel activity or Naᵥ1.8 receptors refers to the administration of the compounds of the present invention to a subject with the disease, disorder or condition. One outcome of treatment may be reducing the disease, disorder or condition mediated by Naᵥ1.8 sodium ion channel activity or Naᵥ1.8 receptors. Another outcome of treatment may be alleviating the disease, disorder or condition mediated by Naᵥ1.8 sodium ion channel activity or Naᵥ1.8 receptors. Another outcome of treatment may be ameliorating the disease, disorder or condition mediated by Naᵥ1.8 sodium ion channel activity or Naᵥ1.8 receptors. Another outcome of treatment may be suppressing the disease, disorder or condition mediated by Naᵥ1.8 sodium ion channel activity or Naᵥ1.8 receptors. Another outcome of treatment may be managing the disease, disorder or condition mediated by Naᵥ1.8 sodium ion channel activity or Naᵥ1.8 receptors.

Another outcome of treatment may be preventing the disease, disorder or condition mediated by Naᵥ1.8 sodium ion channel activity or Naᵥ1.8 receptors.

Prevention of the disease, disorder or condition mediated by Naᵥ1.8 sodium ion channel activity or Naᵥ1.8 receptors refers to the administration of the compounds of the present invention to a subject at risk of the disease, disorder or condition. One outcome of prevention may be reducing the disease, disorder or condition mediated by Naᵥ1.8 sodium ion channel activity or Naᵥ1.8 receptors in a subject at risk of the disease, disorder or condition. Another outcome of prevention may be suppressing the disease, disorder or condition mediated by Naᵥ1.8 sodium ion channel activity or Naᵥ1.8 receptors in a subject at risk of the disease, disorder or condition. Another outcome of prevention may be ameliorating the disease, disorder or condition mediated by Naᵥ1.8 sodium ion channel activity or Naᵥ1.8 receptors in a subject at risk of the disease, disorder or condition. Another outcome of prevention may be alleviating the disease, disorder or condition mediated by Naᵥ1.8 sodium ion channel activity or Naᵥ1.8 receptors in a subject at risk of the disease, disorder or condition. Another outcome of prevention may be managing the disease, disorder or condition mediated by Naᵥ1.8 sodium ion channel activity or Naᵥ1.8 receptors in a subject at risk of the disease, disorder or condition.

One outcome of treatment may be reducing the amount of pain experienced by a subject relative to that subject's pain immediately before the administration of the compounds of the present invention. Another outcome of treatment may be alleviating the amount of pain experienced by a subject relative to that subject's pain immediately before the administration of the compounds of the present invention. Another outcome of treatment may be ameliorating the amount of pain experienced by a subject relative to that subject's pain immediately before the administration of the compounds of the present invention. Another outcome of treatment may be suppressing the amount of pain experienced by a subject relative to that subject's pain immediately before the administration of the compounds of the present invention. Another outcome of treatment may be managing the amount of pain experienced by a subject relative to that subject's pain immediately before the administration of the compounds of the present invention. Another outcome of treatment may be ameliorating the amount of pain experienced by a subject relative to that subject's pain immediately before the administration of the compounds of the present invention.

Another outcome of treatment may be preventing further pain experienced by a subject after the administration of the compounds of the present invention.

Prevention of pain refers to the administration of the compounds of the present invention to reduce the pain of a subject at risk of pain. Prevention includes, but is not limited to, the administration to a subject prior to surgery or other expected painful event. One outcome of prevention may be reducing pain in a subject at risk of pain. Another outcome of prevention may be suppressing pain in a subject at risk of pain. Another outcome of prevention may be ameliorating pain in a subject at risk of pain. Another outcome of prevention may be alleviating pain in a subject at risk of pain. Another outcome of prevention may be managing pain in a subject at risk of pain.

The terms “administration of” and or “administering a” compound should be understood to mean providing a compound of the invention or a prodrug of a compound of the invention to the individual or mammal in need of treatment.

The administration of the compound of structural formula I in order to practice the present methods of therapy is carried out by administering an effective amount of the compound of structural formula I to the mammal in need of such treatment or prophylaxis. The need for a prophylactic administration according to the methods of the present invention is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature.

### Administration and Dose Ranges

Any suitable route of administration may be employed for providing a mammal, especially a human, with an effective dose of a compound of the present invention. For example, oral, intravenous, infusion, subcutaneous, transcutaneous, intramuscular, intradermal, transmucosal, intramucosal, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

In the treatment or prevention of disorders, diseases and/or conditions which require inhibition of Naᵥ1.8 sodium ion channel activity, a suitable dosage level will generally be about 0.0001 to 500 mg per kg patient body weight per day which can be administered in single or multiple doses. In one embodiment, a suitable dosage level may be about 0.001 to 500 mg per kg patient body weight per day. In another embodiment, a suitable dosage level may be about 0.001 to about 250 mg/kg per day. In another embodiment, a suitable dosage level may be about 0.01 to about 250 mg/kg per day. In another embodiment, a suitable dosage level may be about 0.1 to about 100 mg/kg per day. In another embodiment, a suitable dosage level may be about 0.05 to 100 mg/kg per day. In another embodiment, a suitable dosage level may be about 0.1 to 50 mg/kg per day. In another embodiment, a suitable dosage level may be about 0.05 to 0.5 mg/kg per day. In another embodiment, a suitable dosage level may be about 0.5 to 5 mg/kg per day. In another embodiment, a suitable dosage level may be about 5 to 50 mg/kg per day. For oral administration, the compositions are preferably provided in the form of tablets containing 0.01 to 1000 mg of the active ingredient, particularly 0.01, 0.025, 0.05, 0.075, 0.1, 0.25, 0.5, 0.75, 1.0, 2.5, 5.0, 7.5, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 8 times per day; preferably, 1 to 4 times a day; more preferably once or twice per day. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of this invention may be used in pharmaceutical compositions comprising (a) the compound(s) or pharmaceutically acceptable salts thereof, and (b) a pharmaceutically acceptable carrier. The compounds of this invention may be used in pharmaceutical compositions that include one or more other active pharmaceutical ingredients. The compounds of this invention may also be used in pharmaceutical compositions in which the compound of the present invention or a pharmaceutically acceptable salt thereof is the only active ingredient.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

Compounds of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention. In the treatment of patients who have pain conditions, pruritic conditions and cough conditions, more than one drug is commonly administered. The compounds of this invention may generally be administered to a patient who is already taking one or more other drugs for these conditions. Often the compounds will be administered to a patient who is already being treated with one or more anti-pain compounds when the patient's pain is not adequately responding to treatment.

The combination therapy also includes therapies in which the compound of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered in combination with a compound of the present invention, and either administered separately or in the same pharmaceutical composition, include but are not limited to:
(i) an opioid agonist;
(ii) an opioid antagonist;
(iii) a calcium channel antagonist;
(iv) a NMDA receptor agonist;
(v) a NMDA receptor antagonist;
(vi) a COX-2 selective inhibitor;
(vii) a NSAID (non-steroidal anti-inflammatory drug);
(viii) an analgesic;
(ix) a sodium channel inhibitor;
(x) an anti-NGF antibody;
(xi) a Naᵥ1.7 inhibitor;
(xii) a HCN inhibitor;
(xiii) a TRPV1 antagonist;
(xiv) a Naᵥ1.7 biological; and
(xv) a Naᵥ1.8 biological; and
   pharmaceutically acceptable salts thereof.

In another embodiment of the present invention, the pharmaceutical composition comprises:
(1) a compound of claim 1 or a pharmaceutically acceptable salt thereof;
(2) or more compounds, or pharmaceutically acceptable salts thereof, selected from the group consisting of;
   (i) an opioid agonist;
   (ii) an opioid antagonist;
   (iii) a calcium channel antagonist;
   (iv) a NMDA receptor agonist;
   (v) a NMDA receptor antagonist;
   (vi) a COX-2 selective inhibitor;
   (vii) a NSAID (non-steroidal anti-inflammatory drug);
   (viii) an analgesic;
   (ix) a sodium channel inhibitor;
   (x) an anti-NGF antibody;
   (xi) a Naᵥ1.7 inhibitor;
   (xii) a HCN inhibitor;
   (xiii) a TRPV1 antagonist;
   (xiv) a Naᵥ1.7 biological; and
   (xv) a Naᵥ1.8 biological; and
      pharmaceutically acceptable salts thereof; and
(3) a pharmaceutically acceptable carrier.

A Nav 1.7 biological means a protein, including, but not limited to, antibodies, nanobodies and peptides, that inhibits the function of the Nav1.7 channel. A Nav 1.8 biological means a protein, including, but not limited to, antibodies, nanobodies and peptides, that inhibits the function of the Nav1.8 channel.

Specific compounds of use in combination with a compound of the present invention include: sodium channel inhibitors, including but not limited to, lidocaine including the lidocaine patch; tricyclic antidepressants including, but not limited to, amitriptyline; and SRI/NRI drugs, including but not limited to, duloxetine.

Suitable opioid agonists include, but are not limited to, codeine, fentanyl, hydrocodone, hydromorphone, levorphanol, meperidine, methadone, morphine, oxycodone, oxymorphone, buprenorphine, butorphanol, dezocine, nalbuphine, pentazocine, and tramadol.

Suitable opioid antagonists include, but are not limited to, naltrexone and naloxone.

Suitable calcium channel antagonists include, but are not limited to, Amlodipine, Diltiazem, Felodipine, gabapentin. Isradipine, Nicardipine. Nifedipine, Nisoldipine, pregabalin, Verapamil, and ziconitide.

Suitable NMDA receptor antagonists include, but are not limited to, ketamine, methadone, memantine, amantadine, and dextromethorphan.

Suitable COX-2 inhibitors include, but are not limited to, celecoxib, etoricoxib and parecoxib.

Suitable NSAIDs or non-steroidal anti-inflammatory drugs include, but are not limited to, aspirin, diclofenac, diflunisal, etodolac, fenoprofin, flurbiprofen, ibuprofen, indomethacin, ketoprofen, meclofenamic acid, mefenamic acid, meloxicam, naproxen, naproxen sodium, oxaprozin, piroxicam, sulindac, and tolmetin.

Suitable analgesics include, but are not limited to, acetaminophen and duloxetine.

The above combinations include combinations of a compound of the present invention not only with one other active compound, but also with two or more other active compounds. Non-limiting examples include combinations of compounds with two or more active compounds selected from: opioid agonists; opioid antagonists; calcium channel antagonists; NMDA receptor agonists; NMDA receptor antagonists; COX-2 selective inhibitors; NSAIDs (non-steroidal anti-inflammatory drugs); and an analgesic.

The compounds of the present invention, or a pharmaceutically acceptable salt thereof, may also be used in combination with spinal cord stimulation therapy and cutaneous stimulation therapy.

The present invention also provides a method for the treatment or prevention of a Naᵥ1.8 sodium ion channel activity mediated disease, disorder or condition, which method comprises administration to a patient in need of such treatment or at risk of developing a Naᵥ1.8 sodium ion channel activity mediated disease with a therapeutically effective amount of a Naᵥ1.8 sodium ion channel activity inhibitor and an amount of one or more active ingredients, such that together they give effective relief.

In a further aspect of the present invention, there is provided a pharmaceutical composition comprising a Naᵥ1.8 sodium ion channel activity inhibitor and one or more active ingredients, together with at least one pharmaceutically acceptable carrier or excipient.

Thus, according to a further aspect of the present invention there is provided the use of a Naᵥ1.8 sodium ion channel activity inhibitor and one or more active ingredients for the manufacture of a medicament for the treatment or prevention of a Naᵥ1.8 sodium ion channel activity mediated disease, disorder or condition. In a further or alternative aspect of the present invention, there is therefore provided a product comprising a Naᵥ1.8 sodium ion channel activity inhibitor and one or more active ingredients as a combined preparation for simultaneous, separate or sequential use in the treatment or prevention of a Naᵥ1.8 sodium ion channel activity mediated disease, disorder or condition. Such a combined preparation may be, for example, in the form of a twin pack.

It will be appreciated that for the treatment or prevention of pain conditions, pruritic conditions and cough conditions, a compound of the present invention may be used in conjunction with another pharmaceutical agent effective to treat that disease, disorder or condition.

The present invention also provides a method for the treatment or prevention of pain conditions, pruritic conditions and cough conditions, which method comprises administration to a patient in need of such treatment an amount of a compound of the present invention and an amount of another pharmaceutical agent effective to threat that disorder, disease or condition, such that together they give effective relief.

The present invention also provides a method for the treatment or prevention of pain conditions, pruritic conditions and cough conditions, which method comprises administration to a patient in need of such treatment an amount of a compound of the present invention and an amount of another pharmaceutical agent useful in treating that particular condition, disorder or disease, such that together they give effective relief.

The term “therapeutically effective amount” means the amount the compound of structural formula I that will elicit the biological or medical response of a cell, tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with a COX-2 inhibitor the weight ratio of the compound of the Formula I to the COX-2 inhibitor will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following reaction schemes and Examples illustrate methods which may be employed for the synthesis of the compounds of structural formula I described in this invention. These reaction schemes and Examples are provided to illustrate the invention and are not to be construed as limiting the invention in any manner. All substituents are as defined above unless indicated otherwise. Several strategies based upon synthetic transformations known in the literature of organic synthesis may be employed for the preparation of the compounds of structural formula I. The scope of the invention is defined by the appended claims.

### Instrumentation

Reverse phase chromatography was carried out on a Gilson GX-281 equipped with a column selected from the following: Phenomenex Synergi C18 (150 mm×30 mm×4 micron), YMC-Actus Pro C18 (150 mm×30 mm×5 micron), Xtimate C18 (150 mm×25 mm×5 micron), Boston Green ODS (150 mm×30 mm×5 micron), XSELECT C18 (150 mm×30 mm×5 micron), and Waters XSELECT C18 (150 mm×30 mm×5 micron). Conditions included either high pH (0)-100% acetonitrile/water eluent comprising 0.1% v/v 10 mM NH4CO3 or 0.05% NH₄OH) or low pH (0-95% acetonitrile/water eluent comprising 0.1% v/v TFA) and are noted for some examples.

SFC chiral resolution was carried out on a Sepiate Prep SFC 100, Multigram II (MG II), THAR80 prep SFC, or a Waters SFC (80, 200, or 350).

LC/MS determinations were carried out on a Waters Classing Aquity system equipped with TUV and MS detectors and a Waters SQD mass spectrometer, a Shimadzu 20 UV 254 and 220 nM with Shimadzu 2010 or 2020 mass spectrometer, or an Agilent 1200 HPLC quipped with DAD/ELSD and G6110 MSD using one of the following conditions: 1) Ascentis Express C18 (3×50 mm) 2.7 μm column using mobile phase containing A: 0.05% TFA in water and B: 0.05% TFA in acetonitrile with a gradient from 90:10 (A:B) to 5:95 (A:B) over 6 min at a flow rate of 1.8 mL/min, UV detection at 210 nm: 2) Aquity BEH C18, (1.0×50 mm) 1.7 μm column using mobile phase containing A: 0.05% TFA in water and B: 0.05% TFA in acetonitrile with a gradient from 90:10 (A:B) to 5:95 (A:B) over 2 min at a flow rate of 0.3 mL/min, UV detection at 215 nm: 3) Agilent YMC J′Sphere H-80 (3×50 mm) 5 μm column using mobile phase containing A: 0.1% TFA in water and B: acetonitrile with a gradient from 95:5 (A:B) to 0:100 (A:B) over 3.6 min and 0:100 (A:B) for 0.4 min at a flow rate of 1.4 mL/min, UV detection at 254 and 220 nm and Agilent 1100 quadrupole mass spectrometer: 4) an Agilent TC-C18 (2.1×50 mm) 5 μm column using mobile phase containing A: 0.0375% TFA in water and B: 0.01875% TFA in acetonitrile with a gradient from 90:10 (A:B) for 0.4 min to 90:10 to 0:100 (A:B) over 3 min and 10:90 (A:B) for 0.6 min at a flow rate of 0.8 mL/min, UV detection at 254 and 220 nm and Agilent 6110 quadrupole mass spectrometer.

Proton or 1H NMR was acquired using a Varian Unity-Inova 400 MHz NMR spectrometer equipped with a Varian 400 ATB PFG 5 mm, Nalorac DBG 400-5 or a Nalorac IDG 400-5 probe, a Varian-400 MHz MR spectrometer equipped with an Auto X ID PFG Probe 5 mm, a Varian 400 MHz VNMRS spectrometer equipped with a PFG 4Nuc Probe 5 mm, or a Bruker AvanceIII 500 MHz spectrometer equipped with a PABBO Probe 5 mm in accordance with standard analytical techniques, unless specified otherwise, and results of spectral analysis are reported. Chemical shift (8) values are reported in delta (8) units, parts per million (ppm). Chemical shifts for 1H NMR spectra are given relative to signals for residual non-deuterated solvent (CDCl₃ referenced at δ 7.26 ppm; DMSO d-6 referenced at δ 2.50 ppm and CD₃OD referenced at δ 3.31 ppm). Multiples are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

### Abbreviations

AcOH is acetic acid; BAST is bis(2-methoxyethyl)aminosulfur trifluoride; Boc is tert-butoxy carbonyl; Calc'd is calculated; CDI is 1,1′-carbonyldiimidazole, DAST is diethylaminosulfur trifluoride; DIBAL-H is diisobutylaluminum hydride; DCE is dichloroethane; DCM is dichloromethane; DEA is diethanolamine; DIPEA or DIEA is N,N-diisopropylethylamine; DMA is dimethylacetamide; DME is dimethoxyethane; DMF is dimethylformamide; DMSO is dimethylsulfoxide; dppf is 1,1-bis(diphenylphosphino)ferrocene; EDC is 1-ethyl-3-(3-dimethylaminopropyl)carbodiimide; Et₃N is triethyl amine; Et₂₀ is diethyl ether; EtOAc is ethyl acetate; EtOH is ethanol; g is grams; h or hr(s) is hour(s); HATU is 1-[bis(dimethylamino)-methylene]-1H-1,2,3-triazolo[4,5-b]pyridinium-3-oxidehexa-fluorophosphate; Hex is hexanes; HOAt is 1-Hydroxy-7-azabenzotriazole; HPLC is high-performance liquid chromatography; IPA is isopropyl alcohol; iPrMgCl is isopropylmagnesium chloride; iPrMgCl-LiCl is isopropylmagnesium chloride lithium chloride complex; L is liter; LAH is lithium aluminum hydride; LC/MS is liquid chromatography-mass spectrometry; LRMS is low resolution mass spectrometry; M is molar; Me is methyl; MeOH is methanol; MeCN is acetonitrile; mg is milligrams; mL is milliliter; mmol is millimole(s); MPLC is medium pressure liquid chromatography; N is normal; NaHMDS is sodium bis(trimethylsilyl)amide; NH₄OAc is ammonium acetate, NMO is 4-methylmorpholine N-oxide; NMP is N-methylpyrrolidone; PCC is pyridinium chlorochromate; Pd/C is palladium on carbon; Pd(dppf)Cl₂ is [1,1-bis(diphenyl-phosphino)-ferrocene]dichloropalladium(II); Pd(OAc)₂ is palladium(II) acetate; Pd(PPh₃)₄ is tetrakis(triphenylphosphine)-palladium(0); Pd(t-Bu₃P)₂ is Bis(tri-tert-butyl-phosphine)-palladium(0); Pet. ether or PE is petroleum ether; PG is protecting group; ppm is milligrams per liter; Prep. or prep is preparative; psi is pounds per square inch; rt or RT is room temperature; SFC is Supercritical Fluid Chromatography; TBAF is tetrabutylammonium fluoride; TLC is thin layer chromatography; tBuXPhosPd G2 is Chloro(2-dicyclohexylphosphino-2′,4,6′-triiso-propyl-1,1′-biphenyl)[2-(2′-amino-1,1′-biphenyl)]palladium(II); tBuXPhos Pd G3 is [(2-Di-tert-butylphosphino-2,4,6′-triisopropyl-1,1′-biphenyl)-2-(2-amino-1,1′-biphenyl)] palladium(II) methanesulfonate; TEA is triethylamine; TFA is trifluoroacetic acid; THF is tetrahydrofuran; Ti(OEt)₄ is titanium (IV) ethoxide; Ti(OiPr)₄ is titanium (IV) isopropoxide; TLC is thin layer chromatography; UV is ultraviolet; v/v is volume per volume; and xantphos is 4,5-bis(diphenylphosphino)-9,9-dimethylxanthene.

As illustrated in Scheme A, compounds of the invention can be prepared by condensation between an appropriately functionalized aldehyde A-1 and tert-butanesulfinamide, utilizing dehydrating agents such as Ti(OEt)₄ or Ti(OiPr)₄, to afford intermediate A-2. Intermediate A-2 can then be reacted with a variety of organometallic nucleophiles A-3 to give intermediate A-4 which can be deprotected under acidic conditions to give amines of formula A-5. Amine A-5 can then be brought together with a piperazine A-6, utilizing urea coupling conditions (using triphosgene or CDI as coupling regents) to deliver compounds of formula A-7. In some embodiments, a protecting group, such as Boc, may need to be removed throughout the course of synthesis. Aldehydes of type A-1 and organometallics of type A-3 are commercially available or may be synthesized from appropriate starting materials and reagents.

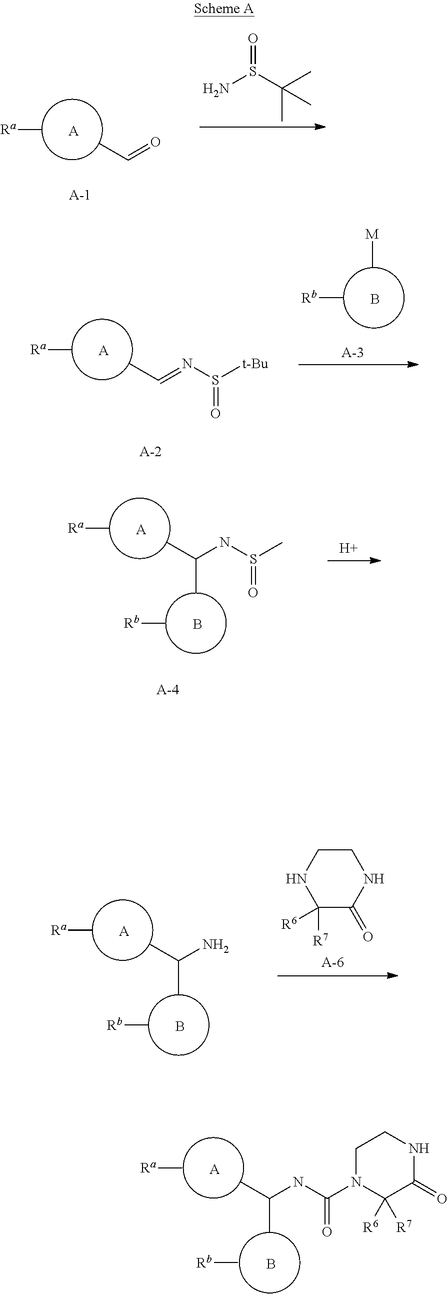

As illustrated in Scheme B, compounds of the invention can be prepared by activation of appropriately functionalized carboxylic acid B-1 with either (COCl)₂ or amide coupling with amine B-2 to give intermediates of B-3. These intermediates are then suitable to for reaction with a variety of organometallic nucleophiles A-3 to give intermediate B-4. Intermediate B-4 can then undergo reductive amination reaction in the presence of an amine source and reductant to yield intermediates of A-5. In some cases, tert-butanesulfinamide was used as the amine source and would require deprotection (in an acidic environment) following reductive amination. Amine A-5 can then be reacted with a piperazine A-6, utilizing urea coupling conditions (using triphosgene or CDI as coupling regents) to deliver compounds of formula A-7. In some embodiments, a protecting group, such as Boc, may need to be removed throughout the course of synthesis. Carboxylic acids of type B-1 and organometallics of type A-3 are commercially available or may be synthesized from appropriate starting materials and reagents.

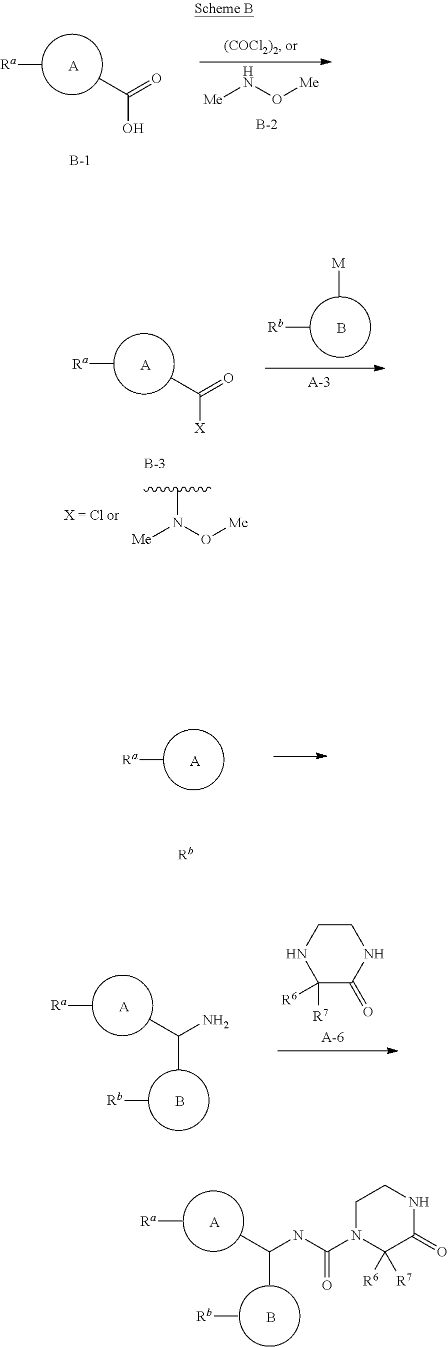

### EXAMPLES

### Examples 1A and 1B

### (2R)—N—((R)(3-chloro-2,4-difluorophenyl)(6-(2,2,2-trifluoroethoxy)pyridin-3-yl)methyl)-2-methyl-3-oxopiperazine-1-carboxamide and (2R)—N—((S)(3-chloro-2,4-difluorophenyl)(6-(2,2,2-trifluoroethoxy)pyridin-3-yl)methyl)-2-methyl-3-oxopiperazine-1-carboxamide

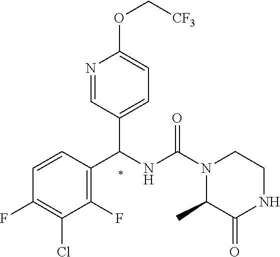 [SMILES: C[C@@H]1C(=O)NCCN1C(=O)NC(c1ccc(OCC(F)(F)F)nc1)c1ccc(F)c(Cl)c1F]

Step 1: (E)-2-methyl-N-((6-(2,2,2-trifluoroethoxy)pyridin-3-yl)methylene)propane-2-sulfinamide To a solution of 6-(2,2,2-trifluoroethoxy)nicotinaldehyde (1.98 g, 9.65 mmol) and 2-methyl propane-2-sulfinamide (1.228 g, 10.13 mmol) in CH₂Cl₂ (8 mL) was added titanium(IV) isopropoxide (6 mL, 20.27 mmol). The mixture was stirred at RT for 20 hours, then H₂O (30 mL) and ethyl acetate (40 mL) were added. The mixture was stirred at RT for 20 minutes, then filtered through a Celite® pad. The separated organic phase was dried over Na₂SO₄, filtered and concentrated under reduced pressure to give the title compound. LRMS m/z (M+H): calculated 308.3, observed 309.2.

Step 2: N-((3-chloro-2,4-difluorophenyl)(6-(2,2,2-trifluoroethoxy)pyridin-3-yl)methyl)-2-methylpropane-2-sulfinamide 1-Bromo-3-chloro-2,4-difluorobenzene (277 mg, 1.218 mmol) was dissolved in anhydrous THF and purged under N₂ for 5 minutes, followed by the addition of 1.3 M isopropylmagnesium chloride-lithium chloride complex in THF (0.938 mL, 1.220 mmol). The mixture was stirred at RT for 5 hours and (E)-2-methyl-N-((6-(2,2,2-trifluoroethoxy)-pyridin-3-yl)methylene)propane-2-sulfinamide (200 mg, 0.649 mmol) was added in one portion. The reaction continued at RT for 20 hours. Then the reaction was quenched with saturated aqueous NH₄Cl and extracted with diethyl ether. The separated organic phase was dried over Na₂SO₄, filtered and concentrated under reduced pressure to give the title compound. LRMS m/z (M+H): calculated 456.9, observed 457.2.

Step 3: (3-chloro-2,4-difluorophenyl)(6-(2,2,2-trifluoroethoxy)pyridin-3-yl)methanamine hydrochloride To a solution of N-((3-chloro-2,4-difluorophenyl)(6-(2,2,2-trifluoroethoxy)-pyridin-3-yl)methyl)-2-methylpropane-2-sulfinamide (296 mg, 0.648 mmol) in CH₂Cl₂ (2 mL) and MeOH (1 mL) was added HCl in 1,4-dioxane (4 M, 2 mL, 8.00 mmol). The mixture was stirred at RT for 2 hours and concentrated under reduced pressure. The resulting residue was treated with diethyl ether (15 mL), and filtered to collect the solid. The solid was washed with diethyl ether and dried under vacuum to give the title compound. LRMS m/z (M+H): calculated 352.7, observed 353.2.

Step 4: examples 1A and 1B To a solution of (3-chloro-2,4-difluorophenyl)(6-(2,2,2-trifluoroethoxy)pyridin-3-yl)methanamine HCl (98 mg, 0.252 mmol) in CH₂Cl₂ (3 mL) at 0° C. were added Et₃N (0.176 mL, 1.259 mmol) and triphosgene (74.7 mg, 0.252 mmol). The mixture was stirred at 0° C. for 1 hour, then (R)-3-methylpiperazin-2-one (43.1 mg, 0.378 mmol) was added. After stirring at 0° C. for 1 hour, the reaction was warmed to RT, stirred for 1 hour, and concentrated under reduced pressure. The resulting residue was purified by column chromatography on silica gel, eluting with (0-4% MeOH/DCM) to give a mixture of isomers which was further separated by SFC (AD-H column, 35% MeOH co-solvent) to give examples 1A (first eluted fraction) and 1B (second eluted fraction).

Example 1A: LRMS m/z (M+H): calculated 492.8, observed 493.3. ¹H NMR δ (ppm) (500 MHz, Chloroform-d): 8.05 (s, 1H), 7.56 (d, J=7.7 Hz, 1H), 7.29 (s, 1H), 7.04 (t, J=7.9 Hz, 1H), 6.87 (d, J=8.2 Hz, 1H), 6.32 (d, J=6.3 Hz, 2H), 5.53-5.31 (m, 1H), 4.77 (q, J=8.5 Hz, 2H), 4.49 (s, 1H), 4.23 (d, J=12.5 Hz, 1H), 3.33 (s, 1H), 3.20 (s, 1H), 1.50 (d, J=5.8 Hz, 3H).

Example 1B: LRMS m/z (M+H): calculated 492.8, observed 493.3. ¹H NMR δ (ppm) (500 MHz, Chloroform-d): 8.04 (s, 1H), 7.54 (d, J=8.5 Hz, 1H), 7.26 (d, J=7.8 Hz, 1H), 7.04 (t, J=8.2 Hz, 1H), 6.87 (d, J=8.5 Hz, 1H), 6.32 (d, J=7.1 Hz, 1H), 6.23 (s, 1H), 5.25 (d, J=7.0 Hz, 1H), 4.76 (q, J=8.5 Hz, 2H), 4.45 (q, J=6.7 Hz, 1H), 4.23 (d, J=12.9 Hz, 1H), 3.57-3.47 (m, 1H), 3.34 (d, J=10.9 Hz, 1H), 3.23 (t, J=10.9 Hz, 1H), 1.52 (d, J=6.9 Hz, 3H).

TABLE 1 The following examples were prepared according to the synthetic procedure for Examples 1A and 1B, using appropriate starting materials and reagents.

| | | | Calc'd | Observed | |
|---|---|---|---|---|---|
| Example | Compound | Name | [M + H]⁺ | [M + H]⁺ | Conditions |
| 2A | 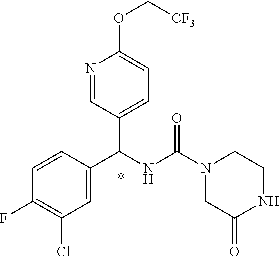 [SMILES: O=C1CN(C(=O)NC(c2ccc(OCC(F)(F)F)nc2)c2ccc(F)c(Cl)c2)CCN1] | N-((R or S)-(3-chloro- 4-fluorophenyl)(6- (2,2,2-trifluoro- ethoxy)pyridin-3- yl)methyl)-3- oxopiperazine-1- carboxamide | 460.8 | 461.0 | SFC: OJ-H Co-solvent: 35% (EtOH + 0.2% DIPEA) peak 1 |
| 2B | 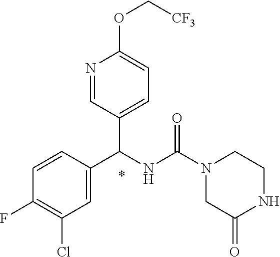 [SMILES: O=C1CN(C(=O)NC(c2ccc(OCC(F)(F)F)nc2)c2ccc(F)c(Cl)c2)CCN1] | N-((S or R)-(3-chloro- 4-fluorophenyl)(6- (2,2,2- trifluoroethoxy)pyridin- 3-yl)methyl)-3- oxopiperazine-1- carboxamide | 460.8 | 461.0 | SFC: OJ-H Co-solvent: 35% (EtOH + 0.2% DIPEA) peak 2 |
| 3A | 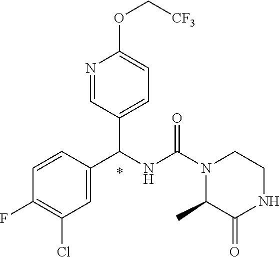 [SMILES: C[C@@H]1C(=O)NCCN1C(=O)NC(c1ccc(OCC(F)(F)F)nc1)c1ccc(F)c(Cl)c1] | (2R)-N-((R or S)-(3- chloro-4- fluorophenyl)(6-(2,2,2- trifluoro- ethoxy)pyridin-3- yl)methyl)-2-methyl-3- oxopiperazine-1- carboxamide | 474.8 | 475.4 | SFC: OJ-H Co-solvent: 20% EtOH peak 1 |
| 3B | 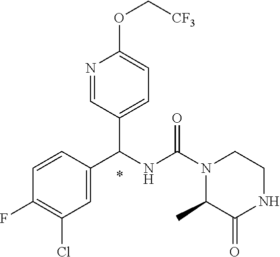 [SMILES: C[C@@H]1C(=O)NCCN1C(=O)NC(c1ccc(OCC(F)(F)F)nc1)c1ccc(F)c(Cl)c1] | (2R)-N-((S or R)-(3- chloro-4- fluorophenyl)(6-(2,2,2- trifluoro- ethoxy)pyridin-3- yl)methyl)-2-methyl-3- oxopiperazine-1- carboxamide | 474.8 | 475.4 | SFC: OJ-H Co-solvent: 20% EtOH peak 2 |
| 4A | 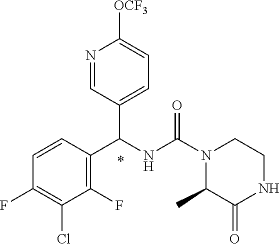 [SMILES: C[C@@H]1C(=O)NCCN1C(=O)NC(c1ccc(OC(F)(F)F)nc1)c1ccc(F)c(Cl)c1F] | (2R)-N-((R or S)-(3- chloro-2,4- difluorophenyl)(6- (trifluoromethoxy) pyridin-3-yl)methyl)-2- methyl-3- oxopiperazine-1- carboxamide | 478.8 | 479.3 | SFC: IC Co-solvent: 20%(EtOH + 0.1%NH₃• H₂O) Peak1 |
| 4B | 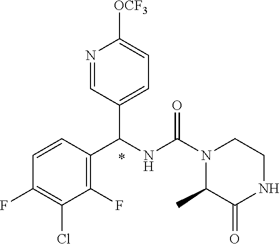 [SMILES: C[C@@H]1C(=O)NCCN1C(=O)NC(c1ccc(OC(F)(F)F)nc1)c1ccc(F)c(Cl)c1F] | (2R)-N-((S or R)-(3- chloro-2,4- difluorophenyl)(6- (trifluoromethoxy) pyridin-3-yl)methyl)-2- methyl-3- oxopiperazine-1- carboxamide | 478.8 | 479.3 | SFC: IC Co-solvent: 20%(EtOH + 0.1%NH₃• H₂O) Peak2 |
| 5A | 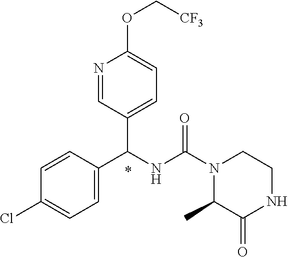 [SMILES: C[C@@H]1C(=O)NCCN1C(=O)NC(c1ccc(Cl)cc1)c1ccc(OCC(F)(F)F)nc1] | (2R)-N-((R or S)-(4- chlorophenyl)(6-(2,2,2- trifluoro- ethoxy)pyridin-3- yl)methyl)-2-methyl-3- oxopiperazine-1- carboxamide | 456.8 | 457.3 | SFC: OJ-H Co-solvent: 30% EtOH Peak 1 |
| 5B | 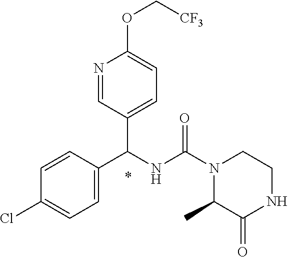 [SMILES: C[C@@H]1C(=O)NCCN1C(=O)NC(c1ccc(Cl)cc1)c1ccc(OCC(F)(F)F)nc1] | (2R)-N-((S or R)-(4- chlorophenyl)(6-(2,2,2- trifluoro- ethoxy)pyridin-3- yl)methyl)-2-methyl-3- oxopiperazine-1- carboxamide | 456.8 | 457.3 | SFC: OJ-H Co-solvent: 30% EtOH Peak 2 |
| 6A | 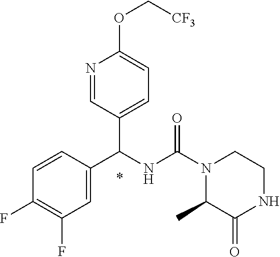 [SMILES: C[C@@H]1C(=O)NCCN1C(=O)NC(c1ccc(OCC(F)(F)F)nc1)c1ccc(F)c(F)c1] | (2R)-N-((R or S)-(3,4- difluorophenyl)(6- (2,2,2-trifluoro- ethoxy)pyridin-3- yl)methyl)-2-methyl-3- oxopiperazine-1- carboxamide | 458.4 | 459.3 | SFC: OJ-H Co-solvent: 20% MeOH peak 1 |
| 6B | 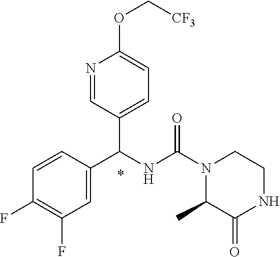 [SMILES: C[C@@H]1C(=O)NCCN1C(=O)NC(c1ccc(OCC(F)(F)F)nc1)c1ccc(F)c(F)c1] | (2R)-N-((S or R)-(3,4- difluorophenyl)(6- (2,2,2-trifluoro- ethoxy)pyridin-3- yl)methyl)-2-methyl-3- oxopiperazine-1- carboxamide | 458.4 | 459.3 | SFC: OJ-H Co-solvent: 20% MeOH peak 2 |
| 7A | 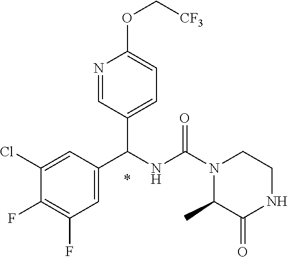 [SMILES: C[C@@H]1C(=O)NCCN1C(=O)NC(c1ccc(OCC(F)(F)F)nc1)c1cc(F)c(F)c(Cl)c1] | (2R)-N-((R or S)-(3- chloro-4,5- difluorophenyl)(6- (2,2,2-trifluoro- ethoxy)pyridin-3- yl)methyl)-2-methyl-3- oxopiperazine-1- carboxamide | 492.8 | 493.4 | SFC: AD-H Co-solvent: 40% EtOH peak 1 |
| 7B | 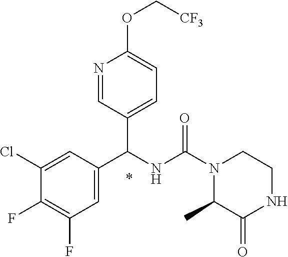 [SMILES: C[C@@H]1C(=O)NCCN1C(=O)NC(c1ccc(OCC(F)(F)F)nc1)c1cc(F)c(F)c(Cl)c1] | (2R)-N-((S or R)-(3- chloro-4,5-difluoro- phenyl)(6-(2,2,2-tri- fluoroethoxy)pyridin-3- yl)methyl)-2-methyl-3- oxo-piperazine-1- carboxamide | 492.8 | 493.4 | SFC: AD-H Co-solvent: 40% EtOH peak 2 |
| 8A | 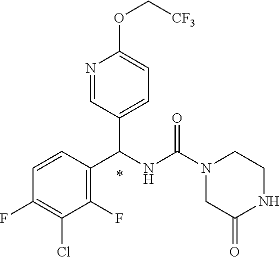 [SMILES: O=C1CN(C(=O)NC(c2ccc(OCC(F)(F)F)nc2)c2ccc(F)c(Cl)c2F)CCN1] | N-((R or S)-(3-chloro- 2,4-difluorophenyl)(6- (2,2,2-trifluoroethoxy) pyridin-3-yl)methyl)-3- oxopiperazine-1- carboxamide | 478.8 | 479.3 | SFC: OJ-H Co-solvent: 35% (EtOH + 0.2% DIPEA) peak 1 |
| 8B | 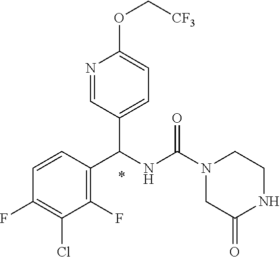 [SMILES: O=C1CN(C(=O)NC(c2ccc(OCC(F)(F)F)nc2)c2ccc(F)c(Cl)c2F)CCN1] | N-((S or R)-(3-chloro- 2,4-difluorophenyl)(6- (2,2,2-trifluoroethoxy) pyridin-3-yl)methyl)-3- oxopiperazine-1- carboxamide | 478.8 | 479.3 | SFC: OJ-H Co-solvent: 35% (EtOH + 0.2% DIPEA) peak 2 |
| 9A | 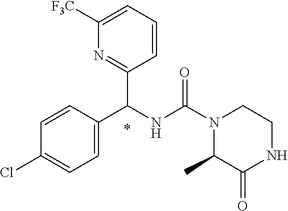 [SMILES: C[C@@H]1C(=O)NCCN1C(=O)NC(c1ccc(Cl)cc1)c1cccc(C(F)(F)F)n1] | (2R)-N-((R or S)-(4- chlorophenyl)(6- (trifluoromethyl)pyridin- 2-yl)methyl)-2-methyl- 3-oxo-piperazine-1- carboxamide | 426.8 | 427.2 | Silica Prep-TLC (3% MeOH/ DCM) polar fraction |
| 9B | 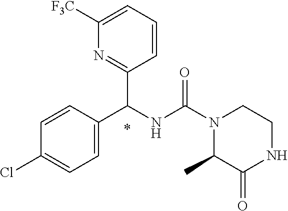 [SMILES: C[C@@H]1C(=O)NCCN1C(=O)NC(c1ccc(Cl)cc1)c1cccc(C(F)(F)F)n1] | (2R)-N-((S or R)-(4- chlorophenyl)(6- (trifluoromethyl)pyridin- 2-yl)methyl)-2-methyl- 3-oxopiperazine-1- carboxamide | 426.8 | 427.2 | Silica Prep-TLC (3% MeOH/ DCM) Less polar fraction |
| 10A | 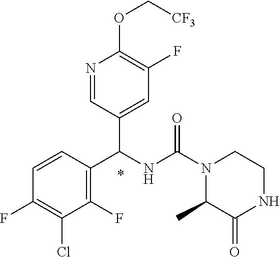 [SMILES: C[C@@H]1C(=O)NCCN1C(=O)NC(c1cnc(OCC(F)(F)F)c(F)c1)c1ccc(F)c(Cl)c1F] | (2R)-N-((R or S)-(3- chloro-2,4- difluorophenyl)(5- fluoro-6-(2,2,2- trifluoroethoxy)pyridin- 3-yl)methyl)-2-methyl- 3-oxopiperazine-1- carboxamide | 510.8 | 511.5 | SFC: OJ-H Co-solvent: 20% MeOH peak 1 |
| 10B | 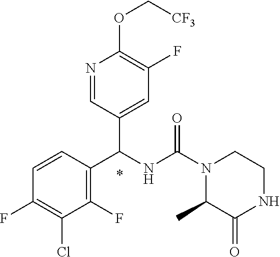 [SMILES: C[C@@H]1C(=O)NCCN1C(=O)NC(c1cnc(OCC(F)(F)F)c(F)c1)c1ccc(F)c(Cl)c1F] | (2R)-N-((S or R)-(3- chloro-2,4- difluorophenyl)(5- fluoro-6-(2,2,2- trifluoroethoxy)pyridin- 3-yl)methyl)-2-methyl- 3-oxopiperazine-1- carboxamide | 510.8 | 511.5 | SFC: OJ-H Co-solvent: 20% MeOH peak 2 |
| 11A | 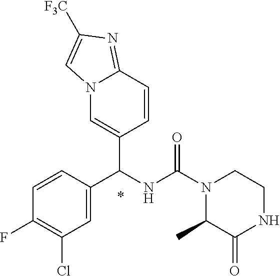 [SMILES: C[C@@H]1C(=O)NCCN1C(=O)NC(c1ccc(F)c(Cl)c1)c1ccc2nc(C(F)(F)F)cn2c1] | (2R)-N-((R or S)-(3- chloro-4-fluoro- phenyl)(2-(trifluoro- methyl)imidazo[1,2- a]pyridin-6-yl)- methyl)-2-methyl-3- oxopiperazine-1- carboxamide | 483.9 | 484.3 | SFC: AD-H Co-solvent: 30% EtOH peak 1 |
| 11B | 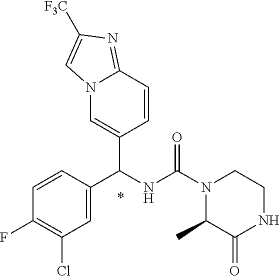 [SMILES: C[C@@H]1C(=O)NCCN1C(=O)NC(c1ccc(F)c(Cl)c1)c1ccc2nc(C(F)(F)F)cn2c1] | (2R)-N-((S oir R)-(3- chloro-4- fluorophenyl)(2- (trifluoromethyl)imidazo [1,2-a]pyridin-6- yl)methyl)-2-methyl-3- oxopiperazine-1- carboxamide | 483.9 | 484.3 | SFC: AD-H Co-solvent: 30% EtOH peak 2 |

### Examples 12A and 12B

### (2R)—N—((R)-(3-chloro-2,4-difluorophenyl)(2-(2,2,2-trifluoroethoxy)pyrimidin-5-yl)methyl)-2-methyl-3-oxopiperazine-1-carboxamide and (2R)—N—((S)-(3-chloro-2,4-difluorophenyl)(2-(2,2,2-trifluoroethoxy)pyrimidin-5-yl)methyl)-2-methyl-3-oxopiperazine-1-carboxamide

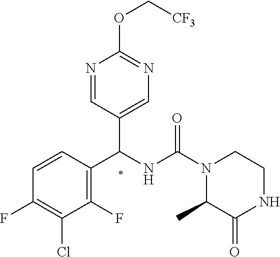 [SMILES: C[C@@H]1C(=O)NCCN1C(=O)NC(c1cnc(OCC(F)(F)F)nc1)c1ccc(F)c(Cl)c1F]

Step 1: (3-chloro-2,4-difluorophenyl)(2-(2,2,2-trifluoroethoxy)pyrimidin-5-yl)methanone To a solution of 2-(2,2,2-trifluoroethoxy)pyrimidine-5-carboxylic acid (870 mg, 3.92 mmol) in DCM (15.00 mL) at 0° C. was added 2 M (COCl)₂ in DCM (3.92 mL, 7.83 mmol) and one drop of DMF. The reaction was warmed to RT for 4 hours, then heated to 40° C. and stirred for 30 minutes. The mixture was concentrated under reduced pressure. The resulting residue was dissolved in THF (4 mL, Solution A). In a different reaction flask, 2-chloro-1,3-difluoro-4-iodobenzene (1397 mg, 5.09 mmol) in anhydrous tetrahydrofuran (15 mL) at 0° C. was added 1.3 M isopropylmagnesium chloride-lithium chloride complex in THF (3.92 mL, 5.09 mmol). The mixture was stirred at 0° C. for 2 h, followed by addition of copper(I) cyanide (526 mg, 5.88 mmol). The mixture was stirred at 0° C. for 30 minutes, then added to solution A. The reaction mixture was maintained at 0° C. for 2 h, then warmed to RT and stirred for 20 hours. The reaction was quenched with 40 mL of saturated aqueous NH₄Cl and extracted with ethyl acetate (2×40) mL). The combined organic phase was dried over Na₂SO₄, filtered and concentrated under reduced pressure to give the title compound. LRMS m/z (M+H): calculated 352.6, observed 353.1.

Step 2: (E)-N-((3-chloro-2,4-difluorophenyl)(2-(2,2,2-trifluoroethoxy)pyrimidin-5-yl)-methylene)-2-methylpropane-2-sulfinamide A microwave tube was charged with 2-methyl-propane-2-sulfinamide (0.516 g, 4.25 mmol), (3-chloro-2,4-difluorophenyl)(2-(2,2,2-trifluoroethoxy)pyrimidin-5-yl)methanone (1.0 g, 2.84 mmol) and titanium (iv) ethoxide (3 mL, 14.31 mmol). The mixture was microwaved at 110° C. for 40 minutes, then the mixture was cooled to RT, poured into brine and ethyl acetate, and filtered through a Celite®; pad. The organic phase was separated, dried over Na₂SO₄, filtered and concentrated under reduced pressure. The resulting residue was purified by column chromatography on silica gel, eluting with (0)-50% ethyl acetate/hexane) to give the title compound. LRMS m/z (M+H): calculated 455.8, observed 456.2.

Step 3: I-((3-chloro-2,4-difluorophenyl)(2-(2,2,2-trifluoroethoxy)pyrimidin-5-yl)methyl)-2-methylpropane-2-sulfinamide To a solution of (E)-N-((3-chloro-2,4-difluorophenyl)(2-(2,2,2-trifluoroethoxy)pyrimidin-5-yl)methylene)-2-methylpropane-2-sulfinamide (800 mg, 1.755 mmol) in EtOH (8 mL) at 0° C. was added NaBH₄ (66.4 mg. 1.755 mmol). The mixture was stirred at 0° C. for 10 minutes, quenched with H₂O and extracted with diethy ether. The separated organic phase was dried over Na₂SO₄, filtered and concentrated under reduced pressure to give the title compound. LRMS m/z (M+H): calculated 457.8, observed 458.3.

Step 4: (3-chloro-2,4-difluorophenyl)(2-(2,2,2-trifluoroethoxy)pyrimidin-5-yl)methanamine To a solution of N-((3-chloro-2,4-difluorophenyl)(2-(2,2,2-trifluoroethoxy)pyrimidin-5-yl)-methyl)-2-methylpropane-2-sulfinamide (800 mg, 1.747 mmol) in CH₂Cl₂ (8 mL) at 0° C. was added 4 M HCl in 1,4-dixoane (3 mL, 12.00 mmol). The resulting mixture was stirred at RT for 30 minutes, then concentrated under reduced pressure. The resulting residue was dissolved in 10 mL DCM, followed by the addition of NH₃ in MeOH (7 N, 5 mL). The mixture was stirred for 1 minute, then concentrated. The resulting residue was purified by column chromatography on silica gel, eluting with (0-5% MeOH/DCM) to give the title compound. LRMS m/z (M+H): calculated 353.7, observed 354.2.

Step 5: Examples 12A and 12B To a solution of (3-chloro-2,4-difluorophenyl)(2-(2,2,2-trifluoroethoxy)pyrimidin-5-yl)methanamine (110 mg, 0.311 mmol) in CH₂Cl₂ (4 mL) at 0° C. were added Et₃N (0.173 mL, 1.244 mmol) and triphosgene (92 mg, 0.311 mmol). The mixture was stirred at 0° C. for 1 hour, then (R)-3-methylpiperazin-2-one (53.3 mg, 0.467 mmol) was added. After stirring at 0° C. for 1 hour, the reaction was warmed to RT and maintained for 1 hour. Then the reaction mixture was concentrated under reduced pressure to give a residue that was purified by column chromatography on silica gel, eluting with (0-4% MeOH/DCM) to give a mixture of isomers, which was further separated by SFC (OD-H column, 30% EtOH co-solvent) to give examples 12A (first eluted fraction) and 12B (second eluted fraction).

Example 12A: LRMS m/z (M+H): calculated 493.8, observed 494.3. ¹H NMR δ (ppm) (500 MHz, Chloroform-d): 8.51 (s, 2H), 7.37 (td, J=8.3, 5.9 Hz, 1H), 7.07 (t, J=8.4 Hz, 1H), 6.42 (d, J=7.5 Hz, 1H), 6.20 (s, 1H), 6.14 (d, J=6.6 Hz, 1H), 4.83 (qd, J=8.3, 2.3 Hz, 2H), 4.62 (q, J=6.9 Hz, 1H), 4.31 (d, J=13.3 Hz, 1H), 3.45 (td, J=11.7, 4.3 Hz, 1H), 3.37-3.27 (m, 1H), 3.22-3.07 (m, 1H), 1.50 (d, J=7.0 Hz, 3H).

Example 12B: LRMS m/z (M+H): calculated 493.8, observed 494.3. ¹H NMR δ (ppm) (500 MHz, Chloroform-d): 8.48 (s, 2H), 7.32 (q, J=8.2 Hz, 1H), 7.06 (t, J=8.3 Hz, 1H), 6.36 (d, J=7.4 Hz, 1H), 6.31 (s, 1H), 5.85 (s, 1H), 4.82 (q, J=8.3 Hz, 2H), 4.57 (q, J=6.8 Hz, 1H), 4.26 (d, J=13.0 Hz, 1H), 3.49 (td, J=11.6, 4.1 Hz, 1H), 3.33 (d, J=11.9 Hz, 1H), 3.27-3.15 (m, 1H), 1.47 (d, J=6.8 Hz, 3H).

TABLE 2 The following examples were prepared according to the synthetic procedure for Examples 12A and 12B, using appropriate starting materials and reagents

| | | | Calc'd | Observed | |
|---|---|---|---|---|---|
| Example | Compound | Name | [M + H]⁺ | [M + H]⁺ | Conditions |
| 13A | 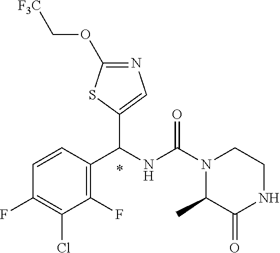 [SMILES: C[C@@H]1C(=O)NCCN1C(=O)NC(c1cnc(OCC(F)(F)F)s1)c1ccc(F)c(Cl)c1F] | (2R)-N-((R or S)-(3- chloro-2,4-difluoro- phenyl)(2-(2,2,2-tri- fluoroethoxy)thiazol-5- yl)methyl)-2-methyl-3- oxopiperazine-1- carboxamide | 498.9 | 499.4 | SFC: OD- H Co- solvent: 25% MeOH peak 1 |
| 13B | 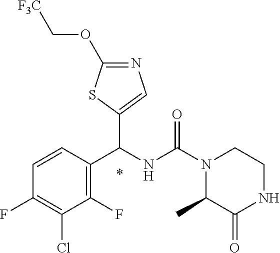 [SMILES: C[C@@H]1C(=O)NCCN1C(=O)NC(c1cnc(OCC(F)(F)F)s1)c1ccc(F)c(Cl)c1F] | (2R)-N-((S or R)-(3- chloro-2,4-difluoro- phenyl)(2-(2,2,2- trifluoroethoxy) thiazol-5-yl)methyl)-2- methyl-3- oxopiperazine-1- carboxamide | 498.9 | 499.4 | SFC: OD- H Co- solvent: 25% MeOH peak 2 |
| 14A | 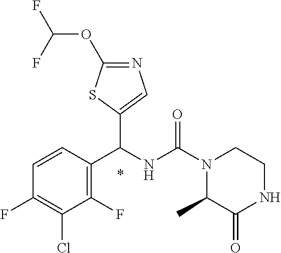 [SMILES: C[C@@H]1C(=O)NCCN1C(=O)NC(c1cnc(OC(F)F)s1)c1ccc(F)c(Cl)c1F] | (2R)-N-((R or S)-(3- chloro-2,4- difluorophenyl)(2- (difluoromethoxy) thiazol-5-yl)methyl)-2- methyl-3- oxopiperazine-1- carboxamide | 466.8 | 467.5 | SFC: AD- H Co- solvent: 15% MeOH peak 1 |
| 14B | 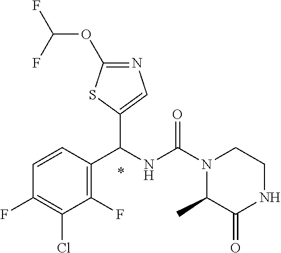 [SMILES: C[C@@H]1C(=O)NCCN1C(=O)NC(c1cnc(OC(F)F)s1)c1ccc(F)c(Cl)c1F] | (2R)-N-((S or R)-(3- chloro-2,4- difluorophenyl)(2- (difluoromethoxy) thiazol-5-yl)methyl)-2- methyl-3- oxopiperazine-1- carboxamide | 466.8 | 467.5 | SFC: AD- H Co- solvent: 15% MeOH peak 2 |

### Examples 15A and 15B

### (2R)—N—((R)-(3-chloro-2,4-difluorophenyl)(6-(trifluoromethyl)pyridin-3-yl)methyl)-2-methyl-3-oxopiperazine-1-carboxamide and (2R)—N—((S)-(3-chloro-2,4-difluorophenyl)(6-(trifluoromethyl)pyridin-3-yl)methyl)-2-methyl-3-oxopiperazine-1-carboxamide

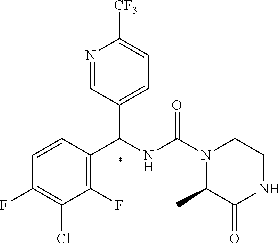 [SMILES: C[C@@H]1C(=O)NCCN1C(=O)NC(c1ccc(C(F)(F)F)nc1)c1ccc(F)c(Cl)c1F]

Step 1: (E)-2-methyl-N-((6-(trifluoromethyl)pyridin-3-yl)methyle)propane-2-sulfinamide 6-(trifluoromethyl)nicotinaldehyde (447 mg, 2.55 mmol), 2-methylpropane-2-sulfinamide (325 mg, 2.68 mmol) and titanium(IV) isopropoxide (2.5 mL, 8.44 mmol) were combined in a microwave tube. The mixture was microwaved at 90° C. for 20 minutes, then cooled to RT and poured into 30 mL brine and 50 mL ethyl acetate. The mixture was filtered through a Celite® pad. The separated organic phase was dried over Na₂SO₄, filtered and concentrated under reduced pressure. The resulting residue was purified by column chromatography on silica gel, eluting with (0-100% ethyl acetate/hexane) to give the title compound. LRMS m/z (M+H): calculated 278.3, observed 279.2.

Step 2: N-((3-chloro-2,4-difluorophenyl)(6-(trifluoromethyl)pyridin-3-yl)methyl)-2-methylpropane-2-sulfinamide 1-Bromo-3-chloro-2,4-difluorobenzene (490 mg, 2.156 mmol) was dissolved in anhydrous THF (8 mL) and purged under N₂ for 5 minutes, followed by addition of isopropylmagnesium chloride-lithium chloride complex in THF (1.3 M, 1.658 mL, 2.156 mmol). The mixture was stirred at RT for 5 hours, then (E)-2-methyl-N-((6-(trifluoromethyl)pyridin-3-yl)methylene) propane-2-sulfinamide (300 mg, 1.078 mmol) was added in one portion. The reaction was stirred at RT for 20 hours, then quenched with saturated aqueous NH₄Cl and extracted with diethyl ether. The organic layer was dried over Na₂SO₄, filtered and concentrated under reduced pressure to give the title compound. LRMS m/z (M+H): calculated 426.8, observed 427.3.

Step 3: (3-chloro-2,4-difluorophenyl)(6-(trifluoromethyl)pyridin-3-yl)methanamine hydrochloride To a solution of N-((3-chloro-2,4-difluorophenyl)(6-(trifluoromethyl)pyridin-3-yl)methyl)-2-methylpropane-2-sulfinamide (460 mg, 1.078 mmol) in CH₂Cl₂ (1.5 mL) and MeOH (0.5 mL) was added HCl in 1,4-dioxane (4 M, 2 mL, 8.00 mmol). The mixture was stirred at RT for 2 hours and then concentrated under reduced pressure. The resulting residue was washed with diethyl ether (2×10 mL) and filtered to give the title compound. LRMS m/z (M+H): calculated 322.7, observed 323.2.

Step 4: examples 15A and 15B To a solution of (3-chloro-2,4-difluorophenyl)(6-(trifluoro-methyl)pyridin-3-yl)methanamine, HCl (120 mg, 0.334 mmol) in CH₂Cl₂ (4 mL) at 0° C. was added Et₃N (0.233 mL, 1.671 mmol) and triphosgene (99 mg, 0.334 mmol). The mixture was stirred at 0° C. for 1 hour, followed by the addition of (R)-3-methylpiperazin-2-one (57.2 mg, 0.501 mmol). The reaction was stirred at 0° C. for 1 hour, then warmed to RT for 1 hour, and concentrated under reduced pressure. The resulting residue was purified by column chromatography on silica gel, eluting with (0-4% MeOH/DCM) to give a mixture of isomers, which was further separated by SFC (AD-H column, 25% MeOH co-solvent) to give Examples 15A (first eluted fraction) and 15B (second eluted fraction).

Example 15A: LRMS m/z (M+H): calculated 462.8, observed 463.3. ¹H NMR δ (ppm) (500 MHz, Chloroform-d): 8.77 (s, 1H), 7.78 (d, J=8.1 Hz, 1H), 7.67 (d, J=8.1 Hz, 1H), 7.36 (q, J=8.2 Hz, 1H), 7.06 (t, J=8.3 Hz, 1H), 6.51 (d, J=7.5 Hz, 1H), 6.25 (s, 1H), 6.12 (s, 1H), 4.61 (q, J=6.7 Hz, 1H), 4.30 (d, J=12.6 Hz, 1H), 3.44 (td, J=11.6, 3.9 Hz, 1H), 3.29 (d, J=11.8 Hz, 1H), 3.22-3.03 (m, 1H), 1.49 (d, J=7.0 Hz, 3H).

Example 15B: LRMS m/z (M+H): calculated 462.8, observed 463.3. ¹H NMR δ (ppm) (500 MHz, Chloroform-d): 8.70 (s, 1H), 7.76 (d, J=8.0 Hz, 1H), 7.69 (d, J=8.1 Hz, 1H), 7.28 (d, J=7.7 Hz, 1H), 7.05 (t, J=8.3 Hz, 1H), 6.46 (d, J=7.3 Hz, 1H), 6.30 (s, 1H), 5.67 (d, J=7.1 Hz, 1H), 4.52 (q, J=7.0 Hz, 1H), 4.26 (d, J=13.7 Hz, 1H), 3.58-3.44 (m, 1H), 3.38-3.30 (m, 1H), 3.27-3.14 (m, 1H), 1.50 (d, J=7.0 Hz, 3H).

TABLE 3 The following examples were prepared according to the synthetic procedure for Examples 15A and 15B, using the appropriate starting materials and reagents

| | | | Calc'd | Observed | |
|---|---|---|---|---|---|
| Example | Compound | Name | [M + H]⁺ | [M + H]⁺ | Conditions |
| 16A | 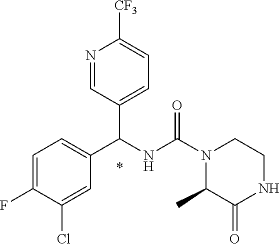 [SMILES: C[C@@H]1C(=O)NCCN1C(=O)NC(c1ccc(C(F)(F)F)nc1)c1ccc(F)c(Cl)c1] | (2R)-N-((R or S)-(3- chloro-4- fluorophenyl)(6- (trifluoromethyl)pyridin- 3-yl)methyl)-2-methyl- 3-oxopiperazine-1- carboxamide | 444.8 | 445.3 | SFC: AD-H Co-solvent: 30% EtOH peak 1 |
| 16B | 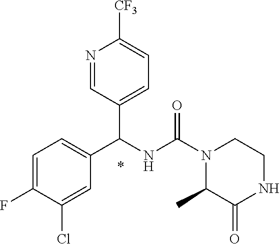 [SMILES: C[C@@H]1C(=O)NCCN1C(=O)NC(c1ccc(C(F)(F)F)nc1)c1ccc(F)c(Cl)c1] | (2R)-N-((S or R)-(3- chloro-4- fluorophenyl)(6- (trifluoromethyl)pyridin- 3-yl)methyl)-2-methyl- 3-oxopiperazine-1- carboxamide | 444.8 | 445.3 | SFC: AD-H Co-solvent: 30% EtOH peak 2 |
| 17A | 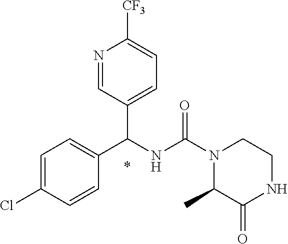 [SMILES: C[C@@H]1C(=O)NCCN1C(=O)NC(c1ccc(Cl)cc1)c1ccc(C(F)(F)F)nc1] | (2R)-N-((R or S)-(4- chlorophenyl)(6- (trifluoromethyl)pyridin- 3-yl)methyl)-2-methyl- 3-oxopiperazine-1- carboxamide | 426.8 | 427.3 | SFC: AD-H Co-solvent: 30% EtOH peak 1 |
| 17B | 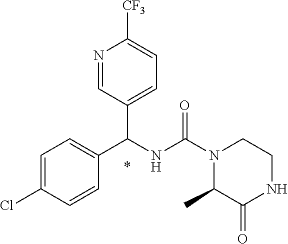 [SMILES: C[C@@H]1C(=O)NCCN1C(=O)NC(c1ccc(Cl)cc1)c1ccc(C(F)(F)F)nc1] | (2R)-N-((S or R)-(4- chlorophenyl)(6- (trifluoromethyl)pyridin- 3-yl)methyl)-2-methyl- 3-oxopiperazine-1- carboxamide | 426.8 | 427.3 | SFC: AD-H Co-solvent: 30% EtOH peak 2 |
| 18A | 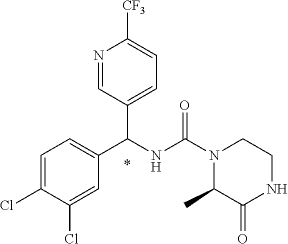 [SMILES: C[C@@H]1C(=O)NCCN1C(=O)NC(c1ccc(C(F)(F)F)nc1)c1ccc(Cl)c(Cl)c1] | (2R)-N-((R or S)-(3,4- dichlorophenyl)(6- (trifluoromethyl)pyridin- 3-yl)methyl)-2-methyl- 3-oxopiperazine-1- carboxamide | 460.1 | 461.3 | SFC: AD-H Co-solvent: 35% EtOH peak 1 |
| 18B | 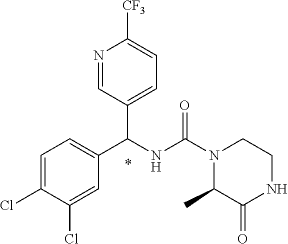 [SMILES: C[C@@H]1C(=O)NCCN1C(=O)NC(c1ccc(C(F)(F)F)nc1)c1ccc(Cl)c(Cl)c1] | (2R)-N-((S or R)-(3,4- dichlorophenyl)(6- (trifluoromethyl)pyridin- 3-yl)methyl)-2-methyl- 3-oxopiperazine-1- carboxamide | 460.1 | 461.3 | SFC: AD-H Co-solvent: 35% EtOH peak 2 |
| 19A | 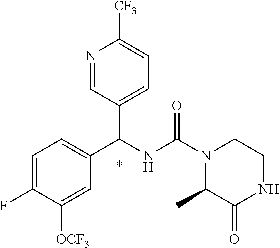 [SMILES: C[C@@H]1C(=O)NCCN1C(=O)NC(c1ccc(C(F)(F)F)nc1)c1ccc(F)c(OC(F)(F)F)c1] | (2R)-N-((R or S)-(4- fluoro-3-(trifluoro- methoxy)phenyl)(6- (trifluoromethyl) pyridin-3-yl)methyl)-2- methyl-3- oxopiperazine-1- carboxamide | 494.4 | 495.4 | SFC: OJ-H Co-solvent: 15% (IPA + 0.2% DIPEA) peak 1 |
| 19B | 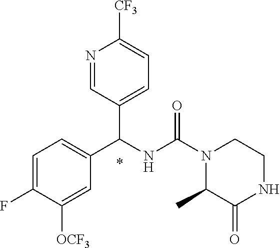 [SMILES: C[C@@H]1C(=O)NCCN1C(=O)NC(c1ccc(C(F)(F)F)nc1)c1ccc(F)c(OC(F)(F)F)c1] | (2R)-N-((S or R)-(4- fluoro-3-(trifluoro- methoxy)phenyl)(6- (trifluoromethyl) pyridin-3-yl)methyl)-2- methyl-3- oxopiperazine-1- carboxamide | 494.4 | 495.4 | SFC: OJ-H Co-solvent: 15% (IPA + 0.2% DIPEA) peak 2 |
| 20A | 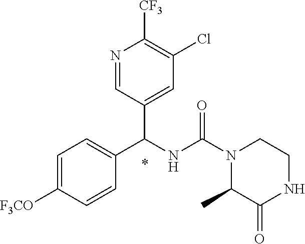 [SMILES: C[C@@H]1C(=O)NCCN1C(=O)NC(c1ccc(OC(F)(F)F)cc1)c1cnc(C(F)(F)F)c(Cl)c1] | (2R)-N-((R or S)-(5- chloro-6-(trifluoro- methyl)pyridin-3-yl)(4- (trifluoromethoxy) phenyl)methyl)-2- methyl-3- oxopiperazine-1- carboxamide | 510.8 | 511.55 | SFC: AD-H Co-solvent: 30% MeOH peak 1 |
| 20B | 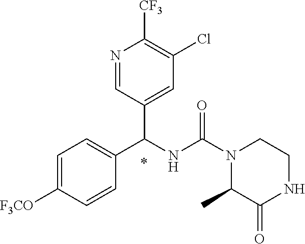 [SMILES: C[C@@H]1C(=O)NCCN1C(=O)NC(c1ccc(OC(F)(F)F)cc1)c1cnc(C(F)(F)F)c(Cl)c1] | (2R)-N-((S or R)-(5- chloro-6-(trifluoro- methyl)pyridin-3-yl)(4- (trifluoromethoxy) phenyl)methyl)-2- methyl-3- oxopiperazine-1- carboxamide | 510.8 | 511.55 | SFC: AD-H Co-solvent: 30% MeOH peak 2 |
| 21A | 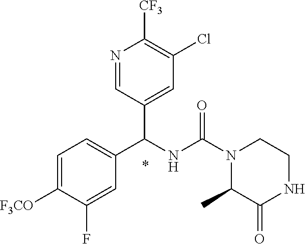 [SMILES: C[C@@H]1C(=O)NCCN1C(=O)NC(c1ccc(OC(F)(F)F)c(F)c1)c1cnc(C(F)(F)F)c(Cl)c1] | (2R)-N-((R or S)-(5- chloro-6-(trifluoro- methyl)pyridin-3-yl)(3- fluoro-4- (trifluoromethoxy) phenyl)methyl)-2- methyl-3- oxopiperazine-1- carboxamide | 528.8 | 529.5 | SFC: OJ-H Co-solvent: 15% MeOH peak 1 |
| 21B | 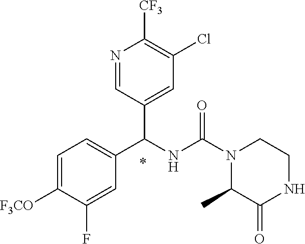 [SMILES: C[C@@H]1C(=O)NCCN1C(=O)NC(c1ccc(OC(F)(F)F)c(F)c1)c1cnc(C(F)(F)F)c(Cl)c1] | (2R)-N-((S or R)-(5- chloro-6-(trifluoro- methyl)pyridin-3-yl)(3- fluoro-4-(trifluoro- methoxy)phenyl) methyl)-2-methyl-3- oxo-piperazine-1- carboxamide | 528.8 | 529.5 | SFC: OJ-H Co-solvent: 15% MeOH peak 2 |
| 22A | 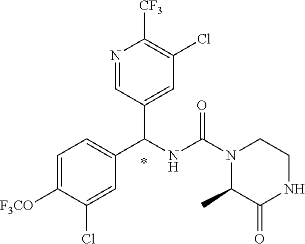 [SMILES: C[C@@H]1C(=O)NCCN1C(=O)NC(c1ccc(OC(F)(F)F)c(Cl)c1)c1cnc(C(F)(F)F)c(Cl)c1] | (2R)-N-((R or S)-(3- chloro-4-(trifluoro- methoxy)phenyl)(5- chloro-6-(trifluoro- methyl)pyridin-3- yl)methyl)-2-methyl-3- oxopiperazine-1- carboxamide | 544.1 | 545.5 | SFC: OJ-H Co-solvent: 15% MeOH peak 1 |
| 22B | 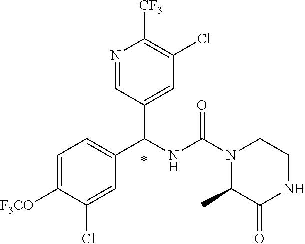 [SMILES: C[C@@H]1C(=O)NCCN1C(=O)NC(c1ccc(OC(F)(F)F)c(Cl)c1)c1cnc(C(F)(F)F)c(Cl)c1] | (2R)-N-((S or R)-(3- chloro-4-(trifluoro- methoxy)phenyl)(5- chloro-6-(trifluoro- methyl)pyridin-3- yl)methyl)-2-methyl-3- oxopiperazine-1- carboxamide | 544.1 | 545.5 | SFC: OJ-H Co-solvent: 15% MeOH peak 2 |
| 23A | 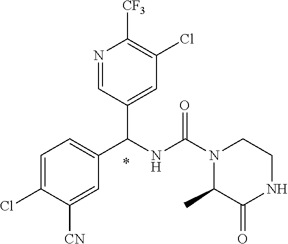 [SMILES: C[C@@H]1C(=O)NCCN1C(=O)NC(c1cnc(C(F)(F)F)c(Cl)c1)c1ccc(Cl)c(C#N)c1] | (2R)-N-((R or S)-(4- chloro-3-cyano- phenyl)(5-chloro-6- (trifluoromethyl) pyridin-3-yl)methyl)-2- methyl-3- oxopiperazine-1- carboxamide | 485.1 | 486.5 | SFC: OJ-H Co-solvent: 25% MeOH peak 1 |
| 23B | 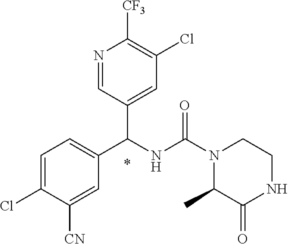 [SMILES: C[C@@H]1C(=O)NCCN1C(=O)NC(c1cnc(C(F)(F)F)c(Cl)c1)c1ccc(Cl)c(C#N)c1] | (2R)-N-((S or R)-(4- chloro-3-cyano- phenyl)(5-chloro-6- (trifluoromethyl) pyridin-3-yl)methyl)-2- methyl-3- oxopiperazine-1- carboxamide | 485.1 | 486.5 | SFC: OJ-H Co-solvent: 25% MeOH peak 2 |
| 24A | 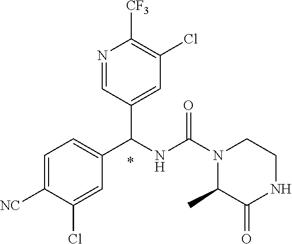 [SMILES: C[C@@H]1C(=O)NCCN1C(=O)NC(c1ccc(C#N)c(Cl)c1)c1cnc(C(F)(F)F)c(Cl)c1] | (2R)-N-((R or S)-(3- chloro-4-cyano- phenyl)(5-chloro-6- (trifluoromethyl) pyridin-3-yl)methyl)-2- methyl-3- oxopiperazine-1- carboxamide | 485.1 | 486.5 | SFC: OD-H Co-solvent: 15% MeOH peak 1 |
| 24B | 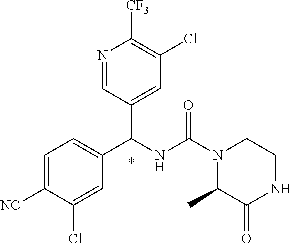 [SMILES: C[C@@H]1C(=O)NCCN1C(=O)NC(c1ccc(C#N)c(Cl)c1)c1cnc(C(F)(F)F)c(Cl)c1] | (2R)-N-((S or R)-(3- chloro-4-cyano- phenyl)(5-chloro-6- (trifluoromethyl) pyridin-3-yl)methyl)-2- methyl-3- oxopiperazine-1- carboxamide | 485.1 | 486.5 | SFC: OD-H Co-solvent: 15% MeOH peak 2 |
| 25A | 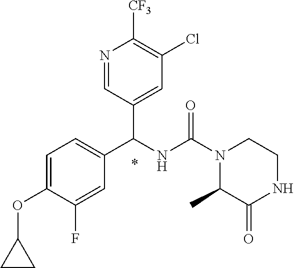 [SMILES: C[C@@H]1C(=O)NCCN1C(=O)NC(c1ccc(OC2CC2)c(F)c1)c1cnc(C(F)(F)F)c(Cl)c1] | (2R)-N-((R or S)-(5- chloro-6-(trifluoro- methyl)pyridin-3-yl)(4- cyclopropoxy-3- fluorophenyl)methyl)- 2-methyl-3- oxopiperazine-1- carboxamide | 500.9 | 501.6 | SFC: AD-H Co-solvent: 35% MeOH peak 1 |
| 25B | 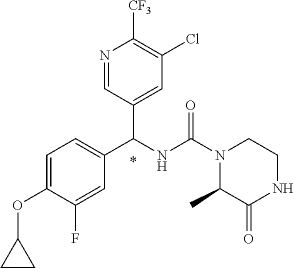 [SMILES: C[C@@H]1C(=O)NCCN1C(=O)NC(c1ccc(OC2CC2)c(F)c1)c1cnc(C(F)(F)F)c(Cl)c1] | (2R)-N-((S or R)-(5- chloro-6-(trifluoro- methyl)pyridin-3-yl)(4- cyclopropoxy-3- fluorophenyl)methyl)- 2-methyl-3- oxopiperazine-1- carboxamide | 500.9 | 501.6 | SFC: AD-H Co-solvent: 35% MeOH peak 2 |
| 26A | 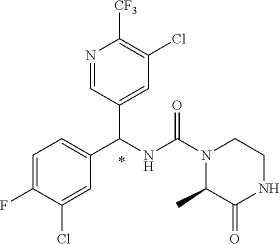 [SMILES: C[C@@H]1C(=O)NCCN1C(=O)NC(c1ccc(F)c(Cl)c1)c1cnc(C(F)(F)F)c(Cl)c1] | (2R)-N-((R or S)-(3- chloro-4-fluoro- phenyl)(5-chloro-6- (trifluoromethyl) pyridin-3-yl)methyl)-2- methyl-3- oxopiperazine-1- carboxamide | 478.1 | 479.3 | SFC: AD-H Co-solvent: 30% MeOH peak 1 |
| 26B | 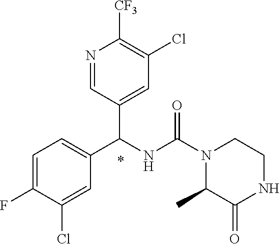 [SMILES: C[C@@H]1C(=O)NCCN1C(=O)NC(c1ccc(F)c(Cl)c1)c1cnc(C(F)(F)F)c(Cl)c1] | (2R)-N-((S or R)-(3- chloro-4-fluoro- phenyl)(5-chloro-6- (trifluoromethyl) pyridin-3-yl)methyl)-2- methyl-3- oxopiperazine-1- carboxamide | 478.1 | 479.3 | SFC: AD-H Co-solvent: 30% MeOH peak 2 |
| 27A | 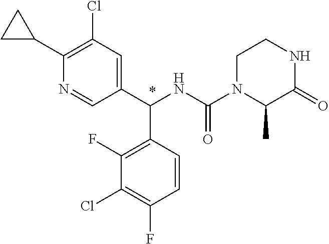 [SMILES: C[C@@H]1C(=O)NCCN1C(=O)NC(c1cnc(C2CC2)c(Cl)c1)c1ccc(F)c(Cl)c1F] | (2R)-N-((R or S)-(3- chloro-2,4-difluoro- phenyl)(5-chloro-6- cyclopropyl pyridin-3- yl)methyl)-2-methyl-3- oxopiperazine-1- carboxamide | 468.1 | 469.1 | SFC: AD-H Co-solvent: 35% EtOH peak 1 |
| 27B | 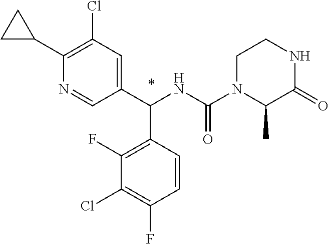 [SMILES: C[C@@H]1C(=O)NCCN1C(=O)NC(c1cnc(C2CC2)c(Cl)c1)c1ccc(F)c(Cl)c1F] | (2R)-N-((S or R)-(3- chloro-2,4- difluorophenyl)(5- chloro-6-cyclopropyl pyridin-3-yl)methyl)-2- methyl-3- oxopiperazine-1- carboxamide | 468.1 | 469.1 | SFC: AD-H Co-solvent: 35% EtOH peak 2 |

### Example 28A

### (2R)—N—((R or S)-(3,4-dichlorophenyl)(2-(trifluoromethyl)pyrimidin-5-yl)methyl)-2-methyl-3-oxopiperazine-1-carboxamide

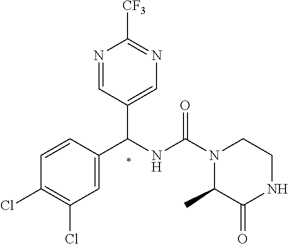 [SMILES: C[C@@H]1C(=O)NCCN1C(=O)NC(c1cnc(C(F)(F)F)nc1)c1ccc(Cl)c(Cl)c1]

Step 1: (R, E)-2-methyl-N-((2-(trifluoromethyl)pyrimidin-5-yl)methylene)propane-2-sulfinamide A microwave tube was charged with 2-(trifluoromethyl)pyrimidine-5-carbaldehyde (1.5 g, 8.52 mmol), (R)-2-methylpropane-2-sulfinamide (1.239 g, 10.22 mmol) and tetraethoxytitanium (5.83 g, 25.6 mmol). The mixture was microwaved at 90° C. for 25 minutes, then cooled to RT, and 30 mL H₂O and 50 mL ethyl acetate were added. The reaction was stirred for 10 minutes, then filtered through a Celite® pad. The separated organic layer was dried over Na₂SO₄, filtered and concentrated under reduced pressure. The resulting residue was purified by column chromatography on silica gel, eluting with (0-50% ethyl acetate/hexane to give the title compound. LRMS m/z (M+H): calculated 279.3, observed 280.3.

Step 2: (R)—N-((3,4-dichlorophenyl)(2-(trifluoromethyl)pyrimidin-5-yl)methyl)-2-methyl propane-2-sulfinamide (isomer A) and (R)—N-((3,4-dichlorophenyl)(2-(trifluoromethyl) pyrimidin-5-yl)methyl)-2-methylpropane-2-sulfinamide (isomer B) To a solution of (R,E)-2-methyl-N-((2-(trifluoromethyl)pyrimidin-5-yl)methylene)propane-2-sulfinamide (300 mg, 1.074 mmol) in THF (8 mL) at −20° C. was added 3,4-dichlorophenylmagnesium bromide in THF (1 M, 1.611 mL, 1.611 mmol). The reaction was stirred at −20° C. for 2 hours, then warmed to 0° C. and quenched with saturated aqueous NH₄Cl. The mixture was extracted with diethyl ether, and the separated organic layer was dried over Na₂SO₄, filtered and concentrated under reduced pressure. The resulting residue was purified via preparative TLC, eluting with (30% ethyl acetate/hexane), to give two fractions: Isomer A (polar fraction), LRMS m/z (M+H): calculated 425.0, observed 426.3; and Isomer B (less polar fraction), LRMS m/z (M+H): calculated 425.0, observed 426.3.

Step 3: (3,4-dichlorophenyl)(2-(trifluoromethyl)pyrimidin-5-yl)methanamine hydrochloride To a solution of (R)—N-((3,4-dichlorophenyl)(2-(trifluoromethyl)pyrimidin-5-yl)methyl)-2-methylpropane-2-sulfinamide (isomer A, 190 mg, 0.446 mmol) in DCM (2 mL) was added HCl in 1,4-dioxane (4 M, 2 mL, 8.00 mmol). The mixture was stirred at RT for 1 hour and concentrated under reduced pressure. The resulting residue was washed with 2×10 mL diethyl ether, and filtered to give the title compound. LRMS m/z (M+H): calculated 321.0, observed 322.2.

Step 4: Example 28A To a solution of ((3,4-dichlorophenyl)(2-(trifluoromethyl)pyrimidin-5-yl)methanamine, HCl (55 mg, 0.153 mmol) in CH₂Cl₂ (2 mL) at 0° C. were added Et₃N (0.107 mL, 0.767 mmol) and triphosgene (45.5 mg, 0.153 mmol). The mixture was stirred at 0° C. for 1 hour, and then (R)-3-methylpiperazin-2-one (26.3 mg, 0.230 mmol) was added. The reaction was stirred at 0° C. for 1 hour, then warmed to RT over 1 hour, and concentrated under reduced pressure. The resulting residue was purified by column chromatography on silica gel, eluting with (0-4% MeOH/DCM) to give Example 28A: LRMS m/z (M+H): calculated 461.1, observed 462.2. ¹H NMR δ (ppm) (500 MHz, Chloroform-d): 8.91 (s, 2H), 7.49 (d, J=8.2 Hz, 2H), 7.19 (dd, J=8.3, 2.1 Hz, 1H), 6.59 (s, 1H), 6.34 (d, J=7.8 Hz, 1H), 6.12 (s, 1H), 4.67 (q, J=6.8 Hz, 1H), 4.37 (dd, J=13.7, 3.1 Hz, 1H), 3.44 (td, J=11.8, 4.3 Hz, 1H), 3.30 (d, J=12.0 Hz, 1H), 3.20-3.09 (m, 1H), 1.49 (d, J=7.0 Hz, 3H).

TABLE 4 The following examples were prepared according to the synthetic procedure for Example 28A, using the appropriate starting materials and reagents

| | | | Calc'd | Observed | |
|---|---|---|---|---|---|
| Example | Compound | Name | [M + H]⁺ | [M + H]⁺ | Conditions |
| 28B | 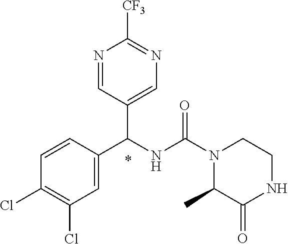 [SMILES: C[C@@H]1C(=O)NCCN1C(=O)NC(c1cnc(C(F)(F)F)nc1)c1ccc(Cl)c(Cl)c1] | (2R)-N-((S or R)-(3,4- dichlorophenyl)(2- (trifluoromethyl) pyrimidin-5-yl) methyl)-2-methyl-3- oxopiperazine-1- carboxamide | 461.1 | 462.2 | In step 3 above, using isomer B as starting material |
| 29A | 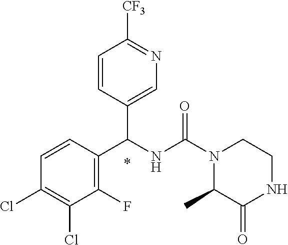 [SMILES: C[C@@H]1C(=O)NCCN1C(=O)NC(c1ccc(C(F)(F)F)nc1)c1ccc(Cl)c(Cl)c1F] | (2R)-N-((R or S)-(3,4- dichloro-2-fluoro- phenyl)(6-(trifluoro- methyl)pyridin-3- yl)methyl)-2-methyl-3- oxopiperazine-1- carboxamide | 478.3 | 479.3 | In step 2 SFC: AD-H Co- solvent: 35% EtOH peak 1 |
| 29B | 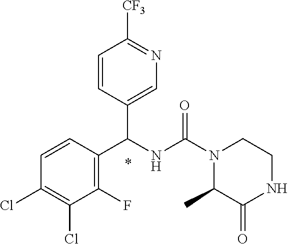 [SMILES: C[C@@H]1C(=O)NCCN1C(=O)NC(c1ccc(C(F)(F)F)nc1)c1ccc(Cl)c(Cl)c1F] | (2R)-N-((3,4-dichloro- 2-fluorophenyl)(6-(tri- fluoromethyl)pyridin-3- yl)methyl)-2-methyl-3- oxopiperazine-1- carboxamide | 478.3 | 479.3 | In step 2 SFC: AD-H Co- solvent: 35% EtOH peak 2 |
| 30A | 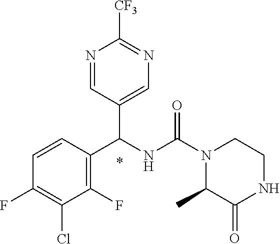 [SMILES: C[C@@H]1C(=O)NCCN1C(=O)NC(c1cnc(C(F)(F)F)nc1)c1ccc(F)c(Cl)c1F] | (2R)-N-((R or S)-(3- chloro-2,4-difluoro- phenyl)(2-(trifluoro- methyl)pyrimidin-5- yl)methyl)-2-methyl-3- oxopiperazine-1- carboxamide | 463.8 | 464.3 | In step 2 SFC: OD-H Co- solvent: 15% EtOH peak 1 |
| 30B | 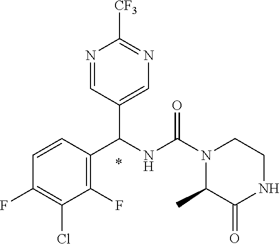 [SMILES: C[C@@H]1C(=O)NCCN1C(=O)NC(c1cnc(C(F)(F)F)nc1)c1ccc(F)c(Cl)c1F] | (2R)-N-((S or R)-(3- chloro-2,4-difluoro- phenyl)(2-(trifluoro- methyl)pyrimidin-5- yl)methyl)-2-methyl-3- oxopiperazine-1- carboxamide | 463.8 | 464.3 | In step 2 SFC: OD-H Co- solvent: 15% EtOH peak 2 |
| 31A | 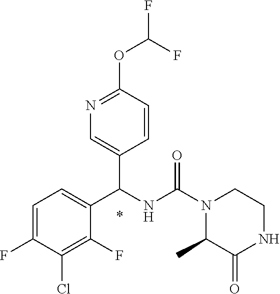 [SMILES: C[C@@H]1C(=O)NCCN1C(=O)NC(c1ccc(OC(F)F)nc1)c1ccc(F)c(Cl)c1F] | (2R)-N-((R or S)-(3- chloro-2,4-difluoro- phenyl)(6-(difluoro- methoxy)pyridin-3- yl)methyl)-2-methyl-3- oxopiperazine-1- carboxamide | 460.8 | 461.4 | In step 2 SFC: AD-H Co- solvent: 15% MeOH peak 1 |
| 31B | 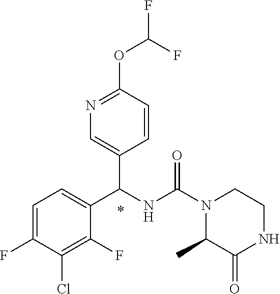 [SMILES: C[C@@H]1C(=O)NCCN1C(=O)NC(c1ccc(OC(F)F)nc1)c1ccc(F)c(Cl)c1F] | (2R)-N-((S or R)-(3- chloro-2,4-difluoro- phenyl)(6-(difluoro- methoxy)pyridin-3- yl)methyl)-2-methyl-3- oxopiperazine-1- carboxamide | 460.8 | 461.4 | In step 2 SFC: AD-H Co- solvent: 15% MeOH peak 2 |
| 32A | 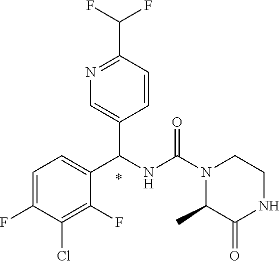 [SMILES: C[C@@H]1C(=O)NCCN1C(=O)NC(c1ccc(C(F)F)nc1)c1ccc(F)c(Cl)c1F] | (2R)-N-((R or S)-(3- chloro-2,4-difluoro- phenyl)(6-(difluoro- methyl)pyridin-3- yl)methyl)-2-methyl-3- oxopiperazine-1- carboxamide | 444.8 | 445.4 | In step 2 SFC: OD-H Co- solvent: 15% MeOH peak 1 |
| 32B | 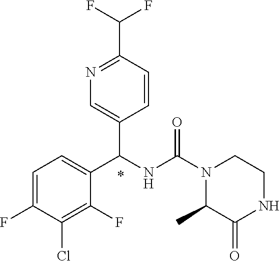 [SMILES: C[C@@H]1C(=O)NCCN1C(=O)NC(c1ccc(C(F)F)nc1)c1ccc(F)c(Cl)c1F] | (2R)-N-((s or R)-(3- chloro-2,4-difluoro- phenyl)(6-(difluoro- methyl)pyridin-3- yl)methyl)-2-methyl-3- oxopiperazine-1- carboxamide | 444.8 | 445.4 | In step 2 SFC: OD-H Co- solvent: 15% MeOH peak 2 |
| 33A | 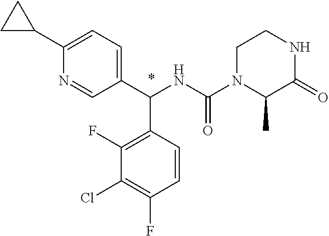 [SMILES: C[C@@H]1C(=O)NCCN1C(=O)NC(c1ccc(C2CC2)nc1)c1ccc(F)c(Cl)c1F] | (2R)-N-((R or S)-(3- chloro-2,4-difluoro- phenyl)(6-cyclo- propylpyridin-3- yl)methyl)-2-methyl-3- oxopiperazine-1- carboxamide | 398.9 | 399.4 | In step 2 silica gel column eluant (0-50% ethyl acetate/ hexane) 1^{st} fraction |
| 33B | 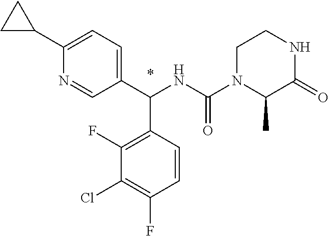 [SMILES: C[C@@H]1C(=O)NCCN1C(=O)NC(c1ccc(C2CC2)nc1)c1ccc(F)c(Cl)c1F] | (2R)-N-((S or R)-(3- chloro-2,4-difluoro- phenyl)(6-cyclo- propylpyridin-3- yl)methyl)-2-methyl-3- oxopiperazine-1- carboxamide | 398.9 | 399.4 | In step 2 silica gel column eluant (0-50% ethyl acetate/ hexane) 2nd fraction |

### Example 34

### (2R)—N—((R or S)-(3-chloro-2,4-difluorophenyl)(5-fluoro-6-(trifluoromethyl)pyridin-3-yl)methyl)-2-methyl-3-oxopiperazine-1-carboxamide

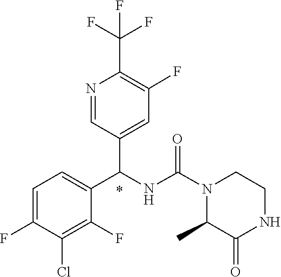 [SMILES: C[C@@H]1C(=O)NCCN1C(=O)NC(c1cnc(C(F)(F)F)c(F)c1)c1ccc(F)c(Cl)c1F]

Step 1: (R,E)-N-((5-fluoro-6-(trifluoromethyl)pyridin-3-yl)methylene)-2-methylpropane-2-sulfinamide A microwave tube was charged with (R)-2-methylpropane-2-sulfinamide (1.378 g, 11.37 mmol), 5-fluoro-6-(trifluoromethyl)nicotinaldehyde (1.83 g, 9.48 mmol), titanium(IV) ethoxide (3.97 mL, 18.95 mmol) and toluene (5 mL). The mixture was microwaved at 100° C. for 20 minutes, cooled to RT, and then 30 mL H₂O and 100 mL ethyl acetate were added. The reaction was stirred for 10 minutes, then filtered through a Celite® pad. The separated organic layer was dried over Na₂SO₄, filtered and concentrated under reduced pressure. The resulting residue was purified by column chromatography on silica gel, eluting with (0-40% ethyl acetate/hexane to give the title compound. LRMS m/z (M+H): calculated 296.3, observed 279.3.

Step 2: N-((3-chloro-2,4-difluorophenyl)(5-fluoro-6-(trifluoromethyl)pyridin-3-yl)methyl)-2-methylpropane-2-sulfinamide (Isomer A) and N-((3-chloro-2,4-difluorophenyl)(5-fluoro-6-(trifluoromethyl)pyridin-3-yl)methyl)-2-methylpropane-2-sulfinamide (Isomer B) To a solution of 2-chloro-1,3-difluoro-4-iodobenzene (915 mg, 3.33 mmol) in anhydrous THF (15 mL) at −78° C. was added isopropylmagnesium chloride-lithium chloride complex in THF (1.3 M, 1.973 mL, 2.57 mmol). The reaction mixture was stirred at −78° C. for 1 hour, then at −20° C. for 1 hour. The reaction was again cooled to −78° C., followed by the addition of (R,E)-N-((5-fluoro-6-(trifluoromethyl)pyridin-3-yl)methylene)-2-methylpropane-2-sulfinamide (760) mg. 2.57 mmol). The mixture was stirred at −78° C. for 1 hour, then gradually warmed to 0° C. over 1 hour. The reaction was then quenched with saturated aqueous NH₄Cl and extracted with ethyl acetate. The separated organic layer was dried over Na₂SO₄, filtered and concentrated under reduced pressure. The resulting residue was purified by column chromatography on silica gel, eluting with (0-40% ethyl acetate/hexane) to give a mixture, which was further separated by SFC (AD-H column, 5% MeOH co-solvent) to give Isomer A (first fraction) and Isomer B (second fraction). Isomer A: LRMS m/z (M+H): calculated 444.8, observed 445.4. Isomer B: LRMS m/z (M+H): calculated 444.8, observed 445.4.

Step 3: (3-chloro-2,4-difluorophenyl)(5-fluoro-6-(trifluoromethyl)pyridin-3-yl)methanamine hydrochloride To a solution of N-((3-chloro-2,4-difluorophenyl)(5-fluoro-6-(trifluoro-methyl)pyridin-3-yl)methyl)-2-methylpropane-2-sulfinamide (Isomer A) (800 mg, 1.798 mmol) in CH₂Cl₂ (2 mL) at 0° C. was added HCl in 1,4-dioxane (4 M, 1 mL, 4.00 mmol). The mixture was stirred at RT for 1 hour and then concentrated under reduced pressure. The resulting residue was washed with 2×10 mL diethyl ether and filtered to give the title compound. LRMS m z (M+H): calculated 340.6, observed 341.3.

Step 4: Example 34 To a solution of (3-chloro-2,4-difluorophenyl)(5-fluoro-6-(trifluoromethyl)pyridin-3-yl)methanamine, HCl (129 mg, 0.342 mmol) in CH₂Cl₂ (3 mL) at 0° C. were added Et₃N (0.191 mL, 1.368 mmol) and triphosgene (81 mg, 0.274 mmol). The mixture was stirred at 0° C. for 1 hour and then (R)-3-methylpiperazin-2-one (54.7 mg, 0.479 mmol) was added. he reaction was stirred at 0° C. for 1 hour, then warmed to RT for 1 hour, and concentrated under reduced pressure. The resulting residue was purified by column chromatography on silica gel, eluting with (0-4% MeOH/DCM) to give Example 34: LRMS m/z. (M+H): calculated 480.8, observed 481.4. ¹H NMR δ (ppm) (500 MHz, Chloroform-d): 8.52 (s, 1H), 7.58 (d, J=10.4 Hz, 1H), 7.37-7.30 (m, 1H), 7.07 (t, J=8.3 Hz, 1H), 6.52 (d, J=7.6 Hz, 1H), 6.31 (s, 1H), 6.13 (s, 1H), 4.65 (q, J=6.7 Hz, 1H), 4.30 (d, J=13.2 Hz, 1H), 3.48 (td, J=11.6, 3.9 Hz, 1H), 3.32 (d, J=11.9 Hz, 1H), 3.23-3.12 (m, 1H), 1.48 (d, J=7.0 Hz, 3H).

TABLE 5 The following examples were prepared according to the synthetic procedure for Example 34, using the appropriate starting materials and reagents

| | | | Calc'd | Observed | |
|---|---|---|---|---|---|
| Example | Compound | Name | [M + H]⁺ | [M + H]⁺ | Conditions |
| 35 | 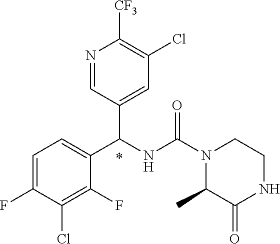 [SMILES: C[C@@H]1C(=O)NCCN1C(=O)NC(c1cnc(C(F)(F)F)c(Cl)c1)c1ccc(F)c(Cl)c1F] | (2R)-N-((R or S)-(3- chloro-2,4-difluoro phenyl)(5-chloro-6- (trifluoromethyl) pyridin-3-yl) methyl)-2-methyl-3- oxopiperazine-1- carboxamide | 496.2 | 497.4 | In step 2 SFC: OD-H Co-solvent: 10% MeOH first fraction taken forward in step 3) |
| 36 | 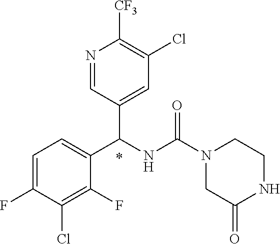 [SMILES: O=C1CN(C(=O)NC(c2cnc(C(F)(F)F)c(Cl)c2)c2ccc(F)c(Cl)c2F)CCN1] | N-((R or S)-(3- chloro-2,4- difluorophenyl)(5- chloro-6-(trifluoro- methyl)pyridin-3- yl)methyl)-3- oxopiperazine-1- carboxamide | 482.0 | 483.4 | In step 2 SFC: OD-H Co-solvent: 10% MeOH first fraction taken forward in step 3) |
| 37A | 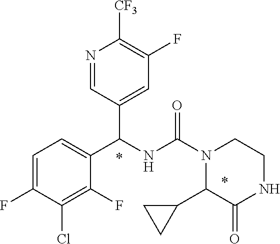 [SMILES: O=C1NCCN(C(=O)NC(c2cnc(C(F)(F)F)c(F)c2)c2ccc(F)c(Cl)c2F)C1C1CC1] | N-(R or S)-(3- chloro-2,4- difluorophenyl)(5- fluoro-6-(trifluoro- methyl)pyridin-3- yl)methyl)-(R or S)- 2-cyclopropyl-3- oxopiperazine-1- carboxamide | 506.8 | 507.5 | In step 3 using isomer A In step 4 SFC: OD-H Co-solvent: 30% EtOH peak 1 |
| 37B | 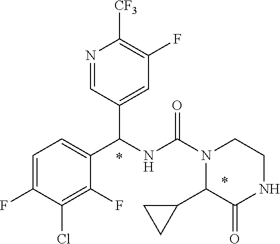 [SMILES: O=C1NCCN(C(=O)NC(c2cnc(C(F)(F)F)c(F)c2)c2ccc(F)c(Cl)c2F)C1C1CC1] | N-((R or S)-(3- chloro-2,4- difluorophenyl)(5- fluoro-6-(trifluoro- methyl)pyridin-3- yl)methyl)-(S or R)- 2-cyclopropyl-3- oxopiperazine-1- carboxamide | 506.8 | 507.5 | In step 3 using isomer A In step 4 SFC: OD-H Co-solvent: 30% EtOH peak 2 |

### Examples 38A and 38B

### N—((R or S)-(3-chloro-2,4-difluorophenyl)(6-(trifluoromethyl)pyridin-3-yl)methyl)-(R)-2-cyclopropyl-3-oxopiperazine-1-carboxamide and N—((R or S)-(3-chloro-2,4-difluorophenyl)(6-(trifluoromethyl)pyridin-3-yl)methyl)-(S)-2-cyclopropyl-3-oxopiperazine-1-carboxamide

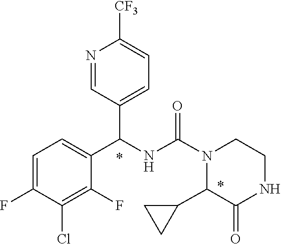 [SMILES: O=C1NCCN(C(=O)NC(c2ccc(C(F)(F)F)nc2)c2ccc(F)c(Cl)c2F)C1C1CC1]

Step 1: (R,E)-2-methyl-N-((6-(trifluoromethyl)pyridin-3-yl)methylene)propane-2-sulfinamide 6-(trifluoromethyl)nicotinaldehyde (2.04 g, 11.65 mmol), (R)-2-methylpropane-2-sulfinamide (1.694 g, 13.98 mmol) and tetraisopropoxytitanium (8.62 mL, 29.1 mmol) were combined in a microwave tube. The mixture was microwaved at 90° C. for 20 minutes, then cooled to RT and poured into 30 mL brine and 100 mL ethyl acetate. The mixture was filtered through a Celite® pad. The separated organic layer was dried over Na₂SO₄, filtered and concentrated under reduce pressure. The resulting residue was purified by column chromatography on silica gel, eluting with (0-100% ethyl acetate/hexane) to give the title compound. LRMS m/z (M+H): calculated 278.3, observed 279.2.

Step 2: (R)—N-((3-chloro-2,4-difluorophenyl)(6-(trifluoromethyl)pyridin-3-yl)methyl)-2-methylpropane-2-sulfinamide (Isomer A) and (R)—N-((3-chloro-2,4-difluorophenyl)(6-(trifluoromethyl)pyridin-3-yl)methyl)-2-methylpropane-2-sulfinamide (Isomer B) To a solution of 2-chloro-1,3-difluoro-4-iodobenzene (4.17 g, 15.20 mmol) in THF (20 mL) at −20° C. was added isopropylmagnesium chloride-lithium chloride complex in THF (1.3 M, 11.69 mL, 15.20 mmol). The mixture was stirred at −20° C. for 2 hours, followed by the addition of (R,E)-2-methyl-N-((6-(trifluoromethyl)pyridin-3-yl)methylene)propane-2-sulfinamide (2.82 g, 10.13 mmol). The reaction was stirred at −20° C. for 2 h, then warmed to RT, quenched with saturated aqueous NH₄Cl and extracted with diethyl ether. The separated organic layer was dried over Na₂SO₄, filtered and concentrated under reduced pressure to give a mixture, which was further separated by SFC (OD-H column, 20% MeOH co-solvent) to give Isomer A (first fraction) and Isomer B (second fraction).

Step 3: (3-chloro-2,4-difluorophenyl)(6-(trifluoromethyl)pyridin-3-yl)methanamine hydrochloride To a solution of (R)—N-((3-chloro-2,4-difluorophenyl)(6-(trifluoromethyl)pyridin-3-yl)methyl)-2-methylpropane-2-sulfinamide (Isomer B, 1.80 g, 4.22 mmol) in DCM (8 mL) and MeOH (2 mL) was added HCl in 1,4-dioxane (4 M, 6 mL, 24.00 mmol). The mixture was stirred at RT for 2 hours and then concentrated under reduced pressure. The resulting residue was washed with diethyl ether (2×20 mL) and filtered to give the title compound. LRMS m/z (M+H): calculated 322.7, observed 323.2.

Step 4: Examples 38A and 38B To a solution of (3-chloro-2,4-difluorophenyl)(6-(trifluoro-methyl)pyridin-3-yl)methanamine HCl (118 mg, 0.329 mmol) in CH₂Cl₂ (4 mL) at 0° C. were added Et₃N (0.229 mL, 1.643 mmol) and triphosgene (97 mg, 0.329 mmol). The mixture was stirred at 0° C. for 1 hour and then 3-cyclopropylpiperazin-2-one (69.1 mg, 0.493 mmol) was added. After stirring at 0° C. for 1 hour, the reaction was warmed to RT over 1 hour, and concentrated under reduced pressure. The resulting residue that was purified by column chromatography on silica gel, eluting with (0-4% MeOH/DCM) to give a mixture, which was further separated by SFC (OJ-H column, 30% MeOH co-solvent) to give Examples 38A (first eluted fraction) and 38B (second eluted fraction).

Example 38A: LRMS m/z (M+H): calculated 488.8, observed 489.5. ¹H NMR δ (ppm) (500 MHz, Chloroform-d): 8.78-8.62 (m, 1H), 7.82-7.73 (m, 1H), 7.68 (d, J=8.2 Hz, 1H), 7.30-7.24 (m, 1H), 7.05 (t, J=8.3 Hz, 1H), 6.57-6.33 (m, 2H), 5.72 (s, 1H), 4.21 (d, J=6.3 Hz, 1H), 4.14 (d, J=12.0 Hz, 1H), 3.54-3.37 (m, 3H), 1.30-1.21 (m, 1H), 0.69 (p, J=9.1 Hz, 1H), 0.59 (dq, J=17.5, 9.6, 7.4 Hz, 2H), 0.46 (d, J=5.3 Hz, 1H).

Example 38B: LRMS m/z (M+H): calculated 488.8, observed 489.5. ¹H NMR δ (ppm) (500 MHz, Chloroform-d): 8.73 (s, 1H), 7.78 (d, J=8.1 Hz, 1H), 7.68 (d, J=8.1 Hz, 1H), 7.36-7.29 (m, 1H), 7.07 (t, J=8.3 Hz, 1H), 6.47 (d, J=7.6 Hz, 1H), 6.28 (s, 1H), 5.78 (s, 1H), 4.28 (d, J=6.1 Hz, 1H), 4.19-4.10 (m, 1H), 3.53-3.35 (m, 3H), 1.24 (d, J=6.1 Hz, 1H), 0.73-0.66 (m, 1H), 0.59 (ddt, J=13.0, 9.0, 4.8 Hz, 2H), 0.47 (dt, J=10.4, 5.3 Hz, 1H).

TABLE 6 The following examples were prepared according to the synthetic procedure for Examples 38A and 38B, using the appropriate starting materials and reagents

| | | | Calc'd | Observed | |
|---|---|---|---|---|---|
| Example | Compound | Name | [M + H]⁺ | [M + H]⁺ | Conditions |
| 39A | 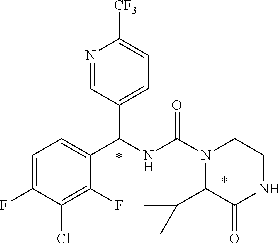 [SMILES: CC(C)C1C(=O)NCCN1C(=O)NC(c1ccc(C(F)(F)F)nc1)c1ccc(F)c(Cl)c1F] | N-((R or S)-(3-chloro- 2,4-difluorophenyl)(6- (trifluoromethyl)pyridin- 3-yl)methyl)-(R or S)-2-isopropyl-3-oxo- piperazine-1- carboxamide | 490.9 | 491.5 | SFC: AD-H Co-solvent: 35% EtOH peak 1 |
| 39B | 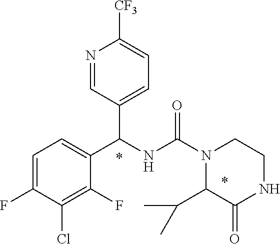 [SMILES: CC(C)C1C(=O)NCCN1C(=O)NC(c1ccc(C(F)(F)F)nc1)c1ccc(F)c(Cl)c1F] | N-((R or S)-(3-chloro- 2,4-difluorophenyl)(6- (trifluoromethyl)pyridin- 3-yl)methyl)-(S or R)-2-isopropyl-3-oxo- piperazine-1- carboxamide | 490.9 | 491.5 | SFC: AD-H Co-solvent: 35% EtOH peak 2 |
| 40A | 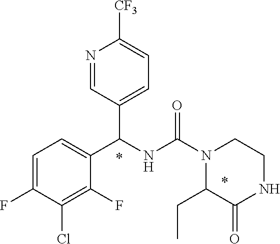 [SMILES: CCC1C(=O)NCCN1C(=O)NC(c1ccc(C(F)(F)F)nc1)c1ccc(F)c(Cl)c1F] | N-((R or S)-(3-chloro- 2,4-difluorophenyl)(6- (trifluoromethyl)pyridin- 3-yl)methyl)-(R or S)-2-ethyl-3- oxopiperazine-1- carboxamide | 476.8 | 477.5 | SFC: OJ-H Co-solvent: 20% MeOH peak 1 |
| 40B | 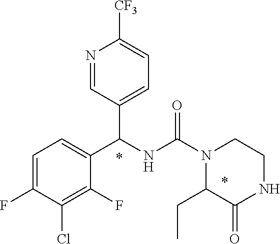 [SMILES: CCC1C(=O)NCCN1C(=O)NC(c1ccc(C(F)(F)F)nc1)c1ccc(F)c(Cl)c1F] | N-((R or S)-(3-chloro- 2,4-difluorophenyl)(6- (trifluoromethyl)pyridin- 3-yl)methyl)-(S or R)-2-ethyl-3- oxopiperazine-1- carboxamide | 476.8 | 477.5 | SFC: OJ-H Co-solvent: 20% MeOH peak 2 |
| 41A | 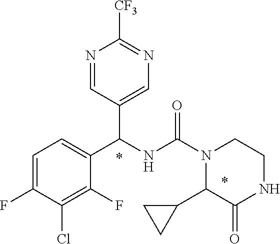 [SMILES: O=C1NCCN(C(=O)NC(c2cnc(C(F)(F)F)nc2)c2ccc(F)c(Cl)c2F)C1C1CC1] | N-((R or S)-(3-chloro- 2,4-difluorophenyl)(2- (trifluoromethyl) pyrimidin- 5-yl)methyl)-(R or S)-2-cyclopropyl-3- oxopiperazine-1- carboxamide | 489.8 | 490.4 | In step 2 SFC: OD-H Co-solvent: 15% EtOH peak 2 In step 4 SFC: OJ-H Co-solvent: 25% MeOH peak 1 |
| 41B | 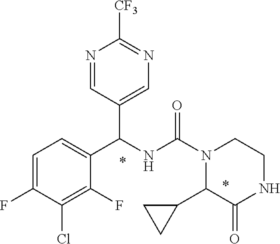 [SMILES: O=C1NCCN(C(=O)NC(c2cnc(C(F)(F)F)nc2)c2ccc(F)c(Cl)c2F)C1C1CC1] | N-((R or S)-(3-chloro- 2,4-difluorophenyl)(2- (trifluoromethyl) pyrimidin- 5-yl)methyl)-(S or R)-2-cyclopropyl-3- oxopiperazine-1- carboxamide | 489.8 | 490.4 | In step 2 SFC: OD-H Co-solvent: 15% EtOH peak 2 In step 4 SFC: OJ-H Co-solvent: 25% MeOH peak 2 |

### Example 42

### N—((R or S)-(3-chloro-4-fluorophenyl)(6-(trifluoromethyl)pyridin-2-yl)methyl)-3-oxopiperazine-1-carboxamide

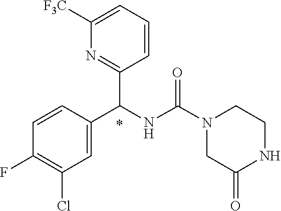 [SMILES: O=C1CN(C(=O)NC(c2ccc(F)c(Cl)c2)c2cccc(C(F)(F)F)n2)CCN1]

Step 1: (R,E)-N-(3-chloro-4-fluorobenzylidene)-2-methylpropane-2-sulfinamide Titanium(IV) ethoxide (1476 μl, 6.99 mmol), 3-chloro-4-fluorobenzaldehyde (554 mg, 3.49 mmol) and (R)-2-methylpropane-2-sulfinamide (423 mg, 3.49 mmol) were combined in a microwave tube. The mixture was microwaved at 70° C. for 20 minutes, then cooled to RT, and poured into 30 mL brine and 50 mL ethyl acetate. The mixture was filtered through a Celite® pad, and the separated organic layer was dried over Na₂SO₄, filtered and concentrated under reduced pressure to give the title compound. LRMS m/z (M+H): calculated 261.7, observed 262.2.

Step 2: (R)—N-((3-chloro-4-fluorophenyl)(6-(trifluoromethyl)pyridin-2-yl)methyl)-2-methylpropane-2-sulfinamide (Isomer B) To a solution of 2-iodo-6-(trifluoromethyl)pyridine (993 mg, 3.64 mmol) in THF (15 mL) at −78° C. was added isopropylmagnesium chloride-lithium chloride complex in THF (1.3 M, 2.97 mL, 3.87 mmol). The mixture was stirred at −78° C. for 2 hours and followed by the addition of (R,E)-N-(3-chloro-4-fluorobenzylidene)-2-methylpropane-2-sulfinamide (880 mg, 3.36 mmol). The mixture was warmed to 0° C. for 2 hours, then warmed to RT, quenched with saturated aqueous NH₄Cl, and extracted with diethyl ether. The separated organic layer was dried over Na₂SO₄, filtered and concentrated under reduced pressure to give a mixture, which was further separated by SFC (Whelk-1 column, 15% MeOH co-solvent) to give Isomer A (first eluted fraction) and Isomer B (second eluted fraction).

Step 3: (3-chloro-4-fluorophenyl)(6-(trifluoromethyl)pyridin-2-yl)methanamine hydrochloride To a solution of (R)—N—((R)-(3-chloro-4-fluorophenyl)(6-(trifluoromethyl)pyridin-2-yl)methyl)-2-methylpropane-2-sulfinamide (Isomer B, 706 mg, 1.727 mmol) in CH₂Cl₂ (10 mL) was added HCl in dioxane (4M, 2 mL, 8.00 mmol). The mixture was stirred at RT for 2 hours and then concentrated under reduced pressure. The resulting residue was washed with diethyl ether (2×10 mL) and filtered to give the title compound. LRMS m/z (M+H): calculated 304.7, observed 305.6.

Step 4: Example 42 To a solution of (R)-(3-chloro-4-fluorophenyl)(6-(trifluoromethyl)pyridin-2-yl)methanamine, HCl (15 mg, 0.044 mmol) in CH₂Cl₂ (1.5 mL) at 0° C. was added Et₃N (0.037 mL, 0.264 mmol) and triphosgene (13.05 mg, 0.044 mmol). The mixture was stirred at 0° C. for 1 hour, followed by the addition of piperazin-2-one (8.80 mg, 0.088 mmol). After stirring at 0° C. for 1 hour, the reaction was warmed to RT for 1 hour, and then concentrated under reduced pressure. The resulting residue that was purified by column chromatography on silica gel, eluting with (0-4% MeOH/DCM) to give Example 42: LRMS m/z (M+H): calculated 430.8, observed 431.4. ¹H NMR δ (ppm) (500 MHz, Chloroform-d): 7.88 (t, J=7.8 Hz, 1H), 7.65 (d, J=7.7 Hz, 1H), 7.42 (d, J=7.9 Hz, 1H), 7.39 (d, J=6.9 Hz, 1H), 7.23 (d, J=2.4 Hz, 1H), 7.10 (t, J=8.7 Hz, 1H), 6.09 (d, J=4.4 Hz, 1H), 4.23-4.04 (m, 2H), 3.80-3.71 (m, 1H), 3.67-3.58 (m, 1H), 3.42 (s, 2H).

TABLE 7 The following examples were prepared according to the synthetic procedure for Example 42, using the appropriate starting materials and reagents

| | | | Calc'd | Observed | |
|---|---|---|---|---|---|
| Example | Compound | Name | [M + H]⁺ | [M + H]⁺ | Conditions |
| 43 | 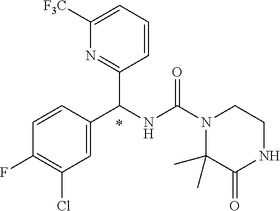 [SMILES: CC1(C)C(=O)NCCN1C(=O)NC(c1ccc(F)c(Cl)c1)c1cccc(C(F)(F)F)n1] | N-((R or S)-(3-chloro-4- fluorophenyl)(6- (trifluoromethyl)pyridin- 2-yl)methyl)-2,2- dimethyl-3-oxopiperazine- 1-carboxamide | 458.8 | 459.4 | In step 4 Using the same amine isomer as example 42 |
| 44 | 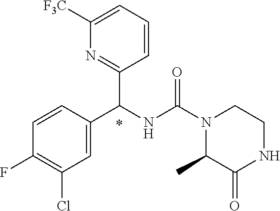 [SMILES: C[C@@H]1C(=O)NCCN1C(=O)NC(c1ccc(F)c(Cl)c1)c1cccc(C(F)(F)F)n1] | (2R)-N-((Ror S)-(3- chloro-4-fluoro-phenyl)(6- (trifluoro-methyl)pyridin- 2-yl)methyl)-2-methyl-3- oxopiperazine-1- carboxamide | 444.8 | 445.4 | In step 4 Using the same amine isomer as example 42 |
| 45 | 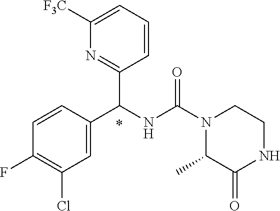 [SMILES: C[C@H]1C(=O)NCCN1C(=O)NC(c1ccc(F)c(Cl)c1)c1cccc(C(F)(F)F)n1] | (2S)-N-((R or S)-(3- chloro-4-fluoro-phenyl)(6- (trifluoro-methyl)pyridin- 2-yl)methyl)-2-methyl-3- oxopiperazine-1- carboxamide | 444.8 | 445.4 | In step 4 Using the same amine isomer as example 42 |
| 46A | 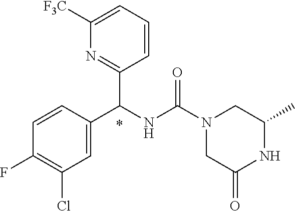 [SMILES: C[C@H]1CN(C(=O)NC(c2ccc(F)c(Cl)c2)c2cccc(C(F)(F)F)n2)CC(=O)N1] | (3S)-N-((R or S)-(3- chloro-4-fluoro-phenyl)(6- (trifluoro-methyl)pyridin- 2-yl)methyl)-3-methyl-5- oxopiperazine-1- carboxamide | 444.8 | 445.4 | In step 4 Using the same amine isomer as example 42 |
| 46B | 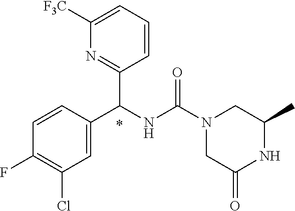 [SMILES: C[C@@H]1CN(C(=O)NC(c2ccc(F)c(Cl)c2)c2cccc(C(F)(F)F)n2)CC(=O)N1] | (3R)-N-((S or R)-(3- chloro-4-fluoro-phenyl)(6- (trifluoro-methyl)pyridin- 2-yl)methyl)-3-methyl-5- oxopiperazine-1- carboxamide | 444.8 | 445.4 | In step 4 Using the same amine isomer as example 42 |
| 47A | 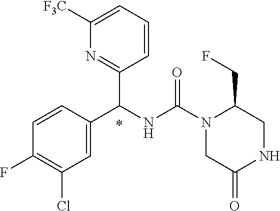 [SMILES: O=C1CN(C(=O)NC(c2ccc(F)c(Cl)c2)c2cccc(C(F)(F)F)n2)[C@@H](CF)CN1] | (2R)-N-((R or S)-(3- chloro-4-fluoro-phenyl)(6- (trifluoro-methyl)pyridin- 2-yl)methyl)-2- (fluoromethyl)-5- oxopiperazine-1- carboxamide | 462.8 | 463.1 | In step 4 Using the same amine isomer as example 42 |
| 47B | 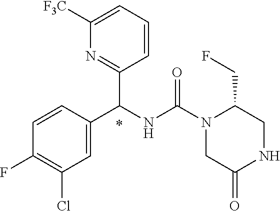 [SMILES: O=C1CN(C(=O)NC(c2ccc(F)c(Cl)c2)c2cccc(C(F)(F)F)n2)[C@H](CF)CN1] | (2S)-N-((R or S)-(3- chloro-4-fluoro-phenyl)(6- (trifluoro-methyl)pyridin- 2-yl)methyl)-2- (fluoromethyl)-5- oxopiperazine-1- carboxamide | 462.8 | 463.1 | In step 4 Using the same amine isomer as example 42 |

### Examples 48A and 48B

### (2R)—N—((R)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide and (2R)—N—((S)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide

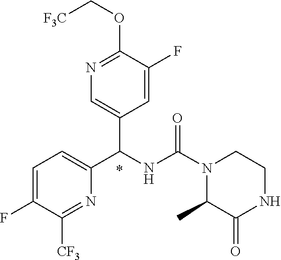 [SMILES: C[C@@H]1C(=O)NCCN1C(=O)NC(c1cnc(OCC(F)(F)F)c(F)c1)c1ccc(F)c(C(F)(F)F)n1]

Step 1: 3-fluoro-2-(trifluoromethyl)-6-vinylpyridine To a solution of 6-chloro-3-fluoro-2-(trifluoromethyl)pyridine (2.20 g, 11.03 mmol) in 1,4-dioxane (15 mL) were added Cs₂CO₃ (7.18 g, 22.05 mmol), vinylboronic acid pinacol ester (2.81 mL, 16.54 mmol) and water (200 μL). The mixture was purged with N₂ for 10 minutes, followed by the addition of 1,1′-bis(di-tert-butylphosphino) ferrocene palladium dichloride (0.359 g, 0.551 mmol). The reaction was heated to 80° C. and stirred for 20 hours, then quenched with H₂O and extracted with diethyl ether. The separated organic layer was dried over Na₂SO₄, filtered and concentrated under reduced pressure to give the title compound. LRMS m/z (M+H): calculated 191.1, observed 192.3.

Step 2: 5-fluoro-6-(trifluoromethyl)picolinaldehyde To a solution of 3-fluoro-2-(trifluoro-methyl)-6-vinylpyridine (2000 mg, 10.46 mmol) in THF (10 mL) at 0° C. was added sequentially water (1 mL), 2.5% osmium tetroxide in t-butanol (3.28 mL, 0.262 mmol), 2,6-dimethylpyridine (2.438 mL, 20.93 mmol) and sodium periodate (8953 mg, 41.9 mmol). The resulting mixture was then warmed to RT, stirred at RT for 3 hours, diluted with 30 mL diethyl ether and filtered. The filtrate was partitioned between diethyl ether and water. The separated organic layer was dried over Na₂SO₄, filtered and concentrated under reduced pressure to give the title compound.

Step 3: (R,E)-N-((5-fluoro-6-(trifluoromethyl)pyridin-2-yl)methylene)-2-methylpropane-2-sulfinamide To a solution of 5-fluoro-6-(trifluoromethyl)picolinaldehyde (2.0 g, 10.36 mmol) and (R)-2-methylpropane-2-sulfinamide (2.008 g, 16.57 mmol) in CH₂Cl₂ (20 mL) was added Cs₂CO₃ (5.40 g, 16.57 mmol). The mixture was stirred at RT for 2 hours and filtered through a Celite® pad to remove the solids. After washing with CH₂Cl₂, the combined filtrate was concentrated under reduced pressure. The resulting residue was purified by column chromatography on silica gel, eluting with (0-20% ethyl acetate/hexane) to give the title compound. LRMS m/z (M+H): calculated 296.3, observed 297.3.

Step 4: (R)—N-((5-fluoro-6-(2,2,2-trifluoroethoxy)pyridin-3-yl)(5-fluoro-6-(trifluoromethyl) pyridin-2-yl)methyl)-2-methylpropane-2-sulfinamide 3-Fluoro-5-iodo-2-(2,2,2-tri-fluoroethoxy)pyridine (399 mg. 1.242 mmol) was dissolved in anhydrous THF (4 mL) and cooled to −78° C., followed by the addition of isopropyl magnesium chloride-lithium chloride complex in THF (1.3 M, 1.015 mL, 1.320 mmol). The mixture was stirred at −78° C. for 30 minutes, then warmed to 0° C. Then (R,E)-N-((5-fluoro-6-(trifluoromethyl)pyridin-2-yl)methylene)-2-methylpropane-2-sulfinamide (230) mg, 0.776 mmol) in THF (1 mL) was added and the reaction was stirred at 0° C. for 1 hour, and then stirred at RT for 1 hour. The reaction mixture was partitioned between ethyl acetate and saturated aqueous NH₄Cl. The separated organic layer was dried over Na₂SO₄, filtered and concentrated under reduced pressure. The resulting residue was purified by column chromatography on silica gel, eluting with (0-20% ethyl acetate/hexane) to give the title compound. LRMS m/z (M+H): calculated 491.4, observed 492.5.

Step 5: (5-fluoro-6-(2,2,2-trifluoroethoxy)pyridin-3-yl)(5-fluoro-6-(trifluoromethyl)pyridin-2-yl)methanamine hydrochloride A solution of (R)—N-((5-fluoro-6-(2,2,2-trifluoroethoxy)pyridin-3-yl)(5-fluoro-6-(trifluoromethyl)pyridin-2-yl)methyl)-2-methylpropane-2-sulfinamide (180 mg, 0.366 mmol) in CH₂Cl₂ (1 mL) was cooled to (° C.), followed by the addition of HCl in 1,4-dioxane (4 M, 1 mL, 4.00 mmol). The reaction was stirred at 0° C. for 2 hours, then concentrated under reduced pressure. The resulting residue was washed with 2×5 mL hexane and filtered to give the title compound. LRMS m/z (M+H): calculated 387.2, observed 388.4.

Step 5: examples 48A and 48B To a solution of (5-fluoro-6-(2,2,2-trifluoroethoxy)pyridin-3-yl)(5-fluoro-6-(trifluoromethyl)pyridin-2-yl)methanamine, HCl (70 mg, 0.165 mmol) in CH₂Cl₂ (2 mL) at 0° C. were added Et₃N (92.14 mL, 0.661 mmol)) and triphosgene (49.0 mg, 0.165 mmol). The mixture was stirred at 0° C. for 1 hour, then (R)-3-methylpiperazin-2-one (28.3 mg, 0.248 mmol) was added. The reaction was stirred at 0° C. for 1 hour, then warmed to RT for 1 hour, and concentrated under reduced pressure. The resulting residue was purified by column chromatography on silica gel, eluting with (0)-4% MeOH/DCM) to give a mixture, which was further separated by SFC (OJ-H column, 10% EtOH with 0.2% DIPEA co-solvent) to give Examples 48A (first eluted fraction) and 48B (second eluted fraction).

Example 48A: LRMS m/z (M+H): calculated 527.4, observed 528.5. ¹H NMR δ (ppm) (500 MHz, Chloroform-d): 7.96 (d, J=2.0 Hz, 1H), 7.65 (t, J=8.9 Hz, 1H), 7.46 (dd, J=8.7, 3.4 Hz, 1H), 7.35 (dd, J=10.1, 2.0 Hz, 1H), 6.68 (d, J=5.9 Hz, 1H), 6.16 (d, J=5.7 Hz, 1H), 5.94 (s, 1H), 4.83 (dddd, J=20.9, 12.5, 8.4, 4.0 Hz, 2H), 4.55-4.49 (m, 1H), 4.24 (d, J=12.2 Hz, 1H), 3.56-3.48 (m, 1H), 3.35 (s, 1H), 3.29-3.20 (m, 1H), 1.52 (d, J=6.8 Hz, 3H).

Example 48B: LRMS m/z (M+H): calculated 527.4, observed 528.5. ¹H NMR δ (ppm) (500 MHz, Chloroform-d): 7.97 (d, J=2.0 Hz, 1H), 7.65 (t, J=8.9 Hz, 1H), 7.49 (dd, J=8.6, 3.4 Hz, 1H), 7.37 (dd, J=10.1, 2.0 Hz, 1H), 6.64 (d, J=5.9 Hz, 1H), 6.11 (d, J=5.8 Hz, 1H), 6.08 (s, 1H), 4.90-4.75 (m, 2H), 4.53 (q, J=7.0 Hz, 1H), 4.11 (d, J=13.6 Hz, 1H), 3.52 (td, J=11.2, 3.7 Hz, 1H), 3.36 (dq, J=11.6, 3.5 Hz, 1H), 3.28 (ddd, J=14.1, 10.8, 3.6 Hz, 1H), 1.56 (d, J=7.1 Hz, 3H).

TABLE 8 The following examples were prepared according to the synthetic procedure for Examples 48A and 48B, using the appropriate starting materials and reagents

| | | | Calc'd | Observed | |
|---|---|---|---|---|---|
| Example | Compound | Name | [M + H]⁺ | [M + H]⁺ | Conditions |
| 49A | 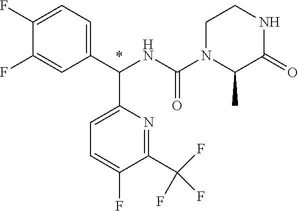 [SMILES: C[C@@H]1C(=O)NCCN1C(=O)NC(c1ccc(F)c(F)c1)c1ccc(F)c(C(F)(F)F)n1] | (2R)-N-((R or S)-(3,4- difluorophenyl)(5-fluoro- 6-(trifluoro- methyl)pyridin-2- yl)methyl)-2-methyl-3- oxopiperazine-1- carboxamide | 446.3 | 447.4 | SFC: AD-H Co-solvent: 15% (IPA + 0.2% DIPEA) peak 1 |
| 49B | 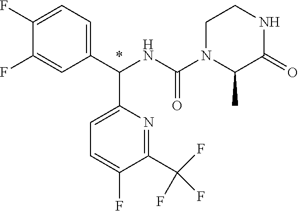 [SMILES: C[C@@H]1C(=O)NCCN1C(=O)NC(c1ccc(F)c(F)c1)c1ccc(F)c(C(F)(F)F)n1] | (2R)-N-((S or R)-(3- chloro-4-fluoro- phenyl)(6-(trifluoro- methyl)pyridin-2- yl)methyl)-2-methyl-3- oxopiperazine-1- carboxamide | 446.3 | 447.4 | SFC: AD-H Co-solvent: 15% (IPA + 0.2% DIPEA) peak 2 |
| 50A | 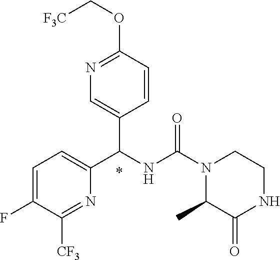 [SMILES: C[C@@H]1C(=O)NCCN1C(=O)NC(c1ccc(OCC(F)(F)F)nc1)c1ccc(F)c(C(F)(F)F)n1] | (2R)-N-((R or S)-(5- fluoro-6-(trifluoro- methyl)pyridin-2-yl)(6- (2,2,2-trifluoro- ethoxy)pyridin-3- yl)methyl)-2-methyl-3- oxopiperazine-1- carboxamide | 509.4 | 510.5 | silica gel preparative TLC (4% MeOH/DCM) polar fraction |
| 50B | 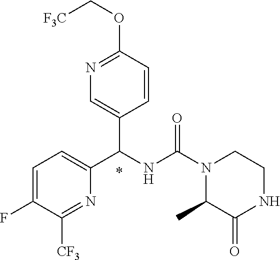 [SMILES: C[C@@H]1C(=O)NCCN1C(=O)NC(c1ccc(OCC(F)(F)F)nc1)c1ccc(F)c(C(F)(F)F)n1] | (2R)-N-((S or R)-(5- fluoro-6-(trifluoro- methyl)pyridin-2-yl)(6- (2,2,2-trifluoro- ethoxy)pyridin-3- yl)methyl)-2-methyl-3- oxopiperazine-1- carboxamide | 509.4 | 510.5 | silica gel preparative TLC (4% MeOH/DCM) less polar fraction |
| 51A | 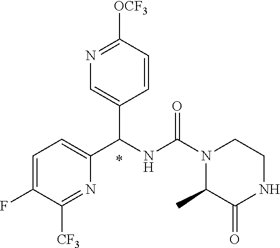 [SMILES: C[C@@H]1C(=O)NCCN1C(=O)NC(c1ccc(OC(F)(F)F)nc1)c1ccc(F)c(C(F)(F)F)n1] | (2R)-N-((R or S)-(5- fluoro-6- (trifluoromethyl)pyridin- 2-yl)(6-(trifluoro- methoxy)pyridin-3- yl)methyl)-2-methyl-3- oxopiperazine-1- carboxamide | 495.4 | 496.5 | silica gel preparative TLC (4% MeOH/DCM) polar fraction |
| 51B | 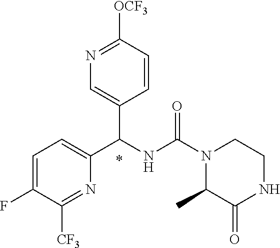 [SMILES: C[C@@H]1C(=O)NCCN1C(=O)NC(c1ccc(OC(F)(F)F)nc1)c1ccc(F)c(C(F)(F)F)n1] | (2R)-N-((S or R)-(5- fluoro-6-(trifluoro- methyl)pyridin-2-yl)(6- (trifluoromethoxy) pyridin-3-yl)methyl)-2- methyl-3-oxopiperazine- 1-carboxamide | 495.4 | 496.5 | silica gel preparative TLC (4% MeOH/DCM) less polar fraction |
| 52A | 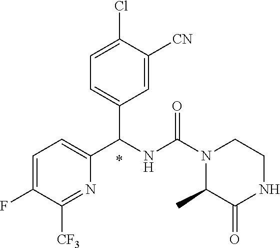 [SMILES: C[C@@H]1C(=O)NCCN1C(=O)NC(c1ccc(Cl)c(C#N)c1)c1ccc(F)c(C(F)(F)F)n1] | (2R)-N-((R or S)-(4- chloro-3-cyano- phenyl)(5-fluoro-6- (trifluoromethyl)pyridin- 2-yl)methyl)-2-methyl-3- oxopiperazine-1- carboxamide | 469.8 | 470.5 | SFC: OJ-H Co-solvent: 25% MeOH peak 1 |
| 52B | 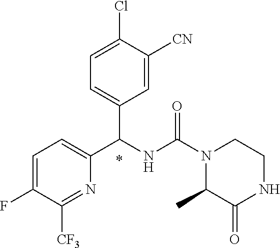 [SMILES: C[C@@H]1C(=O)NCCN1C(=O)NC(c1ccc(Cl)c(C#N)c1)c1ccc(F)c(C(F)(F)F)n1] | (2R)-N-((S or R)-(4- chloro-3-cyano- phenyl)(5-fluoro-6- (trifluoromethyl)pyridin- 2-yl)methyl)-2-methyl-3- oxopiperazine-1- carboxamide | 469.8 | 470.5 | SFC: OJ-H Co-solvent: 25% MeOH peak 2 |
| 53A | 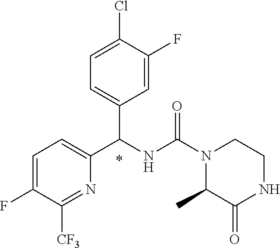 [SMILES: C[C@@H]1C(=O)NCCN1C(=O)NC(c1ccc(Cl)c(F)c1)c1ccc(F)c(C(F)(F)F)n1] | (2R)-N-((R or S)-(4- chloro-3-fluoro- phenyl)(5-fluoro-6- (trifluoromethyl)pyridin- 2-yl)methyl)-2-methyl-3- oxopiperazine-1- carboxamide | 462.8 | 463.5 | SFC: AD-H Co-solvent: 20% (IPA + 0.2% DIPEA) peak 1 |
| 53B | 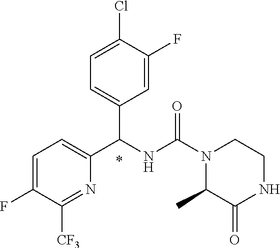 [SMILES: C[C@@H]1C(=O)NCCN1C(=O)NC(c1ccc(Cl)c(F)c1)c1ccc(F)c(C(F)(F)F)n1] | (2R)-N-((S or R)-(4- chloro-3-fluoro- phenyl)(5-fluoro-6- (trifluoromethyl)pyridin- 2-yl)methyl)-2-methyl-3- oxopiperazine-1- carboxamide | 462.8 | 463.5 | SFC: AD-H Co-solvent: 20% (IPA + 0.2% DIPEA) peak 2 |
| 54A | 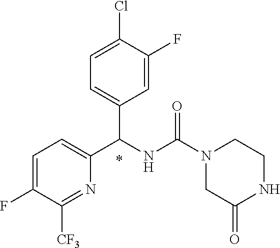 [SMILES: O=C1CN(C(=O)NC(c2ccc(Cl)c(F)c2)c2ccc(F)c(C(F)(F)F)n2)CCN1] | N-((R or S)-(4-chloro-3- fluorophenyl)(5-fluoro-6- (trifluoromethyl)pyridin- 2-yl)methyl)-3- oxopiperazine-1- carboxamide | 448.8 | 449.5 | SFC: OJ-H Co-solvent: 15% EtOH peak 1 |
| 54B | 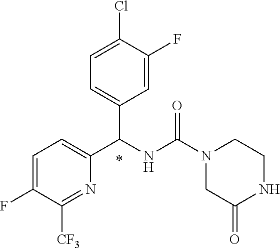 [SMILES: O=C1CN(C(=O)NC(c2ccc(Cl)c(F)c2)c2ccc(F)c(C(F)(F)F)n2)CCN1] | N-((S or R)-(4-chloro-3- fluorophenyl)(5-fluoro-6- (trifluoromethyl)pyridin- 2-yl)methyl)-3- oxopiperazine-1- carboxamide | 448.8 | 449.5 | SFC: OJ-H Co-solvent: 15% EtOH peak 2 |
| 55A | 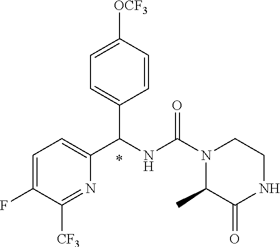 [SMILES: C[C@@H]1C(=O)NCCN1C(=O)NC(c1ccc(OC(F)(F)F)cc1)c1ccc(F)c(C(F)(F)F)n1] | (2R)-N-((R or S)-(5- fluoro-6-(trifluoro- methyl)pyridin-2-yl)(4- (trifluoromethoxy)phenyl) methyl)-2-methyl-3- oxopiperazine-1- carboxamide | 494.4 | 495.5 | silica gel preparative TLC (4% MeOH/ DCM) less polar fraction |
| 55B | 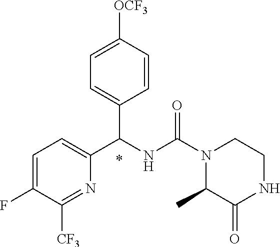 [SMILES: C[C@@H]1C(=O)NCCN1C(=O)NC(c1ccc(OC(F)(F)F)cc1)c1ccc(F)c(C(F)(F)F)n1] | (2R)-N-((S or R)-(5- fluoro-6-(trifluoro- methyl)pyridin-2-yl)(4- (trifluoromethoxy)phenyl) methyl)-2-methyl-3- oxopiperazine-1- carboxamide | 494.4 | 495.5 | silica gel preparative TLC (4% MeOH/ DCM) polar fraction |
| 56A | 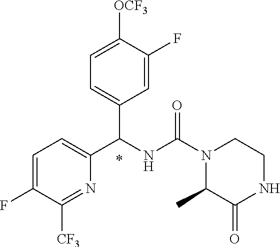 [SMILES: C[C@@H]1C(=O)NCCN1C(=O)NC(c1ccc(OC(F)(F)F)c(F)c1)c1ccc(F)c(C(F)(F)F)n1] | (2R)-N-((R or S)-(3- fluoro-4-(trifluoro- methoxy)phenyl)(5- fluoro-6-(trifluoro- methyl)pyridin-2- yl)methyl)-2-methyl-3- oxopiperazine-1- carboxamide | 512.4 | 513.5 | silica gel preparative TLC (4% MeOH/ DCM) polar fraction |
| 56B | 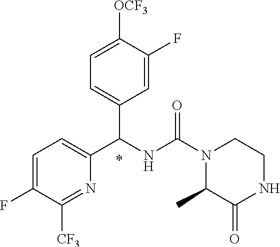 [SMILES: C[C@@H]1C(=O)NCCN1C(=O)NC(c1ccc(OC(F)(F)F)c(F)c1)c1ccc(F)c(C(F)(F)F)n1] | (2R)-N-((S or R)-(3- fluoro-4-(trifluoro- methoxy)phenyl)(5- fluoro-6-(trifluoro- methyl)pyridin-2- yl)methyl)-2-methyl-3- oxopiperazine-1- carboxamide | 512.4 | 513.5 | silica gel preparative TLC (4% MeOH/ DCM) less polar fraction |
| 57A | 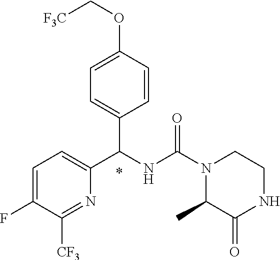 [SMILES: C[C@@H]1C(=O)NCCN1C(=O)NC(c1ccc(OCC(F)(F)F)cc1)c1ccc(F)c(C(F)(F)F)n1] | (2R)-N-((R or S)-(5- fluoro-6-(trifluoro- methyl)pyridin-2-yl)(4- (2,2,2-trifluoro- ethoxy)phenyl)methyl)-2- methyl-3-oxopiperazine- 1-carboxamide | 508.4 | 509.5 | silica gel preparative TLC (3% MeOH/ DCM) less polar fraction |
| 57B | 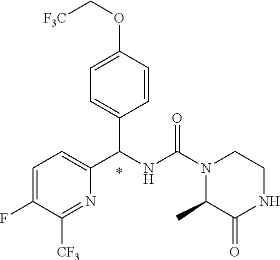 [SMILES: C[C@@H]1C(=O)NCCN1C(=O)NC(c1ccc(OCC(F)(F)F)cc1)c1ccc(F)c(C(F)(F)F)n1] | (2R)-N-((S or R)-(5- fluoro-6-(trifluoro- methyl)pyridin-2-yl)(4- (2,2,2-trifluoro- ethoxy)phenyl)methyl)-2- methyl-3-oxopiperazine- 1-carboxamide | 508.4 | 509.5 | silica gel preparative TLC (3% MeOH/ DCM) polar fraction |
| 58A | 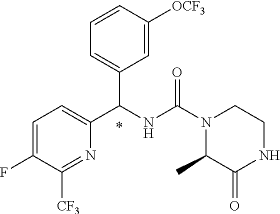 [SMILES: C[C@@H]1C(=O)NCCN1C(=O)NC(c1cccc(OC(F)(F)F)c1)c1ccc(F)c(C(F)(F)F)n1] | (2R)-N-((R or S)-(5- fluoro-6-(trifluoro- methyl)pyridin-2-yl)(3- (trifluoromethoxy)phenyl) methyl)-2-methyl-3- oxopiperazine-1- carboxamide | 494.4 | 495.5 | silica gel preparative TLC (4% MeOH/ DCM) polar fraction |
| 58B | 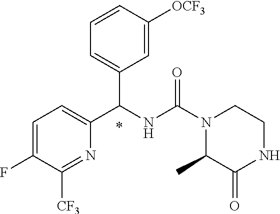 [SMILES: C[C@@H]1C(=O)NCCN1C(=O)NC(c1cccc(OC(F)(F)F)c1)c1ccc(F)c(C(F)(F)F)n1] | (2R)-N-((S or R)-(5- fluoro-6-(trifluoro- methyl)pyridin-2-yl)(3- (trifluoromethoxy)phenyl) methyl)-2-methyl-3- oxopiperazine-1- carboxamide | 494.4 | 495.5 | silica gel preparative TLC (4% MeOH/ DCM) less polar fraction |
| 59A | 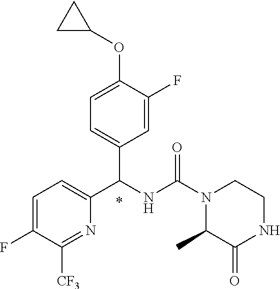 [SMILES: C[C@@H]1C(=O)NCCN1C(=O)NC(c1ccc(OC2CC2)c(F)c1)c1ccc(F)c(C(F)(F)F)n1] | (2R)-N-((R or S)-(4- cyclopropoxy-3- fluorophenyl)(5-fluoro-6- (trifluoromethyl)pyridin- 2-yl)methyl)-2-methyl-3- oxopiperazine-1- carboxamide | 484.4 | 485.6 | silica gel preparative TLC (4% MeOH/ DCM) less polar fraction |
| 59B | 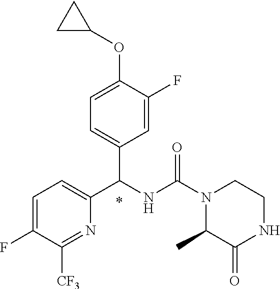 [SMILES: C[C@@H]1C(=O)NCCN1C(=O)NC(c1ccc(OC2CC2)c(F)c1)c1ccc(F)c(C(F)(F)F)n1] | (2R)-N-((S or R)-(4- cyclopropoxy-3- fluorophenyl)(5-fluoro-6- (trifluoromethyl)pyridin- 2-yl)methyl)-2-methyl-3- oxopiperazine-1- carboxamide | 484.4 | 485.6 | silica gel preparative TLC (4% MeOH/ DCM) polar fraction |
| 60A | 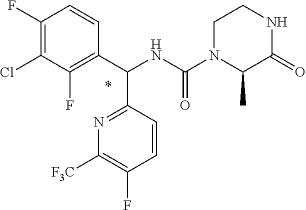 [SMILES: C[C@@H]1C(=O)NCCN1C(=O)NC(c1ccc(F)c(C(F)(F)F)n1)c1ccc(F)c(Cl)c1F] | (2R)-N-((R or S)-(3- chloro-2,4-difluoro- phenyl)(5-fluoro-6- (trifluoromethyl)pyridin- 2-yl)methyl)-2-methyl-3- oxopiperazine-1- carboxamide | 480.1 | 481.1 | SFC: OJ-H Co-solvent: 20% (EtOH with 0.1%NH₃• H₂O) peak 1 |
| 60B | 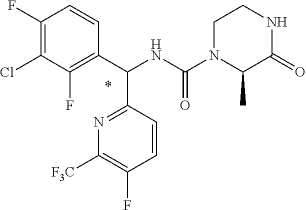 [SMILES: C[C@@H]1C(=O)NCCN1C(=O)NC(c1ccc(F)c(C(F)(F)F)n1)c1ccc(F)c(Cl)c1F] | (2R)-N-((S or R)-(3- chloro-2,4-difluoro- phenyl)(5-fluoro-6- (trifluoromethyl)pyridin- 2-yl)methyl)-2-methyl-3- oxopiperazine-1- carboxamide | 484.4 | 485.6 | SFC: OJ-H Co-solvent: 20% (EtOH with 0.1%NH₃• H₂O) peak 2 |

### Examples 61A and 61B

### (2R)—N—((R)-(5-cyano-6-(trifluoromethyl)pyridin-2-yl)(3-fluoro-4-(trifluoromethoxy)phenyl)methyl)-2-methyl-3-oxopiperazine-1-carboxamide and (2R)—N—((S)-(5-cyano-6-(trifluoromethyl)pyridin-2-yl)(3-fluoro-4-(trifluoromethoxy)phenyl)methyl)-2-methyl-3-oxopiperazine-1-carboxamide

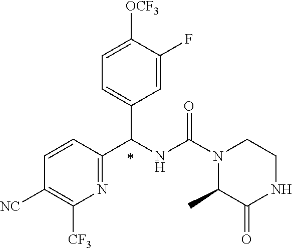 [SMILES: C[C@@H]1C(=O)NCCN1C(=O)NC(c1ccc(OC(F)(F)F)c(F)c1)c1ccc(C#N)c(C(F)(F)F)n1]

Step 1: 5-chloro-N-methoxy-N-methyl-6-(trifluoromethyl)picolinamide To a solution of 5-chloro-6-(trifluoromethyl)picolinic acid (1.0 g, 4.43 mmol) in CH₂Cl₂ (15 mL) were added N,O-dimethylhydroxylamine HCl (0.649 g, 6.65 mmol), HATU (2.53 g, 6.65 mmol) and DIPEA (2.323 mL, 13.30 mmol). The resulting mixture was stirred at RT for 20 hours and then concentrated under reduced pressure. The resulting residue was purified by column chromatography on silica gel, eluting with (0)-3% MeOH/DCM) to give the title compound. LRMS m/z (M+H): calculated 268.6, observed 269.3.

Step 2: 5-cyano-N-methoxy-N-methyl-6-(trifluoromethyl)picolinamide To a solution of 5-chloro-N-methoxy-N-methyl-6-(trifluoromethyl)picolinamide (320 mg, 1.191 mmol) in DMA (5 ml) under N₂ were added zinc cyanide (280 mg, 2.383 mmol) and (2-dicyclohexylphosphino-2′,6′-dimethoxy biphenyl) [2-(2′-amino-1,1′-biphenyl)]palladium(ii) methanesulfonate (93 mg, 0.119 mmol). The mixture was heated to 100° C. and stirred for 20 hours, then cooled to RT and partitioned between ethyl acetate and water. The separated organic layer was dried over Na₂SO₄, filtered and concentrated under reduce pressure. The resulting residue was purified by column chromatography on silica gel, eluting with (0)-40% ethyl acetate/hexane) to give the title compound. LRMS m/z (M+H): calculated 259.2, observed 260.3.

Step 3: 6-(3-fluoro-4-(trifluoromethoxy)benzoyl)-2-(trifluoromethyl)nicotinonitrile A solution of 4-bromo-2-fluoro-1-(trifluoromethoxy)benzene (385 mg, 1.487 mmol) in anhydrous THF (5 mL) was purged with N₂ for 10 minutes, followed by he addition of isopropylmagnesium chloride lithium chloride complex in THF (1.3 M, 1.144 mL, 1.487 mmol). The mixture, under N₂, was heated to 40° C. and stirred for 1 hour. Then the mixture was cooled to 0° C. and 5-cyano-N-methoxy-N-methyl-6-(trifluoromethyl)picolinamide (257 mg, 0.992 mmol) was added in one portion. The reaction was stirred at 0° C. under N₂ for 3 hours, then quenched with 30 mL saturated aqueous NH₄Cl, and extracted with diethyl ether (2×20 mL). The combined organic layers were dried over Na₂SO₄, filtered and concentrated under reduced pressure to give the title compound. LRMS m/z (M+H): calculated 378.2, observed 379.0.

Step 4: (R,E)-N-((5-cyano-6-(trifluoromethyl)pyridin-2-yl)(3-fluoro-4-(trifluoromethoxy)phenyl) methylene)-2-methylpropane-2-sulfinamide To a solution of 6-(3-fluoro-4-(trifluoromethoxy)-benzoyl)-2-(trifluoromethyl)nicotinonitrile (370 mg, 0.978 mmol) and (R)-2-methylpropane-2-sulfinamide (237 mg, 1.957 mmol) in toluene (1 mL) was added titanium(IV) ethoxide (0.410 mL, 1.957 mmol). The mixture was heated to 100° C., stirred for 1 hour, and then cooled to RT. H₂O (20 mL) and diethyl ether (50 mL) were added, and the resulting mixture was stirred for 10 minutes, and then filtered through a Celite®; pad. The organic layer was separated, dried over Na₂SO₄, filtered and concentrated under reduced pressure to give the title compound. LRMS m z (M+H): calculated 481.4, observed 482.4.

Step 5: (R)—N-((5-cyano-6-(trifluoromethyl)pyridin-2-yl)(3-fluoro-4-(trifluoromethoxy)phenyl) methyl)-2-methylpropane-2-sulfinamide A solution of (R,E)-N-((5-cyano-6-(trifluoromethyl)pyridin-2-yl)(3-fluoro-4-(trifluoromethoxy)phenyl) methylene)-2-methylpropane-2-sulfinamide (471 mg, 0.978 mmol) in THF (8 mL) and water (0.5 mL) was cooled to 0° C., followed by the addition of NaBH₄ (111 mg, 2.94 mmol). The mixture was stirred at 0° C. for 3 hours and then partitioned between diethyl ether and saturated aqueous NaHCO₃. The separated organic layer was dried over Na₂SO₄, filtered and concentrated under reduced pressure. The resulting residue was purified by column chromatography on silica gel, eluting with (0-50% ethyl acetate/hexane) to give the title compound. LRMS m/z (M+H): calculated 483.4, observed 484.4.

Step 6: 6-(amino(3-fluoro-4-(trifluoromethoxy)phenyl)methyl)-2-(trifluoromethyl)nicotinonitrile hydrochloride To a solution of (R)—N-((5-cyano-6-(trifluoromethyl)pyridin-2-yl)(3-fluoro-4-(trifluoromethoxy) phenyl)methyl)-2-methylpropane-2-sulfinamide (100 mg, 0.207 mmol) in CH₂Cl₂ (500 μL) was added HCl in 1,4-dioxane (4 M, 500 μL, 2.000 mmol). The mixture was stirred at RT for 2 hours and then concentrated under reduced pressure. The resulting residue was washed with hexane (2×5 ml) and filtered to give the title compound. LRMS m/z (M+H): calculated 379.2, observed 380.2.

Step 7: Examples 61A and 61B To a solution of 6-(amino(3-fluoro-4-(trifluoromethoxy)-phenyl)methyl)-2-(trifluoromethyl) nicotinonitrile HCl (80 mg, 0.192 mmol) in anhydrous acetonitrile (1.5 mL) was added CDI (62.4 mg, 0.385 mmol). The reaction was stirred at RT under N₂ for 1 hour, followed by the addition of (R)-3-methylpiperazin-2-one (43.9 mg, 0.385 mmol). The reaction mixture was stirred at RT for 2 hours, then concentrated under reduced pressure. The resulting residue was purified by silica gel preparative TLC, eluting with (4% MeOH/DCM), to give two fractions: Example 61A (less polar fraction): LRMS m/z (M+H): calculated 519.4, observed 520.5. ¹H NMR δ (ppm) (500 MHz, Chloroform-d): 8.19 (d, J=8.2 Hz, 1H), 7.61 (d, J=8.2 Hz, 1H), 7.32 (t, J=7.8 Hz, 1H), 7.22 (dd, J=10.2, 2.0 Hz, 1H), 7.17 (d, J=8.5 Hz, 1H), 6.53 (d, J=5.9 Hz, 1H), 6.42 (s, 1H), 6.27 (d, J=6.1 Hz, 1H), 4.53 (q, J=7.0 Hz, 1H), 4.23 (d, J=13.6 Hz, 1H), 3.51 (td, J=12.1, 11.4, 3.9 Hz, 1H), 3.41-3.30 (m, 1H), 3.24 (ddd, J=14.1, 11.1, 3.5 Hz, 1H), 1.60 (d, J=7.0 Hz, 3H); and

Example 61B (polar fraction): LRMS m/z (M+H): calculated 519.4, observed 520.5. ¹H NMR δ (ppm) (500 MHz, Chloroform-d): 8.20 (d, J=8.1 Hz, 1H), 7.63 (d, J=8.2 Hz, 1H), 7.32 (t, J=7.7 Hz, 1H), 7.21 (dd, J=10.2, 1.9 Hz, 1H), 7.19-7.16 (m, 1H), 6.46 (d, J=5.2 Hz, 1H), 6.26 (s, 1H), 6.21 (d, J=5.9 Hz, 1H), 4.53 (q, J=7.0 Hz, 1H), 4.12 (d, J=13.3 Hz, 1H), 3.52 (td, J=12.0, 11.4, 3.9 Hz, 1H), 3.41-3.34 (m, 1H), 3.29 (ddd, J=14.0, 10.8, 3.5 Hz, 1H), 1.56 (d, J=7.1 Hz, 3H).

### Examples 62A and 62B

### (2R)—N—((R)-(3-chloro-2,4-difluorophenyl)(2-(trifluoromethyl)thiazol-5-yl)methyl)-2-methyl-3-oxopiperazine-1-carboxamide and (2R)—N—((S)-(3-chloro-2,4-difluorophenyl)(2-(trifluoromethyl)thiazol-5-yl)methyl)-2-methyl-3-oxopiperazine-1-carboxamide

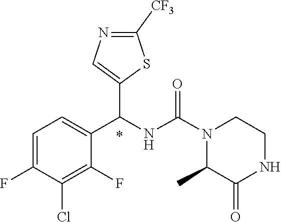 [SMILES: C[C@@H]1C(=O)NCCN1C(=O)NC(c1cnc(C(F)(F)F)s1)c1ccc(F)c(Cl)c1F]

Step 1: (R,E)-2-methyl-N-((2-(trifluoromethyl)thiazol-5-yl)methylene)propane-2-sulfinamide To a solution of (R)-(+)-2-methyl-2-propanesulfinamide (0.803 g, 6.62 mmol) and 2-(trifluoromethyl)-1,3-thiazole-5-carbaldehyde (1.0 g, 5.52 mmol) in toluene (4 mL) was added titanium(IV) ethoxide (2.315 mL, 11.04 mmol). The mixture was heated to 80° C. for 3 hours and then cooled to RT, followed by the addition of H₂O (30 mL) and ethyl acetate (50 mL). The mixture was stirred for 10 min, and then filtered through a Celite® pad. The separated organic layer was dried over Na₂SO₄, filtered and concentrated under reduced pressure to give the title compound. LRMS m/z (M+H): calculated 284.3, observed 285.3.

Step 2: (R)—N-((3-chloro-2,4-difluorophenyl)(2-(trifluoromethyl)thiazol-5-yl)methyl)-2-methylpropane-2-sulfinamide To a solution of 2-chloro-1,3-difluoro-4-iodobenzene (420 mg, 1.530 mmol) in anhydrous THF (3 mL) at 0° C. was added isopropylmagnesium chloride lithium chloride complex in THF (1.3M, 1.177 mL, 1.530 mmol). The mixture was stirred at 0° C. for 1 hour, followed by the addition of (R,E)-2-methyl-N-((2-(trifluoromethyl)thiazol-5-yl)methylene) propane-2-sulfinamide (290 mg, 1.020 mmol). The reaction mixture was stirred at 0° C. for 1 hour, then warmed to RT for 1 hour. The reaction was then quenched with saturated aqueous NH₄Cl and extracted with diethyl ether. The separated organic layer was dried over Na₂SO₄, filtered and concentrated under reduced pressure to give the title compound. LRMS m/z (M+H): calculated 432.9, observed 433.4.

Step 3: (3-chloro-2,4-difluorophenyl)(2-(trifluoromethyl)thiazol-5-yl)methanamine hydrochloride To a solution of (R)—N-((3-chloro-2,4-difluorophenyl)(2-(trifluoromethyl)thiazol-5-yl)methyl)-2-methylpropane-2-sulfinamide (442 mg, 1.021 mmol) in CH₂Cl₂ (1 mL) and MeOH (0.2 mL) was added HCl in 1,4-dioxane (4 M, 1 mL, 4.00 mmol). The mixture was stirred at RT for 30 minutes and then concentrated under reduced pressure. The resulting residue was washed with 2×5 mL diethyl ether, and filtered to give the title compound. LRMS m/z (M+H): calculated 328.7, observed 329.3.

Step 4: Example 62A and 62B To a solution of (3-chloro-2,4-difluorophenyl)(2-(trifluoro-methyl)thiazol-5-yl)methanamine HCl (180 mg, 0.493 mmol) in CH₂Cl₂ (8 mL) at 0° C. was added triphosgene (146 mg, 0.493 mmol) and Et₃N (0.069 mL, 0.493 mmol). The mixture was stirred at 0° C. for 1 hour, and then (R)-3-methylpiperazin-2-one (73.2 mg, 0.641 mmol) was added. After stirring at 0° C. for 1 additional hour, the reaction warmed to RT and stirred at RT for 1 hour. Then the reaction mixture was concentrated under reduced pressure. The resulting residue was purified by column chromatography on silica gel, eluting with (0-4% MeOH/DCM), to give a mixture, which was further separated by SFC (OJ-H column, 10% MeOH co-solvent) to give Examples 62A (first eluted fraction) and 62B (second eluted fraction).

Example 62A: LRMS m/z (M+H): calculated 468.8, observed 469.4. ¹H NMR δ (ppm) (500 MHz, Chloroform-d): 7.59 (s, 1H), 7.36 (q, J=8.0 Hz, 1H), 7.08 (t, J=8.3 Hz, 1H), 6.62 (d, J=7.9 Hz, 1H), 6.32 (s, 1H), 5.81 (s, 1H), 4.48 (s, 1H), 4.24 (d, J=13.0 Hz, 1H), 3.63 (s, 1H), 3.49 (d, J=15.6 Hz, 1H), 3.35 (d, J=10.8 Hz, 1H), 1.53 (d, J=6.1 Hz, 3H).

Example 62B: LRMS m/z (M+H): calculated 468.8, observed 469.4. ¹H NMR δ (ppm) (500 MHz, Chloroform-d): 7.64 (s, 1H), 7.43-7.35 (m, 1H), 7.12-7.04 (m, 1H), 6.65 (d, J=7.8 Hz, 1H), 6.28 (s, 1H), 6.06 (s, 1H), 4.55 (q, J=7.0 Hz, 1H), 4.24 (d, J=13.2 Hz, 1H), 3.54-3.45 (m, 1H), 3.37-3.28 (m, 1H), 3.19 (ddd, J=14.3, 11.3, 3.7 Hz, 1H), 1.47 (d, J=7.0 Hz, 3H).

TABLE 9 The following examples were prepared according to the synthetic procedure for Examples 62A and 62B, using the appropriate starting materials and reagents

| | | | Calc'd | Observed | |
|---|---|---|---|---|---|
| Example | Compound | Name | [M + H]⁺ | [M + H]⁺ | Conditions |
| 63A | 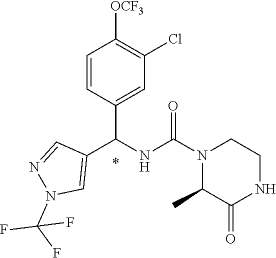 [SMILES: C[C@@H]1C(=O)NCCN1C(=O)NC(c1ccc(OC(F)(F)F)c(Cl)c1)c1cnn(C(F)(F)F)c1] | (2R)-N-((R or S)-(3- chloro-4-(trifluoro- methoxy)phenyl)(1- (trifluoromethyl)-1H- pyrazol-4-yl)methyl)-2- methyl-3-oxopiperazine- 1-carboxamide | 499.8 | 500.4 | SFC: OJ-H Co-solvent: 25% MeOH peak 1 |
| 63B | 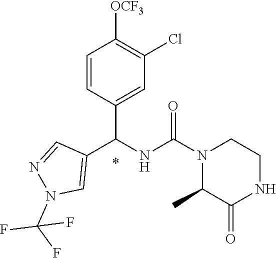 [SMILES: C[C@@H]1C(=O)NCCN1C(=O)NC(c1ccc(OC(F)(F)F)c(Cl)c1)c1cnn(C(F)(F)F)c1] | (2R)-N-((S or R)-(3- chloro-4-(trifluoro- methoxy)phenyl)(1- (trifluoromethyl)-1H- pyrazol-4-yl)methyl)-2- methyl-3-oxopiperazine- 1-carboxamide | 499.8 | 500.4 | SFC: OJ-H Co-solvent: 25% MeOH peak 2 |
| 64A | 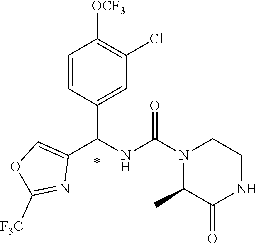 [SMILES: C[C@@H]1C(=O)NCCN1C(=O)NC(c1ccc(OC(F)(F)F)c(Cl)c1)c1coc(C(F)(F)F)n1] | (2R)-N-((R or S)-(3- chloro-4-(trifluoro- methoxy)phenyl)(2- (trifluoromethyl)oxazol- 4-yl)methyl)-2-methyl- 3-oxopiperazine-1- carboxamide | 500.8 | 501.4 | SFC: OJ-H Co-solvent: 10% MeOH peak 1 |
| 64B | 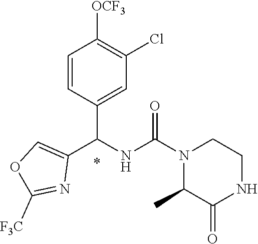 [SMILES: C[C@@H]1C(=O)NCCN1C(=O)NC(c1ccc(OC(F)(F)F)c(Cl)c1)c1coc(C(F)(F)F)n1] | (2R)-N-((S or R)-(3- chloro-4-(trifluoro- methoxy)phenyl)(2- (trifluoromethyl)oxazol- 4-yl)methyl)-2-methyl- 3-oxopiperazine-1- carboxamide | 500.8 | 501.4 | SFC: OJ-H Co-solvent: 10% MeOH peak 2 |
| 65A | 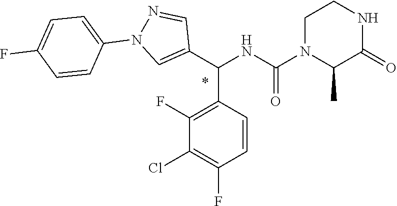 [SMILES: C[C@@H]1C(=O)NCCN1C(=O)NC(c1cnn(-c2ccc(F)cc2)c1)c1ccc(F)c(Cl)c1F] | (2R)-N-((R or S)-(3- chloro-2,4-difluoro- phenyl)(1-(4-fluoro- phenyl)-1H-pyrazol-4- yl)methyl)-2-methyl-3- oxopiperazine-1- carboxamide | 477.9 | 478.5 | SFC: OJ-H Co-solvent: 40% MeOH peak 1 |
| 65B | 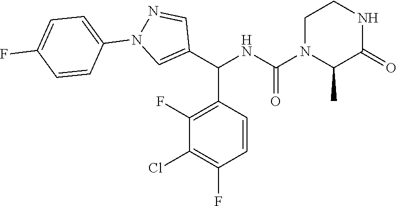 [SMILES: C[C@@H]1C(=O)NCCN1C(=O)NC(c1cnn(-c2ccc(F)cc2)c1)c1ccc(F)c(Cl)c1F] | (2R)-N-((S or R)-(3- chloro-2,4-difluoro- phenyl)(1-(4-fluoro- phenyl)-1H-pyrazol-4- yl)methyl)-2-methyl-3- oxopiperazine-1- carboxamide | 477.9 | 478.5 | SFC: OJ-H Co-solvent: 40% MeOH peak 2 |

### Examples 66A and 66B

### (2R)—N—((R)-(3-chloro-4-fluorophenyl)(1-(difluoromethyl)-1H-pyrazol-3-yl)methyl)-2-methyl-3-oxopiperazine-1-carboxamide and (2R)—N—((S)-(3-chloro-4-fluorophenyl)(1-(difluoromethyl)-1H-pyrazol-3-yl)methyl)-2-methyl-3-oxopiperazine-1-carboxamide

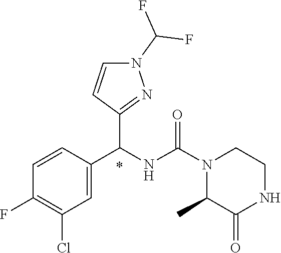 [SMILES: C[C@@H]1C(=O)NCCN1C(=O)NC(c1ccc(F)c(Cl)c1)c1ccn(C(F)F)n1]

Step 1: 1-(difluoromethyl)-N-methoxy-N-methyl-1H-pyrazole-3-carboxamide To a solution of 1-(difluoromethyl)-1H-pyrazole-3-carboxylic acid (1.4 g, 8.64 mmol) in DCM (30 mL) was added CDI (1.400 g, 8.64 mmol) at 20° C. The reaction was stirred for 1 hour, then TEA (2.408 mL, 17.27 mmol) and N,O-dimethylhydroxylamine hydrochloride (0.842 g, 8.64 mmol) were added. The resulting mixture was stirred at 20° C. for another 12 h, then water (30 mL) was added and the mixture was extracted with DCM (2×5 mL). The combined organic layers were dried over Na₂SO₄, filtered and concentrated under reduced pressure. The resulting residue was purified by flash silica gel chromatography (ISCO®: 20 g SepaFlash® Silica Flash Column, eluent of 38% ethyl acetate/pet. ether) to give the title compound. LRMS m/z (M+H): calculated 205.1, observed 206.0.

Step 2: (3-chloro-4-fluorophenyl)(1-(difluoromethyl)-1H-pyrazol-3-yl)methanone To a solution of 1-(difluoromethyl)-N-methoxy-N-methyl-1H-pyrazole-3-carboxamide (500 mg, 2.437 mmol) in THF (10 mL) was added (4-chloro-3-fluorophenyl)magnesium bromide in THF (0.5 M, 14.62 mL, 7.31 mmol) at −78° C. over 5 minutes. Then the mixture was stirred at 20° C. for 1 hour, followed by the addition of saturated aqueous NH₄Cl (5 mL) and extractraction with ethyl acetate. The combined organic layers was dried over Na₂SO₄, filtered and concentrated under reduced pressure. The resulting residue was purified by flash silica gel chromatography (ISCO®; 12 g SepaFlash® Silica Flash Column, eluent of 2.9% ethyl acetate/petroleum ether) to give the title compound. ¹H NMR (500 MHz, CD₃OD) δ 8.44-8.50 (m, 1H), 8.31-8.39 (m, 1H), 8.20-8.26 (m, 1H), 7.55-7.81 (m, 1H), 7.42 (t, J=9.0 Hz, 1H), 7.09-7.15 (m, 1H).

Step 3: (3-chloro-4-fluorophenyl)(1-(difluoromethyl)-1H-pyrazol-3-yl)methanamine To a mixture of (3-chloro-4-fluorophenyl)(1-(difluoromethyl)-1H-pyrazol-3-yl)methanone (500 mg, 1.821 mmol) and ammonium acetate (2105 mg, 27.3 mmol) in EtOH (8 mL) was added sodium cyanoborohydride (172 mg, 2.73 mmol) at 20° C. The mixture was stirred under microwaves (Biotage Initiator) at 130° C. for 10 min. Then the reaction mixture was concentrated to remove most of the EtOH, treated with 2 N NaOH until pH>10, and extracted with ethyl acetate (3×15 mL). The combined organic layers were dried over Na₂SO₄, filtered and concentrated under reduced pressure to give the title compound. LRMS m/z (M+H): calculated 275.6, observed 276.3.

Step 4: Examples 66A and 66B A mixture of CDI (176 mg, 1.088 mmol) and (3-chloro-4-fluorophenyl)(1-(difluoromethyl)-1H-pyrazol-3-yl)methanamine (150 mg, 0.544 mmol) in DMF (2 mL) was stirred at 20° C. for 1 h. Then (R)-3-methylpiperazin-2-one (74.5 mg, 0.653 mmol) was added. The resulting mixture was stirred at 20° C. for 1 h. Then the resulting solid was filtered off and the filtrate was purified by Prep-HPLC (64:36 to 34:66; water (0.1% TFA):MeCN (0.1% TFA)) to give a mixture, which was further separated by Chiral-SFC (OJ-H column 25% (0.1% NH₃H₂O+EtOH) co-solvent) to give Examples 66A (first eluted fraction) and 66B (second eluted fraction).

Example 66A: LRMS m/z (M+H): calculated 415.8, observed 416.1. ¹H NMR δ (ppm) (400 MHz, CD3CN0): 7.88-7.94 (m, 1H), 7.13-7.50 (m, 4H), 6.31-6.43 (m, 2H), 6.05-6.13 (m, 2H), 4.34-4.44 (m, 1H), 3.88-3.99 (m, 1H), 3.25-3.36 (m, 1H), 3.14-3.24 (m, 2H), 1.31-1.37 (m, 3H).

Example 66B: LRMS m/z (M+H): calculated 415.8, observed 416.1. ¹H NMR δ (ppm) (400 MHz, CD3CN): 7.88-7.94 (m, 1H), 7.11-7.51 (m, 4H), 6.35-6.41 (m, 2H), 6.01-6.16 (m, 2H), 4.41 (q, J=7.2 Hz, 1H), 3.93-4.01 (m, 1H), 3.25-3.34 (m, 1H), 3.13-3.23 (m, 2H), 1.33-1.39 (m, 3H).

TABLE 10 The following examples were prepared according to the synthetic procedure for Examples 66A and 66B, using the appropriate starting materials and reagents

| | | | Calc'd | Observed | |
|---|---|---|---|---|---|
| Example | Compound | Name | [M + H]⁺ | [M + H]⁺ | Conditions |
| 67A | 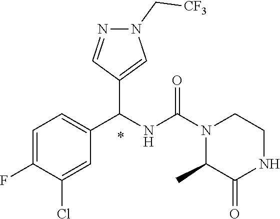 [SMILES: C[C@@H]1C(=O)NCCN1C(=O)NC(c1ccc(F)c(Cl)c1)c1cnn(CC(F)(F)F)c1] | (2R)-N-((R orS)-(3- chloro-4-fluorophenyl)(1- (2,2,2-trifluoroethyl)-1H- pyrazol-4-yl)methyl)-2- methyl-3-oxopiperazine- 1-carboxamide | 447.1 | 448.1 | SFC: YMC Amylose-C Co-solvent: 45% (EtOH + 0.05% DEA) peak 1 |
| 67B | 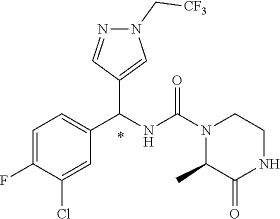 [SMILES: C[C@@H]1C(=O)NCCN1C(=O)NC(c1ccc(F)c(Cl)c1)c1cnn(CC(F)(F)F)c1] | (2R)-N-((S or R)-(3- chloro-4-fluorophenyl)(1- (2,2,2-trifluoroethyl)-1H- pyrazol-4-yl)methyl)-2- methyl-3-oxopiperazine- 1-carboxamide | 447.1 | 448.1 | SFC: YMC Amylose-C Co-solvent: 45% (EtOH + 0.05% DEA) peak 2 |

### Examples 68A and 68B

### (2R)—N—((R)-(4-chlorophenyl)(2-(trifluoromethyl)pyrimidin-4-yl)methyl)-2-methyl-3-oxopiperazine-1-carboxamide and (2R)—N—((S)-(4-chlorophenyl)(2-(trifluoromethyl)pyrimidin-4-yl)methyl)-2-methyl-3-oxopiperazine-1-carboxamide

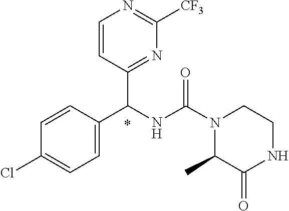 [SMILES: C[C@@H]1C(=O)NCCN1C(=O)NC(c1ccc(Cl)cc1)c1ccnc(C(F)(F)F)n1]

Step 1: (E)-2-methyl-N-((2-(trifluoromethyl)pyrimidin-4-yl)methylene)propane-2-sulfinamide To a mixture of 2-(trifluoromethyl)pyrimidine-4-carbaldehyde (400 mg, 2.271 mmol) and 2-methylpropane-2-sulfinamide (330 mg, 2.73 mmol) in THF (8 mL) was added tetraisopropoxytitanium (1291 mg, 4.54 mmol) at 20° C. The resulting mixture was stirred at 20° C. for 2 hours, then poured into water (20 mL) and filtered. The filtrate was extracted with ethyl acetate (3×20 mL). The combined organic layers were washed with brine (10 mL), dried over anhydrous sodium sulfate and filtered. The filtrate was concentrated under reduced pressure to give a residue, which was purified by flash silica gel chromatography (ISCO®; 4 g SepaFlash® Silica Flash Column, eluent of 18% petroleum ether/ethyl acetate) to give the title compound. ¹H NMR (400 MHz, CDCl₃) δ 9.06 (d, J=5.2 Hz, 1H), 8.72 (s, 1H), 8.11 (d, J=5.2 Hz, 1H), 1.29 (s, 9H).

Step 2: N-((4-chlorophenyl)(2-(trifluoromethyl)pyrimidin-4-yl)methyl)-2-methylpropane-2-sulfinamide To a stirred solution of (E)-2-methyl-N-((2-(trifluoromethyl)pyrimidin-4-yl)methylene)propane-2-sulfinamide (300 mg, 1.074 mmol) in THF (15 mL) was added (4-chlorophenyl) magnesium-bromide in THF (1 M, 2.6 mL, 2.60 mmol) at 0° C. The reaction was stirred at 0° C. for 2 hours, then quenched with saturated NH₄Cl aqueous solution (10 mL) and extracted with EtOAc (2×10 mL). The combined organic layers were dried over anhydrous sodium sulfate and filtered. The filtrate was concentrated under reduced pressure to give the title compound. LRMS m/z (M+H): calculated 391.1, observed 392.1.

Step 3: (4-chlorophenyl)(2-(trifluoromethyl)pyrimidin-4-yl)methanamine hydrochloride To a stirred solution of N-((4-chlorophenyl)(2-(trifluoromethyl)pyrimidin-4-yl)methyl)-2-methyl-propane-2-sulfinamide (100 mg, 0.255 mmol) in MeOH (1.0 mL) was added HCl MeOH (4 M, 1.0 mL) at 20° C. The reaction was stirred at 20° C. for 12 hours, then the solvent was removed under reduced pressure to give the title compound. LRMS m/z (M+H): calculated 287.0, observed 288.0.

Step 4: Examples 68A and 68B To a stirred solution of CDI (51 mg, 0.315 mmol) in DMF (2.0 mL) was added (4-chlorophenyl) (2-(trifluoromethyl)pyrimidin-4-yl)methanamine hydrochloride (60 mg, 0.209 mmol) in DMF (1.0 mL) at 20° C. Then the reaction was stirred at 20° C. for 30 minutes and (R)-3-methylpiperazin-2-one (36 mg, 0.315 mmol) was added. The reaction mixture was stirred at 15° C. for 2 hours. Then the reaction mixture was purified via Prep-HPLC (60:40 to 30:70; water (0.1% TFA):MeCN (0.1% TFA)) to give a mixture, which was further separated by chiral SFC (OD-H column, cosolvent: 30% EtOH with 0.1% NH₃H₂O) to give Examples 68A (first eluted fraction) and 68B (second eluted fraction).

Example 68A: LRMS m/z (M+H): calculated 427.8, observed 428.2. ¹H NMR δ (ppm) (400 MHz, CD₃OD): 8.88 (d, J=5.2 Hz, 1H), 7.64 (d, J=5.2 Hz, 1H), 7.32-7.39 (m, 4H), 6.15 (s, 1H), 4.60 (q, J=7.2 Hz, 1H), 4.03-4.11 (m, 1H), 3.37-3.45 (m, 2H), 3.21-3.28 (m, 1H), 1.47 (d, J=6.8 Hz, 3H).

Example 68B: LRMS m/z (M+H): calculated 427.8, observed 428.2. ¹H NMR δ (ppm) (400 MHz, CD₃OD): 8.89 (d, J=5.2 Hz, 1H), 7.68 (d, J=5.2 Hz, 1H), 7.31-7.37 (m, 4H), 6.14 (s, 1H), 4.60 (q, J=7.2 Hz, 1H), 4.02-4.09 (m, 1H), 3.33-3.37 (m, 2H), 3.22-3.29 (m, 1H), 1.40 (d, J=6.8 Hz, 3H).

TABLE 11 The following examples were prepared according to the synthetic procedure for Examples 68A and 68B, using the appropriate starting materials and reagents

| | | | Calc'd | Observed | |
|---|---|---|---|---|---|
| Example | Compound | Name | [M + H]⁺ | [M + H]⁺ | Conditions |
| 69A | 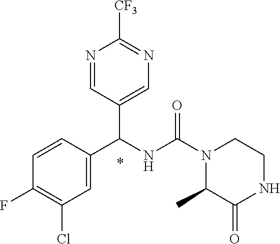 [SMILES: C[C@@H]1C(=O)NCCN1C(=O)NC(c1cnc(C(F)(F)F)nc1)c1ccc(F)c(Cl)c1] | (2R)-N-((R or S)-(3- chloro-4-fluorophenyl)(2- (trifluoromethyl)pyrimidin- 5-yl)methyl)-2-methyl-3- oxopiperazine-1- carboxamide | 445.1 | 446.1 | SFC: WHELK- O1 Co-solvent: 0-40% (EtOH with 0.1% NH₃H₂O) peak 1 |
| 69B | 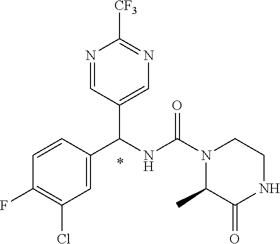 [SMILES: C[C@@H]1C(=O)NCCN1C(=O)NC(c1cnc(C(F)(F)F)nc1)c1ccc(F)c(Cl)c1] | (2R)-N-((S or R)-(3- chloro-4-fluorophenyl)(2- (trifluoromethyl)pyrimidin- 5-yl)methyl)-2-methyl-3- oxopiperazine-1- carboxamide | 445.1 | 446.1 | SFC: WHELK- O1 Co-solvent: 0-40% (EtOH with 0.1% NH₃H₂O) peak 2 |

### Examples 70A and 70B

### (2R)—N—((R)-(3-chloro-4-fluorophenyl)(5-fluoro-6-(2,2,2-trifluoroethoxy)pyridin-2-yl)methyl)-2-methyl-3-oxopiperazine-1-carboxamide and (2R)—N—((S)-(3-chloro-4-fluorophenyl)(5-fluoro-6-(2,2,2-trifluoroethoxy)pyridin-2-yl)methyl)-2-methyl-3-oxopiperazine-1-carboxamide

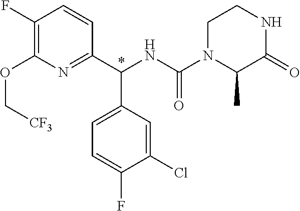 [SMILES: C[C@@H]1C(=O)NCCN1C(=O)NC(c1ccc(F)c(Cl)c1)c1ccc(F)c(OCC(F)(F)F)n1]

Step 1: 6-chloro-5-fluoro-N-methoxy-N-methylpicolinamide To a mixture of 6-chloro-5-fluoropicolinic acid (5 g, 28.5 mmol) in DCM (20 mL) was added CDI (5.54 g, 34.2 mmol) at 20° C. The mixture was stirred at 20° C. for 1 hour under N₂. Then N,O-dimethylhydroxylamine hydrochloride (3.33 g, 34.2 mmol) and triethylamine (12.35 mL, 85 mmol) were added, and the mixture was stirred at 20° C. for 16 hours under N₂. The reaction mixture was then diluted with water (50 mL), extracted with DCM (3×30 mL). The combined organic layers were washed with brine (50 mL), dried over anhydrous Na₂SO₄, filtered and concentrated. The resulting residue was purified by flash silica gel chromatography (ISCO®: 20 g SepaFlash® Silica Flash Column, eluent of [0˜30]% ethyl acetate/pet. ether) to give the title compound. LRMS m z (M+H): calculated 218.1, observed 219.0.

Step 2: 5-fluoro-N-methoxy-N-methyl-6-(2,2,2-trifluoroethoxy)picolinamide To a mixture of 6-chloro-5-fluoro-N-methoxy-N-methylpicolinamide (3 g, 13.72 mmol), tBuXPhosPd G2 (1.068 g, 1.372 mmol) and Cs₂CO₃ (9.39 g, 28.8 mmol) in toluene (20 mL) was added 2,2,2-trifluoro-ethanol (1.098 g, 10.98 mmol) at 20° C. The mixture was stirred at 80° C. for 16 hours under N₂. Then the mixture was filtered, and filtrate was concentrated under reduced pressure. The resulting residue was purified by flash silica gel chromatography (ISCO®; 20 g SepaFlash® Silica Flash Column, eluent of [0˜30]% ethyl acetate/petroleum ether) to give the title compound. LRMS m/z (M+H): calculated 282.1, observed 283.1.

Step 3: (3-chloro-4-fluorophenyl)(5-fluoro-6-(2,2,2-trifluoroethoxy)pyridin-2-yl)methanone To a solution of 4-bromo-2-chloro-1-fluorobenzene (2.449 g, 11.69 mmol) in THF (5 mL) was added isopropylmagnesium chloride in THF (1.3 M, 6.54 mL, 8.50 mmol) at 0° C. and the mixture was stirred at 20° C. for 1 h. Then a solution of 5-fluoro-N-methoxy-N-methyl-6-(2,2,2-trifluoroethoxy)picolinamide (1.5 g. 5.32 mmol) in THF (5 mL) was added, and the resulting mixture was stirred at 20° C. for 16 hours. Then saturated aqueous NH₄Cl (20 mL) was added to the reaction, and the mixture was extracted with EtOAc (3×50 mL). The combined organic layers were dried by Na₂SO₄, filtered and evaporated under reduced pressure. The resulting residue was purified by flash silica gel chromatography (ISCO®: 20 g SepaFlash® Silica Flash Column, eluent of 30% ethyl acetate/petroleum ether) to give the title compound. LRMS m/z. (M+H): calculated 351.0, observed 352.1.

Step 4: (3-chloro-4-fluorophenyl)(5-fluoro-6-(2,2,2-trifluoroethoxy)pyridin-2-yl)methanamine NH₄OAc (986 mg. 12.80 mmol) and NaBH—CN (80 mg, 1.280 mmol) were added to a solution of (3-chloro-4-fluorophenyl)(5-fluoro-6-(2,2,2-trifluoroethoxy)pyridin-2-yl)methanone (300 mg. 0.853 mmol) in EtOH (5 mL) in a 30 mL microwave vial. The mixture was stirred and heated at 130° C. for 10 minutes in a microwave reactor. Then the reaction mixture was concentrated to remove most of the EtOH, treated with 2 N NaOH until pH>10, and extracted with EtOAc (2×20 mL). The combined organic layers were dried over Na₂SO₄, filtered, and concentrated under reduced pressure. The resulting residue was purified by flash silica gel chromatography (ISCO®; 20 g SepaFlash® Silica Flash Column, eluent of [0˜30]% ethyl acetate/petroleum ether) to give the title compound. ¹H NMR (500 MHz, CD₃OD-d₄) δ 7.61-7.71 (m, 2H), 7.43-7.45 (m, 1H), 7.33 (t, J=8.5 Hz, 1H), 7.07 (dd, J=2.5, 8.0 Hz, 1H), 5.63 (s, 1H), 4.98-5.24 (m, 2H).

Step 5: Examples 70A and 70B A mixture of CDI (101 mg. 0.624 mmol) and (3-chloro-4-fluorophenyl)(5-fluoro-6-(2,2,2-trifluoroethoxy)pyridin-2-yl)methanamine (220 mg. 0.624 mmol) in DCM (2 mL) was stirred at 20° C. for 1 hour. Then (R)-3-methylpiperazin-2-one (71.2 mg. 0.624 mmol) was added. The resulting mixture was stirred at 20° C. for 16 hours, then the mixture was dissolved in water (20 mL) and DCM (20 mL). The organic layer was separated and the aqueous layer was back extracted with DCM (3×20 mL). The combined organic layers were washed with brine (20 mL), dried over anhydrous Na₂SO₄, filtered and the filtrate was evaporated under reduced pressure. The resulting residue was purified by Prep-HPLC (73:27 to 43:57; water (0.1% TFA):MeCN(0.1% TFA)) to give a mixture of isomers, which was further separated by Chiral-SFC (Column AD-H, Co solvent: 0-43% EtOH with 0.05% DEA) to give Examples 70A (first eluted fraction) and 70B (second eluted fraction).

Example 70A: LRMS m/z (M+H): calculated 492.1, observed 493.1. ¹H NMR δ (ppm) (400 MHz, CD₃OD-d₄): 7.57 (dd, J=8.0, 10.0 Hz, 1H), 7.45 (dd, J=2.0, 7.2 Hz, 1H), 7.28-7.29 (m, 1H), 7.16-7.24 (m, 1H), 7.07 (dd, J=2.8, 8.4 Hz, 1H), 6.06 (s, 1H), 4.91-4.98 (m, 2H), 4.61 (q, J=7.2 Hz, 1H), 4.02-4.14 (m, 1H), 3.24-3.43 (m, 3H), 1.43 (d, J=7.2 Hz, 3H).

Example 70B: LRMS m/z (M+H): calculated 492.1, observed 493.1. ¹H NMR δ (ppm) (400 MHz, CD₃OD-d₄): 7.57 (dd, J=8.0, 10.0 Hz, 1H), 7.46 (dd, J=2.4, 7.04 Hz, 1H), 7.28-7.29 (m, 1H), 7.16-7.24 (m, 1H), 7.05 (dd, J=2.4, 8.0 Hz, 1H), 6.06 (s, 1H), 4.91-4.98 (m, 2H), 4.61 (q, J=7.2 Hz, 1H), 4.01-4.15 (m, 1H), 3.22-3.43 (m, 3H), 1.44 (d, J=7.2 Hz, 3H).

TABLE 12 The following examples were prepared according to the synthetic procedure for Examples 70A and 70B, using the appropriate starting materials and reagents

| | | | Calc'd | Observed | |
|---|---|---|---|---|---|
| Example | Compound | Name | [M + H]⁺ | [M + H]⁺ | Conditions |
| 71A | 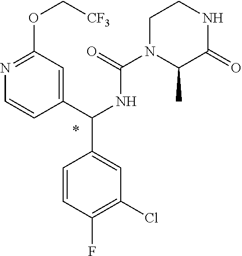 [SMILES: C[C@@H]1C(=O)NCCN1C(=O)NC(c1ccnc(OCC(F)(F)F)c1)c1ccc(F)c(Cl)c1] | (2R)-N-((R or S)-(3- chloro-4-fluorophenyl)(2- (2,2,2-trifluoro- ethoxy)pyridin-4- yl)methyl)-2-methyl-3- oxopiperazine-1- carboxamide | 474.1 | 475.2 | SFC: WHELK-O1 Co-solvent: 0-40% (EtOH with 0.1% NH3H2O) peak 1 |
| 71B | 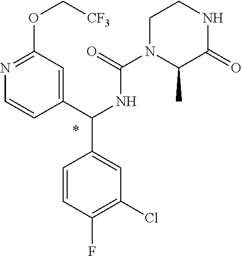 [SMILES: C[C@@H]1C(=O)NCCN1C(=O)NC(c1ccnc(OCC(F)(F)F)c1)c1ccc(F)c(Cl)c1] | (2R)-N-((S or R)-(3- chloro-4-fluorophenyl)(2- (2,2,2-trifluoro- ethoxy)pyridin-4- yl)methyl)-2-methyl-3- oxopiperazine-1- carboxamide | 474.1 | 475.2 | SFC: WHELK-O1 Co-solvent: 0-40% (EtOH with 0.1% NH3H2O) peak 2 |

### Examples 72A and 72B

### (2R)—N—((R)-(3-chloro-4-fluorophenyl)(6-(difluoromethoxy)pyridin-2-yl)methyl)-2-methyl-3-oxopiperazine-1-carboxamide and (2R)—N—((S)-(3-chloro-4-fluorophenyl)(6-(difluoromethoxy)pyridin-2-yl)methyl)-2-methyl-3-oxopiperazine-1-carboxamide

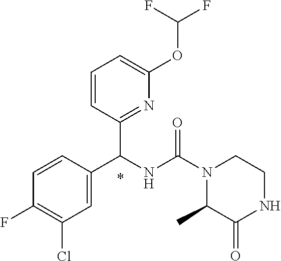 [SMILES: C[C@@H]1C(=O)NCCN1C(=O)NC(c1ccc(F)c(Cl)c1)c1cccc(OC(F)F)n1]

Step 1: 2-chloro-6-(difluoromethoxy)pyridine To a mixture of 6-chloropyridin-2-ol in MeCN (15 mL) was added sodium hydride (0.617 g, 15.44 mmol) at 0° C. The reaction was stirred for 1 hour, then 2,2-difluoro-2-(fluorosulfonyl)acetic acid (2.062 g, 11.58 mmol) was added at 0° C. The reaction mixture was stirred at 20° C. for 12 hour, then water (40 mL) was added, and the mixture was extracted with ethyl acetate (3×15 mL). The combined organic layers were washed with brine (20 mL), dried over Na₂SO₄, filtered, and the filtrate was evaporated under reduced pressure. The crude product was purified by flash silica gel chromatography (ISCO®: 12 g SepaFlash® Silica Flash Column, Eluent of 2.5% petroleum ether/ethyl acetate) to give the title compound. LRMS m/z (M+H): calculated 179.5, observed 180.0.

Step 2: 6-(difluoromethoxy)-N-methoxy-N-methylpicolinamide To a mixture of 2-chloro-6-(difluoromethoxy)pyridine (2 g, 11.14 mmol) and N,O-dimethylhydroxylamine hydrochloride (1.630 g, 16.71 mmol) in toluene (15 mL) were added triethylamine (3.38 g, 33.4 mmol), xantphos (0.645 g, 1.114 mmol), and Pd(OAc)₂ (0.150 g, 0.668 mmol). The reaction vessel was degassed and backfilled with CO (three times). The resulting mixture was stirred under CO (pressure: 15 atm) at 80° C. for 18 hour, then the mixture was cooled to room temperature. To the reaction mixture was added water (250 mL), and the mixture was extracted with ethyl acetate (3×20 mL). The combined organic layers were washed with brine (50 mL), dried over Na₂SO₄, filtered, and the filtrate was evaporated under reduced pressure. The crude product was purified by flash silica gel chromatography (ISCO®: 20 g SepaFlash® Silica Flash Column, eluent of 1% petroleum ether/ethyl acetate) to give the title compound. LRMS m/z (M+H): calculated 232.1, observed 233.2.

Step 3: 6-(difluoromethoxy)picolinaldehyde To a mixture of 6-(difluoromethoxy)-N-methoxy-N-methylpicolinamide (400 mg, 1.723 mmol) in THF (5 mL) was added DIBAL-H in toluene (1 M, 3.45 mL, 3.45 mmol) at (° C.) The resulting mixture was stirred at 0° C. for 1 hour. Then water (30 mL) was added and the mixture was extracted with DCM (4×15 mL). The combined organic layers were washed with brine (30 mL), dried over Na₂SO₄, filtered and the filtrate was evaporated under reduced pressure to give the title compound. LRMS m/z (M+H): calculated 173.1.0, observed 174.1.

Step 4: (R,E)-N-((6-(difluoromethoxy)pyridin-2-yl)methylene)-2-methylpropane-2-sulfinamide To a mixture of 6-(difluoromethoxy)picolinaldehyde (200 mg crude) in THF (5 mL) was added titanium (IV) ethoxide (0.570 ml, 2.77 mmol) at 0° C. The resulting mixture was stirred at 20° C. for 1 hour, then diluted with ethyl acetate (20 mL) and brine (100 mL), and filtered. The filtrate was extracted with ethyl acetate (3×15 mL). The combined organic layers were evaporated under reduced pressure. The resulting crude product was purified by flash silica gel chromatography (ISCO®: 4 g SepaFlash® Silica Flash Column, Eluent of 28% petroleum ether/ethyl acetate) to give the title compound LRMS m/z. (M+H): calculated 276.1, observed 277.0.

Step 5: (R)—N-((3-chloro-4-fluorophenyl)(6)-(difluoromethoxy)pyridin-2-yl)methyl)-2-methylpropane-2-sulfinamide To a mixture of (R,E)-N-((6-(difluoromethoxy)pyridin-2-yl)methylene)-2-methylpropane-2-sulfinamide (100 mg, 0.362 mmol) in toluene (1 mL) was added (3-chloro-4-fluorophenyl) magnesium bromide in THF (0.5 M, 2.172 mL, 1.086 mmol) at −45° C. The resulting mixture was stirred at −45° C. for 90 minutes. To the mixture was added saturated aqueous NH₄Cl (10 mL), and the resulting mixture was extracted with ethyl acetate (3×8 mL). The combined organic layers were dried over Na₂SO₄, filtered, and the filtrate was evaporated under reduced pressure. The resulting residue was purified by prep-TLC (SiO₂, petroleum ether:ethyl acetate=1:1) to give the title compound LRMS m/z (M+H): calculated 406.1, observed 407.1.

Step 6: (3-chloro-4-fluorophenyl)(6-(difluoromethoxy)pyridin-2-yl)methanamine hydrochloride To a mixture of (R)—N-((3-chloro-4-fluorophenyl)(6-(difluoromethoxy)pyridin-2-yl)methyl)-2-methylpropane-2-sulfinamide (100 mg, 0.246 mmol) in MeOH (2 ml) was added HCl/MeOH (3 mL, 0.246 mmol). The resulting mixture was stirred at 15° C. for 1 hour, then evaporated under reduced pressure to give the title compound. LRMS m/z (M+H): calculated 302, observed 303.

Step 7: Examples 72A and 72B To a mixture of (3-chloro-4-fluorophenyl)(6-(difluoro-methoxy)pyridin-2-yl)methanamine hydrochloride (100 mg crude) and CDI (96 mg, 0.590 mmol) in DMF (1.5 mL) was added DIEA (0.103 mL, 0.590 mmol). The reaction was stirred at 20° C. for 2 hours, then (R)-3-methylpiperazin-2-one (40.4 mg, 0.354 mmol) was added. The resulting mixture was stirred at 20° C. for 1 hour and then purified by reverse phase HPLC (55:45 to 25:75; water (0.1% TFA):MeCN (0.1% TFA)), followed by lyophilization to give a mixture of isomers, which was further separated by Chiral-SFC (Column AS-H, Co solvent: 0-43% EtOH with 0.1% NH₃H₂O) to give Examples 72A (first eluted fraction) and 72B (second eluted fraction).

Example 72A: LRMS m/z (M+H): calculated 442.8, observed 443.2. ¹H NMR δ (ppm) (500 MHz, CD₃OD-d₄): 7.86 (t, J=8.0 Hz, 1H), 7.49 (dd, J=2.0, 7.0 Hz, 1H), 7.40-7.74 (m, 1H), 7.29-7.35 (m, 1H), 7.19-7.26 (m, 2H), 6.91 (d, J=8.0 Hz, 1H), 6.09 (s, 1H), 4.63 (q, J=7.0 Hz, 1H), 4.05-4.14 (m, 1H), 3.37-3.46 (m, 1H), 3.27-3.33 (m, 2H), 1.46 (d, J=7.0 Hz, 3H).

Example 72B: LRMS m/z (M+H): calculated 442.8, observed 443.2. ¹H NMR δ (ppm) (500 MHz, CD₃OD-d₄): 7.86 (t, J=8.0 Hz, 1H), 7.44-7.77 (m, 2H), 7.29-7.36 (m, 1H), 7.17-7.26 (m, 2H), 6.92 (d, J=8.0 Hz, 1H), 6.09 (s, 1H), 4.64 (q, J=7.0 Hz, 1H), 4.05-4.17 (m, 1H), 3.37-3.45 (m, 1H), 3.26-3.33 (m, 2H), 1.47 (d, J=7.0 Hz, 3H).

TABLE 13 The following examples were prepared according to the synthetic procedure for Examples 72A and 72B, using the appropriate starting materials and reagents

| | | | Calc'd | Observed | |
|---|---|---|---|---|---|
| Example | Compound | Name | [M + H]⁺ | [M + H]⁺ | Conditions |
| 73A | 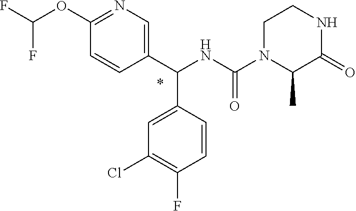 [SMILES: C[C@@H]1C(=O)NCCN1C(=O)NC(c1ccc(OC(F)F)nc1)c1ccc(F)c(Cl)c1] | (2R)-N-((R or S)-(3- chloro-4- fluorophenyl)(6- (difluoromethoxy) pyridin-3-yl)methyl)-2- methyl-3- oxopiperazine-1- carboxamide | 442.1 | 443.1 | SFC: AD-H Co-solvent: 0-35% (EtOH with 0.05% DEA) peak 1 |
| 73B | 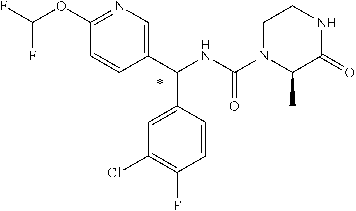 [SMILES: C[C@@H]1C(=O)NCCN1C(=O)NC(c1ccc(OC(F)F)nc1)c1ccc(F)c(Cl)c1] | (2R)-N-((R or S)-(3- chloro-4-fluoro- phenyl)(6-(difluoro- methoxy)pyridin-3- yl)methyl)-2-methyl-3- oxopiperazine-1- carboxamide | 442.1 | 443.1 | SFC: AD-H Co-solvent: 0-35% EtOH with 0.05% DEA) peak 2 |
| 74A | 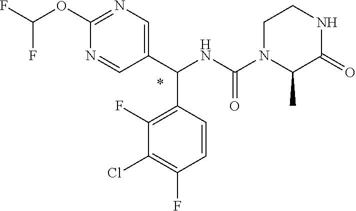 [SMILES: C[C@@H]1C(=O)NCCN1C(=O)NC(c1cnc(OC(F)F)nc1)c1ccc(F)c(Cl)c1F] | (2R)-N-((R or S)-(3- chloro-2,4-difluoro- phenyl)(2-(difluoro- methoxy)pyrimidin-5- yl)methyl)-2-methyl-3- oxopiperazine-1- carboxamide | 461.1 | 462.0 | SFC: OJ-H Co-solvent: 0-35% (EtOH with 0.1% NH3H2O) peak 1 |
| 74B | 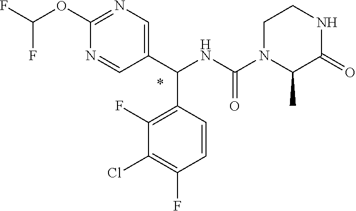 [SMILES: C[C@@H]1C(=O)NCCN1C(=O)NC(c1cnc(OC(F)F)nc1)c1ccc(F)c(Cl)c1F] | (2R)-N-((S or R)-(3- chloro-2,4- difluorophenyl)(2- (difluoromethoxy) pyrimidin- 5-yl)methyl)-2- methyl-3- oxopiperazine-1- carboxamide | 461.1 | 462.0 | SFC: OJ-H Co-solvent: 0-35% (EtOH with 0.1% NH3H2O) peak 2 |

### Examples 75A and 75B

### (2R)—N—((R)-(3-chloro-2,4-difluorophenyl)(6-(1,1-difluoroethyl)pyridin-3-yl)methyl)-2-methyl-3-oxopiperazine-1-carboxamide and (2R)—N—((S)-(3-chloro-2,4-difluorophenyl)(6-(1,1-difluoroethyl)pyridin-3-yl)methyl)-2-methyl-3-oxopiperazine-1-carboxamide

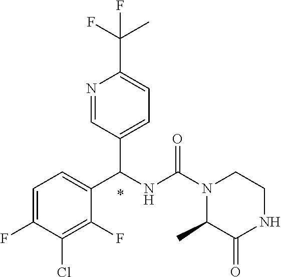 [SMILES: C[C@@H]1C(=O)NCCN1C(=O)NC(c1ccc(C(C)(F)F)nc1)c1ccc(F)c(Cl)c1F]

Step 1: 5-bromo-2-(1,1-difluoroethyl)pyridine A solution of 1-(5-bromopyridin-2-yl)ethan-1-one (6 g, 30.0 mmol) in BAST (60 mL) was stirred at 70° C. for 2 hours. The reaction mixture was quenched by the addition of saturated aqueous. NaHCO₃ (100 mL) and extracted with DCM (3×50 mL). The combined organic layers were dried over Na₂SO₄, filtered, and the filtrate was evaporated under reduced pressure. The resulting crude product was purified by flash silica gel chromatography (ISCO: 40 g Agela Silica Flash column, eluent of 0˜2% EtOAc/petroleum ether) to give the title compound. ¹H NMR (CD₃OD, 400 MHz) δ 8.71 (d, J=4.0 Hz, 1H), 8.13 (dd, J=8.0 Hz, 8.0 Hz, 1H), 7.64 (d, J=8.0 Hz, 1H), 1.97 (t, J=8.0 Hz, 3H).

Step 2: 2-(1,1-difluoroethyl)-5-vinylpyridine To a mixture of 5-bromo-2-(1,1-difluoroethyl)-pyridine (2.8 g, 12.61 mmol) and 1,4-dioxane (60 mL) in water (12 mL) were added K₂CO₃ (3.49 g, 25.2 mmol), potassium trifluoro(vinyl)borate (3.38 g, 25.2 mmol) and Pd(dppf)Cl₂ (0.923 g, 1.261 mmol). The reaction mixture was stirred at 80° C. for 18 hours under N₂. Then the reaction was quenched with water (100 mL), extracted with DCM (3×80 mL). The combined organic layers were dried over Na₂SO₄, and filtered. The filtrate was concentrated in vacuo, and the resulting residue was purified by Prep. MPLC (ISCO®: 40 g SepaFlash® Silica Flash Column, eluent of 0˜1% EtOAc/petroleum ether) to give the title compound. LRMS m z (M+H): calculated 169.2, observed 170.1.

Step 3: 6-(1,1-difluoroethyl)nicotinaldehyde To a solution of 2-(1,1-difluoroethyl)-5-vinylpyridine (1.7 g. 10.05 mmol) in 1,4-dioxane (30 mL) and water (10 mL) was added sodium periodate (4.30 g, 20.10 mmol) and osmium (VIII) oxide (0.128 g, 0.502 mmol). The reaction was stirred at 20° C. for 18 hours. Then the mixture was diluted with water (50 mL), and extracted with EtOAc (3×50 mL). The combined organic layers were washed with brine (50) mL), dried over Na₂SO₄, filtered, and the filtrate was concentrated in vacuo. The resulting residue was purified by Prep. MPLC (ISCO®: 20 g SepaFlash® Silica Flash Column, eluent of (˜1% EtOAc/petroleum ether) to give the title compound. ¹H NMR (CD₃OD, 400 MHz) δ 10.15 (s, 1H), 8.69 (s, 1H), 8.02 (dd, J=1.6 Hz, 8.0 Hz, 1H), 7.68-7.71 (m, 1H), 1.97 (t, J=18.0 Hz, 3H).

Step 4: (R,E)-N-((6-(1,1-difluoroethyl)pyridin-3-yl)methylene)-2-methylpropane-2-sulfinamide To a solution of 6-(1,1-difluoroethyl)nicotinaldehyde (1.7 g. 9.93 mmol) in THF (30 mL) were added tetraethoxytitanium (2.266 g. 9.93 mmol) and (R)-2-methylpropane-2-sulfinamide (1.204 g. 9.93 mmol). The reaction mixture was stirred at 70° C. for 2 hours and then cooled to RT. The mixture was then diluted with ethyl acetate (20 mL) and brine (100 mL), and filtered. The filtrate was extracted with ethyl acetate (3×50 mL), and the combined organic layers were dried over Na₂SO₄ and filtered. The filtrate was evaporated under reduced pressure to give the title compound. ¹H NMR (CD₃OD, 400 MHz): δ 9.09 (s, 1H), 8.71 (s, 1H), 8.45 (dd, J=8.0 Hz, 4.0 Hz, 1H), 7.86 (d, J=8.0 Hz, 1H), 2.02 (t. J=18.0 Hz, 3H), 1.29 (d, J=4.0 Hz, 9H).

Step 5: (R)—N-((3-chloro-2,4-difluorophenyl)(6-(1,1-difluoroethyl)pyridin-3-yl)methyl)-2-methylpropane-2-sulfinamide To a solution of isopropylmagnesium chloride in THF (2 M, 3.83 mL, 7.66 mmol) was added 1-bromo-3-chloro-2,4-difluorobenzene (1741 mg, 7.66 mmol) at 0) ° C. over 3 hours. The resulting mixture was added to a solution of (R,E)-N-((6-(1,1-difluoroethyl)pyridin-3-yl)methylene)-2-methylpropane-2-sulfinamide (700 mg, 2.55 mmol) in THF (5 mL). The reaction mixture was stirred at 25° C. for 18 hours, then water (20 mL) was added, and the mixture was extracted with ethyl acetate (3×20 mL). The combined organic layers were washed with brine (20 mL), dried over Na₂SO₄, filtered, and the filtrate was evaporated under reduced pressure. The resulting crude product was purified by flash silica gel chromatography (ISCO: 12 g Agela Silica Flash Column, Eluent of 0˜50% EtOAc/pet.ether) to to give the title compound. LRMS m/z (M+H): calculated 422.8, observed 423.4.

Step 6: (3-chloro-2,4-difluorophenyl)(6-(1,1-difluoroethyl)pyridin-3-yl)methanamine hydrochloride To a solution of (R)—N-((3-chloro-2,4-difluorophenyl)(6-(1,1-difluoroethyl)-pyridin-3-yl)methyl)-2-methylpropane-2-sulfinamide (140 mg, 0.331 mmol) in MeOH (1 mL) was added HCl-MeOH (2M, 1 mL). The reaction mixture was stirred at 25° C. for 2 hours, then evaporated under reduced pressure to give the title compound.

Step 7: Examples 75A and 75B To a solution of (3-chloro-2,4-difluorophenyl)(6-(1,1-difluoroethyl)pyridin-3-yl)methanamine hydrochloride (118 mg crude) in DMF (2 mL) was added CDI (108 mg, 0.664 mmol). The reaction mixture was stirred at 25° C. for 1 hour, then (R)-3-methylpiperazin-2-one (49.3 mg, 0.432 mmol) was added. The reaction mixture was stirred at 25° C. for 18 hours, then purified by prep-HPLC (80:20 to 50:50; water (0.1% TFA):MeCN (0.1% TFA)) to give a mixture of isomers, which was further separated by Chiral-SFC (Column AD-H, Co solvent: 0-30% EtOH with 0.1% NH₃H₂O) to give Examples 75A (first eluted fraction) and 75B (second eluted fraction).

Example 75A: LRMS m/z (M+H): calculated 458.8, observed 459.1. ¹H NMR δ (ppm) (400 MHz, CD₃OD-d4): 8.52 (s, 1H), 7.80 (dd, J=2.0, 8.4 Hz, 1H), 7.70 (d, J=8.4 Hz, 1H), 7.31 (dt, J=6.4, 8.4 Hz, 1H), 7.17 (dt, J=1.6, 8.8 Hz, 1H), 6.47 (s, 1H), 4.59 (q, J=6.8 Hz, 1H), 4.02-4.11 (m, 1H), 3.34-3.43 (m, 1H), 3.21-3.30 (m, 2H), 1.97 (t, J=18.8 Hz, 3H), 1.42 (d, J=7.2 Hz, 3H).

Example 75B: LRMS m/z (M+H): calculated 458.8, observed 459.1. ¹H NMR δ (ppm) (400 MHz, CD₃OD-d₄): 8.54 (d, J=2.0 Hz, 1H), 7.84 (dd, J=2.0, 8.0 Hz, 1H), 7.72 (d, J=8.0 Hz, 1H), 7.28-7.32 (m, 1H), 7.16-7.23 (m, 1H), 6.48 (s, 1H), 4.56-4.65 (m, 1H), 4.04-4.14 (m, 1H), 3.37-3.46 (m, 1H), 3.25-3.32 (m, 2H), 1.99 (t, J=18.4 Hz, 3H), 1.45 (d, J=6.8 Hz, 3H).

### Examples 76A and 76B

### (2R)—N—((R)-(5-chloro-6-(trifluoromethyl)pyridin-2-yl)(4-cyanophenyl)methyl)-2-methyl-3-oxopiperazine-1-carboxamide and (2R)—N—((S)-(5-chloro-6-(trifluoromethyl)pyridin-2-yl)(4-cyanophenyl)methyl)-2-methyl-3-oxopiperazine-1-carboxamide

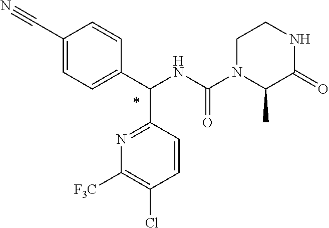 [SMILES: C[C@@H]1C(=O)NCCN1C(=O)NC(c1ccc(C#N)cc1)c1ccc(Cl)c(C(F)(F)F)n1]

Step 1: 3-chloro-2-(trifluoromethyl)-6-vinylpyridine To a mixture of 3,6-dichloro-2-(trifluoromethyl)pyridine (2 g. 9.26 mmol), potassium trifluoro(vinyl)borate (1.861 g. 13.89 mmol) and K₂CO₃ (2.56 g, 18.52 mmol) in THF (30 mL) and water (3 mL) was added Pd(dppf)Cl₂ (0.339 g. 0.463 mmol) at 20° C. under N₂. The reaction was stirred at 80° C. for 12 hours, then water (3 mL) was added and the mixture was extracted with DCM (3×15 mL). The combined organic layers were dried over Na₂SO₄ and filtered. The filtrate was concentrated under vacuum to give the title compound. LRMS m/z (M+H): calculated 207.5, observed 208.0. Step 2: 5-chloro-6-(trifluoromethyl)picolinaldehyde A mixture of 3-chloro-2-(trifluoromethyl)-6-vinylpyridine (1.922 g. 9.26 mmol), NMO (2.169 g, 18.52 mmol) and OsO₄ (4.63 mL. 0.463 mmol) in THF (10 mL) and water (5 mL) was stirred at 20° C. for 12 hours. Then NaIO₄ (5.94 g, 27.8 mmol) was added, and the mixture was stirred at 20° C. for 2 hours. Then water (60 mL) was added, and the mixture was extracted by DCM (3×40 mL). The combined organic layers were dried over Na₂SO₄, filtered, and the filtrate was concentrated under vacuum to give the title compound. LRMS m/z (M+H): calculated 209.6, observed 210.0.

Step 3: (R,E)-N-((5-chloro-6-(trifluoromethyl)pyridin-2-yl)methylene)-2-methylpropane-2-sulfinamide To a mixture of 5-chloro-6-(trifluoromethyl)picolinaldehyde (1.5 g crude) and (R)-2-methylpropane-2-sulfinamide (1.041 g. 8.59 mmol) in THF (5 mL) was added Ti(OEt)₄ (2.94 mL, 14.32 mmol) at 15° C. The reaction mixture was stirred at 80° C. for 1 hour, then diluted with ethyl acetate (60 mL) and brine (150 mL), and filtered. The filtrate was extracted with EtOAc (75 mL). The organic layer was dried over Na₂SO₄, filtered and concentrated. The resulting residue was purified by flash silica gel chromatography (ISCO®: 24 g SepaFlash® Silica Flash column, eluent of 15% ethyl acetate/pet. ether) to give the title compound. ¹H NMR (400 MHz, CDCl₃) δ 8.73 (s, 1H), 8.19 (d, J=8.4 Hz, 1H), 7.99 (d, J=8.4 Hz, 1H), 1.30 (s, 9H).

Step 4: (R)—N-((5-chloro-6-(trifluoromethyl)pyridin-2-yl)(4-cyanophenyl)methyl)-2-methylpropane-2-sulfinamide To a solution of 4-bromobenzonitrile (640 mg. 3.52 mmol) in THF (3 mL) was added isopropylmagnesium lithium chloride in THF (1.3 M, 2.460 mL. 3.20 mmol) at (° C.) The reaction was stirred at 20° C. for 1 hour, then added to a mixture of (R,E)-N-((5-chloro-6-(trifluoromethyl)pyridin-2-yl)methylene)-2-methylpropane-2-sulfinamide (500) mg, 1.599 mmol) in THF (2 mL). The resulting mixture was stirred at 20° C. for 1 hour, then 30) saturated aqueous NH₄Cl (5 mL) was added, and the mixture was extracted with EtOAc (3×5 mL). The combined organic layers were dried over Na₂SO₄, filtered, and the filtrate was evaporated under reduced pressure. The resulting residue was purified by flash silica gel chromatography (ISCO®: 12 g SepaFlash® Silica Flash Column, eluent of 60% ethyl acetate/pet. ether) to give the title compound. LRMS m/z (M+H): calculated 415.1, observed 416.1.

Step 5: 4-(amino(5-chloro-6-(trifluoromethyl)pyridin-2-yl)methyl)benzonitrile hydrochloride To a solution of (R)—N-((5-chloro-6-(trifluoromethyl)pyridin-2-yl)(4-cyanophenyl)methyl)-2-methylpropane-2-sulfinamide (570 mg, 1.371 mmol) in MeOH (2 mL) was added HCl/MeOH (2M, 5 mL). The resulting mixture was stirred at 20° C. for 2 hours, then concentrated under reduced pressure to give the title compound. LRMS m/z (M+H): calculated 311.0, observed 312.0.

Step 6: Examples 76A and 76B A mixture of CDI (104 mg, 0.642 mmol) and 4-(amino(5-chloro-6-(trifluoromethyl)pyridin-2-yl)methyl)benzonitrile hydrochloride (100 mg, 0.321 mmol) in DMF (2 mL) was stirred at 20° C. for 1 hour. Then (R)-3-methylpiperazin-2-one (40.3 mg, 0.353 mmol) was added, and the resulting mixture was stirred at 20° C. for 1 hour. The resulting solid was filtered off, and the filtrate was purified by prep-HPLC (80:20 to 50:50; water (0.1% TFA):MeCN (0.1% TFA)) to give a mixture of isomers, which was further separated by Chiral-SFC (Column OJ-H, Co solvent: 0-30% EtOH with 0.1% NH₃H₂O) to give Examples 76A (first eluted fraction) and 76B (second eluted fraction).

Example 76A: LRMS m/z (M+H): calculated 451.1, observed 452.2. ¹H NMR δ (ppm) (500 MHz, CD₃OD-d4): 8.03-8.09 (m, 1H), 7.69-7.75 (m, 2H), 7.58-7.64 (m, 1H), 7.51-7.57 (m, 2H), 6.27 (s, 1H), 4.60 (q, J=7.0 Hz, 1H), 4.05-4.11 (m, 1H), 3.36-3.44 (m, 1H), 3.25-3.30 (m, 2H), 1.44-1.50 (m, 3H).

Example 76B: LRMS m/z (M+H): calculated 451.1, observed 452.2. ¹H NMR δ (ppm) (500 MHz, CD₃OD-d₄): 8.04-8.10 (m, 1H), 7.69-7.75 (m, 2H), 7.63-7.69 (m, 1H), 7.49-7.55 (m, 2H), 6.26 (s, 1H), 4.59 (q, J=6.5 Hz, 1H), 4.03-4.11 (m, 1H), 3.34-3.40 (m, 1H), 3.25-3.30 (m, 2H), 1.41-1.47 (m, 3H).

TABLE 14 The following examples were prepared according to the synthetic procedure for Example 76A and 76B, using the appropriate starting materials and reagents

| | | | Calc'd | Observed | |
|---|---|---|---|---|---|
| Example | Compound | Name | [M + H]⁺ | [M + H]⁺ | Conditions |
| 77A | 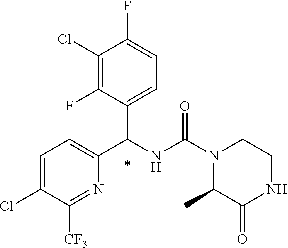 [SMILES: C[C@@H]1C(=O)NCCN1C(=O)NC(c1ccc(Cl)c(C(F)(F)F)n1)c1ccc(F)c(Cl)c1F] | (2R)-N-((R or S)-(3- chloro-2,4-difluoro phenyl)(5-chloro-6- (trifluoromethyl)pyridin- 2-yl)methyl)-2-methyl-3- oxopiperazine-1- carboxamide | 496.1 | 497.1 | SFC: AD-H Co-solvent: 25% (IPA + 0.1% NH3.H2O) peak 1 |
| 77B | 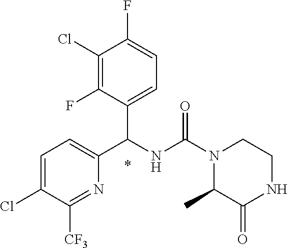 [SMILES: C[C@@H]1C(=O)NCCN1C(=O)NC(c1ccc(Cl)c(C(F)(F)F)n1)c1ccc(F)c(Cl)c1F] | (2R)-N-((S or R)-(3- chloro-2,4-difluoro phenyl)(5-chloro-6- (trifluoromethyl)pyridin- 2-yl)methyl)-2-methyl-3- oxopiperazine-1- carboxamide | 496.1 | 497.1 | SFC: AD-H Co-solvent: 25% (IPA + 0.1% NH3.H2O) Peak 2 |
| 78A | 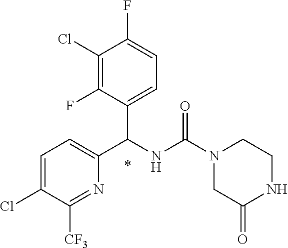 [SMILES: O=C1CN(C(=O)NC(c2ccc(Cl)c(C(F)(F)F)n2)c2ccc(F)c(Cl)c2F)CCN1] | N-((R or S)-(3-chloro- 2,4-difluorophenyl)(5- chloro-6-(trifluoro- methyl)pyridin-2- yl)methyl)-3- oxopiperazine-1- carboxamide | 482.0 | 482.9 | SFC: AD-H Co-solvent: 25% (IPA + 0.1% NH3.H2O) peak 1 |
| 78B | 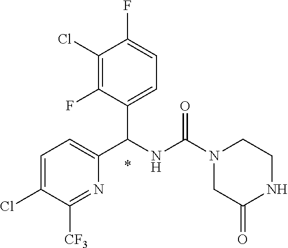 [SMILES: O=C1CN(C(=O)NC(c2ccc(Cl)c(C(F)(F)F)n2)c2ccc(F)c(Cl)c2F)CCN1] | N-((S or R)-(3-chloro- 2,4-difluorophenyl)(5- chloro-6-(trifluoro- methyl)pyridin-2- yl)methyl)-3- oxopiperazine-1- carboxamide | 482.0 | 482.9 | SFC: AD-H Co-solvent: 25% (IPA + 0.1%NH3. H2O) Peak 2 |
| 79 | 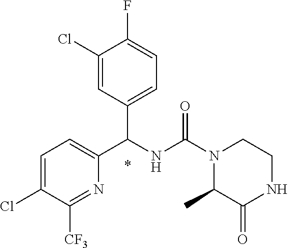 [SMILES: C[C@@H]1C(=O)NCCN1C(=O)NC(c1ccc(F)c(Cl)c1)c1ccc(Cl)c(C(F)(F)F)n1] | (2R)-N-((R or S)-(3- chloro-4-fluoro- phenyl)(5-chloro-6- (trifluoromethyl)pyridin- 2-yl)methyl)-2-methyl-3- oxopiperazine-1- carboxamide | 478.0 | 479.0 | SFC: AD-H Co-solvent: 25% (IPA + 0.1% NH3.H2O) Peak 2 |
| 80 | 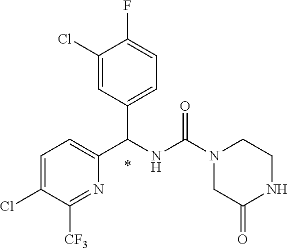 [SMILES: O=C1CN(C(=O)NC(c2ccc(F)c(Cl)c2)c2ccc(Cl)c(C(F)(F)F)n2)CCN1] | N-((R or S)-(3-chloro-4- fluorophenyl)(5-chloro-6- (trifluoromethyl)pyridin- 2-yl)methyl)-3- oxopiperazine-1- carboxamide | 464.0 | 465.0 | SFC: AD-H Co-solvent: 25% IPA + 0.1% NH3.H2O) Peak 2 |
| 81A | 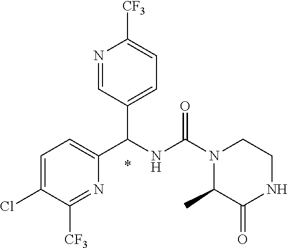 [SMILES: C[C@@H]1C(=O)NCCN1C(=O)NC(c1ccc(C(F)(F)F)nc1)c1ccc(Cl)c(C(F)(F)F)n1] | (2R)-N-((R or S)-(5- chloro-6- (trifluoromethyl)pyridin- 2-yl)(6-(trifluoro-methyl) pyridin-3-yl)methyl)-2- methyl-3-oxopiperazine- 1-carboxamide | 495.1 | 496.1 | SFC: OJ-H Co-solvent: 20% EtOH + 0.1% NH₃.H₂O) Peak 1 |
| 81B | 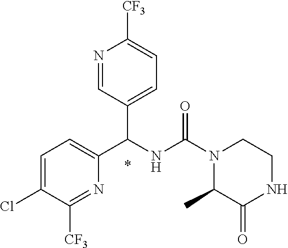 [SMILES: C[C@@H]1C(=O)NCCN1C(=O)NC(c1ccc(C(F)(F)F)nc1)c1ccc(Cl)c(C(F)(F)F)n1] | (2R)-N-((S or R)-(5- chloro-6- (trifluoromethyl)pyridin- 2-yl)(6-(trifluoro-methyl) pyridin-3-yl)methyl)-2- methyl-3-oxopiperazine- 1-carboxamide | 495.1 | 496.1 | SFC: OJ-H Co-solvent: 20% EtOH + 0.1% NH₃.H₂O) Peak 2 |

### Examples 82A and 82B

### ((2R)—N—((R)-(5-chloro-6-(trifluoromethyl)pyridin-3-yl)(5-fluoro-6-(trifluoromethyl)pyridin-2-yl)methyl)-2-methyl-3-oxopiperazine-1-carboxamide and ((2R)—N—((S)-(5-chloro-6-(trifluoromethyl)pyridin-3-yl)(5-fluoro-6-(trifluoromethyl)pyridin-2-yl)methyl)-2-methyl-3-oxopiperazine-1-carboxamide

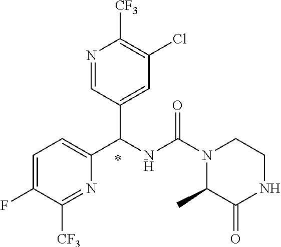 [SMILES: C[C@@H]1C(=O)NCCN1C(=O)NC(c1cnc(C(F)(F)F)c(Cl)c1)c1ccc(F)c(C(F)(F)F)n1]

Step 1: 3-chloro-5-(4,4,5,5-tetramethyl-1,3,2-dioxaborolan-2-yl)-2-(trifluoromethyl)pyridine To a solution of 3-chloro-2-(trifluoromethyl)pyridine (2.0 g, 11.02 mmol) and 4,4,4′,4′,5,5,5′,5′-octamethyl-2,2′-bi(1,3,2-dioxaborolane) (4.20 g, 16.53 mmol) in hexane (30 mL) were added 4,4′-di-tert-butyl-2,2′-bipyridine (0.296 g, 1.102 mmol) and bis(1,5-cyclooctadiene)rhodium(i) tetrafluoroborate (0.365 g, 0.551 mmol). The mixture was stirred at 65° C. for 18 hours, then diluted with water (30 mL), and extracted with DCM (3×10 mL). The combined organic layers were washed with brine (20 mL), dried over Na₂SO₄, filtered and the filtrate was concentrated under reduced pressure. The resulting residue was purified by MPLC (ISCO®: 12 g SepaFlash® Silica Flash Column, eluent of 0)˜8% pet. ether/EtOAc) to give the title compound. ¹H NMR (400 MHz, CDCl₃) δ 8.84 (s, 1H), 8.21 (s, 1H), 1.37 (s, 12H).

Step 2: 3-chloro-5-iodo-2-(trifluoromethyl)pyridine To a solution of 3-chloro-5-(4,4,5,5-tetramethyl-1,3,2-dioxaborolan-2-yl)-2-(trifluoromethyl) pyridine (1 g. 3.25 mmol) in DME (15 mL) were added 1-iodopyrrolidine-2,5-dione (2.195 g, 9.76 mmol), copper(I) iodide (0.062 g, 0.325 mmol), 1,10-phenanthroline (0.059 g, 0.325 mmol) and K₂CO₃ (0.905 g, 6.50 mmol) under N₂. The mixture was stirred at 50° C. for 12 hours, then cooled to RT, diluted with water (20 mL), and extracted with DCM (3×10 mL). The combined organic layers were washed with brine (20 mL), dried over Na₂SO₄, filtered, and the filtrate was concentrated in vacuo. The resulting residue was purified by Prep. TLC (ISCO®: 12 g SepaFlash® Silica Flash Column, eluent of 0˜1% petroleum ether/EtOAc) to give the title compound. LRMS m/z (M+H): calculated 306.9, observed 307.9.

Step 3: (R)—N-((5-chloro-6-(trifluoromethyl)pyridin-3-yl)(5-fluoro-6-(trifluoromethyl)pyridin-2-yl)methyl)-2-methylpropane-2-sulfinamide To a solution of 3-chloro-5-iodo-2-(trifluoro-methyl)pyridine (280 mg, 0.911 mmol) in toluene (3 mL) was added isopropylmagnesium lithium chloride in THF (1.3 M, 0.654 mL, 0.851 mmol) at −40° C. The mixture was stirred at −40° C. for 1 hour, then (R,E)-N-((5-fluoro-6-(trifluoromethyl) pyridin-2-yl)methylene)-2-methylpropane-2-sulfinamide (Intermediate from step 3 for Examples 48A and 48B, 180 mg, 0.608 mmol) in toluene (2 mL) was added. The mixture was stirred at −40° C. and then slowly warmed to 29° C. Then the reaction mixture was stirred at 29° C. for 4 hours, quenched with saturated aqueous NH₄Cl (10 mL), and extracted with EtOAc (2×10 mL). The combined organic layers were washed with brine (20 mL), dried over Na₂SO₄, filtered, and the filtrate was concentrated under reduced pressure. The resulting residue was purified by Prep. TLC (SiO₂, petroleum ether:EtOAc=2:1) to give the title compound. LRMS m/z (M+H): calculated 477.1, observed 478.1

Step 4: (5-chloro-6-(trifluoromethyl)pyridin-3-yl)(5-fluoro-6-(trifluoromethyl)pyridin-2-yl)methanamine hydrochloride To a solution of (R)—N-((5-chloro-6-(trifluoromethyl)pyridin-3-yl)(5-fluoro-6-(trifluoromethyl) pyridin-2-yl)methyl)-2-methylpropane-2-sulfinamide (240) mg, 0.502 mmol) in MeOH (2 mL) was added HCl/MeOH (4 N, 2 mL). The mixture was stirred at 27° C. for 11 hours, then concentrated under reduced pressure to give the title compound. LRMS m z (M+H): calculated 373.1, observed 374.1

Step 5: examples 82A and 82B To a solution of (5-chloro-6-(trifluoromethyl)pyridin-3-yl)(5-fluoro-6-(trifluoromethyl)pyridin-2-yl)methanamine hydrochloride (100 mg crude) in DMF (3 mL) was added CDI (56.8 mg, 0.351 mmol). The mixture was stirred at 27° C. for 1 hour, then (R)-3-methylpiperazin-2-one (22.01 mg, 0.193 mmol) was added. The reaction mixture was stirred at 27° C. for 2 hours, then diluted with MeCN (1 mL) and purified by prep-HPLC (62:38 to 32:68; water (0.1% TFA):MeCN (0.1% TFA)) to give a mixture of isomers, which was further separated by chiral-SFC (Column OJ-H, Co solvent: 0-30% EtOH with 0.1% NH₃H₂O) to give examples 82A (first eluted fraction) and 82B (second eluted fraction).

Example 82A: LRMS m/z (M+H): calculated 513.8.1, observed 514.2. ¹H NMR δ (ppm) (400 MHz, CD₃OD-d₄): 8.61 (d, J=1.6 Hz, 1H), 8.08 (s, 1H), 7.83-7.93 (m, 2H), 6.36 (s, 1H), 4.54-4.62 (m, 1H), 4.00-4.08 (m, 1H), 3.32-3.43 (m, 2H), 3.25-3.29 (m, 1H), 1.44 (d, J=7.2 Hz, 3H).

Example 82B: LRMS m/z (M+H): calculated 513.8.1, observed 514.2. ¹H NMR δ (ppm) (400 MHz, CD₃OD-d₄): 8.61 (d, J=2.0 Hz, 1H), 8.10 (d, J=1.2 Hz, 1H), 7.86-7.93 (m, 1H), 7.79-7.84 (m, 1H), 6.37 (s, 1H), 4.57-4.62 (m, 1H), 3.97-4.18 (m, 1H), 3.32-3.43 (m, 2H), 3.24-3.29 (m, 1H), 1.45 (d, J=7.2 Hz, 3H).

TABLE 15 The following examples were prepared according to the synthetic procedure for Examples 82A and 82B, using the appropriate starting materials and reagents

| | | | Calc'd | Observed | |
|---|---|---|---|---|---|
| Example | Compound | Name | [M + H]⁺ | [M + H]⁺ | Conditions |
| 83A | 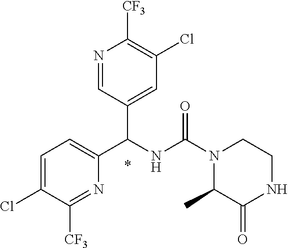 [SMILES: C[C@@H]1C(=O)NCCN1C(=O)NC(c1cnc(C(F)(F)F)c(Cl)c1)c1ccc(Cl)c(C(F)(F)F)n1] | (2R)-N-((R or S)-(3- chloro-2,4- difluorophenyl)(5- chloro-6- (trifluoromethyl)pyridin- 2-yl)methyl)-2-methyl- 3-oxopiperazine-1- carboxamide | 529.1 | 530.1 | SFC: AD-H Co-solvent: 25% (IPA + 0.1% NH3.H2O) peak 1 |
| 83B | 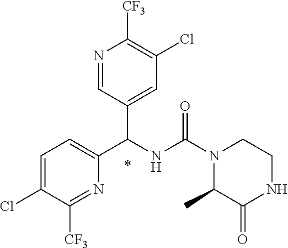 [SMILES: C[C@@H]1C(=O)NCCN1C(=O)NC(c1cnc(C(F)(F)F)c(Cl)c1)c1ccc(Cl)c(C(F)(F)F)n1] | (2R)-N-((S or R)-(3- chloro-2,4- difluorophenyl)(5- chloro-6- (trifluoromethyl)pyridin- 2-yl)methyl)-2-methyl- 3-oxopiperazine-1- carboxamide | 529.1 | 530.1 | SFC: AD-H Co-solvent: 25% (IPA + 0.1% NH3.H2O) Peak 2 |
| 84A | 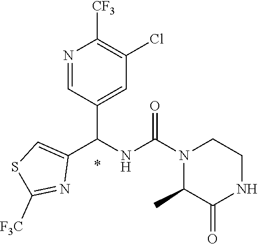 [SMILES: C[C@@H]1C(=O)NCCN1C(=O)NC(c1cnc(C(F)(F)F)c(Cl)c1)c1csc(C(F)(F)F)n1] | (2R)-N-((R or S)-(5- chloro-6-(trifluoro methyl)pyridin-3-yl)(2- (trifluoromethyl)thiazol- 4-yl)methyl)-2-methyl- 3-oxopiperazine-1- carboxamide | 501.1 | 502.1 | SFC: OD-H Co-solvent: 15% (EtOH + 0.1% NH3.H2O) peak 1 |
| 84B | 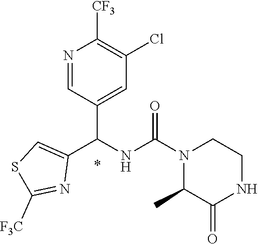 [SMILES: C[C@@H]1C(=O)NCCN1C(=O)NC(c1cnc(C(F)(F)F)c(Cl)c1)c1csc(C(F)(F)F)n1] | (2R)-N-((S or R)-(5- chloro-6-(trifluoro methyl)pyridin-3-yl)(2- (trifluoromethyl)thiazol- 4-yl)methyl)-2-methyl- 3-oxopiperazine-1- carboxamide | 501.1 | 502.1 | SFC: OD-H Co-solvent: 15% EtOH + 0.1% NH3.H2O) Peak 2 |

### Examples 85A and 85B

### (2R)—N—((R)-(3-chloro-4-fluorophenyl)(3-(trifluoromethyl)-1H-pyrazol-5-yl)methyl)-2-methyl-3-oxopiperazine-1-carboxamide and (2R)—N—((S)-(3-chloro-4-fluorophenyl)(3-(trifluoromethyl)-1H-pyrazol-5-yl)methyl)-2-methyl-3-oxopiperazine-1-carboxamide

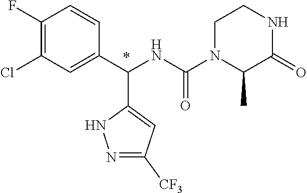 [SMILES: C[C@@H]1C(=O)NCCN1C(=O)NC(c1ccc(F)c(Cl)c1)c1cc(C(F)(F)F)n[nH]1]

Step 1: N-methoxy-N-methyl-5-(trifluoromethyl)-1H-pyrazole-3-carboxamide To a solution of 5-(trifluoromethyl)-1H-pyrazole-3-carboxylic acid (1.5 g, 8.33 mmol) in DMF (30 mL) was added DIEA (4.36 mL, 24.99 mmol) and HATU (6.33 g, 16.66 mmol) at 0° C. over 30 minutes. N,O-dimethylhydroxylamine hydrochloride (1.219 g, 12.49 mmol) was added, and the resulting mixture was stirred at 25° C. for 2 hours. Then water (150 mL) was added, and the mixture was extracted with ethyl acetate (3×15 mL). The combined organic layers were washed with brine (100 mL), dried over Na₂SO₄, filtered, and the filtrate was evaporated under reduced pressure. The resulting crude product was purified by flash silica gel chromatography (ISCO®; 12 g SepaFlash® Silica Flash Column, Eluent of 9% petroleum ether/ethyl acetate) to give the title compound. LRMS m/z (M+H): calculated 223.1, observed 223.9.

Step 2: (3-chloro-4-fluorophenyl)(5-(trifluoromethyl)-1H-pyrazol-3-yl)methanone To mixture of N-methoxy-N-methyl-5-(trifluoromethyl)-1H-pyrazole-3-carboxamide (600 mg, 2.69 mmol) in THF (3 mL) was added (3-chloro-4-fluorophenyl)magnesium bromide (13.44 mL, 13.44 mmol, 1 M in THF). The mixture was stirred at 0° C. for 2 hours, then aqueous NH₄Cl (20 mL) was added, and the mixture was extracted with ethyl acetate (3×10 mL). The combined organic layers were washed with brine (20 mL), dried over Na₂SO₄, filtered, and the filtrate was evaporated under reduced pressure to give the title compound. ¹H NMR (400 MHz, CDCl₃) δ 8.10 (dd, J=2.0, 6.8 Hz, 1H), 7.90-7.99 (m, 1H), 7.35 (t, J=8.4 Hz, 1H), 7.08 (s, 1H).

Step 3: (R,Z)—N-((3-chloro-4-fluorophenyl)(5-(trifluoromethyl)-1H-pyrazol-3-yl)methylene)-2-methylpropane-2-sulfinamide To a microwave tube charged with (3-chloro-4-fluorophenyl)(5-(trifluoromethyl)-1H-pyrazol-3-yl)methanone (400 mg. 1.367 mmol), (R)-2-methylpropane-2-sulfinamide (249 mg, 2.050 mmol) and toluene (3 mL) was added titanium(IV) ethoxide (0.562 mL, 2.73 mmol). The reaction mixture was microwaved at 105° C. for 30 minutes and then cooled to RT. The resulting crude product (400 mg, crude) was used directly in the next step without further purification.

Step 4: (R)—N-((3-chloro-4-fluorophenyl)(5-(trifluoromethyl)-1H-pyrazol-3-yl)methyl)-2-methylpropane-2-sulfinamide A solution of (R,Z)—N-((3-chloro-4-fluorophenyl)(5-(trifluoro-methyl)-1H-pyrazol-3-yl)methylene)-2-methylpropane-2-sulfinamide (400 mg, crude, from Step 3) in THF (5 mL) and water (0.01 mL) was cooled to −78° C., followed by the addition of NaBH₄ (57.4 mg, 1.516 mmol). The reaction mixture was stirred at −78° C. for 1 hour, then gradually warmed to 0° C. over 1 hour and maintained at 0° C. for 1 hour. The reaction mixture was warmed to room temperature, then aqueous NaHCO₃ (50 mL) was added, and the mixture was extracted with ethyl acetate (3×10 mL). The combined organic layers were washed with brine (50 mL), dried over Na₂SO₄, filtered, and the filtrate was evaporated under reduced pressure. The resulting residue was purified by prep-TLC (SiO₂, petroleum ether:ethyl acetate=1:1) to give the title compound. LRMS m/z (M+H): calculated 397.1, observed 398.0.

Step 5: (3-chloro-4-fluorophenyl)(5-(trifluoromethyl)-1H-pyrazol-3-yl)methanamine hydrochloride To a mixture of (R)—N-((3-chloro-4-fluorophenyl)(5-(trifluoromethyl)-1H-pyrazol-3-yl)methyl)-2-methylpropane-2-sulfinamide (300 mg, 0.754 mmol) in MeOH (1 mL) was added HCl in methanol (3 M, 3 mL, 0.754 mmol) at 25° C. The resulting mixture was stirred at 25° C. for 1 hour. Then the was mixture evaporated under reduced pressure to give the title compound. LRMS m/z (M+H): calculated 293.1, observed 294.0.

Step 6: Examples 85A and 85B A mixture of (3-chloro-4-fluorophenyl)(5-(trifluoromethyl)-1H-pyrazol-3-yl)methanamine hydrochloride (100 mg crude) and CDI (98 mg, 0.606 mmol) in DMF (1 mL) was stirred at 20° C. for 10 minutes, then (R)-3-methylpiperazin-2-one (41.5 mg, 0.364 mmol) in DMF (0.5 mL) was added. The resulting mixture was stirred at 20° C. for 1 hour and then purified by reverse phase HPLC (57:43 to 27:73; water (0.1% TFA):MeCN (0.1% TFA)), followed by lyophilization to give a mixture of isomers, which was further separated by Chiral-SFC (Column AD-H, Co solvent: 25% IPA with 0.1% NH₃:H₂O) to give Examples 85A (first eluted fraction) and 85B (second eluted fraction).

Example 85A: LRMS m/z (M+H): calculated 433.1, observed 434.1. ¹H NMR δ (ppm) (400 MHz, CD₃OD-d₄): 7.47-51 (m, 1H), 7.22-7.35 (m, 2H), 6.31-6.35 (m, 1H), 6.26 (s, 1H), 4.60 (q, J=7.2 Hz, 1H), 3.99-4.12 (m, 1H), 3.34-3.47 (m, 1H), 3.20-3.30 (m, 2H), 1.43 (d, J=7.2 Hz, 3H).

Example 85B: LRMS m/z (M+H): calculated 433.1, observed 434.1. ¹H NMR δ (ppm) (400 MHz, CD₃OD-d₄): 7.45-7.49 (m, 1H), 7.22-7.37 (m, 2H), 6.30-6.34 (m, 1H), 6.18-6.29 (m, 1H), 4.56 (q, J=6.8 Hz, 1H), 4.04-4.09 (m, 1H), 3.94-4.19 (m, 1H), 3.33-3.43 (m, 1H), 3.22-3.30 (m, 2H), 1.42 (d, J=7.2 Hz, 3H).

TABLE 16 The following examples were prepared according to the synthetic procedure for Example 85A and 85B, using the appropriate starting materials and reagents

| | | | Calc'd | Observed | |
|---|---|---|---|---|---|
| Example | Compound | Name | [M + H]⁺ | [M + H]⁺ | Conditions |
| 86A | 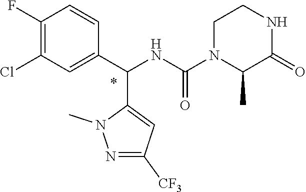 [SMILES: C[C@@H]1C(=O)NCCN1C(=O)NC(c1ccc(F)c(Cl)c1)c1cc(C(F)(F)F)nn1C] | (2R)-N-((R or S)-(3- chloro-4-fluoro- phenyl)(1-methyl-3- (trifluoro-methyl)-1H- pyrazol-5-yl)methyl)-2- methyl-3- oxopiperazine-1- carboxamide | 447.1 | 448.1 | SFC: AD-H Co-solvent: 25% (IPA + 0.1% NH₃.H₂O) peak 1 |
| 86B | 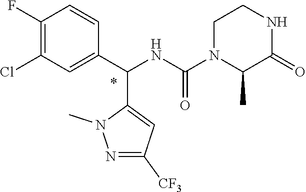 [SMILES: C[C@@H]1C(=O)NCCN1C(=O)NC(c1ccc(F)c(Cl)c1)c1cc(C(F)(F)F)nn1C] | (2R)-N-((S or R)-(3- chloro-4-fluoro- phenyl)(1-methyl-3- (trifluoro-methyl)-1H- pyrazol-5-yl)methyl)-2- methyl-3-oxo- piperazine-1- carboxamide | 447.1 | 448.1 | SFC: AD-H Co-solvent: 25% (IPA + 0.1% NH₃.H₂O) Peak 2 |
| 87A | 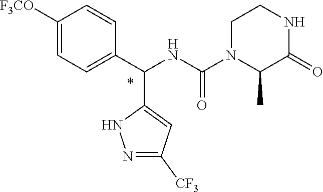 [SMILES: C[C@@H]1C(=O)NCCN1C(=O)NC(c1ccc(OC(F)(F)F)cc1)c1cc(C(F)(F)F)n[nH]1] | (2R)-2-methyl-3-oxo- N-((R or S)-(4- (trifluoro methoxy) phenyl)(3- (trifluoromethyl)-1H- pyrazol-5- yl)methyl)piperazine-1- carboxamide | 465.1 | 466.1 | SFC: AD-H Co-solvent: 25% (IPA + 0.1% NH₃.H₂O) peak 1 |
| 87B | 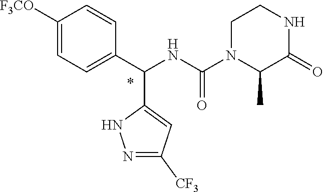 [SMILES: C[C@@H]1C(=O)NCCN1C(=O)NC(c1ccc(OC(F)(F)F)cc1)c1cc(C(F)(F)F)n[nH]1] | (2R)-2-methyl-3-oxo- N-((S or R)-(4- (trifluoro methoxy) phenyl)(3- (trifluoromethyl)-1H- pyrazol-5- yl)methyl)piperazine-1- carboxamide | 465.1 | 466.1 | SFC: AD-H Co-solvent: 25% (IPA + 0.1% NH₃.H₂O) Peak 2 |

### EXAMPLE OF A PHARMACEUTICAL COMPOSITION

As a specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

### Biological Assays

### Qube® Assay Experimental Procedure

Compounds were tested on human NaV 1.8 and NaV1.5 channels stably expressed in human embryo kidney (HEK) 293 cells. Sodium current measurements on Qube® were conducted as follows: automated 384-well patch-clamp assays on the Qube® platform (Sophion Biosciences) were used to measure the inhibition of sodium flow through human NaV1.8 and NaV1.5 channels. Whole-cell voltage-clamp recordings were performed in QChips® (Sophion Biosciences) at room temperature. NaV1.8 current measurements on Qube® were obtained as follows: NaV1.8 currents were elicited with a 10 second 1 Hertz (Hz) pulse train from a holding potential of −90 millivolts (mV), delivered to the cells once per minute in the control condition (DMSO only) and after compound addition. The 1 hertz pulse train stimulation consisted of ten test pulses to 10 millivolt (mV) for 20 milliseconds (ms), each of which was followed by a 980 millisecond repolarization to −67 millivolts. At the end of the 10 second pulse train stimulation, a 5 second hyperpolarization step to −100 millivolt (mV) was used to recover NaV1.8 from fast inactivation. The peak currents elicited by the 1st and 10th test pulses were used to determine IC50 values for resting inhibition and inactivated state inhibition. NaV1.5 current measurements on Qube® were obtained as follows: NaV1.5 currents were elicited with a 20 second 3 Hertz pulse train in the control condition (DMSO only) and after compound addition. The pulse train consisted of sixty 20 millisecond test pulses to 0 millivolt from a holding potential of −80 millivolt (mV). The average peak currents elicited by the last 3 test pulses were used to determine IC₅₀ values for NaV1.5 inhibition.

The following buffers were used for the Qube® recordings: External buffer for NaV1.8 Qube® recording: 150 NaCl, 2 CaCl₂, 5 KCl, 1 MgCl₂, 10 HEPES, 12 Dextrose; External buffer for Qube® NaV1.5 recording: 120 N-Methyl-D-Glucamine, 40 NaCl, 1 KCl, 2.7 CaCl₂), 5 HEPES, 0.5 MgCl₂; and Internal buffer for Qube® recording: 120 CsF, 30 CsCl, 10 EGTA, 5 HEPES, 5 NaF, 2 MgCl₂.

For all Qube® experiments offline analysis was used to determine percent inhibition as a function of drug concentration. IC₅₀ values were determined by fitting to the Hill equation. The compounds of the present invention have Nav1.8 IC₅₀ values in the Qube® Assay of less than 5 micromolar. Specific IC₅₀ values of the compounds of Examples 1A-87B in the Qube® Assay are listed in Table I.

TABLE I
IC₅₀ values (nM) for Examples in the Nav1.8 Qube ® Assay

| Example | IC₅₀ (nM) |
|---|---|
| 1A | 1.09 |
| 1B | 30.9 |
| 2A | 8.98 |
| 2B | 122.8 |
| 3A | 2.22 |
| 3B | 21.9 |
| 4A | 3.9 |
| 4B | 25.7 |
| 5A | 17.6 |
| 5B | 28.88 |
| 6A | 20.02 |
| 6B | 127.8 |
| 7A | 10.77 |
| 7B | 19.62 |
| 8A | 4.46 |
| 8B | 136.1 |
| 9A | 61.6 |
| 9B | 8.03 |
| 10A | 1.18 |
| 10B | 27.99 |
| 11A | 67.44 |
| 11B | 196.5 |
| 12A | 9.24 |
| 12B | 500.8 |
| 13A | 2.13 |
| 13B | 9.42 |
| 14A | 5.57 |
| 14B | 100.5 |
| 15A | 4.74 |
| 15B | 59.88 |
| 16A | 13.46 |
| 16B | 31.68 |
| 17A | 101 |
| 17B | 34.58 |
| 18A | 3.89 |
| 18B | 4.69 |
| 19A | 41.1 |
| 19B | 72.56 |
| 20A | 4.01 |
| 20B | 4 |
| 21A | 1.45 |
| 21B | 7.71 |
| 22A | 0.31 |
| 22B | 2.88 |
| 23A | 16.96 |
| 23B | 6.06 |
| 24A | 22.31 |
| 24B | 2.84 |
| 25A | 3.51 |
| 25B | 15.8 |
| 26A | 1.89 |
| 26B | 3.81 |
| 27A | 52.58 |
| 27B | 2.88 |
| 28A | 10.75 |
| 28B | 43.69 |
| 29A | 21.43 |
| 29B | 3.42 |
| 30A | 506 |
| 30B | 9.18 |
| 31A | 3.47 |
| 31B | 16.14 |
| 32A | 96.83 |
| 32B | 13.02 |
| 33A | 14.39 |
| 33B | 514.1 |
| 34 | 2.12 |
| 35 | 1.13 |
| 36 | 6.97 |
| 37A | 1.85 |
| 37B | 187.6 |
| 38A | 558.6 |
| 38B | 2.22 |
| 39A | 2628 |
| 39B | 15.24 |
| 40A | 728.8 |
| 40B | 10.51 |
| 41A | 2021 |
| 41B | 4.94 |
| 42 | 32.17 |
| 43 | 229.9 |
| 44 | 8.55 |
| 45 | 1698 |
| 46A | 923.6 |
| 46B | 506.5 |
| 47A | 2305 |
| 47B | 3757 |
| 48A | 4.78 |
| 48B | 208.7 |
| 49A | 258.3 |
| 49B | 8.65 |
| 50A | 73.69 |
| 50B | 8.55 |
| 51A | 128.6 |
| 51B | 13.19 |
| 52A | 5.79 |
| 52B | 64.5 |
| 53A | 68.89 |
| 53B | 1.38 |
| 54A | 284.5 |
| 54B | 4.14 |
| 55A | 2.68 |
| 55B | 15.66 |
| 56A | 11.75 |
| 56B | 1.31 |
| 57A | 6.79 |
| 57B | 125 |
| 58A | 3.6 |
| 59A | 3.33 |
| 59B | 31.8 |
| 60A | 2.19 |
| 60B | 8.29 |
| 61A | 1.02 |
| 61B | 3.83 |
| 62A | 197.4 |
| 62B | 2.45 |
| 63A | 1.122 |
| 63B | 13.45 |
| 64A | 4.96 |
| 64B | 3.5 |
| 65A | 8.05 |
| 65B | 89.33 |
| 66A | 125 |
| 66B | 82.32 |
| 67A | 890.4 |
| 67B | 41.95 |
| 68A | 34.91 |
| 68B | 760.9 |
| 69A | 136 |
| 69B | 23.02 |
| 70A | 51.38 |
| 70B | 6.87 |
| 71A | 82.6 |
| 71B | 2.76 |
| 72A | 195.1 |
| 72B | 25.28 |
| 73A | 7.01 |
| 73B | 20.58 |
| 74A | 71.15 |
| 74B | 1322 |
| 75A | 89.47 |
| 75B | 2.95 |
| 76A | 5.18 |
| 76B | 167.1 |
| 77A | 4.59 |
| 77B | 1.06 |
| 78A | 15.3 |
| 78B | 3.543 |
| 79 | 0.58 |
| 80 | 1.90 |
| 81A | 7.996 |
| 81B | 110.1 |
| 82A | 17.44 |
| 82B | 3.76 |
| 83A | 6.51 |
| 83B | 3.14 |
| 84A | 16.16 |
| 84B | 201.6 |
| 85A | 57.78 |
| 85B | 5.47 |
| 86A | 3.59 |
| 86B | 153.6 |
| 87A | 123.7 |
| 87B | 10.8 |

The scope of the claims should not be limited by the preferred embodiments set forth in the examples, but should be given the broadest interpretation consistent with the description as a whole.

While the invention has been described and illustrated with reference to certain particular embodiments thereof, those skilled in the art will appreciate that various adaptations, changes, modifications, substitutions, deletions, or additions of procedures and protocols may be made without departing from the scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention.

1. A compound of structural Formula I: 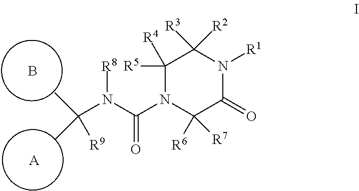
or a pharmaceutically acceptable salt thereof, wherein
one of A and B is selected from:
(1) aryl, and
(2) heteroaryl,
wherein each aryl and heteroaryl is unsubstituted or substituted with one to five substituents selected from R^{a}, and
the other of A and B is selected from:
(1) aryl, and
(2) heteroaryl,
wherein B is unsubstituted or substituted with one to five substituents selected from R^{b}, provided that when A is aryl then B is not aryl;
R¹ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)ₜC(O)R^{j},
(9) —(CH₂)ₜC(O)NR^{e}R^{j},
(10) —(CH₂)ₙNR^{e}C(O)R^{j},
(11) —(CH₂)ₙNR^{e}C(O)OR^{j},
(12) —(CH₂)ₙNR^{e}C(O)N(R^{e})₂,
(13) —(CH₂)ₙNR^{e}C(O)NR^{e}R^{j},
(14) —(CH₂)ₙNR^{e}S(O)ₘR^{j},
(15) —(CH₂)ₙNR^{e}S(O)ₘN(R^{e})₂,
(16) —(CH₂)ₙNR^{e}S(O)ₘNR^{e}R^{j}, and
(17) —(CH₂)ₙNR^{e}R^{j},
wherein each CH₂, alkyl, alkenyl, alkynyl, cycloalkyl and cycloheteroalkyl is unsubstituted or substituted with one to five substituents selected from R^{c};
R² is selected from:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{d},
wherein R² and R³ and the carbon atoms they are connected to can from a —C₃₋₅cycloalkyl ring, and wherein R² and R⁴ and the carbon atoms they are connected to can from a —C₃₋₅cycloalkyl ring;
R³ is selected from:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{d};
R⁴ is selected from:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{f}, and
wherein R⁴ and R⁵ and the carbon atoms they are connected to can from a —C₃₋₅cycloalkyl ring;
R⁵ is selected from the group consisting of:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{f}, and
wherein R⁵ and R⁷ and the carbon atoms they are attached to may form a 4-, 5- or 6-membered saturated ring;
R⁶ is selected from:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)_{S}C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{g}, and wherein R⁶ and R⁷ and the carbon atoms they are connected to can from a —C₃₋₅cycloalkyl ring;
R⁷ is selected from:
(1) hydrogen,
(2) —C₁₋₆alkyl,
(3) —C₂₋₆alkenyl,
(4) —C₂₋₆alkynyl,
(5) —C₃₋₆cycloalkyl,
(6) —C₂₋₆cycloheteroalkyl,
(7) —C₁₋₆alkyl-O—C₁₋₆alkyl-,
(8) —(CH₂)§ C(O)R^{j},
(9) —(CH₂)_{S}C(O)NR^{e}R^{j},
(10) —(CH₂)_{S}NR^{e}C(O)R^{j},
(11) —(CH₂)_{S}NR^{e}C(O)OR^{j},
(12) —(CH₂)_{S}NR^{e}C(O)N(R^{e})₂,
(13) —(CH₂)_{S}NR^{e}C(O)NR^{e}R^{j},
(14) —(CH₂)_{S}NR^{e}S(O)ₘR^{j},
(15) —(CH₂)_{S}NR^{e}S(O)ₘN(R^{e})₂,
(16) —(CH₂)_{S}NR^{e}S(O)ₘNR^{e}R^{j}, and
(17) —(CH₂)_{S}NR^{e}R^{j},
wherein each CH₂, alkyl, alkenyl, alkynyl, cycloalkyl, and cycloheteroalkyl is unsubstituted or substituted with one to five substituents selected from R^{g};
R⁸ is selected from:
(1) hydrogen,
(2) —C₁₋₆alkyl,
(3) —C₃₋₆cycloalkyl, and
(4) —C₂₋₆cycloheteroalkyl,
wherein each alkyl, cycloalkyl and cycloheteroalkyl is unsubstituted or substituted with one to five substituents selected from R^{e};
R⁹ is selected from:
(1) hydrogen,
(2) —C₁₋₆alkyl,
(3) —C₂₋₆alkenyl, and
(4) —C₂₋₆alkynyl,
wherein each alkyl, alkenyl and alkynyl is unsubstituted or substituted with one to five substituents selected from halogen;
each R^{a} is independently selected from:
(1) CN,
(2) OXO,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —C₂₋₆alkenyl-C₃₋₆cycloalkyl,
(17) —C₂₋₆alkenyl-C₂₋₆cycloheteroalkyl,
(18) —C₂₋₆alkenyl-aryl,
(19) —C₂₋₆alkenyl-heteroaryl,
(20) —C₂₋₆alkynyl-C₃₋₆cycloalkyl,
(21) —C₂₋₆alkynyl-C₂₋₆cycloheteroalkyl,
(22) —C₂₋₆alkynyl-aryl,
(23) —C₂₋₆alkynyl-heteroaryl,
(24) —OH,
(25) —(CH₂)ₚ—OC₁₋₆alkyl,
(26) —(CH₂)ₚ—OC₂₋₆alkenyl,
(27) —(CH₂)ₚ—OC₂₋₆alkynyl,
(28) —(CH₂)ₚ—OC₃₋₆cycloalkyl,
(29) —(CH₂)ₚ—OC₂₋₆cycloheteroalkyl,
(30) —(CH₂)ₚ—O-aryl,
(31) —(CH₂)ₚ—O-heteroaryl,
(32) —OC₁₋₆alkyl-C₃₋₆cycloalkyl,
(33) —OC₁₋₆alkyl-C₂₋₆cycloheteroalkyl,
(34) —OC₁₋₆alkyl-aryl,
(35) —OC₁₋₆alkyl-heteroaryl,
(36) —S(O)ᵣR^{h},
(37) —C₁₋₆alkyl-S(O)ᵣR^{h},
(38) —N(R^{k})₂,
(39) —C(O)R^{L}, and
(40) —NR^{k}R^{L},
wherein each R^{a} is unsubstituted or substituted with one to six substituents selected from halogen, CF₃, OH, C₁₋₆alkyl, and OC₁₋₆alkyl;
each R^{b} is independently selected from the group consisting of:
(1) CN,
(2) OXO,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —C₂₋₆alkenyl-C₃₋₆cycloalkyl,
(17) —C₂₋₆alkenyl-C₂₋₆cycloheteroalkyl,
(18) —C₂₋₆alkenyl-aryl,
(19) —C₂₋₆alkenyl-heteroaryl,
(20) —C₂₋₆alkynyl-C₃₋₆cycloalkyl,
(21) —C₂₋₆alkynyl-C₂₋₆cycloheteroalkyl,
(22) —C₂₋₆alkynyl-aryl,
(23) —C₂₋₆alkynyl-heteroaryl,
(24) —OH,
(25) —(CH₂)ₚ—OC₁₋₆alkyl,
(26) —(CH₂)ₚ—OC₂₋₆alkenyl,
(27) —(CH₂)ₚ—OC₂₋₆alkynyl,
(28) —(CH₂)ₚ—OC₃₋₆cycloalkyl,
(29) —(CH₂)ₚ—OC₂₋₆heterocycloalkyl,
(30) —(CH₂)ₚ—O-aryl,
(31) —(CH₂)ₚ—O-heteroaryl,
(32) —OC₁₋₆alkyl-C₃₋₆cycloalkyl,
(33) —OC₁₋₆alkyl-C₂₋₆heterocycloalkyl,
(34) —OC₁₋₆alkyl-aryl,
(35) —OC₁₋₆alkyl-heteroaryl,
(36) —S(O)ᵣRⁱ,
(37) —C₁₋₆alkyl-S(O)ᵣRⁱ,
(38) —N(R^{k})₂,
(39) —C(O)R^{L}, and
(40) —NR^{K}R^{L},
wherein each R^{b} is unsubstituted or substituted with one to six substituents selected from halogen, CF₃, OCF₃, CN, CH₂CF₃, CF₂CH₃, —C₁₋₆alkyl, and O—C₁₋₆alkyl;
R^{c} is selected from:
(1) —C₁₋₆alkyl,
(2) OH,
(3) halogen, and
(4) —OC₁₋₆alkyl,
wherein alkyl is unsubstituted or substituted with one to three halogens;
R^{d} is selected from:
(1) —C₁₋₆alkyl,
(2) OH,
(3) halogen, and
(4) —OC₁₋₆alkyl,
wherein alkyl is unsubstituted or substituted with one to three halogens;
R^{e} is selected from:
(1) hydrogen, and
(2) C₁₋₆alkyl;
R^{f} is selected from:
(1) —C₁₋₆alkyl,
(2) OH,
(3) halogen, and
(4) —OC₁₋₆alkyl,
wherein alkyl is unsubstituted or substituted with one to three halogens;
R^{g} is selected from:
(1) —C₁₋₆alkyl,
(2) OH,
(3) halogen, and
(4) —OC₁₋₆alkyl,
wherein alkyl is unsubstituted or substituted with one to three halogens;
R^{h} is selected from:
(1) hydrogen,
(2) C₁₋₆alkyl,
(3) C₃₋₆cycloalkyl,
(4) aryl, and
(5) heteroaryl;
Rⁱ is selected from:
(1) hydrogen,
(2) C₁₋₆alkyl,
(3) C₃₋₆cycloalkyl,
(4) aryl, and
(5) heteroaryl;
R^{j} is selected from:
(1) hydrogen,
(2) C₁₋₆alkyl,
(3) C₃₋₆alkenyl,
(4) C₃₋₆alkynyl,
(5) C₃₋₆cycloalkyl,
(6) C₂₋₅cycloheteroalkyl,
(7) aryl, and
(8) heteroaryl;
R^{k} is selected from:
(1) hydrogen, and
(2) C₁₋₆alkyl;
R^{L} is selected from:
(1) hydrogen,
(2) C₁₋₆alkyl,
(3) C₃₋₆cycloalkyl,
(4) aryl, and
(5) heteroaryl;
m is independently selected from 0, 1 and 2;
n is independently selected from 2, 3, 4, 5 and 6;
p is independently selected from 0, 1, 2 and 3;
q is independently selected from 0, 1, 2 and 3;
r is independently selected from 0, 1 and 2;
s is independently selected from 0, 1, 2, 3, 4, 5, and 6; and
t is is independently selected from 0, 1, 2, 3, 4, 5, and 6.

2. The compound according to claim 1 wherein A is selected from:
(1) aryl, and
(2) heteroaryl,
wherein each aryl and heteroaryl is unsubstituted or substituted with one to five substituents selected from R^{a}; or a pharmaceutically acceptable salt thereof.

3. The compound according to claim 1 wherein A is selected from:
(1) phenyl,
(2) pyridine,
(3) pyrazole,
(4) oxazole, and
(5) thiazole,
wherein A is unsubstituted or substituted with one to five substituents selected from R^{a};
or a pharmaceutically acceptable salt thereof.

4. The compound according to claim 1 wherein A is selected from:
(1) phenyl, and
(2) pyridine,
wherein phenyl and pyridine are unsubstituted or substituted with one to five substituents selected from R^{a}; or a pharmaceutically acceptable salt thereof.

5. The compound according to claim 1 wherein B is independently selected from:
(1) phenyl,
(2) pyridine,
(3) pyrimidine,
(4) pyrazole,
(5) thiazole,
(6) imidazo[1,2-a]pyridine,
(7) oxazole,
(8) benzofuran,
(9) benzoxazole,
(10) indazole, and
(11) thiazolopyridine,
wherein B is unsubstituted or substituted with one to five substituents selected from R^{b};
or a pharmaceutically acceptable salt thereof.

6. The compound according to claim 1 wherein B is heteroaryl, wherein heteroaryl is unsubstituted or substituted with one to five substituents selected from R^{b}; or a pharmaceutically acceptable salt thereof.

7. The compound according to claim 6 wherein B is independently selected from:
(1) pyridine,
(2) pyrimidine,
(3) pyrazole,
(4) thiazole, and
(5) imidazo[1,2-a]pyridine,
wherein B is unsubstituted or substituted with one to five substituents selected from R^{b}; or a pharmaceutically acceptable salt thereof.

8. The compound according to claim 1 wherein
R¹ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{c};
R² is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R³ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R⁴ is selected from:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁵ is selected from:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁶ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g}; and
R⁷ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
or a pharmaceutically acceptable salt thereof.

9. The compound according to claim 1 wherein R⁸ is selected from:
(1) hydrogen, and
(2) —C₁₋₆alkyl,
wherein alkyl is unsubstituted or substituted with one to five substituents selected from R^{e}; and
R⁹ is selected from:
(1) hydrogen, and
(2) —C₁₋₆alkyl,
wherein each alkyl is unsubstituted or substituted with one to five substituents selected from halogen;
or a pharmaceutically acceptable salt thereof.

10. The compound according to claim 1 wherein A is selected from:
(1) aryl, and
(2) heteroaryl,
wherein each aryl and heteroaryl is unsubstituted or substituted with one to five substituents selected from R^{a}; or a pharmaceutically acceptable salt thereof.

11. The compound according to claim 1 wherein R¹, R², R³, R⁴, R⁵, R⁸, and R⁹ are hydrogen; or a pharmaceutically acceptable salt thereof.

12. The compound according to claim 1 wherein each R^{a} is independently selected from:
(1) CN,
(2) OXO,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —OH,
(17) —OC₁₋₆alkyl,
(18) —OC₃₋₆cycloalkyl, and
(19) —OC₂₋₆cycloheteroalkyl,
wherein each R^{a} is unsubstituted or substituted with one to six substituents selected from halogen, CF₃, OH, C₁₋₆alkyl, and —OC₁₄alkyl; or a pharmaceutically acceptable salt thereof.

13. The compound according to claim 1 wherein each R^{a} is independently selected from:
(1) CN,
(2) halogen,
(3) —C₁₋₆alkyl,
(4) —C₁₋₆alkenyl,
(5) —C₃₋₆cycloalkyl,
(6) aryl,
(7) —OC₁₋₆alkyl, and
(8) —OC₃₋₆cycloalkyl,
wherein each R^{a} is unsubstituted or substituted with one to six substituents selected from halogen, CF₃, OH, C₁₋₆alkyl, and —OC₁₋₆alkyl; or a pharmaceutically acceptable salt thereof.

14. The compound according to claim 1 wherein each R^{b} is independently selected from:
(1) CN,
(2) OXO,
(3) halogen,
(4) —S(O)₂C₁₋₆alkyl,
(5) —C₁₋₆alkyl,
(6) —C₁₋₆alkenyl,
(7) —C₃₋₆cycloalkyl,
(8) —C₂₋₆cycloheteroalkyl,
(9) aryl,
(10) heteroaryl,
(11) —OH,
(12) —OC₁₋₆alkyl,
(13) —OC₃₋₆cycloalkyl, and
(14) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl; or a pharmaceutically acceptable salt thereof.

15. The compound according to claim 1 wherein each R^{b} is independently selected from:
(1) CN,
(2) halogen,
(3) —C₁₋₆alkyl,
(4) —C₁₋₆alkenyl,
(5) —C₃₋₆cycloalkyl,
(6) —C₂₋₆cycloheteroalkyl,
(7) aryl,
(8) heteroaryl,
(9) —OC₁₋₆alkyl,
(10) —OC₃₋₆cycloalkyl, and
(11) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl; or a pharmaceutically acceptable salt thereof.

16. The compound according to claim 1 wherein
A is selected from:
(1) phenyl,
(2) pyridine,
(3) pyrazole,
(4) oxazole, and
(5) thiazole,
wherein A is unsubstituted or substituted with one to five substituents selected from R^{a};
B is independently selected from:
(1) aryl, and
(2) heteroaryl,
wherein B is unsubstituted or substituted with one to five substituents selected from R^{b};
R¹ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{c};
R² is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R³ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{d};
R⁴ is selected from:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁵ is selected from:
(1) hydrogen,
(2) —C₁₋₆alkyl, and
(3) —C₃₋₆cycloalkyl,
wherein alkyl and cycloalkyl is unsubstituted or substituted with one to five substituents selected from R^{f};
R⁶ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁷ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁸ is selected from:
(1) hydrogen, and
(2) —C₁₋₆alkyl,
wherein alkyl is unsubstituted or substituted with one to five substituents selected from R^{e};
R⁹ is selected from:
(1) hydrogen, and
(2) —C₁₋₆alkyl,
wherein each alkyl is unsubstituted or substituted with one to five substituents selected from halogen;
each R^{a} is independently selected from:
(1) CN,
(2) OXO,
(3) halogen,
(4) —S(O)₂C₁₋₆alkyl,
(5) —C₁₋₆alkyl,
(6) —C₁₋₆alkenyl,
(7) —C₂₋₆alkynyl,
(8) —C₃₋₆cycloalkyl,
(9) —C₂₋₆cycloheteroalkyl,
(10) aryl,
(11) heteroaryl,
(12) —C₁₋₆alkyl-aryl,
(13) —C₁₋₆alkyl-heteroaryl,
(14) —C₁₋₆alkyl-C₃₋₆cycloalkyl,
(15) —C₁₋₆alkyl-C₂₋₆cycloheteroalkyl,
(16) —OH,
(17) —OC₁₋₆alkyl,
(18) —OC₃₋₆cycloalkyl, and
(19) —OC₂₋₆cycloheteroalkyl,
wherein each R^{a} is unsubstituted or substituted with one to six substituents selected from halogen, CF₃, OH, C₁₋₆alkyl, and —OC₁₄alkyl; and
each R^{b} is independently selected from:
(1) CN,
(2) oxo,
(3) halogen,
(4) —S(O)₂C₁₋₆alkyl,
(5) —C₁₋₆alkyl,
(6) —C₁₋₆alkenyl,
(7) —C₃₋₆cycloalkyl,
(8) —C₂₋₆cycloheteroalkyl,
(9) aryl,
(10) heteroaryl,
(11) —OH,
(12) —OC₁₋₆alkyl,
(13) —OC₃₋₆cycloalkyl, and
(14) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
or a pharmaceutically acceptable salt thereof.

17. The compound according to claim 1 wherein
A is selected from:
(1) phenyl, and
(2) pyridine,
wherein phenyl and pyridine are unsubstituted or substituted with one to five substituents selected from R^{a};
B is heteroaryl, wherein heteroaryl is unsubstituted or substituted with one to five substituents selected from R^{b};
R¹, R², R³, R⁴ and R⁵ are hydrogen;
R⁶ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁷ is selected from:
(1) hydrogen,
(2) —C₁₋₆alkyl, and
(3) —C₃₋₆cycloalkyl,
wherein each alkyl and cycloalkyl is unsubstituted or substituted with one to five substituents selected from R^{g};
R⁸ and R⁹ are hydrogen;
each R^{a} is independently selected from:
(1) CN,
(2) halogen,
(3) —C₁₋₆alkyl,
(4) —C₁₋₆alkenyl,
(5) —C₃₋₆cycloalkyl, aryl,
(6) —OC₁₋₆alkyl, and
(7) —OC₃₋₆cycloalkyl,
wherein each R^{a} is unsubstituted or substituted with one to six substituents selected from halogen, CF₃, OH, C₁₋₆alkyl, and —OC₁₋₆alkyl; and
each R^{b} is independently selected from:
(1) CN,
(2) halogen,
(3) —C₁₋₆alkyl,
(4) —C₁₋₆alkenyl,
(5) —C₃₋₆cycloalkyl,
(6) —C₂₋₆cycloheteroalkyl,
(7) aryl,
(8) heteroaryl,
(9) —OC₁₋₆alkyl,
(10) —OC₃₋₆cycloalkyl, and
(11) —OC₂₋₆heterocycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
or a pharmaceutically acceptable salt thereof.

18. The compound according to claim 1 selected from:
(1) (2R)—N—((R)(3-chloro-2,4-difluorophenyl)(6-(2,2,2-trifluoroethoxy)pyridin-3-yl)methyl)-2-methyl-3-oxopiperazine-1-carboxamide;
(2) (2R)—N—((S)(3-chloro-2,4-difluorophenyl)(6-(2,2,2-trifluoroethoxy)pyridin-3-yl)methyl)-2-methyl-3-oxopiperazine-1-carboxamide;
(3) N—((R or S)-(3-chloro-4-fluorophenyl)(6-(2,2,2-trifluoro-ethoxy)pyridin-3-yl)methyl)-3-oxopiperazine-1-carboxamide;
(4) N—((S or R)-(3-chloro-4-fluorophenyl)(6-(2,2,2-trifluoroethoxy)pyridin-3-yl)methyl)-3-oxopiperazine-1-carboxamide;
(5) (2R)—N—((R or S)-(3-chloro-4-fluorophenyl)(6-(2,2,2-trifluoroethoxy)pyridin-3-yl)methyl)-2-methyl-3-oxopiperazine-1-carboxamide;
(6) (2R)—N—((S or R)-(3-chloro-4-fluorophenyl)(6-(2,2,2-trifluoroethoxy)pyridin-3-yl)methyl)-2-methyl-3-oxopiperazine-1-carboxamide;
(7) (2R)—N—((R or S)-(3-chloro-2,4-difluorophenyl)(6-(trifluoromethoxy)pyridin-3-yl)methyl)-2-methyl-3-oxopiperazine-1-carboxamide;
(8) (2R)—N—((S or R)-(3-chloro-2,4-difluorophenyl)(6-(trifluoromethoxy)pyridin-3-methyl-3-oxopiperazine-1-carboxamide;
(9) (2R)—N—((R)-(4-chlorophenyl)(6-(2,2,2-trifluoroethoxy)pyridin-3-yl)methyl)-2-methyl-3-oxopiperazine-1-carboxamide;
(10) (2R)—N—((S)-(4-chlorophenyl)(6-(2,2,2-trifluoroethoxy)pyridin-3-yl)methyl)-2-methyl-3-oxopiperazine-1-carboxamide;
(11) (2R)—N—((R)-(3,4-difluorophenyl)(6-(2,2,2-trifluoroethoxy)pyridin-3-yl)methyl)-2-methyl-3-oxopiperazine-1-carboxamide;
(12) (2R)—N—((S)-(3,4-difluorophenyl)(6-(2,2,2-trifluoroethoxy)pyridin-3-yl)methyl)-2-methyl-3-oxopiperazine-1-carboxamide;
(13) (2R)—N—((R)-(3-chloro-4,5-difluorophenyl)(6-(2,2,2-trifluoroethoxy)pyridin-3-yl)methyl)-2-methyl-3-oxopiperazine-1-carboxamide;
(14) (2R)—N—((S)-(3-chloro-4,5-difluorophenyl)(6-(2,2,2-trifluoroethoxy)pyridin-3-yl)methyl)-2-methyl-3-oxopiperazine-1-carboxamide;
(15) N—((R)-(3-chloro-2,4-difluorophenyl)(6-(2,2,2-trifluoroethoxy) pyridin-3-yl)methyl)-3-oxopiperazine-1-carboxamide;
(16) N—((S)-(3-chloro-2,4-difluorophenyl)(6-(2,2,2-trifluoroethoxy) pyridin-3-yl)methyl)-3-oxopiperazine-1-carboxamide;
(17) (2R)—N—((R)-(4-chlorophenyl)(6-(trifluoromethyl)pyridin-2-yl)methyl)-2-methyl-3-oxopiperazine-1-carboxamide;
(18) (2R)—N—((S)-(4-chlorophenyl)(6-(trifluoromethyl)pyridin-2-yl)methyl)-2-methyl-3-oxopiperazine-1-carboxamide;
(19) (2R)—N—((R)-(3-chloro-2,4-difluorophenyl)(5-fluoro-6-(2,2,2-trifluoroethoxy)pyridin-3-yl)methyl)-2-methyl-3-oxopiperazine-1-carboxamide;
(20) (2R)—N—((S)-(3-chloro-2,4-difluorophenyl)(5-fluoro-6-(2,2,2-trifluoroethoxy)pyridin-3-yl)methyl)-2-methyl-3-oxopiperazine-1-carboxamide;
(21) (2R)—N—((R)-(3-chloro-4-fluorophenyl)(2-(trifluoromethyl)imidazo[1,2-a]pyridin-6-yl)methyl)-2-methyl-3-oxopiperazine-1-carboxamide;
(22) (2R)—N—((S)-(3-chloro-4-fluorophenyl)(2-(trifluoromethyl)imidazo[1,2-a]pyridin-6-yl)methyl)-2-methyl-3-oxopiperazine-1-carboxamide;
(23) (2R)—N—((R)-(3-chloro-2,4-difluorophenyl)(2-(2,2,2-trifluoroethoxy)pyrimidin-5-methyl-3-oxopiperazine-1-carboxamide;
(24) (2R)—N—((S)-(3-chloro-2,4-difluorophenyl)(2-(2,2,2-trifluoroethoxy)pyrimidin-5-methyl-3-oxopiperazine-1-carboxamide;
(25) (2R)—N—((R)-(3-chloro-2,4-difluorophenyl)(2-(2,2,2-trifluoroethoxy)thiazol-5-yl)methyl)-2-methyl-3-oxopiperazine-1-carboxamide;
(26) (2R)—N—((S)-(3-chloro-2,4-difluorophenyl)(2-(2,2,2-trifluoroethoxy)thiazol-5-yl)methyl)-2-methyl-3-oxopiperazine-1-carboxamide;
(27) (2R)—N—((R)-(3-chloro-2,4-difluorophenyl)(2-(difluoromethoxy)thiazol-5-yl)methyl)-2-methyl-3-oxopiperazine-1-carboxamide;
(2R)—N—((S)-(3-chloro-2,4-difluorophenyl)(2-(difluoromethoxy)thiazol-5-yl)methyl)-2-methyl-3-oxopiperazine-1-carboxamide;
(28) methyl-3-oxopiperazine-1-carboxamide;
(29) (2R)—N—((R)-(3-chloro-2,4-difluorophenyl)(6-(trifluoromethyl)pyridin-3-yl)methyl)-2-methyl-3-oxopiperazine-1-carboxamide;
(30) (2R)—N—((S)-(3-chloro-2,4-difluorophenyl)(6-(trifluoromethyl)pyridin-3-yl)methyl)-2-methyl-3-oxopiperazine-1-carboxamide;
(31) (2R)—N—((R)-(3-chloro-4-fluorophenyl)(6-(trifluoromethyl)pyridin-3-yl)methyl)-2-methyl-3-oxopiperazine-1-carboxamide;
(32) (2R)—N—((S)-(3-chloro-4-fluorophenyl)(6-(trifluoromethyl)pyridin-3-yl)methyl)-2-methyl-3-oxopiperazine-1-carboxamide;
(33) (2R)—N—((R)-(4-chlorophenyl)(6-(trifluoromethyl)pyridin-3-yl)methyl)-2-methyl-3-oxopiperazine-1-carboxamide;
(34) (2R)—N—((S)-(4-chlorophenyl)(6-(trifluoromethyl)pyridin-3-yl)methyl)-2-methyl-3-oxopiperazine-1-carboxamide;
(35) (2R)—N—((R)-(3,4-dichlorophenyl)(6-(trifluoromethyl)pyridin-3-yl)methyl)-2-methyl-3-oxopiperazine-1-carboxamide;
(36) (2R)—N—((S)-(3,4-dichlorophenyl)(6-(trifluoromethyl)pyridin-3-yl)methyl)-2-methyl-3-oxopiperazine-1-carboxamide;
(37) (2R)—N—((R)-(4-fluoro-3-(trifluoromethoxy)phenyl)(6-(trifluoromethyl) pyridin-3-yl)methyl)-2-methyl-3-oxopiperazine-1-carboxamide;
(38) (2R)—N—((S)-(4-fluoro-3-(trifluoromethoxy)phenyl)(6-(trifluoromethyl) pyridin-3-methyl-3-oxopiperazine-1-carboxamide;
(39) (2R)—N—((R)-(5-chloro-6-(trifluoromethyl)pyridin-3-yl)(4-(trifluoromethoxy)-phenyl)methyl)-2-methyl-3-oxopiperazine-1-carboxamide;
(40) (2R)—N—((S)-(5-chloro-6-(trifluoromethyl)pyridin-3-yl)(4-(trifluoromethoxy)-phenyl)methyl)-2-methyl-3-oxopiperazine-1-carboxamide;
(41) (2R)—N—((R)-(5-chloro-6-(trifluoromethyl)pyridin-3-yl)(3-fluoro-4-(trifluoromethoxy)-phenyl)methyl)-2-methyl-3-oxopiperazine-1-carboxamide;
(42) (2R)—N—((S)-(5-chloro-6-(trifluoromethyl)pyridin-3-yl)(3-fluoro-4-(trifluoromethoxy)-phenyl)methyl)-2-methyl-3-oxo-piperazine-1-carboxamide;
(43) (2R)—N—((R)-(3-chloro-4-(trifluoromethoxy)phenyl)(5-chloro-6-(trifluoromethylpyridin-3-yl)methyl)-2-methyl-3-oxopiperazine-1-carboxamide;
(44) (2R)—N—((S)-(3-chloro-4-(trifluoromethoxy)phenyl)(5-chloro-6-(trifluoro-methyl)pyridin-3-yl)methyl)-2-methyl-3-oxopiperazine-1-carboxamide;
(45) (2R)—N—((R)-(4-chloro-3-cyanophenyl)(5-chloro-6-(trifluoromethyl) pyridin-3-yl)methyl)-2-methyl-3-oxopiperazine-1-carboxamide;
(46) (2R)—N—((R)-(3-chloro-4-cyanophenyl)(5-chloro-6-(trifluoromethyl) pyridin-3-yl)methyl)-2-methyl-3-oxopiperazine-1-carboxamide;
(47) (2R)—N—((S)-(3-chloro-4-cyanophenyl)(5-chloro-6-(trifluoromethyl) pyridin-3-yl)methyl)-2-methyl-3-oxopiperazine-1-carboxamide;
(48) (2R)—N—((R)-(5-chloro-6-(trifluoromethyl)pyridin-3-yl)(4-cyclopropoxy-3-fluorophenyl)methyl)-2-methyl-3-oxopiperazine-1-carboxamide;
(49) (2R)—N—((S)-(5-chloro-6-(trifluoromethyl)pyridin-3-yl)(4-cyclopropoxy-3-fluorophenyl)methyl)-2-methyl-3-oxopiperazine-1-carboxamide;
(50) (2R)—N—((R)-(3-chloro-4-fluorophenyl)(5-chloro-6-(trifluoromethyl) pyridin-3-yl)methyl)-2-methyl-3-oxopiperazine-1-carboxamide;
(51) (2R)—N—((S)-(3-chloro-4-fluorophenyl)(5-chloro-6-(trifluoromethyl) pyridin-3-yl)methyl)-2-methyl-3-oxopiperazine-1-carboxamide;
(52) (2R)—N—((R)-(3-chloro-2,4-difluorophenyl)(5-chloro-6-cyclopropyl pyridin-3-yl)methyl)-2-methyl-3-oxopiperazine-1-carboxamide;
(53) (2R)—N—((S)-(3-chloro-2,4-difluorophenyl)(5-chloro-6-cyclopropyl pyridin-3-yl)methyl)-2-methyl-3-oxopiperazine-1-carboxamide;
(54) (2R)—N—((R)-(3,4-dichlorophenyl)(2-(trifluoromethyl)pyrimidin-5-yl)methyl)-2-methyl-3-oxopiperazine-1-carboxamide;
(55) (2R)—N—((S)-(3,4-dichlorophenyl)(2-(trifluoromethyl)pyrimidin-5-yl)methyl)-2-methyl-3-oxopiperazine-1-carboxamide;
(56) (2R)—N—((R)-(3,4-dichloro-2-fluorophenyl)(6-(trifluoro-methyl)pyridin-3-yl)methyl)-2-methyl-3-oxopiperazine-1-carboxamide;
(57) (2R)—N—((S)-(3,4-dichloro-2-fluorophenyl)(6-(trifluoro-methyl)pyridin-3-yl)methyl)-2-methyl-3-oxopiperazine-1-carboxamide;
(58) (2R)—N—((R)-(3-chloro-2,4-difluorophenyl)(2-(trifluoro-methyl)pyrimidin-5-yl)methyl)-2-methyl-3-oxopiperazine-1-carboxamide;
(59) (2R)—N—((S)-(3-chloro-2,4-difluorophenyl)(2-(trifluoro-methyl)pyrimidin-5-yl)methyl)-2-methyl-3-oxopiperazine-1-carboxamide;
(60) (2R)—N—((R)-(3-chloro-2,4-difluorophenyl)(6-(difluoro-methoxy)pyridin-3-yl)methyl)-2-methyl-3-oxopiperazine-1-carboxamide;
(61) (2R)—N—((S)-(3-chloro-2,4-difluorophenyl)(6-(difluoro-methoxy)pyridin-3-yl)methyl)-2-methyl-3-oxopiperazine-1-carboxamide;
(62) (2R)—N—((R)-(3-chloro-2,4-difluorophenyl)(6-(difluoro-methyl)pyridin-3-yl)methyl)-2-methyl-3-oxopiperazine-1-carboxamide;
(63) (2R)—N—((S)-(3-chloro-2,4-difluorophenyl)(6-(difluoro-methyl)pyridin-3-yl)methyl)-2-methyl-3-oxopiperazine-1-carboxamide;
(64) (2R)—N—((R)-(3-chloro-2,4-difluorophenyl)(6-cyclo-propylpyridin-3-yl)methyl)-2-methyl-3-oxopiperazine-1-carboxamide;
(65) (2R)—N—((S)-(3-chloro-2,4-difluorophenyl)(6-cyclo-propylpyridin-3-yl)methyl)-2-methyl-3-oxopiperazine-1-carboxamide;
(66) (2R)—N—((R)-(3-chloro-2,4-difluorophenyl)(5-fluoro-6-(trifluoromethyl)pyridin-3-yl)methyl)-2-methyl-3-oxopiperazine-1-carboxamide;
(67) (2R)—N—((S)-(3-chloro-2,4-difluorophenyl)(5-fluoro-6-(trifluoromethyl)pyridin-3-yl)methyl)-2-methyl-3-oxopiperazine-1-carboxamide;
(68) (2R)—N—((R)-(3-chloro-2,4-difluorophenyl)(5-chloro-6-(trifluoromethyl)pyridin-3-yl)methyl)-2-methyl-3-oxopiperazine-1-carboxamide;
(69) (2R)—N—((S)-(3-chloro-2,4-difluorophenyl)(5-chloro-6-(trifluoromethyl)pyridin-3-yl)methyl)-2-methyl-3-oxopiperazine-1-carboxamide;
(70) N—((R)-(3-chloro-2,4-difluorophenyl)(5-chloro-6-(trifluoro-methyl)pyridin-3-yl)methyl)-3-oxopiperazine-1-carboxamide;
(71) N—((S)-(3-chloro-2,4-difluorophenyl)(5-chloro-6-(trifluoro-methyl)pyridin-3-yl)methyl)-3-oxopiperazine-1-carboxamide;
(72) N—(R)-(3-chloro-2,4-difluorophenyl)(5-fluoro-6-(trifluoro-methyl)pyridin-3-yl)methyl)-(R or S)-2-cyclopropyl-3-oxopiperazine-1-carboxamide;
(73) N—((S)-(3-chloro-2,4-difluorophenyl)(5-fluoro-6-(trifluoro-methyl)pyridin-3-yl)methyl)-(S or R)-2-cyclopropyl-3-oxopiperazine-1-carboxamide;
(74) N—((R)-(3-chloro-2,4-difluorophenyl)(6-(trifluoromethyl)pyridin-3-yl)methyl)-(R)-2-cyclopropyl-3-oxopiperazine-1-carboxamide;
(75) N—((S)-(3-chloro-2,4-difluorophenyl)(6-(trifluoromethyl)pyridin-3-yl)methyl)-(S)-2-cyclopropyl-3-oxopiperazine-1-carboxamide;
(76) N—((R)-(3-chloro-2,4-difluorophenyl)(6-(trifluoromethyl)pyridin-3-yl)methyl)-(R or S)-2-isopropyl-3-oxo-piperazine-1-carboxamide;
(77) N—((S)-(3-chloro-2,4-difluorophenyl)(6-(trifluoromethyl)pyridin-3-yl)methyl)-(S or R)-2-isopropyl-3-oxo-piperazine-1-carboxamide;
(78) N—((R)-(3-chloro-2,4-difluorophenyl)(6-(trifluoromethyl)pyridin-3-yl)methyl)-(R or S)-2-ethyl-3-oxopiperazine-1-carboxamide;
(79) N—((S)-(3-chloro-2,4-difluorophenyl)(6-(trifluoromethyl)pyridin-3-yl)methyl)-(R or S)-2-ethyl-3-oxopiperazine-1-carboxamide;
(80) N—((R)-(3-chloro-2,4-difluorophenyl)(2-(trifluoromethyl)pyrimidin-5-yl)methyl)-(R or S)-2-cyclopropyl-3-oxopiperazine-1-carboxamide;
(81) N—((S)-(3-chloro-2,4-difluorophenyl)(2-(trifluoromethyl)pyrimidin-5-yl)methyl)-(S or R)-2-cyclopropyl-3-oxopiperazine-1-carboxamide;
(82) N—((R)-(3-chloro-4-fluorophenyl)(6-(trifluoromethyl)pyridin-2-yl)methyl)-3-oxopiperazine-1-carboxamide;
(83) N—((S)-(3-chloro-4-fluorophenyl)(6-(trifluoromethyl)pyridin-2-yl)methyl)-3-oxopiperazine-1-carboxamide;
(84) N—((R)-(3-chloro-4-fluorophenyl)(6-(trifluoromethyl)pyridin-2-yl)methyl)-2,2-dimethyl-3-oxopiperazine-1-carboxamide;
(85) N—((S)-(3-chloro-4-fluorophenyl)(6-(trifluoromethyl)pyridin-2-yl)methyl)-2,2-dimethyl-3-oxopiperazine-1-carboxamide;
(86) (2R)—N—((R)-(3-chloro-4-fluorophenyl)(6-(trifluoro-methyl)pyridin-2-yl)methyl)-2-methyl-3-oxopiperazine-1-carboxamide;
(87) (2R)—N—((S)-(3-chloro-4-fluorophenyl)(6-(trifluoro-methyl)pyridin-2-yl)methyl)-2-methyl-3-oxopiperazine-1-carboxamide;
(88) (2S)—N—((R)-(3-chloro-4-fluorophenyl)(6-(trifluoro-methyl)pyridin-2-yl)methyl)-2-methyl-3-oxopiperazine-1-carboxamide;
(89) (2S)—N—((S)-(3-chloro-4-fluorophenyl)(6-(trifluoro-methyl)pyridin-2-yl)methyl)-2-methyl-3-oxopiperazine-1-carboxamide;
(90) (3S)—N—((R)-(3-chloro-4-fluorophenyl)(6-(trifluoro-methyl)pyridin-2-yl)methyl)-3-methyl-5-oxopiperazine-1-carboxamide;
(91) (3R)—N—((S)-(3-chloro-4-fluorophenyl)(6-(trifluoro-methyl)pyridin-2-yl)methyl)-3-methyl-5-oxopiperazine-1-carboxamide;
(92) (2R)—N—((R)-(3-chloro-4-fluorophenyl)(6-(trifluoro-methyl)pyridin-2-yl)methyl)-2-(fluoromethyl)-5-oxopiperazine-1-carboxamide;
(93) (2S)—N—((S)-(3-chloro-4-fluorophenyl)(6-(trifluoro-methyl)pyridin-2-yl)methyl)-2-(fluoromethyl)-5-oxopiperazine-1-carboxamide;
(94) (2R)—N—((R)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide;
(95) (2R)—N—((S)-(5-fluoro-6-(2,2,2-trifluoroethoxy)pyridin-3-yl)(5-fluoro-6-(trifluoromethyl)pyridin-2-yl)methyl)-2-methyl-3-oxopiperazine-1-carboxamide;
(96) (2R)—N—((R)-(3,4-difluorophenyl)(5-fluoro-6-(trifluoro-methyl)pyridin-2-yl)methyl)-2-methyl-3-oxopiperazine-1-carboxamide;
(97) (2R)—N—((S)-(3,4-difluorophenyl)(5-fluoro-6-(trifluoro-methyl)pyridin-2-yl)methyl)-2-methyl-3-oxopiperazine-1-carboxamide;
(98) (2R)—N—((R)-(5-fluoro-6-(trifluoro-methyl)pyridin-2-yl)(6-(2,2,2-trifluoro-ethoxy)pyridin-3-yl)methyl)-2-methyl-3-oxopiperazine-1-carboxamide;
(99) (2R)—N—((S)-(5-fluoro-6-(trifluoro-methyl)pyridin-2-yl)(6-(2,2,2-trifluoro-ethoxy)pyridin-3-yl)methyl)-2-methyl-3-oxopiperazine-1-carboxamide;
(100) (2R)—N—((R)-(5-fluoro-6-(trifluoromethyl)pyridin-2-yl)(6-(trifluoro-methoxy)pyridin-3-yl)methyl)-2-methyl-3-oxopiperazine-1-carboxamide;
(101) (2R)—N—((S)-(5-fluoro-6-(trifluoro-methyl)pyridin-2-yl)(6-(trifluoromethoxy)pyridin-3-yl)methyl)-2-methyl-3-oxopiperazine-1-carboxamide;
(102) (2R)—N—((R)-(4-chloro-3-cyanophenyl)(5-fluoro-6-(trifluoromethyl) pyridin-2-yl)methyl)-2-methyl-3-oxopiperazine-1-carboxamide;
(103) (2R)—N—((S)-(4-chloro-3-cyanophenyl)(5-fluoro-6-(trifluoromethyl) pyridin-2-yl)methyl)-2-methyl-3-oxopiperazine-1-carboxamide;
(104) (2R)—N—((R)-(4-chloro-3-fluorophenyl)(5-fluoro-6-(trifluoromethyl) pyridin-2-yl)methyl)-2-methyl-3-oxopiperazine-1-carboxamide;
(105) (2R)—N—((S)-(4-chloro-3-fluoro-phenyl)(5-fluoro-6-(trifluoromethyl) pyridin-2-yl)methyl)-2-methyl-3-oxopiperazine-1-carboxamide;
(106) N—((R)-(4-chloro-3-fluorophenyl)(5-fluoro-6-(trifluoromethyl) pyridin-2-yl)methyl)-3-oxopiperazine-1-carboxamide;
(107) N—((S)-(4-chloro-3-fluorophenyl)(5-fluoro-6-(trifluoromethyl) pyridin-2-yl)methyl)-3-oxopiperazine-1-carboxamide;
(108) (2R)—N—((R)-(5-fluoro-6-(trifluoromethyl)pyridin-2-yl)(4-(trifluoromethoxy)-phenyl)methyl)-2-methyl-3-oxopiperazine-1-carboxamide;
(109) (2R)—N—((S)-(5-fluoro-6-(trifluoromethyl)pyridin-2-yl)(4-(trifluoromethoxy)-phenyl)methyl)-2-methyl-3-oxopiperazine-1-carboxamide;
(110) (2R)—N—((R)-(3-fluoro-4-(trifluoromethoxy)phenyl)(5-fluoro-6-(trifluoro-methyl)pyridin-2-yl)methyl)-2-methyl-3-oxopiperazine-1-carboxamide;
(111) (2R)—N—((S)-(3-fluoro-4-(trifluoromethoxy)phenyl)(5-fluoro-6-(trifluoro-methyl)pyridin-2-yl)methyl)-2-methyl-3-oxopiperazine-1-carboxamide;
(112) (2R)—N—((R)-(5-fluoro-6-(trifluoromethyl)pyridin-2-yl)(4-(2,2,2-trifluoro-ethoxy)phenyl)methyl)-2-methyl-3-oxopiperazine-1-carboxamide;
(113) (2R)—N—((S)-(5-fluoro-6-(trifluoromethyl)pyridin-2-yl)(4-(2,2,2-trifluoro-ethoxy)phenyl)methyl)-2-methyl-3-oxopiperazine-1-carboxamide;
(114) (2R)—N—((R)-(5-fluoro-6-(trifluoromethyl)pyridin-2-yl)(3-(trifluoromethoxy)-phenyl)methyl)-2-methyl-3-oxopiperazine-1-carboxamide;
(115) (2R)—N—((S)-(5-fluoro-6-(trifluoromethyl)pyridin-2-yl)(3-(trifluoromethoxy)-phenyl)methyl)-2-methyl-3-oxopiperazine-1-carboxamide;
(116) (2R)—N—((R)-(4-cyclopropoxy-3-fluorophenyl)(5-fluoro-6-(trifluoromethyl) pyridin-2-yl)methyl)-2-methyl-3-oxopiperazine-1-carboxamide;
(117) (2R)—N—((S)-(4-cyclopropoxy-3-fluorophenyl)(5-fluoro-6-(trifluoromethyl) pyridin-2-yl)methyl)-2-methyl-3-oxopiperazine-1-carboxamide;
(118) (2R)—N—((R)-(3-chloro-2,4-difluorophenyl)(5-fluoro-6-(trifluoromethyl)pyridin-2-yl)methyl)-2-methyl-3-oxopiperazine-1-carboxamide;
(119) (2R)—N—((S)-(3-chloro-2,4-difluorophenyl)(5-fluoro-6-(trifluoromethyl)pyridin-2-yl)methyl)-2-methyl-3-oxopiperazine-1-carboxamide;
(120) (2R)—N—((R)-(5-cyano-6-(trifluoromethyl)pyridin-2-yl)(3-fluoro-4-(trifluoromethoxy)-phenyl)methyl)-2-methyl-3-oxopiperazine-1-carboxamide;
(121) (2R)—N—((S)-(5-cyano-6-(trifluoromethyl)pyridin-2-yl)(3-fluoro-4-(trifluoromethoxy)-phenyl)methyl)-2-methyl-3-oxopiperazine-1-carboxamide;
(122) (2R)—N—((R)-(3-chloro-2,4-difluorophenyl)(2-(trifluoromethyl)thiazol-5-yl)methyl)-2-methyl-3-oxopiperazine-1-carboxamide;
(123) (2R)—N—((S)-(3-chloro-2,4-difluorophenyl)(2-(trifluoromethyl)thiazol-5-yl)methyl)-2-methyl-3-oxopiperazine-1-carboxamide;
(124) (2R)—N—((R)-(3-chloro-4-(trifluoromethoxy)phenyl)(1-(trifluoromethyl)-1H-pyrazol-4-yl)methyl)-2-methyl-3-oxopiperazine-1-carboxamide;
(125) (2R)—N—((S)-(3-chloro-4-(trifluoromethoxy)phenyl)(1-(trifluoromethyl)-1H-pyrazol-4-yl)methyl)-2-methyl-3-oxopiperazine-1-carboxamide;
(126) (2R)—N—((R)-(3-chloro-4-(trifluoromethoxy)phenyl)(2-(trifluoromethyl) oxazol-4-yl)methyl)-2-methyl-3-oxopiperazine-1-carboxamide;
(127) (2R)—N—((S)-(3-chloro-4-(trifluoromethoxy)phenyl)(2-(trifluoromethyl) oxazol-4-yl)methyl)-2-methyl-3-oxopiperazine-1-carboxamide;
(128) (2R)—N—((R)-(3-chloro-2,4-difluorophenyl)(1-(4-fluorophenyl)-1H-pyrazol-4-yl)methyl)-2-methyl-3-oxopiperazine-1-carboxamide;
(129) (2R)—N—((S)-(3-chloro-2,4-difluorophenyl)(1-(4-fluorophenyl)-1H-pyrazol-4-yl)methyl)-2-methyl-3-oxopiperazine-1-carboxamide;
(130) (2R)—N—((R)-(3-chloro-4-fluorophenyl)(1-(difluoromethyl)-1H-pyrazol-3-yl)methyl)-2-methyl-3-oxopiperazine-1-carboxamide;
(131) (2R)—N—((S)-(3-chloro-4-fluorophenyl)(1-(difluoromethyl)-1H-pyrazol-3-yl)methyl)-2-methyl-3-oxopiperazine-1-carboxamide;
(132) (2R)—N—((R)-(3-chloro-4-fluorophenyl)(1-(2,2,2-trifluoroethyl)-1H-pyrazol-4-yl)methyl)-2-methyl-3-oxopiperazine-1-carboxamide;
(133) (2R)—N—((S)-(3-chloro-4-fluorophenyl)(1-(2,2,2-trifluoroethyl)-1H-pyrazol-4-yl)methyl)-2-methyl-3-oxopiperazine-1-carboxamide;
(134) (2R)—N—((R)-(4-chlorophenyl)(2-(trifluoromethyl)pyrimidin-4-yl)methyl)-2-methyl-3-oxopiperazine-1-carboxamide;
(135) (2R)—N—((S)-(4-chlorophenyl)(2-(trifluoromethyl)pyrimidin-4-yl)methyl)-2-methyl-3-oxopiperazine-1-carboxamide;
(136) (2R)—N—((R)-(3-chloro-4-fluorophenyl)(2-(trifluoromethyl)pyrimidin-5-yl)methyl)-2-methyl-3-oxopiperazine-1-carboxamide;
(137) (2R)—N—((S)-(3-chloro-4-fluorophenyl)(2-(trifluoromethyl)pyrimidin-5-yl)methyl)-2-methyl-3-oxopiperazine-1-carboxamide;
(138) (2R)—N—((R)-(3-chloro-4-fluorophenyl)(5-fluoro-6-(2,2,2-trifluoroethoxy)pyridin-2-yl)methyl)-2-methyl-3-oxopiperazine-1-carboxamide;
(139) (2R)—N—((S)-(3-chloro-4-fluorophenyl)(5-fluoro-6-(2,2,2-trifluoroethoxy)pyridin-2-yl)methyl)-2-methyl-3-oxopiperazine-1-carboxamide;
(140) (2R)—N—((R)-(3-chloro-4-fluorophenyl)(2-(2,2,2-trifluoroethoxy)pyridin-4-yl)methyl)-2-methyl-3-oxopiperazine-1-carboxamide;
(141) (2R)—N—((S)-(3-chloro-4-fluorophenyl)(2-(2,2,2-trifluoroethoxy)pyridin-4-yl)methyl)-2-methyl-3-oxopiperazine-1-carboxamide;
(142) (2R)—N—((R)-(3-chloro-4-fluorophenyl)(6-(difluoromethoxy)pyridin-2-yl)methyl)-2-methyl-3-oxopiperazine-1-carboxamide;
(143) (2R)—N—((S)-(3-chloro-4-fluorophenyl)(6-(difluoromethoxy)pyridin-2-yl)methyl)-2-methyl-3-oxopiperazine-1-carboxamide;
(144) (2R)—N—((R)-(3-chloro-4-fluorophenyl)(6-(difluoromethoxy)pyridin-3-yl)methyl)-2-methyl-3-oxopiperazine-1-carboxamide;
(145) (2R)—N—((S)-(3-chloro-4-fluorophenyl)(6-(difluoromethoxy)pyridin-3-yl)methyl)-2-methyl-3-oxopiperazine-1-carboxamide;
(146) (2R)—N—((R)-(3-chloro-2,4-difluorophenyl)(2-(difluoromethoxy)pyrimidin-5-yl)methyl)-2-methyl-3-oxopiperazine-1-carboxamide;
(147) (2R)—N—((S)-(3-chloro-2,4-difluorophenyl)(2-(difluoromethoxy)pyrimidin-5-yl)methyl)-2-methyl-3-oxopiperazine-1-carboxamide;
(148) (2R)—N—((R)-(3-chloro-2,4-difluorophenyl)(6-(1,1-difluoroethyl)pyridin-3-yl)methyl)-2-methyl-3-oxopiperazine-1-carboxamide;
(149) (2R)—N—((S)-(3-chloro-2,4-difluorophenyl)(6-(1,1-difluoroethyl)pyridin-3-yl)methyl)-2-methyl-3-oxopiperazine-1-carboxamide;
(150) x(2R)—N—((R)-(5-chloro-6-(trifluoromethyl)pyridin-2-yl)(4-cyanophenyl)methyl)-2-methyl-3-oxopiperazine-1-carboxamide;
(151) (2R)—N—((S)-(5-chloro-6-(trifluoromethyl)pyridin-2-yl)(4-cyanophenyl)methyl)-2-methyl-3-oxopiperazine-1-carboxamide;
(152) (2R)—N—((R)-(3-chloro-2,4-difluorophenyl)(5-chloro-6-(trifluoromethyl)pyridin-2-yl)methyl)-2-methyl-3-oxopiperazine-1-carboxamide;
(153) (2R)—N—((S)-(3-chloro-2,4-difluorophenyl)(5-chloro-6-(trifluoromethyl)pyridin-2-yl)methyl)-2-methyl-3-oxopiperazine-1-carboxamide;
(154) N—((R)-(3-chloro-2,4-difluorophenyl)(5-chloro-6-(trifluoromethyl)pyridin-2-yl)methyl)-3-oxopiperazine-1-carboxamide;
(155) N—((S)-(3-chloro-2,4-difluorophenyl)(5-chloro-6-(trifluoromethyl)pyridin-2-yl)methyl)-3-oxopiperazine-1-carboxamide;
(156) (2R)—N—((R)-(3-chloro-4-fluorophenyl)(5-chloro-6-(trifluoromethyl) pyridin-2-yl)methyl)-2-methyl-3-oxopiperazine-1-carboxamide;
(157) (2R)—N—((S)-(3-chloro-4-fluorophenyl)(5-chloro-6-(trifluoromethyl) pyridin-2-yl)methyl)-2-methyl-3-oxopiperazine-1-carboxamide;
(158) N—((R)-(3-chloro-4-fluorophenyl)(5-chloro-6-(trifluoromethyl) pyridin-2-yl)methyl)-3-oxopiperazine-1-carboxamide;
(159) N—((S)-(3-chloro-4-fluorophenyl)(5-chloro-6-(trifluoromethyl) pyridin-2-yl)methyl)-3-oxopiperazine-1-carboxamide;
(160) (2R)—N—((R)-(5-chloro-6-(trifluoromethyl)pyridin-2-yl)(6-(trifluoromethyl) pyridin-3-yl)methyl)-2-methyl-3-oxopiperazine-1-carboxamide;
(161) (2R)—N—((S)-(5-chloro-6-(trifluoromethyl)pyridin-2-yl)(6-(trifluoromethyl) pyridin-3-yl)methyl)-2-methyl-3-oxopiperazine-1-carboxamide;
(162) ((2R)—N—((R)-(5-chloro-6-(trifluoromethyl)pyridin-3-yl)(5-fluoro-6-(trifluoromethyl)pyridin-2-yl)methyl)-2-methyl-3-oxopiperazine-1-carboxamide;
(163) ((2R)—N—((S)-(5-chloro-6-(trifluoromethyl)pyridin-3-yl)(5-fluoro-6-(trifluoromethyl)pyridin-2-yl)methyl)-2-methyl-3-oxopiperazine-1-carboxamide;
(164) (2R)—N—((R)-(3-chloro-2,4-difluorophenyl)(5-chloro-6-(trifluoromethyl)pyridin-2-yl)methyl)-2-methyl-3-oxopiperazine-1-carboxamide;
(165) (2R)—N—((S)-(3-chloro-2,4-difluorophenyl)(5-chloro-6-(trifluoromethyl)pyridin-2-yl)methyl)-2-methyl-3-oxopiperazine-1-carboxamide;
(166) (2R)—N—((R)-(5-chloro-6-(trifluoromethyl)pyridin-3-yl)(2-(trifluoromethyl)thiazol-4-yl)methyl)-2-methyl-3-oxopiperazine-1-carboxamide;
(167) (2R)—N—((S)-(5-chloro-6-(trifluoromethyl)pyridin-3-yl)(2-(trifluoromethyl)thiazol-4-yl)methyl)-2-methyl-3-oxopiperazine-1-carboxamide;
(168) (2R)—N—((R)-(3-chloro-4-fluorophenyl)(3-(trifluoromethyl)-1H-pyrazol-5-yl)methyl)-2-methyl-3-oxopiperazine-1-carboxamide;
(169) (2R)—N—((S)-(3-chloro-4-fluorophenyl)(3-(trifluoromethyl)-1H-pyrazol-5-yl)methyl)-2-methyl-3-oxopiperazine-1-carboxamide;
(170) (2R)—N—((R)-(3-chloro-4-fluorophenyl)(1-methyl-3-(trifluoromethyl)-1H-pyrazol-5-yl)methyl)-2-methyl-3-oxopiperazine-1-carboxamide;
(171) (2R)—N—((S)-(3-chloro-4-fluorophenyl)(1-methyl-3-(trifluoromethyl)-1H-pyrazol-5-yl)methyl)-2-methyl-3-oxopiperazine-1-carboxamide;
(172) (2R)-2-methyl-3-oxo-N—((R)-(4-(trifluoromethoxy)phenyl)(3-(trifluoromethyl)-1H-pyrazol-5-yl)methyl)piperazine-1-carboxamide; and
(173) (2R)-2-methyl-3-oxo-N—((S)-(4-(trifluoro methoxy)phenyl)(3-(trifluoromethyl)-1H-pyrazol-5-yl)methyl)piperazine-1-carboxamide;
or a pharmaceutically acceptable salt thereof.

19. A pharmaceutical composition comprising a compound of claim 1, or a pharmaceutically acceptable salt thereof, and a pharmaceutically acceptable carrier.

20-24. (canceled)

25. A method of treating or preventing a disorder, condition or disease that is responsive to the inhibition of Naᵥ1.8 channel activity in a patient in need thereof comprising administration of a therapeutically effective amount of a compound according to claim 1, or a pharmaceutically acceptable salt thereof.

26. The method of claim 25 wherein the disorder is selected from: pain disorder, a cough disorder, an acute itch disorder or chronic itch disorder.

27. The method of claim 25 wherein the disorder is a pain disorder.

28. The method of claim 27 wherein the pain disorder is selected from: acute pain, inflammatory pain, or neuropathic pain.
